SEQUENCE LISTING (136 sequences)

SEQ ID NO: 1 - DNA, 9188 bp, Canine hepacivirus 1
ctgcgttgtcagcgttttgcgcttgcatgcgctacacgcgtcgtccaacgcggagggaacttcacatcaccatgtgtcactccccctatggagggttccaccccgcttacacggaaatgggttaaccatacccaaagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggacatgagtaataaatctaaaaaccaaaaacccaaaccacaacgaggaccacggggtagggtcaggggtcagtcgcggtctggtcctgtagtattcccttccggcgctgtcctcgtgggggggaggtatatcccccccccccaaaaggccatccgcgggcctcggagaggtctggtgcaagctcctaagtcatcggagcgtacttccccccgaaaaagacgccagcctccgccacaaactgacagctcgtggcgaaagtatttctcaaaattctggggggataggggatacccatggccttatgttgatcccgttctccagtggggggcctgggggtcttctcctggcgcttatcgcactaggtggggaccccgcgaccctcgccacaagtcgcgcaacttggggcgcgtcattgacactcttacctgtggagttgccgatctagctgggtatgtcccagttctcggagctcccgcaggcgcgttgtgtcggggcgcggctcatcttgtacggtttgttgaggacggtgccaatttcatcactggcaacattcctggcatgggtttctctatcttcttgcttgctctcttctcggctgtatctttcggagaggcctctgttgtgcggaacggtggacacgttgtcagtaacgattgtaattcctctcagattttatgggcatcctccgactgggccatccacgaggttgggtgtattccatgcgtggatggtgtctgttgggtgccgttaacatctagtatatctgtcaggaatgaatctgttattgttcgtgggctcggttcgcatatagatgtcttgtccgctatggcttctgtttgttctactctgggaattggcgaggcctgtggtgctgcgactctcacttacattactttcttgtctcgattctttatgcctcttaacttgactaatgattgtgagtgttttctctatcctggcgccatttctacctttgagtttactatgagagctttgcaatccatgatgcccaatttgtccggttttctttcgatgttcagtggacttccaaatactctgttcaccatctttactaacgggcattggggcgttatacttgctctttgtctctatggcacaactaacaactactttaaactttgcttgttgcttttagcttattcaggcttagtttcttgtgatgattaccttaatgtgtctttgtcttgcaacttcactgttaaggagatgtggggctggacgtttttcccaaaatgggcactactcaacggacagaggctaaattgcactgaggggtccycatacaaccctaaatgcaaaggcccttttgatttcaatgtcacaactgatccgtacattgcctatagcgggactcgttcacatccgccctgcccctatcatgtgtctagaccttgctccgtccttgatgcatccagagtgtgtggcaaacctacctgcttcggtcctgcaccgattgaggtgggtgtcactgatcgggatggaaagcttgcttcttggaatgattctggtcagtttttctttgatctgcggtctcctcaccgcccacctcgtggacggtggtatgggtgtgtttggcttaattcgactggctgggtcaagcaatgtggcgctcctccttgtaacatgagactcatgtccaacaagagcaagccttttgtttgccccaccgattgttttaggcaaaaccccaaagccacttatcaactttgtggccaaggaccgtggatcactcattcttgcttgattgattatactgataggtatcttcacttcccgtgcactgagaacttcacagtctaccctgtccgcatgattcttggcgacggcgctagggatgttagagtggcgtgcaaatttaacagatcggtcagctgtaggactgaagataggttgagagcgagtatcgtctcccttctttacagtgtgactaccgcagctgtcccaccatgtcatttttcgccgctacctgctttcactactgggctcatccacttagatcgcaatctgagtgatgtccagtatgtttgggctatgactcccagcgctgtcaatatcttcttgaggctagagtgggctgtgtttttcttgcttcttcttatggatgcgaaggtatgtgcaattctctggttctgtctgtgcttagctttgcaagctgaggcttatctttcagatactatgcgattgatcgcactctcctacattgctgatgattctttgctgtgggctctggttttttactgtgtaatctacttcaccccaagcagggtcccgcccttttgtgtgtttgtatattactggaagtttgccttggcttttatggttctggctttaccacatcgtgcttgggcttttgataatgctagtgctgttacggcagcttttagcatcgctcttttctgcttgtatattacttgcttgtcttgctataaaaaactgtttatgctcgtcaagtggtggctggaatattgggatgttagaattgaatgtgcttggcgctatctcggccctagggtaaatcctagggacgagaagcttgcttttgccttactcttttcattctttcacccttccttgttccgctgtatatacttgcctttagctgttatatgtgggtccttttccatgatcaacaaacgtgttcaaaagatctcgtatcttcgacgagctgaggtcttggttcgggtcctttctatctgcagagatgtttatggtagtaaatgggttcaatggtgtatcttatggttagcttctcatcttggcactttcctttatgaccaccttaccccaatagatacttgggccgcgcctggcttgagagaccttatgcattcgttggagcctataactctttcacccatggagaggaaagttgtcaagtggggcgcgaggaagattgcttgcggcgacattcttcgcggcctgcccgtatctgcgaggcttggtagagaaatctgccttggccccgctgataagctcacaagcaagggctggagattgttatcaccaatcaccgcgaccgtcaccaagacgcgcggtattccttcagctattgtttgttgccttacgggtagggataagtatccccacagaggtcattgttacatcttaacctctttgactaagactttcatgggcaccgtgtgtaaaggtgtgctgtggtcggttcatcatggtggcggcactgctactcttgcttctgataaatcatctttgcttcaagtgttgtgttctcccggtgacgatcttgtggcatggcctgcccccgccgggtccaagtcttttcagccgtgcacttgtggctcggctgatgtgtttttggtcacccgtacagggcaggtcgtcccagcaagaaagacctccgaaaaggatgcttcacttatttctcccttacctatttcatcactgaaaggcagttcgggtggcccagtcctgtgtaaggatggggatcttgtaggtatcttctgttctgcttcggttactaggggggtggctaagcgcatacacttcgcagatatgcggacgcggagcgtttctagttgccctcccaagtacactgatttagattctcctcctgctgttccttcatcttatcaggtctctttccttcatgctcccactggtagcggcaaatccaccaagatgccgctgtcctatgtggagctgggatatcatgtcttagttctcaacccttcagtcgcctcgactctcagttttgggccttacatggataagacctatggggaatgtccaaacatcaggacaggcgcaagctgcaagactacaggctctaaacttacttattccacctatggtaagttcttagctgacgggggtgtttctgctggtgcctatgacattataatatgtgatgagtgccatagcacagactccacatccgtactaggaataggctccgtccttgatggggcagagtctaaaggtgtaaagcttgttgtacttgctactgctacgccacccgggtctcagactgtccctcatcctaacatcgatgaggaggccctcactcagagcggtgacatacccttttacgggaagatgttgaagtcgtctttgctcctaagtgggagacatcttatcttctgccattcgaagaagaagtgtgaagaggtcgcacttctcctgagaaaggccggagctaatgctgtaacctactatcggggtttggatgtttccgtcataccgaatgagggtaatgtcgtcgttgttgccacggacgcccttatgacagggtattctggcaattttgacaccgttactgactgtaacaccgctgtggaactggacattgagttttcccttgacccaacattttctatggttaccactcctaaaccatcagatgccgtttgcagaacacagcgcagggggaggactggtagaggccgcaggggcacttactactatgtaaatagtggtgagcgcccatctggggtcctatcgtcatccgtcctgtgcgagtgctatgacagcggcttggcttggtttggcctgagtccagcgcaggtaactgtgctactccaggcttaccttaagcaacctggccttcccaccggacttgaccacaccgaattttgggagagcgtcttcattgggttacctacagtcgacgctttctttttgtcccagctaaaacaacagggtgtaactttcccttatctaactgccatccaagcaactgtatgccttaacgcccaagctaaggcgcccagcaaagacgagcgctggaaggtgctgtcacggtatattaccacaaaccgtactccaacgcctttgttgtacagacttgaggacacccacgacgatcttacttttactcacccagtgaccaagtatattcaggcctgtatggaggccgaaattgacacccaaactaatgcttgggttatagcgggtgggtgtgttgcagctttggttgctgtcgctgcccttacgggtagcgttgcgatcatagctgaggtacacgtaaatgagaaagttgtggtagtccctcacaagggcgttctctacgccgattttgacgagatggaggaatgttttgatcaccatcaatacatccaacaaggatacgaatgggcgtcgcgtgcagcgcagaaaatccgcgaagtagcggcgtccattgaccctccaactggtcaagcacaaccattgctatcggctgttgagaaattttggaatcagcacatgtggaacatcttatcaggtgtgcagtatttggcaggtcttacaacactcccttataatccatcagtagcttgtctaatgggttttgtttcaggtctcactacaggattgccgaggccagctatggctttcctcaccattctaggtggttgggcggcaagcatggtggcccctccccaggcagcatccacctttgtcggcgctgggctggctggcatcgctattggtgcagtcggcttcactgacgtcatcgttggcctgcttgcggggtacggggccggtgtggctggcgcccttactgccttcaagatcttgagtggtgtcaccccgagtggtgaagacctaattaacctcttgccatctttgctcaaccctggcgcgcttgctgtaggtgtgggcgccgcattcatcctaaaaagatacacaggcggcagtgaaggactcgtcgcatgggtcaatcggctcatagcgttttgttctagggggaaccacgtctcccccgatcactatgtccaacaacagcaggtggtcagggatgtcattgcttgtcttgaatctttaaccttaactagactagtgaaaactatccataactttgttacaagtgaaaatgaccaaaattgtgattttactgccatttatttctttatccaatggttaatgaaaatactttatgattgtttcacctgggcaaagggaataattcttcctcatcttccaggtttcccgatcatatcgtgtgattcaggctactctggccggtgggctggagatggtctcgtttccactcggtgtggttgtggtaacttgatcactggaaatgtgaggaatgaaagaattaggattacaggctcgcggaagtgccgtaacgtctggctcaataccttcccaattaactctactactactggtgggccacggcctaatccatacgatgtgtggaaaacggctgttttgagaataacttccacagagtatgttgagttcaagagggaggggactgctgtcagagttactggcgcaaccgctgacaaattacgtatcccttgccaggtgcctgaacctgatttgatgacctttatagatggagttaggatacataggctggctccaaacccaaaaccaatgttgcgcgatgaggtggtggtcctaattggcaatcacacctacccagtcggcgcgacacttccatgcactccagaaccagatgttgacactgtatcttccttactaactgatccaggacatgtcactgcagagactgcagcaagacggctcaggcgcgggcgtactgttgatgttgagtcctcgagcggctccgagctgtcggcggtttcgcgaggcgcagcttctcgtgtgtcagaagaacatgagatggctggcggccctgtgagaccgctgaccggcgaagacgagctcgcgtggattcgatcattttacggacgctctgtgacgattgaggttgacgacaaggttatcaactttgactcgtggaccatcaattctgggtctgagggggagcactcccgagaatcagtccatgcgccagacgacgatcgggtggtggtggctgcaccgcctccgccgcctggtcctgcctggatgcgcaaagactatgtcccagctctcgtttccgggtgtcccatcaagcccggaagtgctactcctgagcccagtgagccctcggctacagaatctgccccagttgaggaaaaggaggaacctaaagtcgacgataagggtgaggaaacagaccctgacatgccccccctcgaaggtgaagagcctgaagagggggatgacagtcaatgggagactacttctgaaaagggagagtcatgctccttatcctattcgtggaccggtgccctcgtcacggcaacccgccgcgaggagcggcgtcatcctataggtcccctttccaacacactaattactaaacacaacctcgtgtaccagacaacaacagcgtctgctagcgcgaggatggctaaagtgacaattgatagagaacaggtccttgacaaattctactttgatactgtcacccaagtcaaaaagaaagcttcagaggtggctgctgacctgcttacgtgggatgaagttgcgcgcctcactccaaagaacacggctcgcgcgaaatcgggcctttccggttccgatattcgccaacttacgcgagctgcgaggcgggaactccagtcaacgtggcaggacctcttgtcaacctctgatgaacctatccctactacaattatggcaaagaacgaggttttcgtctcgtcgccaacttctcgcaaaccagccaggctgatcgtctaccctgatttgcctgtgcgtgcctgtgagaagagggcgatgtacgacctcttccaaaaattaccctatgctgtcatggggaaggcctatggatttcaatacactcctcgccagcgtgtcgatagactattagacatgtggcggcatttcaagaatcctatgggcttttcttacgacaccaaatgttttgactccactgtcactccgcatgatatagacactgagagggacatctttctaagtgccaatctccctgatgaggccaagacagtcattaagaacttgacgtctaggctttatagagggtcccctatgtacaactcacgtggggacctggttgggagaagggagtgccgtgcttccggggttttcccgacgagcatgggcaacactcttactaacttcatcaaagcctctgcagctgccaaagctgctgggtttgctgaccctcagttcttaatctgtggagatgatcttgtttgtgtcacctccagtaaaggcgttgaggaagatgaacaggcgttgcgtgaattcacaaacgccatgacaaagtactccgccattcctggagatttcccaaagccatactatgacttggagcaaataacatcttgttcctctaatgtgactgttgctcaagacagaaacgggcgaccttattatttcctcactcgtgatccaacaacaccgctggctcgcgcgagttgggagacaattagtcacagtcctgttaacagttggttaggaaacataattgcttttgctcctacggtgtgggttagactcgttttcctcactcatttcttcggtctgctgctccaacaggatgctgtggatcgaaattatgagtttgagatgtacggatcaacctactccgtaaacccacttgacttaccagcaataatttataagctccatggccctgaggcctttgatcttacaaactattctccttacgaggtccagagggtggccgcggcgctccagaagttggggtcacctccgcttcgggcttggaagcgcagagctaagctcgtgcgctctaagctcaaggtgcggggaggccgctactccgttgtcgcggactaccttttcggcttcgcttctgcttacaaaccaaagaggcctgcacctcccggtgtcaatacgattgacgtgtctggatggttttccattggagatgactccattggggacatttacaggcagttaccgcttgtcactgggaggtggatccctcttctccttttgcttcctttattggctgcaatcctttacttcaa

SEQ ID NO: 2 - protein, 2941 aa, Canine hepacivirus 1
  MOD_RES (433)..(433): Any amino acid
MSNKSKNQKPKPQRGPRGRVRGQSRSGPVVFPSGAVLVGGRYIPPPQKAIRGPRRGLVQAPKSSERTSPRKRRQPPPQTDSSWRKYFSKFWGDRGYPWPYVDPVLQWGAWGSSPGAYRTRWGPRDPRHKSRNLGRVIDTLTCGVADLAGYVPVLGAPAGALCRGAAHLVRFVEDGANFITGNIPGMGFSIFLLALFSAVSFGEASVVRNGGHVVSNDCNSSQILWASSDWAIHEVGCIPCVDGVCWVPLTSSISVRNESVIVRGLGSHIDVLSAMASVCSTLGIGEACGAATLTYITFLSRFFMPLNLTNDCECFLYPGAISTFEFTMRALQSMMPNLSGFLSMFSGLPNTLFTIFTNGHWGVILALCLYGTTNNYFKLCLLLLAYSGLVSCDDYLNVSLSCNFTVKEMWGWTFFPKWALLNGQRLNCTEGSXYNPKCKGPFDFNVTTDPYIAYSGTRSHPPCPYHVSRPCSVLDASRVCGKPTCFGPAPIEVGVTDRDGKLASWNDSGQFFFDLRSPHRPPRGRWYGCVWLNSTGWVKQCGAPPCNMRLMSNKSKPFVCPTDCFRQNPKATYQLCGQGPWITHSCLIDYTDRYLHFPCTENFTVYPVRMILGDGARDVRVACKFNRSVSCRTEDRLRASIVSLLYSVTTAAVPPCHFSPLPAFTTGLIHLDRNLSDVQYVWAMTPSAVNIFLRLEWAVFFLLLLMDAKVCAILWFCLCLALQAEAYLSDTMRLIALSYIADDSLLWALVFYCVIYFTPSRVPPFCVFVYYWKFALAFMVLALPHRAWAFDNASAVTAAFSIALFCLYITCLSCYKKLFMLVKWWLEYWDVRIECAWRYLGNPHVNPRDEKLAFALLFSFFHPSLFRCIYLPLAVICGSFSMINKRVQKISYLRRAEVLVRVLSICRDVYGSKWVQWCILWLASHLGTFLYDHLTPIDTWAAPGLRDLMHSLEPITLSPMERKVVKWGARKIACGDILRGLPVSARLGREICLGPADKLTSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMLKSSLLLSGRHLIFCHSKKKCEEVALLLRKAGANAVTYYRGLDVSVIPNEGNVVVVATDALMTGYSGNFDTVTDCNTAVELDIEFSLDPTFSMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHVNEKVVVVPHKGVLYADFDEMEECFDHHQYIQQGYEWASRAAQKIREVAASIDPPTGQAQPLLSAVEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPSGEDLINLLPSLLNPGALAVGVGAAFILKRYTGGSEGLVAWVNRLIAFCSRGNHVSPDHYVQQQQVVRDVIACLESLTLTRLVKTIHNFVTSENDQNCDFTAIYFFIQWLMKILYDCFTWAKGIILPHLPGFPIISCDSGYSGRWAGDGLVSTRCGCGNLITGNVRNERIRITGSRKCRNVWLNTFPINSTTTGGPRPNPYDVWKTAVLRITSTEYVEFKREGTAVRVTGATADKLRIPCQVPEPDLMTFIDGVRIHRLAPNPKPMLRDEVVVLIGNHTYPVGATLPCTPEPDVDTVSSLLTDPGHVTAETAARRLRRGRTVDVESSSGSELSAVSRGAASRVSEEHEMAGGPVRPLTGEDELAWIRSFYGRSVTIEVDDKVINFDSWTINSGSEGEHSRESVHAPDDDRVVVAAPPPPPGPAWMRKDYVPALVSGCPIKPGSATPEPSEPSATESAPVEEKEEPKVDDKGEETDPDMPPLEGEEPEEGDDSQWETTSEKGESCSLSYSWTGALVTATRREERRHPIGPLSNTLITKHNLVYQTTTASASARMAKVTIDREQVLDKFYFDTVTQVKKKASEVAADLLTWDEVARLTPKNTARAKSGLSGSDIRQLTRAARRELQSTWQDLLSTSDEPIPTTIMAKNEVFVSSPTSRKPARLIVYPDLPVRACEKRAMYDLFQKLPYAVMGKAYGFQYTPRQRVDRLLDMWRHFKNPMGFSYDTKCFDSTVTPHDIDTERDIFLSANLPDEAKTVIKNLTSRLYRGSPMYNSRGDLVGRRECRASGVFPTSMGNTLTNFIKASAAAKAAGFADPQFLICGDDLVCVTSSKGVEEDEQALREFTNAMTKYSAIPGDFPKPYYDLEQITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYSVVADYLFGFASAYKPKRPAPPGVNTIDVSGWFSIGDDSIGDIYRQLPLVTGRWIPLLLLLPLLAAILYF

SEQ ID NO: 3 - DNA, 9217 bp, Unknown
  Description of Unknown Non-primate hepacivirus NZP-1-GBX2 polynucleotide isolated from horses
cctccgtgctaggcacggtgcgttgtcagcgttttgcgcttgcatgcgctacacgcgtcgtccaacgcggagggaacttcacatcaccatgtgtcactccccctatggagggttccaccccgcttacacggaaatgggttaaccatacccaaagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggacatgagtaataaatctaaaaaccaaaaacccaaaccacaacgaggaccacggggtagggtcaggggtcagtcgcggtctggtcctgtagtattcccttccggcgctgtcctcgtgggggggaggtatatcccccccccaaagaaggccatccgcgggcctcggagaggtctggtgcaagctcctaagtcatcggagcgtatttccccccgaaaaaaacgccagcctccgccacaaactgacagctcgtggcgaaagtatttctcaaaattctggggggataggggatacccatggccttatgttgatcccgttctccagtggggggcctgggggtcttctcctggcgcttatcgcactaggtggggaccccgcgaccctcgccacaagtcgcgcaacttagggcgcgtcattgacactcttacctgtggagttgccgatctagctgggtatgtcccagttctcggagctcccgcaggcgcgttgtgtcggggcgcggctcatcttgtacggtttgttgaggacggtgccaatttcatcactggcaacattcctggcatgggtttctctatcttcttgcttgctctcttctcggctgtatctttcggagaggcctctgttgtgcggaacggtggacacgttgtcagtaacgattgtaattcctctcagattttatgggcatcctccgactgggccatccacgaggttgggtgcattccatgcgtgggtggtgtctgttgggtgccgttaacatctagtatatctgtcagtaatgaatctgttattgttcgtgggctcggttcgcatatagatgtcttgtccgctatggcttctgtttgttctactctgggaattggcgaggcctgtggtgctgcgactctcacttacattactttcttgtctcgattctttatgcctcttaacttgactaatgattgtgagtgttttctctatcctggcgccatttctacctttgagtttactatgagagctttgcaatccatgatgcccaatttgtccggttttctttcgatgttcagtggacttccaaatactctgttcaccatctttactaacgggcattggggcgttatacttgctctttgtctctatggcacaactaacaactactttaaactttgcttgttgcttttagcttattcaggcttagtttcttgtgataattaccttaatgtgtctttgtcttgcaacttcactgttaaggagatgtggggctggacgtttttcccaaaatgggcactactcaacggacagaggctaaattgcactgaggggtccccatacaaccctaaatgcaaaggcccttttgatttcaatgtcacaactgatccgtacattgcctatagcgggactcgttcacatccgccctgcccctatcatgtgtctagaccttgctccgtccttgatgcatccagagtgtgtggcaaacccacctgcttcggtcctgcaccgattgaggtgggtgtcactgatcgggatggaaagcttgcttcttggaatgattctggtcagtttttctttgatctgcggtctcctcaccgcccacctcgtggacggtggtatgggtgtgtttggcttaattcgactggctgggtcaagcaatgtggcgctcctccttgtaacatgagactcatgtccaacaagagcaagccttttgtttgccccaccgattgttttaggcaaaaccccaaagccacttatcaactttgtggccaaggaccgtggatcactcattcttgcttgattgattatactgataggtatcttcacttcccgtgcactgagaacttcacagtctaccctgtccgcatgattcttggcgacggcgctagggatgttagagtggcgtgcaaatttaacagatcggtcagctgtaggactgaagataggttgagagcgagtatcgtctcccttctttacagtgtgactaccgcagctgtcccaccatgtcatttttcgccgctacctgctttcactactgggctcatccacttagatcgcaatctgagtgatgtccagtatgtttgggctatgactcccagcgctgtcaatatcttcttgaggctagagtgggctgtgtttttcttgcttcttcttatggatgcgaaggtatgtgcaattctctggttctgtctgtgcttagctttgcaagctgaggcttatctttcagatactatgcgattgatcgcactctcctacattgctgatgattctttgctgtgggctctggttttttactgtgtaatctacttcaccccaagcagggtcccgcccttttgtgtgtttgtatattactggaagtttgccttggcgtttatggttctggctttaccacatcgtgcttgggcttttgataatgctagtgctgttacggcagcttttagcatcgctcttttctgcttgtatattacttgcttgtcttgctataaaaaactgtttatgctcgtcaagtggtggctggaatattgggatgttagaattgaatgtgcttggcgctatctcggccctagggtaaatcctagggacgagaagcttgcttttgccttactcttttcattctttcacccttccttgttccgctgtatatacttgcctttagctgttatatgtgggtccttttccatgatcaacaaacgtgttcaaaagatctcgtatcttcgacgcgctgaggtcttggttcgggtcctttctatctgcagagatgtttatggtagtaaatgggttcaatggtgtatcttatggttagcttctcatcttggcactttcctttatgaccaccttaccccaatagatacttgggccgcgcctggcttgagagaccttatgcattcgttggagcccataactctttcacccatggagaggaaagttgtcaagtggggcgcgaggaagattgcttgcggcgacattcttcgcggcctgcccgtatctgcgaggcttggtagagaaatctgccttggccccgctgataagctcacaagcaagggctggagattgttatcaccaatcaccgcgaccgtcactaagacgcgcggtattccttcagctattgtttgttgccttacgggtagggataagtatccccacagaggtcattgttacatcttaacctctttgactaagactttcatgggcaccgtgtgtaaaggtgtgctgtggtcggttcatcatggtggcggcactgctactcttgcttctgataaatcatctttgcttcaagtgttgtgttctcccggtgacgatcttgtggcatggcctgcccccgccgggtccaagtcttttcagccgtgcacttgtggctcggctgatgtgtttttggtcacccgtacagggcaggtcgtcccagcgagaaagacctccgaaaaggatgcttcacttatttctcccttacctatttcatcactgaaaggcagttcgggtggcccagtcctgtgtaaggatggggatcttgtaggtatcttctgttctgcttcggttactaggggggtggctaagcgcatacacttcgcagatatgcggacgcggagcgtttctagttgccctcccaagtacactgatttagattctcctcctgctgttccttcatcttatcaggtctctttccttcacgctcccactggtagcggcaaatccaccaagatgccgctgtcctatgtggagctgggatatcatgtcttagttcttaacccttcagtcgcctcgactctcagttttgggccttacatggataagacctatggggaatgtccaaacatcaggacaggcgcaagctgcaagactacaggctctaaacttacttattccacctatggtaagttcttagctgacgggggtgtttctgctggtgcctatgacattataatatgtgatgagtgccatagcacagactccacatccgtactaggaataggctccgtccttgatggggcagagtctaaaggtgtaaagcttgttgtacttgctactgctacgccacccgggtctcagactgtccctcatcctaacatcgatgaggaggccctcactcagagcggtgacatacccttttacgggaagatgttgaagtcgtctttgctcctaagtgggagacatcttatcttctgccattcgaagaagaagtgtgaagaggtcgcacttctcctgagaaaggccggagctaatgctgtaacctactatcggggtttggatgtttccgtcataccgaatgagggtaatgtcgtcgttgttgccacggacgcccttatgacagggtattctggcaattttgacaccgttactgactgtaacaccgctgtggaactggacattgagttttcccttgacccaacattttctatggttaccactcctaaaccatcagatgccgtttgcagaacacagcgcagggggaggactggtagaggccgcaggggcacttactactatgtaaatagtggtgagcgcccatctggggtcctatcgtcatccgtcctgtgcgagtgctatgacagcggcttggcttggtttggcctgagtccagcgcaggtaactgtgctactccaggcttaccttaagcaacctggccttcccaccggacttgaccacaccgaattttgggagagcgtcttcattgggttacctacagtcgacgctttctttttgtcccagctaaaacaacagggtgtaactttcccttatttaactgccatccaagcaactgtatgccttaacgcccaagctaaggcgcccagcaaagacgagcgctggaaggtgctgtcacggtatattaccacaaaccgtactccaacgcctttgttgtacagacttgaggacacccacgacgatcttacttttactcacccagtgaccaagtatattcaggcctgtatggaggccgaaattgacacccaaactaatgcttgggttattgcgggtgggtgtgttgcagctttggttgctgtcgctgcccttacgggtagcgttgcgatcatagctgaggtacacgtaaatgagaaagttgtggtagtccctcacaagggcgttctctacgccgattttgacgagatggaggaatgttttgatcaccatcaatacatccaacaaggatacgaatgggcgtcgcgtgcagcgcagaaaatccgcgaagtagcggcgtccattgaccctccaactggtcaagcacaaccattgctatcggctgttgagaaattttggaatcagcacatgtggaacatcttatcaggtgtgcagtatttggcaggtcttacaacactcccttataatccatcagtagcttgtctaatgggttttgtttcaggtctcactacaggattgccgagaccagctatggctttcctcaccattctaggtggttgggcggcaagcatggtggcccctccccaggcagcatccacctttgtcggcgctgggctggctggcatcgctatcggtgcagtcggcttcactgacgtcatcgttggcctgcttgcggggtacggggccggtgtggctggcgcccttactgccttcaagatcttgagtggtgtcaccccgagtggtgaagacctaattaacctcttgccatctttgctcaaccctggcgcgcttgctgtaggtgtgggcgccgcattcatcctaaaaagatacacaggcggcagtgaaggactcgtcgcatgggtcaatcggctcatagcgttttgttctagggggaaccacgtctcccccgatcactatgtccaacaacagcaggtggtcagggatgtcattgcttgtcttgaatctttaaccttaactagattagtgaaaactatccataactttgttacaagtgaaaatgaccaaaattgtgattttactgccatttatttctttatccaatggttaatgaaaatactttatgattgtttcacctgggcaaagggaataattcttcctcatcttccaggtttcccgatcatatcgtgtgattcaggctactctggccggtgggctggagatggtctcgtttccactcggtgtggttgtggtaacttgatcactggaaatgtgaggaatgaaagaattaggattacaggctcgcggaagtgccgtaacgtctggctcaataccttcccaattaactctactactactggtgggccacggcctaatccatacgatgtgtggaaaacggctgttttgagaataacttccacagagtatgttgagttcaagagggaggggactgctgtcagagttactggcgcaaccgctgacaaattacgtatcccttgccaggtgcctgaacctgatttgatgacctttatagatggagttaggatacataggctggctccaaacccaaaaccaatgttgcgcgatgaggtggtggtcctaattggcaatcacacctacccagtcggcgcgacacttccatgcactccagaaccagatgttgacactgtatcttccttactaactgatccaggacatgtcactgcagagactgcagcaagacggctcaggcgcgggcgtactgttgatgttgagtcctcgagcggctccgagctgtcggcggtttcgcgaggcgcagcttctcgtgtgtcagaagaacatgagatggctggcggccctgtgagaccgctgaccggcgaagacgagctcgcgtggattcgatcattttacggacgctctgtgacgattgaggttgacgacaaggttatcaactttgactcgtggaccatcaattctgggtctgagggggagcactcccgagaatcagtccatgcgccagacgacgatcgggtggtggtggctgcaccgcctccgccgcctggtcctgcctggatgcgcaaagactatgtcccagctctcgtttccgggtgtcccatcaagcccggaagtgctactcctgagcccagtgagccctcggctacagaatctgccccagttgaggaaaaggaggaacctaaagtcgacgataagggtgaggaaacagaccctgacatgccccccctcgaaggtgaagagcctgaagagggggatgacagtcaatgggagactacttctgaaaagggagagtcatgctccttatcctattcgtggaccggtgccctcgtcacggcaacccgccgtgaggagcggcgtcatcctataggtcccctttccaacacactaattactaaacacaacctcgtgtaccagacaacgacagcgtctgctagcgcgaggatggctaaagtgacaattgatagagaacaggtccttgacaaattctactttgatactgtcacccaagtcaaaaagaaagcttcagaggtgactgctgacctgcttacgtgggatgaagttgcgcgcctcactccaaagaacacggctcgcgcgaaatcgggcctttccggttccgatattcgccaacttacgcgagctgcgaggcgggaactccagtcaacgtggcaggacctcttgtcaacctctgatgaacctattcctactacaattatggcaaagaacgaggttttcgtctcgtcgccaacttctcgcaaaccagccaggctgatcgtctaccctgatttgcctgtgcgtgcctgtgagaagagggcgatgtacgacctcttccaaaaattaccctatgccgtcatggggaaggcctatggatttcaatacactcctcgccagcgtgtcgatagactattagacatgtggcggcatttcaggaatcctatgggcttttcttacgacaccaaatgttttgactccactgtcactccgcatgatatagacactgagagggacatctttctaagtgccaatctccctgatgaggccaagacagtcattaagaacttgacgtctaggctttatagagggtcccctatgtacaactcacgtggggacctggttgggagaagggagtgccgtgcttccggggttttcccgacgagcatgggcaacactcttactaacttcatcaaagcctctgcagctgccaaagctgctgggtttgctgaccctcagttcttaatctgtggagatgatcttgtttgtgtcacctccagtaaaggcgttgaggaagatgaacaggcgttgcgtgaattcacaaacgccatgacaaagtactccgccattcctggagatttcccaaagccatactatgacttggagcaaataacatcttgttcctctaatgtgactgttgctcaagacagaaacgggcgaccttattatttcctcactcgtgatccaacaacaccgctggctcgcgcgagttgggagacaattagtcacagtcctgttaacagttggttaggaaacataattgcttttgctcctacggtgtgggttagactcgttttcctcactcatttcttcggtctgctgctccaacaggatgctgtggatcgaaattatgagtttgagatgtacggatcaacctactccgtaaacccacttgacttaccagcaataatttataagctccatggccctgaggcctttgatcttacaaactattctccttacgaggtccagagggtggccgcggcactccagaagttggggtcacctccgcttcgggcttggaagcgcagagctaagctcgtgcgctctaagctcaaggtgcggggaggccgctactccgttgtcgcggactaccttttcggcttcgcttctgcttacaaaccaaagaggcctgcacctcccggtgtcaatacgattgacgtgtctggatggttttccattggagatgactccattggggacatttacaggcagttaccgcttgtcactgggaggtggatccctcttctccttttgcttcctttattggctgcaatcctttacttcaataaataaaaaaa

SEQ ID NO: 4 - DNA, 9249 bp, Unknown
  Description of Unknown Non-primate hepacivirus G1-073-GBX2 polynucleotide isolated from horses
cctccgtgctaggcacggtgcgttgtcagcgtattgcgcttgcatgcgctccacgcgtcgtccaacgcggagggaacttcacattaccatgtgtcactccccctatggagggatccacccgtctacacggaaatgggttaaccatacccagagtacgggtattcgggtcctcctagggcccccccggcaggtcgagggagctggaattcgtgaatctgtgagtacacggaaatcgcggcttgaacgtctttggaccttcgaagccgaaatttgggcgtgccccacgaaggaaggcgggggcggtgttgggccgccgccccccttgtcccacggtctgataggatgcttgcgagggcacctgccggtctcgtagaccataggacatgggcaacaactctaaaaaccaaaaaccaaaaccccaaagaggaccacggaacagggttaggggccagtcgcggtctggccctgtagtattcccgtccggcgctgttctcgtcggaggaaggtatattcctccgcccaagaaggccatccgcgggcctcgtagaggtttggtgcagactcccaagtcatcggagcggggttccccccggaaaaagcgccaacctccgccacagactgacagctcgtggcgaaagtatttccctaaattttggggggacaggggatatccatggccatacgttgatcccgttcttcagtggagtgcatggggtacctcacctggtgcataccgtactaggtggggcccccgtgaccctcgacacaagtcgcgcaaccttggacgcgtcattgatactatcacctgtggggttgctgaccttgccggatatgttccggtagtcggcgctcccgctggcgcgttgtgtcgtggcgctgctcatcttgtgcggtttgtagaggacggggccaatttcatcactggcaacatccctggcatgggtttctccatctttatcttggctcttctctcagctttgtcattcggcgaagcgtctgtcgtgcgtaacgggggccacgttgttagcaatgactgcaatcactcccagattttgtgggctgcttccgactgggctattcacgaagtgggctgtgtgccctgcgtgaacagttcttgttgggtgcctctcacttcgagtatttctgtgaggaatgagtctgttatcgtacgtggtctcggctcgcacatagatgttcttgctgctatggcgtctgtctgttctactcttggtattggtgaggcttgtggtactgctactctaacttacatcaccttcttatctagatttttcatgtccttaaatcttacaaatgattgtgaatgttttctttaccctggccccatctctacctttcagttcactatgagagctttgcaatctatgatgcccaacctgtccggctttgtgtctatgtttagtggggttcctaataccctacttaccatctttacaaacgggcattggggcgtcattcttgctctatgtctttatggcacaactaataattacttcaagctttgcctgttgctcttagcatattctggcttagtttcctgtgaccaaactgaatatcttaatgtttctttgtcctgtaactttactgttaagcagatgtggggctggacgtttttcccgaagtgggctttattgaatgatcagaggttgaattgcaccgagggttcgccttacaacccgaagtgtaagggaccaatggattttaacatcactgatgatccgttcattgcctataccgggacgcgttcacacccaccctgtccttaccatgtgtctagaccttgctctgtcctaaatgcgtccagagtctgtggcaaaccgacctgtttcggccctgcgcctatagaggttggcgtcactgaccgggatggaaaactagcttcttggaatgatactggccaatttttctttgatcttcggtccccccaccgaccccctcggggccgttggtatgggtgtgtttggctcaactctaccggatgggtaaagcaatgtggcgctcctccgtgtaacatgagactcgtgtctaacaggagcagaacatttgtttgcccgtccgactgctttaggcaaaaccctaaggccacttatcaactgtgtggtcaaggaccttggatcagctataattgtttaattgattacactgataggtacctccatttcccttgtacggagaatttcaccgtttatccggtccggatggttcttggtgatggcgctcgggatgtgagggtggcttgtaaatacaacagatcggtcagctgcagtactgaggataggttgagagcgagtatcgtctccctactctacagtgtgactactgccgctgtacctccttgccacttctctcccttacccgccttcactactggtcttattcacttggaccgaaatctgagtgatgtccagtatgtttgggcgatgactcccagtgctgttaacattttcctgaggctcgagtgggcggtgtttttcctacttctgctcatggatgctaaggtctgtgctatcctctggttttgtttgtgcttagccttgcaggctgaggcttacctttcagatactatgaggcttattgctttgtcatacatagctgatgattctttgctttatgctctgctcttctactgtgtgatttactttaccccaagtagagtacctcctttttgtgtcttcatgtattactggaagttttccttggcctttatggtgttggctctaccacaccgagcttgggcttttgataatactagtgctgttactgctgcctttagtatagctcttttctgcttgtatgtaacctgtttgtcttgttataaaaaactctttatgcttgttaaatggtggcttgaatattgggatgttagggtcgaaagcgcttggcgatatctcggccctagggttaatccaaagagtgatgaaaaacttgctttcgcgttggtcttcacttttctttacccctctttgtttcgtgctgtctatttgcctctggcggtagtctgtggttccttttccatgatcaacaagcgcatacagaaggtacaataccttaggcgcgctgaggtcttggtgagggtcctcactatctgcagggacatttacggtagcaagtgggtgcagtggtgtgttttgtgggttgcttctcattttgggactttcctctatgatcatctaactcctattgacacctgggctgccccagggttgagggacctcctgcattccctagagcccataaccctctctcctatggagaggaaggtagtcaaatggggtgcaaggaaagtcgcttgcggcgacatcttgcatggtctgcctgtatcggcgaggctgggcagagaaatctgtctaggccccgccgaccgtctcacgagcaaaggctggaggttgttatcacccataactgccactgtaaccaagacacgtggcataccctcagcaattgtttgttgtctgaccggcagagacaaatacccccacagaggacattgttatatcttgacgtccttgactaaaaccttcatgggcactgtttgtaagggcgtgctgtggtccgtccaccacggtggcggcactgctacccttgcttctgacaaatcttccctgttgcaagtcttgtgctctccgggcgatgaccttgtcgcatggcctgctccagccgggtccaagtctttccagccttgcacgtgtggctcggctgatgtgtatctggttacccggactgggcaggttgtaccggcacggaaaacgtccgaaaaagatgcttctctcatttcccctttgccaatttcttcgcttaaaggtagctcgggaggtccagtcctgtgcaaggatggggatcttgtgggcattttctgttcagcctccgttactaggggagttgcaaaacggatacacttcgcggacatgcggacgcgaagtgtttcctcctgccccccaaagtacacggatctggactccccacctgcagttccttcttcatatcaggtttctttcctccatgctcctactggcagtggcaaatccaccaagatgccattgtcttatgtagaattgggttatcatgttctagttctcaatccgtcggtcgcctccactcttagtttcgggccatacatggataagacctatggtgagtgtccaaacatcaggactggcgctagctgtaaaactacaggctctaaactaacatattccacctatggaaaattcctagcggatggtggggtttctgcgggtgcttacgacataatcatttgtgatgagtgccatagcacagactccacgtccgttttggggattggatctgtccttgacggggcagaatctaaaggtgtaaagcttgttgttcttgctactgccaccccaccagggtcccaaaccgttcctcatccgaacatcgatgaggaggcccttacccagaatggagatatccccttttacggaaagatgttgaagtcgtccttgctccttagcgggaggcatcttatcttctgccattcaaagaagaagtgcgaagaagtcgcgctccttttgagaaaggctggagctaatgctgtcacctactatcgaggcttggatgtttccgtcatcccgaatgaggggaacgttgttgttgtggccacggacgcccttatgacggggtactctggcaacttcgacactgtgactgactgcaacactgctgtagagctggacattgagttctctcttgacccaactttctccatggttaccactccaaaaccatctgatgccgtctgtcggacacagcgcaggggaaggactggcaggggccgcaggggtacttactactatgtaaatagtggtgaacgcccctccggagtactgtcgtcatccaccctgtgcgaatgttacgatagtggcttggcatggtttggccttagtcccgcgcaggtcactgtgctgctacaggcctatttaaagcaacctggactccccaccggattggaccatactgagttttgggagagcgtcttcataggcttgcctacagttgatgctttctttctatctcagctaaaacaacaaggagttacattcccctatcttacagcaatacaagccactgtctgccttaatgctcaagctaaggctcccagcaaggatgaacgctggaaggtgctgtcccggtatattacaactaaccggactccaacgcccctgttgtacaggcttgaggacacacacgatgacttgacgtttacacaccctgtgaccaagtatatccaggcctgtatggaggccgagattgatacacaaacaaatgcttgggtcatagcgggtggctgcgtcgcggctctggttgccgtcgcagccttaactggcagcgtcgcaatcattgccgaagtgcatgtcaacgaaaaagtcgttgttgttcctcataagggcgttctctatgctgattttgacgagttggaggaatgctttgatcatcatcaatacattcaacaaggttacgagtgggcgtcgcgcgcagcacaaaagatccgcgaggtcgccacgtccattgaacctcctactggtcaagcacaacctatgctatcagcaatagaaaaattttggaatcagcacatgtggaacattctatcaggagtacagtacttggcaggacttactacacttccttataatccatctgtggcttgcttgatgggctttgtttcaggtcttaccacaggattgccacggccagctatggctttcctcaccattttaggcggatgggctgccagcatggtcgctccaccgcaagcagcctccacattcgttggggccggactagccggtattgctatcggcgctgtcgggtttaccgatgttatcgtggggcttcttgctgggtatggggctggcgttgcaggcgcccttaccgcttttaagatcttaagtggtgtcaccccgagtggtgaggatctgattaatttgctcccatctttgctcaaccctggcgccctcgctgtgggcgtgggagccgcttttatcctgaaaaggtatacaggcggcagtgaagggcttgttgcttgggttaatcggctcatcgccttttgttcccggggcaatcatgtctcccctgatcactacgtccaacagcagcaggtcgtcaaggatgtcattgcctgccttgagtccctaaccctaaccagattagtgaaaactatacataattttgttacaagtgaaaatgatcaaaattgtgattttactgccatttatttcttcattcaatggctaatgaaagctctttatgattgtttcacctgggcaaagggaattatcctcccacacctcccgggtttcccaatcatatcgtgtgacacgggctactccggccgttgggccggggagggtttagtgactacgcggtgtggttgtgggaacatgatcaccgggaatgtgaggaatgaaaagataaggataacaggctcgcggaagtgccggaacctttggctcaatgctttcccaatcaattcgacgactactggtgggcctcgacccaatccttatgacacctggaaaacagctgttttgaggatcacgtccactgagtatgttgagtttgagaggaagggcactgcagtaaaggttataggcgctactgctgacaaattaaggattccctgccaggttcctgaacctgatttaatgacttacatagatggagttcggatccacagattggctccgactccaaaacctatgttgcgtgacgaggtggtggtcttaattggcaatcacacctaccccgtgggcgcaactcttccatgcactccagaaccagatgtagacactgtatcttctcttttaactgatccaggacatgtcaccgcagagactgcagctagacggctcagacgcggacgtaccgtggacgttgagtcctcgagcggctccgagctgtcggccgtctcgcggggtgccgcgtctcgtgtgtcagaagaacatgagatgcaaggtggccccgtgagaccgctgactggcgaagacgagctggcgtggattcgttcgttctacggacgttctgtgacgattgaagttgatgataaagttattaacttcgattcatggaccatcaattctgggtccgagggagagcactctcgagagtcagtcgtcgctccagacaacgaccaggtcgtggtggcacaacctccaccacctccaggtccggcttggatgcgaaaagactacgtcccagccctcgtatccgggtgtcccatcaagcccggaagtgctacccctgagcccagtgagccttctgctacagaatctgctcccgtggaggagaaagaggaacttagggtcgacgaggccgatgtcgagacagaccccgaaatgcccccccttgaaggtgaagagcctgaaggggacgatgacggggaatgggagactacgtctgaaaaggcggagtcgtgctcgctgtcgtactcgtggaccggcgccctggtcacggctactcgccgtgaggagaggcgccatcccataggacctctctccaacactctcattaccaaacacaatctcgtctatcagacaacaacagcttctgctagtgcgagaatggctaaagtgacaattgacagagaacaaatttttgacaaacattactttgacactgtcaccgcagtcaagaagagggcttcggaggtgacggcggacctgctcacgtgggatgaagttgctcgtttgacgcccaagaacacagctaaagcaaagtcggggcttagcggctccgatgtgcggaagctcacaagggcggccaggcgggaactaaattccatgtggcaggaccttttgtcaggctttgaagagcccattccaacaacagtcatggcaaaaaatgaggttttcgtgtcatctccgaccgctcgcaagccagctaggctgatcgtctatcccgacctgcctgtgcgtgcctgtgagaaaagggccatgtatgacctcttccaaaaacttccctatgccattatggggaaggcctatggatttcaatacactccccgccagcgtgttgagagactgcttgacatgtggcgacattttaagaatcccatgggcttttcctatgatacaaaatgctttgactctaccgtcactcctcatgatatagatactgagagggacattttccttagtgctaacctacctgatgaagccaaaactgtaatcaaaaatttaacatcaaggctttacaggggatctcccatgtataattcgcgtggggatcttgttggaaagagggaatgtcgcgcttccggggttttcccgacaagcatgggcaacactctgaccaactacattaaggctaccgcggctgccaaagccgctggtttaagtgaccctcagtttctaatttgtggggatgaccttgtatgcatcacctccagcaaaggcgttgaggaagatgagcaagcgctgcgaaacttcaccagcgcaatgacaaagtactccgccattcctggagaccttcccaaaccatactatgacttagaggaaataacatcttgctcctcaaatgtgactgtcgcccaggatagaaatgggcgaccctattatttcctaactcgtgatcccacgacaccactcgcgagagcgagttgggaaacaattagtcacagtcctgtaaatagctggctagggaacataattgcttttgcacccactgtctgggttaggctcgttttcctcactcattttttcggtctattgcttcaacaggatgccgtggatcggaattatgagttcgagatgtatgggtcaacctattcagtaaacccgcttgatttacctgcgataatttataaacttcatggccccgaggcctttgatcttacaaattattctccttacgaagttcagagggtggctgcagcactccagaagttgggatctcccccactacgggcttggaagcggcgagctaagctcgtccgctcaaagctcaaggtacgggggggtcgctacgctgttgtcgctgactacctttttggcttcgcttctgcctatcgtccgaagaggccagcacctcctggcgtaaactcgatcgacgtttccggttggttctccattggagatgactcgattggagacatttaccggcgacttccggtcgttgccgggaggtggatacctcttctgcttttgctacctcttttggctgcaattctgtatttcaataaatagacaaaaaaaaaaaaaaaaaaaaaaaaaa

SEQ ID NO: 5 - DNA, 9219 bp, Unknown
  Description of Unknown Non-primate hepacivirus A6-006-GBX2 polynucleotide isolated from horses
cctccgtgcttggcacggtgcgttgtcagcgttttgcgcctgcatgcgctacacgcgtcgtccaacgcgggggagttccacactaccatgtgtcactccccctatggagggttccaccccgcttacacggaaatgggttaaccacacccatagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggacatgagtaacaaatctaaaaatcaaaaacccaaaccacagcgaggaccacggagtagggtcaggggccagtcgcggtctggtcctgtagtattcccttccggcgcagtccttgtgggggggaggtatatcccccccccaaagaaggccatccgcgggcctcggagaggtctggtgcaagctcccaaatcatcggagcgcacttccccccggaaaaaacgccagcctccgccacaaaccgacagctcgtggcgaaagtattttccaaaattctggggggataggggttatccatggccttatgttgatcccgttctccaatggggggcctgggggtcctctcctggtgcttatcgcactaggtgggggcccagagaccctcgtcacaagtcgcgcaatctggggcgcgtcattgacactctcacctgtggggttgccgacctcgctgggtatgtcccggttctcggcgcacccgcgggcgcactgtgtcgtggcgcggctcaccttgtacggtttgtcgaggacggtgccaatttcatcactggcaacattcctggcatgggtttctctatctttttgcttgctctcttttcggctgtgtctttcggagaggcttctgttgtgcggaacggtggacacgttgtcagtaacgattgcaattcttcgcagatcttatgggcgtcctctgactgggctatccacgaggttggatgtgttccatgcgtggacagtgcttgttgggtgccgctaacatctagcatatctgttaggaatgaatctgttattgttcgtgggctcggttcgcatatagatgtcttatctgctatggcttccgtctgttctactctgggtattggtgaagcgtgtggtactgcaactctcacttatattactttcttgtctcgattctttatgtctcttaacttgactaatgattgtgagtgttttctctatcctggcgccatctctacctttgagtttaccttgagagctttgcaatctatgatgcccaatttgtccggtttcctctcgatgtttagtggacttcctaatactttgttcaccatctttactaacgggcactggggcgtcatactcgctctttgtctctatggcacaactaacaattactttaaactttgcttactgcttttagcttattcgggcttagtttcttgtgatgattacattaatgtgtctttgtcctgtaacttcactgttaagcagatgtggggctggacgtttttcccaaagtgggcgttacttaacggacagaggctgaattgcactgaggggtctccatataaccctaaatgcaaagggccttttgatttcaacattacagctgatccgtttatcgcctatactgggactcgttcacatccaccctgcccttatcatgtgtctagaccttgttctgttattagcgcatccaaagtgtgtggcaaacctacttgtttcggtcctgcaccgattgaggtgggtatcactgatcggaatggaaaacttgcttcttggaatgagtctggtaagttctattttgacttacggtctcctcaccgcccgcctcgcggacggtggtatggatgtgtttggcttaattcaaccggttgggtcaaacaatgtggtgctcccccttgtaacatgccattcatgtctaacaggagcaggacttttgtttgccccaccgattgttttaggcaaaaccccaaagccacctaccagctttgtggccagggaccgtggatcactcactcttgtttgattgattatactgataggtatcttcatttcccgtgcactgaaaatttcacagtctaccctgttcgcatgattcttggtgacggtgctagggacgttagggtggcgtgcaaatataatagatcggtcagctgtacgactgaaaaaaggttgagagcgagtatcgtctcccttctttacagtgtgactactgcagctgtcccaccatgtcatttttcgccgctacctgccttcactactgggcttatccacttagatcgtaatctgagtgacgtccagtatgtttgggctatgactcccagcgctgtcaacatcttcttgaggctagagtgggctgtgtttttcttgcttcttctcatggatgctaagatatgcgcaatcctctggttctgtttgtgcttagctttgcaagctgaggcttatctctcagatactatgcgattaattgcactctcttatattgctgatgattctctgctgtgggcattagctttctattgtgtgatctattttactccaagcagagttccccctttttgtgtatttgtatattattggaaatttgccttagcttttatggttttggctttaccacatcgtgcttgggcctttgataatactagtgctgttacagcagctttcagcatagctcttttttgcttgtatattacctgtttgtcttgctataaaaaattatttttgcttgttaagtggtggttggaatattgggatgttagaattgaatgtgcttggcgctatcttggccctagggtaaaccctagggacgagaagcttgctttcgctttacttttctcattcttttacccgtccttgtttcgctgtatatatttgcctttagctgttatatgtgggtccttttccatgatcaataagcgtgtccaaaagatatcatatcttcggcgcgctgaggtcttggttcgggttctttgcatttgtagagatatttatggtagtaaatgggttcagtggtgtattctctggttagcttctcactttggtactttcctatatgatcaccttaccccaatagacacctgggccgcacctggcttaagagatcttatgcattctttggagcccataactctttcacctatggagaggaaagttgtcaaatggggcgcgaggaaggttgcttgtggcgacattcttcgcggcctgcccgtatccgcgaggcttggtaaagaaatttgccttggccccgctgataagcttacgagcaagggctggaggttattatcaccgatcactgcgaccgtcactaagacgcgcggtattccttcagctattgtttgttgccttacgggtagggacaagtatccccatagaggtcattgttatatcttaacctctttgactaagactttcatgggcaccgtgtgtaaaggtgtgctgtggtcggtccatcatggtggtggtactgctactcttgcttctgataaaacatctctgcttcaagtgttgtgttctcccggtgacgatctggtggcatggcctgctcctgctgggtcaaaatcttttcagccatgcacttgtggctcggctgatgtgtttttggtcacccgtacggggcaggttgtcccagccagaaagacctccgaaaaggacgcttcactcatttcccccttacctatttcatcactgaaaggcagttcaggtggcccggtcctgtgtaaggatggggatcttgtaggtatattctgttctgcttcggttactaggggggtggctaagcgcatacacttcgcagacatgcgaacgcggagcgtttctagttgccctcccaagtatactgatttagactctcctcctgctgtaccctcatcttatcaggtctcttttcttcatgctcccacaggtagcggcaaatccaccaagatgccgctgtcctatgtggagttgggatatcatgtcttagttcttaacccttcagttgcttcaactcttagtttcgggccttatatggacaagacctacggggaatgtcctaacatcaggacgggtgcaagctgtaagactacaggctccaaactcacctattccacctatggtaagttcttagctgacgggggtgtttctgctggcgcttatgacattattatatgtgatgaatgccacagtacagactctacttccgtactaggaataggctccgtccttgatggagcagaatctaaaggtgtgaagcttgttgtacttgctactgctacgccgcccgggtctcagactgtccctcatcccaacatcgatgaggaggcccttactcagagcggtgacatacccttttacgggaagatgttgaagtcgtctttactcctaagtgggagacatcttatcttctgccattcgaagaagaagtgtgaagaggtcgcgcttctcctgagaaaagccggagctaatgctgtaacctactaccggggtttggatgtttccgtcataccgaatgagggtaatgtcgtcgtcgttgccacggacgcacttatgacagggtattctggcaatttcgacaccgttactgattgcaacactgccgtagaactggacattgaattttcccttgacccaactttttctatggttactactcctaaaccatcagacgccgtctgtagaacgcagcgcagggggaggactggtaggggccgcaggggcacttactactatgtaaatagtggcgagcgtccatctggggtcctatcgtcttccgtcctgtgcgagtgttacgacagcggcttggcttggtttggcctgagtccagcgcaggtaactgtgctacttcaggcttaccttaagcaacctggccttcccaccggacttgaccacactgaattttgggagagcgtcttcatcggcttgcctacagttgacgctttctttttgtctcagctgaaacaacaaggtgtaactttcccttatctaactgccatccaagcaactgtatgccttaacgctcaagctaaggcgcccagcaaagacgaacgctggaaggtgctgtcacggtatattaccacaaaccgtactccaacgccgttgctatacagacttgaggacacccacgatgatctcacttttacccacccagtgaccaagtatattcaggcctgcatggaggctgaaattgatacccagactaatgcttgggttatagcgggtgggtgtgttgcagctttggttgctgtcgctgccctcacgggtagcgttgcgatcatagctgaggtacacataaatgagaaagttgtggtagtccctcacaagggcgttctctacgccgattttgacgagctggaggaatgttttgatcaccatcaatacatccagcaaggatacgaatgggcgtcacgtgcagcgcagaagatccgcgaagttgcggcgtccatcgatcctccagctggtcaggcacaaccattgctatcggccgttgagaaattttggaatcagcacatgtggaacatcctatcaggtgtgcagtatttagcaggtcttacaacactcccctacaatccatcagtagcttgtctaatgggttttgtctcaggtctcactacaggtttaccaaggccagctatggcttttctcaccattctaggtggttgggcggcgagcatggtggcccctccccaggcagcatccacctttgtcggcgctgggctggctggcatcgctattggtgcagtcggcttcactgacgtcatcgttggcctgcttgcgggatatggggccggcgtggctggcgcccttactgcctttaagatcctgagtggtgtcactccaagtggtgaagacctaattaacctcttgccatctctgctcaaccctggcgcgcttgctgtaggcgtaggtgccgcctttattctgaagaggtatacaggcggcagtgagggactcgtcgcatgggtcaaccggctcatagcgttttgttccagggggaatcacgtctcccccgatcactacgtccaacaacagcaggtggtcagggatgtcattgcttgtcttgagtctctaactttaactagactggtgaaaactattcataattttgttacaagtgagaataatcaagattgtgattttactgccatctatttctttattcaatggttgatcaaaacactttatgattgtttcacctgggcaaaggggatcatccttccccaccttccaggtttcccgatcatatcgtgtgattcaggctactctggccgatgggccggagatggtctcgtttctactcggtgtggttgtggtaacatcatcactggaaacgtgaggaatgaaaggattcgaatcacaggatcgcggaagtgccgtaacgtctggctcaataccttcccaattaactcatctactactggtggaccacggcctaatccatacgatgtgtggaaaacagccgttttaagaataacttccacagagtacgttgagttcaagagggaggggactgctgtcagagttaccggcgcgaccgctgacaaattacgtattccctgccaggtgcctgaacctgatttgatgacattcatagatggagtccggatacataggctggctccagatccaaaaccaatgttgcgcgatgaggtggtggtcttaattggcaatcacacttacccggtcggcgcgacactcccatgcactccagaaccagatgttgacactgtatcttccctgctaactgatccaggacatgtcactgcagagactgcggcaagacggctcaggcgtgggcgcactgttgatgtcgagtcctcgagcggctccgagctatcggcggtttcgcgaggcgcagcttctcgtgtgtcagaagagcatgagatggctggtggccctgtgagaccgctgaccggcgaagacgagctcgcgtggattcgatcattttacgggcgctcagtgacgattgaggtcgacgacaaggttattaactttgactcgtggacgatcaattctgggtctgagggggagcactctcgagaatcagtcgttgcaccagacaatgatcaggtggtggtggctgcaccgcctccgccgcccggtcctgcctggatgcgcaaagactatgtcccagctctcgtttccgggtgtcccatcaagcccggaagtgctactcctgaacccagtgagccctcagctacagagtctgccacagttgaggaaaaggaggaacctaaacccgacgagaagggtgaagaaacagaccctgacatgccccccctcgaaggtgaagaacctgaagaggaggatgacagtcagtgggagactacgtctgaaaaggcggagtcttgttctttgtcctactcgtggactggtgccctcgtcacggcgactcgccgcgaggagcggcgtcatcccataggtcccctttccaacacactaatcaccaaacataacctcgtgtatcagacaacaacagcgtctgctagcgcgaggatggctaaagtgacaattgatagagaacaagtccttgacaaattctattttgacactgtcacccaagtcaaaaagaaagcctcagaggtgactgctgacctgcttgcgtgggacgaagttgctcgccttactccaaagaacacagctcgcgcaaaatcgggcctctccggctccgatattcgccaactcacacgagctgcgaggcgggaactccaatcaacgtggcaggaccttctgtcatcctctgatgaacctattcctacaacaattatggcaaagaatgaggttttcgtctcgtcgccaactgctcgcaagccagccaggctaatcgtctaccctgacttgcctgtgcgtgcctgtgagaagagggcgatgtacgatctcttccaaaaattaccctatgttgtcatggggaaagcctatggatttcaatacactcctcgccaacgtgtcgacaggctcttagacatgtggcggcatttcaagaatcctatgggcttctcttacgacactaaatgttttgactccactgtcactcctcacgatatagatactgagagggatgtcttcctgagtgccaacctccctgatgaggccaagacagtcatcaaaaacctgacgtctaggctttacagaggatcccccatgtacaactcacgcggggacttggttggaagaagggagtgccgtgcttccggggttttcccgacaagcatgggtaacacccttactaattacatcaaggcctctgcagctgctaaagctgcggggtttgctgaccctcagttcttaatctgtggggatgaccttgtttgtgtcacctccagtaaaggcgttgaggaagatgaacaggcgttgcgtgaatttacgaacgccatgacaaagtactccgccatccctggagacctcccaaagccatactatgacttggagcaaataacatcttgttcctctaatgtgactgttgctcaagacagaaacgggcgaccttattatttcctcactcgcgacccaacaacaccgctggctcgcgcgagttgggagacaattagtcacagtcctgttaacagttggttaggaaacataattgcttttgctcctacagtgtgggttagactcgttttcctcactcatttcttcggcctactgctccagcaggatgctgttgatcggaattatgagtttgagatgtatgggtcgacctactctgtgaatccacttgatttgccagcaataatctataagcttcatggccctgaggcttttgaccttacaaactattctccttacgaggtccaaagagtggccgcagcacttcagaagttggggtcgcctccgcttcgggcttggaagcgcagggctaagctcgtgcgctctaagctcaaggtgcggggtggccgctactctgtcgtcgcggactatcttttcggcttcgcttctgcttacagacctaagaggcctgcgcctcccggtgtcaactcgattgacgtgtctggatggttctccattggagatgactccattggggacatttacaggcagttaccacttgtcaccgggaggtggatccctcttcttcttttgcttcctttactggctgcagtcctttatttcaataaataaaaaaaaaa

SEQ ID NO: 6 - DNA, 9247 bp, Unknown
  Description of Unknown Non-primate hepacivirus B10-022-GBX2 polynucleotide isolated from horses
cctccgtgctaagcacggtgcgttgtcagcgttttgcgcttgcatgcgctacacgcgtcgtccaacgcggagggaatttcacattaccatgtgtcactccccctatggagggtctcaccccgctcacacggaaatgggttaaccatacccatagtacgggtgtgcgggtcctcctagggcccccccggcaggtcgagggagctggaattcgtgaatccgtgagtacacggaaatcgcggcttgaacgtcatacgtgaccttcggagccgaaatttgggcgcgccccacgaaggaaggcgggggcggtgttgggccgccgctccctttatcccacggtctgataggatgcttgcgagggcacctgccggtctcgtagaccataggacatgagtaacaaatctaaaaaccaaaaaccaaaaccccaaagaggaccacggaatagggtgaagggccagtcgcggtctggtcccgtagtattcccgtccggcgctgtcctcgtgggggggagatacatccccccccccaagaaggccatccgcgggcctcgaagaggtttggtgcaggctcctaagtcatcggagcgaactgccccccggaaaaggcgccaacctccgccacaaaccgacacctcgtggcgaaagtactttccaaaattctggggggacaagggatacccatggccttacattgaccccgtgcttcagtggggggcgtggggatcctctcctggtgcttaccgcactaggtggggtccacgcgacccccgacacaagtcgcgcaacttgggtcgggtcatcgacactctcacttgtggtgttgccgatctcgctgggtatgtcccagtgctcggcgctcccgcaggtgctctgtgccggggcgcggctcatcttgtgcggtttgttgaggacggagccaactttatcactggcaatattccaggcatgggtttctctatctttctcttagctctcttttcagctgtatctcttggtgaagcatctgtggtgcggaacggaggtcacgttgtcagtaatgattgtaattcttcgcagattctttgggctgcttccgactgggctatccacgaagtgggatgcgtcccttgcgtggagagttcctgttgggtgccgctcacttccagcatttctgttaggaatgaatctgtgattgttcatggtctcggctcgcacatagatgttcttgccgctatggcttctgtatgctctactttgggaattggcgaggcgtgtggtgctgcaactgtaacttatatcactttcttgtctcgtttctttatgaaccttgacttgaccaatgactgtgaatgctttctttaccccggcgccatttccacctttgagtttactatgagagctttgcaatctatgatgcccaatttgtccggtttcgtttctatgctcagcggagtaccaaatactctgtttaccatttttacgaacgggcattggggcgtcatccttgctctttgtctctatggcacaactaataattactttaaactatgtttgttacttttagcttactctggcttagtttcttgtgaaccagaaaagtcggagtatattaatgtttctttagcttgtaactttactgttaaggagctgtggggctggacgtttttcccaaagtgggcactgctgaacggtcggaggctcaattgtaccgagggctcgccatataacccgaagtgcaaagggcctatggacttcaacaccacaaccgatccggttattgcccacactgggatgcgttcacacccgccttgcccttatcatgtttcaagaccttgctcagtcattaatgcctccagggtttgtggcaaacccacctgtttcggcccagcgccgatcgaggtgggtgtcactgacagggatggtaatctagcttcttggaatgatactggccaatttttctttgatcttcggtctccccatcgcccacctcgtggacggtggtatgggtgcgtttggcttaattcaaccggttgggtcaagcaatgtggcgctccgccttgcaacatgcaacttgagtccaagcctgacaagacatttgtttgcccgtctgattgttttaggcagaaccccaaagccacttatcaactttgtggccaaggaccgtggattacccatagttgcctgattgattacactgatagatatctccatttcccatgcactgaaaatttcacagtctaccctgtccgcatgattcttgggaacggtgctcgggatgttcgtgtggcatgtaagtacaatagatctgtcagctgtaggactgaagataggttgagagcgagtatcgtctccctactctacagtgtgactactgcggctgttccaccttgtcacttctcgccactccctgccttcactactggcctcattcacttggatcgaaatctgagtgatgtccagtatgtttgggcaacgactcccagcgctgtcaatattttcctgaggcttgagtgggctgtcttctttttgcttttgcttatggacgctaaagtctgcgcaatcctttggttctgtttgtgtttggctttgcaagctgaggcttatctttccgatactatgaggctcatagctttgtcttacattgctgatgattctctgctttgggctttagtcttttactgtgtaatttactttaccccaagyagaattcctcctttttgtgtctttgtatattattggaagtttgctttagcttttatggttttggctttgccacatcgtgcttgggcctttgataataccagtgctgttactgctgcttttagtatagctttattttgcttgtatattacctgtttgtcttgctataagaggcttttcttactagtaaggtggtggttggaatattgggatgttagggttgaatgcgcttggcgctatctcggccctagggttaatccgaaagatgaaaaacttgcttttgctttgctattcgctttcttttacccttccctctttcgttgtgtgtatctacctttagcagtggtatgtgggtccttttccatgatcaacaaacgtgtccagaagataacttatcttcgacgcgctgaggtcttggttagggttttgactttatgcagggacctttatggtagcaaatgggtgcagtggtgtgtgctttgggtagcttcttattttggtactttcctttataatcatcttacccctattgacacttgggccgcccccgggttgagggacctcatgcactctttggagcccattactctctctcctatggagaggaaaatagttaagtggggtgcgaggaaagtcgcgtgtggcgacatcctccagggcctgcctgtttcggctcggctcggtagagaaatctgcctgggccctgctgacagacttagcagcaagggttggagactcttatcaccgatcaccgcaaccgtcacgaagacgcgcggcatcccttccgctattgtctgttgtcttacaggtagggacaaatatcctcacagaggccattgttacatcctgacctctttaaccaaaacctttatgggcactgtttgcaaaggcgtgctttggtccgttcaccatggtggcggtactgctactcttgcttctgacaaatcatctctgcttcaagtgttgtgttctcccggtgacgatttggtggcctggcctgctccggctggttctaagtcatttcaaccttgtacttgcggctcggctgacgtgttcttagtcaccaggacgggtcaggtcgttcctgccagaaagacttccgaaaaggacgcttcacttatctctcctttacccatttcatcacttaaaggtagctcaggtggcccggtcctttgtaaggatggggatcttgtaggtattttctgttctgcttctgtcactcgtggggttgctaagcgcatacatttcgcagacatgcgaactcgtagcgtttcgtcttgccccccgaaatacacagacttagatactcctcctgctgttccttcttcttaccaggtttcctttcttcacgctcccactggcagcggcaagtccaccaaaatgccgttgtcatatgtggagttaggttatcatgttctagttctcaatccatctgtagcatcaactcttagtttcgggccttacatggataagacttacggtgagtgccctaacatcaggacgggtgctagttgcaagactacgggctctaagctaacttattccacctatggaaagttcctagcagatgggggtgtctctgcgggcgcttatgacattattatttgtgatgagtgccatagcacagactcgacttctgttttagggattgggtccgtccttgatggtgcggaatccaaaggtgttaagcttgttgtccttgctactgccactccgcctggctctcaaaccgttcctcaccctaacatcgatgaggaggcccttacacagagtggggacatccccttttatgggaagatgytgaagtcgtccacgctccttagtgggaggcaccttatcttctgccactcaaagaagaaatgcgaggaagtcgcgttrcttctgcggaaggcgggagctaatgctgtcacctactaccggggtttggatgtctcagtcatccctaatgaggggaatgtcgttgtggtcgccacggatgccttgatgacagggtaytccggcaacttcgacaccgtaactgactgcaacactgctgtagagctagacattgagttttctttagaccctacttttactatggtcaccactcctaaaccatctgatgccgtttgtcgtacgcagcgcagggggaggactggtaggggccggaggggtacttactactatgttaatagtggtgagcgcccttctggagtcctgtcgtcctccgttctgtgtgaatgctacgatagcggcttggcttggtttggcttgagcccagctcaggtcactgttttactgcaagcctatcttaagcaacctggacttcccactggacttgaccacactgaattttgggagagcgtcttcatagggttacctactgtcgatgctttcttcctgtctcaattaaaacaacaaggagttacattcccatatctgactgctatccaggcaactgtttgcttaaatgcccaggccaaagctcccagcaaagacgaacgctggaaggtgctgtcccggtatattacaactaaccggactcctacacctttgctgtataggcttgaggacacccatgacgatcttactttcacgcacccagtaacaaaatacatccaagcctgcatggaggctgagattgacacccaaactaatgcttgggttattgcgggtggttgtgtcgcagctctggtcgctgtcgctgccttgaccggcagcgtcgctattatagctgaggtacatgtgaatgagaaagtcgtggttgttcctcataaaggtgtcctatatgccgattttgacgagttggaggaatgttttgaccatcatcaatacatccaacaagggtacgaatgggcggcgcgcgctgcgcagaaaatccgcgaggtcgcgtcttccatcgatcctcctactggtcaagcacaaccaatgctatcggctatagaaaagttttggaatcagcacatgtggaacattttgtcaggtgttcaatacttggcagggcttacaacactcccctacaatccatctgtagcttgccttatgggatttgtttctggtctcaccacaggtttgcctagaccagctatggctttcctcaccattctaggcgggtgggctgcgagcatggtagctcctcctcaggcagcgtccacatttgtcggtgctggactggctggcatcgctattggtgctgtcggtttcactgatgtaatcgtgggcctgctagcggggtatggggctggcgttgctggagcccttaccgcctttaagatcttgagtggtgtttctccaagtggtgaagaccttgttaatcttttaccagctttgctcaaccctggtgctcttgctgttggtgtaggcgctgcctttatcctgaaaaggtatactggcggcagtgaagggcttgttgcgtgggtcaacagactcattgcattttgctctcgggggaaccatgtctccccagatcactatgtccagcagcagcaggtcgtcagggatgtcattgcctgccttgaatccttaactcttaccagattagtcaaaactatccataattttgtcacaagtgaaaatgaccaaaactgtgattttaccaccatttatttctttatacaatggctaatgaaagctcttcatgattgtttcacttgggcaaagggaatcatccttccccacctcccaggactcccaatcatatcgtgtgattatggctactctggccgatgggccggagatggtattatcacgacgcggtgtggttgtggaaatatgatctccgggactgtgagaaatgaaagaattaggatcacaggctcgcggaagtgccgtaacgtctggctcaacaccttcccaattaactcgtcaactactggtggaccacggccaaatccttacgacacctggaaaacggccgtcttaagaattacttccacagagtacgttgagtttaagagggagggcactgctgtcagagtcatcggcgctaccgcagacaaattacgtatcccctgtcaagtacctgagcctgatttcatgacattcatagacggggtcagaattcatcggctggctccgtctccaaaacctatgttgcgcgacgaggtggtggtcctaatcggaaaccatacctacccggtcggcgcaacccttccttgcactccagaaccagatgtagatactgtgtcttctttattgactgacccaggacatgtcactgcagaaaccgctgcccgacggctcaggagagggcgtaccgttgacatcgagtcctcgagcggctctgagctgtcagccgtttcgcggggcgctgcctctcgtgtgtcagaagaacacgagatggccggcggcccagtgagaccgttgactggcgaggacgagctggcgtggattaggtcgttttacggacgttctgttacgattgaagtcgatgacaaagttatcaacttcgattcatggaccatcaattctgggtcggaaggagagcactctcgagaatcagtcgacgctccagacgatgatcgggtggtggtggctgaaccacctccacctcccggtccggcttggatgcggaaagactatgtcccagctctcgtttccgggtgtcccatcaagcccggtagtgctactcctgaacccagtgagccttcggccacggaatctgcccctgtggaagagacggaggagcccatggttgacgagaagggcgaggagacagaccctgacatgccccctctcgagggtgaagaacctgaggaaggggatgacagtcagtgggaaactacgtctgaaaaggcggagtcgtgctctttgtcctactcgtggactggcgcactcgtcacggcaacacgccgtgaggagaggcgccaccctataggccctctgtccaacactcttatcaccaggcataatctcgtctatcagaccacaacagcgtcagctagtgcgagaatggccaaagtgacaattgacagagaacaaattttagacaaatactactttgatactgtcacccaagtcaagaaccgggcgaaggaggtgacggctgacctgctatcctgggatgaagtggcccgcttaacaccaaagaacacagctcgagctcgatccggtttatccggatccgatgtgcgcaagctgactcgatcagctaggcgggaactaaattccatgtggcaggatcttctatcatcctctgaggaacccatccctacaacagtcatggctaagaacgaggtgttcgtgtcgtcgccaactgctcggaagccagccaggctgatcgtttaccccgatttgcctgtgcgggcttgcgagaagcgggcgatgtacgacctcttccaaaaattaccctatgctgttatggggaaggcctatgggtttcaatacactccccgccaacgtgtttccaggttactggacatgtggcggacttttaaaaaccctatgggcttttcctatgataccaagtgttttgactccacggtcactccccatgatatagaaactgagagagacatttttcttagtgccaacctccctgatgaagctaggactgttatcaagaacttaacatctaggctttatagagggtcccccatgtacaactcacgtggggatctggttggaaagagagaatgccgtgcttctggggttttccctacaagcatgggcaacactctgactaattacatcaaagcaacggccgctgctaaggcagctggccttgctgaccctcagttccttatctgcggagatgatcttgtttgtgtcacatccagtaaaggcgtcgaggaagacgagcaagcattacgtgatttcactaatgctatgacgaagtactccgccatacctggagaccttcccaaaccatattatgacttggagcagataacatcttgttcctctaatgtgactgttgcccaagacaggaatgggcgaccttattatttccttacccgcgacccgacaacaccacttgctcgggcgagttgggaaacaattagtcatagtcctgtaaacagctggttagggaacataattgcctttgctcctactttgtgggttaggctcgttttcctcactcatttctttggcctactgcttcaacaagatgcggtggatcaaaattatgagttcgagatgtacgggtctacctactctgtaaatcctcttgatttgccggcaataatttataagcttcatggccctgaggcctttgatcttaccaattattctccttacgaggtccagagagtggctgctgcactccagaagttggggtcaccacctctccgagcttggaagcgtagagctaagctcgttcgctcaaagcttaaagtacgtggtggtcgctactctgttgtggcggactatctctttggcttcgcttctgcttatcgaccgaagaggcctgcgcctcctggcgtaaattcgattgatgtttccggttggttctccattggagatgactccattggggatatctaccgacaattaccaatcgttgctgggaggtggattcctcttcttcttttgcttcctttattggctgcaattctttacttcaacaaataagaaagaaaagaaaaataatt

SEQ ID NO: 7 - DNA, 9221 bp, Unknown
  Description of Unknown Non-primate hepacivirus F8-068-GBX2 polynucleotide isolated from horses
cctccgtgcttggcacggtgcgttgtcagcgtgttgcgcttgcatgcgctctacgcgtcgtccaacgcggagggaatttcacattaccatgtgtcactccccctatggagggatccacccgtctacacggaaacgggttaaccatactctgagtacgggtattcgggtcctcctagggcccccccggcaggtcgagggagctggaattcgtgaatccgtgagtacacggaaatcgcggcttgaacgtcttaggaccttcggagccgaaatttgggcgtgccccacgaaggaaggcgggggcggtgttgggccgccgcccccctagtcccacggtctgataggatgcttgcgagggcacctgccggtctcgtagaccataggacatgggcaacaactctaaaaatcaaaaaccaaaaccccaaagaggaccacggaatagggttaggggtcagtcgcggtctggccctgtagtattcccgtccggcgctgttctcgttggaggaaggtacattcctccgccaaagaaggccatccgcgggcctcgtaaaggtttggtgcaggctcccaagtcatcggagcgtggctccccccggaaaaagcgccaacctccgccacaaactgacagctcgtggcgaaagtatttccctaaattctggggggacaagggatatccgtggccatatgttgatcccgtcctccagtggagcgcatggggtacctcacctggtgcataccgcaccaggtggggcccccgtgaccctcgacacaagtcgcgcaaccttgggcgcgtcattgatactctcacgtgtggggtcgccgaccttgccggatatgttccggtactcggcgctcccgctggtgcattgtgtcgtggcgctgctcatcttgtgcggtttgttgaggacggggccaatttcatcactggcaacatccctggcatgggtttctctatctttatcttggctcttctttcagttctgtcattcggcgaagcgtctgttgtgcgtgctggaggccatattgtcagtaacgactgcaactcttctcagattttgtgggctgcttccgactgggctatccacgaagtgggctgcgtgccctgcgtggagagcacttgttgggtgcctctcacttcaagcatttctgtcaagaacgagtctgttattgtacgtggtcttggttcgcatatagacgtccttgctgctatggcgtctgtctgttctactcttggtattggtgaggcttgtggtgctgctactctaggttacattacttttctatccagatttttcatgtccttaaatcttacaaatgattgtgagtgcttcctctaccctggcgccatctctacctttgagtttactatgagagctctacagtctatgatgcctaacctgtccggttttgtgtctatgttcagtggggttcctaataccctatttaccatctttacaaacgggcattggggcgtcattcttgctctatgtctttatggcacaacaaataattactttaagctttgcctgttgctcctagcatattctggcttagtttcttgtgaatctgactatctcaatgtttctttgtcctgtaatttcactgttaaacagatgtggggctggacatttttcccgaagtgggctatattgaatgatcagaggttgaattgcaccgagggttcgccttacaacccgaagtgtaagggaccaatggacttcaatatcactgctgacccggtcattggctactccgggacacgttcgcacccaccctgtccttaccatgtgtctaggccttgctctatcttagatgcgtccagagtctgtggcaaaccgacctgtttcggccctgcgccgattgaggttggcgtcactgaccaggatggaaacctagtttcttggaatgattctggcaaattcttttttgatcttcggtccccccaccgaccccctcggggccgttggtacgggtgtgtttggcttaattctaccgggtgggtaaagcaatgtggcgctcctccgtgtaacatggcactcatgtctggtaagggcaaaacatttgtttgcccgtccgattgttttaggcaaaaccctaaggccacttatcaactatgtggccaaggaccgtggatcagttataattgtttaattgattacactgataggtatctccatttcccctgtacggaaaatttcaccgtttacccggtccggatggttcttggtgatggcgctcgggatgtgagggtggcgtgtaaatacaataggtcagaaagatgcaggactgaagataggttgagagcgagtatcgtctccctactctacagtgtgactactgccgctgtaccgccttgccacttctcccctttacccgccttcactactggccttattcacttggaccgaaatctgagtgatgtccagtatgtttgggcgatgactcccagtgctgtcaacgttttcctgaggctcgagtgggcggtgtttttcctacttctgctcatggatgctaaagtttgcgctattctctggttttgtttgtgtttagccttgcaggctgaggcttatctttcacatactatgaggcttattgctttatcatacatagctgatgattctttgctttatgctttacttttctattgtgtgatttactataccgaaagtagagtgcctcctttttgtgtctttatgtattactggaagttttccttggcttttctggtgttggctttaccacaccgagcttgggcttttgataacgctagtgctgttactgctgcttttagtatagctcttttttgcttgtatgtaacttgtttatcttgctataaaaagctctttatgcttgttaaatggtggcttgaatattgggatgttagggtcgagtgcgcttggcgacatctcggccccggggtttctccacatagtgaaagatttgcttttatattggtctttgcttttctttacccccccttgtttcgtgctgtttatttgcctctggctgtggtgtgtggttccttttccatgatcaacaagcgcgtacagaaaatacagtaccttaggcgcgcggaggtcttggtgagggtcctcactatctgcagagacgtttatggtagcaagtgggtgcagtggtgcgttctgtgggttgcttctcattttggtactttcctttatgatcatttaactcctattgacacctgggctgccccagggttgagggacctcatgcattccttagagcctataactctctctcctatggagaggatggtagtcaagtggggtgcaagaaaaatcgcttgtggcgatatcttgcatggcctgcctgtatcggcgaggctgggcagagaaatctgcctaggccccgccgaccgacttacgagcaagggctggaggttgttatcacctataactgccaccgtaaccaagacacgtggcatcccttcggcaattgtttgttgtctgaccggcagagacaaatatccccatagaggacactgttacatcttaacgtccttgactaaaaccttcatgggcactgtttgtaaaggtgtgctgtggtccgtccaccacggcggcggcactgccactcttgcttctgataaatcttctctgttgcaagtcctgtgctctccgggcgatgatcttgtcgcatggcctgctccagccgggtccaaatctttccagccttgcacgtgtggatcggctgatgtgtacttggttacccggactgggcaggttgtgccggcacggaaaacgtccgaaaaagatgcttctctcatttctcctttgccaatttcatcgcttaaaggtagctcgggaggtccagtcctgtgtaaggacggggatctcgtgggtattttctgttcagcctccgttaccaggggagttgcaaaacggatacacttcgcggacatgcggacgcgcagtgtttcctcttgccccccgaagtacacggatctggactccccgcctgcagtcccttcttcataccaggtttcttttctccatgcccctactggcagtggcaaatccaccaagatgccattgtcttatgtagaattgggttatcatgttctagttctcaatccgtcggtcgcctccactcttagctttgggccatacatggataagacctatggtgagtgtccgaacatcaggactggtgctagttgcaaaactacaggctccaaactcacatattccacctatggaaagttcctagcggatggtggggtttctgcgggcgcttatgacataatcatttgtgatgagtgccatagcacagactccacgtccgttttgggaattggatctgtccttgacggggcagaatctaaaggtgtgaagcttgttgttcttgccactgccaccccaccggggtcccaaaccgttcctcatcctaacatcgatgaggaggctcttacccagaatggagacatccccttctacgggaagatgttgaagtcatccttgctcctcagtggaaggcatcttatcttctgccattcaaagaagaaatgcgaggaagtcgcacttcttctgagaaaggctggggctaatgctgtcacctactaccggggcttggaggtttccgtcattccgaatgaggggaatgttgttgtcgttgccacggatgcccttatgacggggtattctggtaacttcgacactgtgactgactgcaacaccgctgtagagctggacattgagttttctcttgacccaactttttccatggttaccactccaaaaccatctgatgccgtctgtcggacacagcgcagggggaggactggcagaggccgcaggggtacttactactatgtaaatagtggtgagcgcccctccggagtactgtcgtcatccgtcatgtgtgagtgttacgatagtggcttggcatggtttggccttagtcctgcgcaggttactgtgctgctacaggcctatttaaagcaacctggactccccaccggattggaccatactgagttttgggagagtgtcttcataggcttgcctacagttgatgctttctttttatctcagctaaaacaacaaggagttacattcccttatcttactgcaatacaagctactgtttgtcttaatgctcaagctaaagctcccagcaaggatgaacgttggaaggtgctgtctcggtacatcacaactaaccgtactccaacgcccctgttgtacaggcttgaggacacacacgatgacttgacgtttacgcaccctgtgaccaagtatattcaggcctgtatggaggccgagattgatacacaaacaaatgcttgggtcatagcgggtggttgcgtcgcggctctggtcgcygtcgcagccttaactggcagcgtcgcgatcattgccgaggtgcatgtcaacgaaaaagtggtcgttgttcctcataagggcgttctctatgctgatttcgacgagttggaggaatgctttgatcaccatcaatacattcaacaaggttatgagtgggcgtcgcgcgcagcacaaaaaatccgtgaggtcgccacgtccattgaacctcctactggtcaaacacagcctatgctatcagcaatagagaaattttggaatcagcacatgtggaacattctatcaggagtacagtacttggcaggacttactacacttccttataatccatctgtagcttgcttaatgggctttgtctcaggtcttaccacaggattgccacggccagctatggctttcctcaccattctaggcggatgggctgccagcatggtggccccaccacaagcagcctccacattcgttggggccggactagccggtatagctatcggcgctgtcgggtttaccgatgttatcgtggggcttcttgctggatatggggctggcgttgccggtgcccttaccgcttttaagatcttgagtggtgtcactccaaatggtgaagatctgattaatttgcttccatctttgcttaaccctggcgctctcgctgtgggcgtgggagctgcctttatcctgaaaaggtatacaggcggcagtgaagggcttgttgcctgggtcaatcggctcatcgccttttgctcccgaggcaatcatgtctcccctgatcactacgtccaacagcagcaggtcgtcaaggatgtcattgcttgccttgagtccttaaccctaaccagattagtgaaaactatacataattttgttacaagtgaaaatgatcaaaattgtgattttactgccatctatttttttattcaatggctaatgaaagctctttatgattgtttcacctgggccaagggaattattcttccacacctcccaggttttccactcatatcgtgtgacacgggctactccggccgttgggccggggagggtttggtgactacgcggtgtggttgtgggaacatgatcactgggaacgtgaggaatgaaagaattaggataacgggctcgcggaagtgccggaacctctggctcaatgctttcccaatcaactcgacaactactggtgggcctcgacccaatccttatgacacctggaaaacagctgttctgaggatcacgtccactgagtatgttgagtttgaaaggaagggcactgcagtaagggttattggcgctaccgctgacaaattgagaattccctgccaggtccctgaacctgatcttatgacctacatagatggagttcggattcacagattggctccgactccaaaacctatgttgcgtgatgaggtggtggtcttaattggcaatcacacctaccccgtgggcgcaactcttccatgcactccagaaccagacgtagacactgtatcttcccttttaactgatccaggacatgtcacagcagagactgcagccagacggctcagacgcggacgtaccgtggacgtcgagtcctcgagcggctccgagctgtcggccgtttcgcggggtgccgtgtctcgtgtgtcagaagaacatgagatgcaaggtggccccgtgagaccgctgactggcgaagacgagctggcgtggattcgttcgttttacggacgttccgtgacaattgaagttgatgacaaggttattaactttgattcgtggaccatcaattctgggtccgagggagggcactcgcgagagtcagtcgtcgccccagacaacgaccaggtcgtggtggcagaacccccgccacctccaggtccggcttggatgcgaaaagactatgtcccagctcttgtatccgggtgtcccatcaagcccggaagtgctacccctgagcccagtgagccttctgctacagaatctgcccgcgtggaggagaaagaggaacctaaggttgacgaggacggtgccgagacagaccccgaaatgcctcctctcgaaggtgaagagcctgaaggggatgatgacggggaatgggagacagacccctctgggtctgaggcgccggcagtcgacgccaactgctcgctatcgtactcgtggacgggggctctcgtcacggctacccgccgtgaggagaggcgccatcccataggacctctctccaacactctcatcaccaaacacaatctcgtctaccagacaacaacagcttctgctagtgcgagaatggctaaagtgacaattgacagagaacaaatttttgacaaacattactttgacactgtcaccgcagtcaagaagagggcttcggaggtggcggcggacctgcttacgtgggatgaagttgctcgcttgacgcccaagaacacagctaaagcaaagtcggggcttagtggctccgacgtgcggaagctcacaagggcggccaggcgagagttgaattccatgtggcaggaccttctgtcagactctgaagagcccattccaacaacagttatggcaaaaaatgaggtctttgtgtcatctccgactgctcgcaagccagctaggctgatcgtctatcccgacctgcctgtgcgtgcctgtgagaaaagggccatgtatgacctcttccaaaaacttccctatgccattatggggaaggcctatggatttcaatacactcctcgccaacgtgttgagagattacttgacatgtggcggcattttaagaatcctatgggcttttcctatgatacaaaatgctttgactccactgtcactcctcatgatatagatactgagagggacattttccttagtgccaacttacctgatgaagccaaaattgtaatcaaaaatctaacatcaaggctttacaggggatctcccatgtataattcgcgtggggatcttgttggaaagagggaatgtcgtgcttccggggttttcccgacaagcatgggcaatactctgaccaactacattaaggccgccgcggccgccaaagccgctggcttaagtgaccctcagtttctaatttgtggggatgaccttgtctgcatcacctccagcaaaggcgttgaggaagatgagcaagcgctgcgagacttcaccagcgcaatgacaaagtactccgccattcctggggaccttcccaaaccatactatgacctagagcagataacatcttgctcttcaaatgtgactgttgcccaggatagaaatgggcgaccttattatttcctgactcgtgatcccacaacaccacttgcgagagcgagttgggaaacgattagtcacagtcctgtaaatagctggctagggaacataattgcttttgctcccactgtctgggttaggctcgttttcctcactcatttttttggtctattactccaacaggatgccgtggaccggaattatgagtttgagatgtacgggtcaacctattcagtgaatccgcttgatttacctgcaataatttataaacttcatggccctgaggcttttgatcttacaaattattctccttacgaggttcagagggtggctgcagcactccagaagttggggtctcccccactgcgggcttggaaacgtcgagctaagctcgtccgctcgaagctcaaagtgcggggaggccgctacgctgttgttgctgactacctctttggcttcgcttctgcctatcgcccgaagaggcccgctcctcctggcgtaaattcgatcgatgtttccggttggttctccattggggatgactccattggagatatttatcggcaacttccggtcgttgctgggaagtggatccctcttctgcttttgctacctcttttggctgcaattctttat

SEQ ID NO: 8 - DNA, 9223 bp, Unknown
  Description of Unknown Non-primate hepacivirus G5-077-GBX2 polynucleotide isolated from horses
ggcacggtgcgttgtcagcgttttgcgcttgcatgcgctacacgcgtcgtccaacgcggagggaatttcacattaccatgtgtcactccccctatggagggttccaccccgcttacacggaaatgggttaaccatacccagagtacgggtatgcgggtcctcctagggcccccccggcaggtcgagggagctggaattcgtgaattcgtgagtacacgaaaatcgcggcttggacgtcattgacctccggagccgaaatttgggcgtgccccacgaaggaaggcgggggcggtgttgggccgccgccccctttatcccacggtctgataggatgcttgcgagggcacctgccggtctcgtagaccataggacatgagtaacaaatctaaaaaccaaaaacccaaaccacaacgaggaccacggaatagggtcaggggccagtcgcggtctggtcctgtagtattcccttccggcgcagtccttgtgggggggaggtatatcccccccccaaagaaggccatccgcgggcctcggagaggtctggtgcaagctccaaaatcatcggagcgtacttccccccggaaaaaacgccagcctccgccacaaactgacagctcgtggcgaaagtactttccaaagttctggggggacaagggatatccatggccttatgttgatcccgtcctacaatggggggcctgggggtcctctcccggtgcttatcgcactagatggggaccacgcgacccccgccacaaatcgcgcaatctgggacgtgtcatcgatactctcacctgtggggttgccgatctcgctgggtatgtcccagttgtcggcgcacccgcaggtgcactttgtcgtggcgcggctcatcttgtacggtttgttgaggacggtgctaatttcataactggcaacattcctggcatgggtttctctatctttttgcttgctctcttttcagctgtgtctttcggagaggcctctgttgtgcggaacggtggacacgttgtcagtaacgattgcaactcctcgcagattttgtgggcgtcctccgactgggctatccatgaggttggatgtgttccatgcgtggacagtgcttgttgggtgccgcttacatctagtatatctgttaggaatgaatctgttattgttcgtggactcggttcgcacatagatgtcttatctgccatggcttctgtttgctctactttaggtattggcgaggcatgtggtactgcaactctcacttacattactttcttgtctaaattctttatgtctttaaacctgactaatgattgtgaatgttttttgtatcctggtgctatctctacctttgagtttactttgagagctttgcaatctatgatgcccaatctgtccggttttgtttcgatgtttagtggacttcccaacactctatttaccatcttcactaacgggcactggggcgttatacttgctctttgtctctatggcacaaccaataattactttaaactttgcttattgcttttagcttattcggcattagtttcttgtgatgattctacctaccttaatgtttccttggcatgtaattttactgttaagcagatgtggggctggacgtttttcccaaagtgggcgttacttaacggacagaggctaaactgcactgaggggtctccttataaccctaagtgcaaagagccttttgattttaacatcacaactgatccggttatcgcctatagtgggactcgttcacatccgccctgcccctatcatgtgtctagaccttgctctgttcttaatgcatccagagtgtgtggcaaacctacctgtttcggacctgctccgattgaggtgggagtcactgatcgggatggtaatcttgcttcctggaatgattctggccaattttattttgatctacggtcgcctcaccgcccacctcgtggacggtggtatgggtgtgtttggcttaattcgaccggttgggtcaagcagtgtggcgctcctccttgtaacatgaaacttatgtccaacaggagcaagacatttgtttgccctaccgactgttttaggcagaaccccaaagccacttatcaactttgtggccaagggccgtggatcactcattcttgcttgattgactatactgataggtaccttcacttcccgtgcactgaaaatttcacagtttaccctgtccgcatgattcttggtgacggcgctagggatgtcagggtggcatgcaaatttaacagatcgcgcagctgtaggactgaagacaggttgagagcaagtatcgtctccctgctttacagtgtgactaccgcagctgttccgccatgtcatttttcgccgttacctgcattcaccactgggctaatccacttggatcgtaatctgagtgacgtccagtatgtgtgggctatgactcccagcgctgtcaacatcttcttgaggctggagtgggctgtgtttttcctgcttcttcttatggatgctaaggtatgcgcaattctttggttctgcttgtgcttagctttgcaagctgaggcttatctttcagacactatgcgattgattgcactttcttatgttgctgatgactccttgctttgggctttggtcttttactgtgtaatatactttaccccaagcagggttcctcccttttgtgtatttgtgtattactggaagtttgctttagctttcatggttttggctttaccacatcgtgcttgggcttttgataatgccagtgctgtcacagcagcttttagcatagctcttttctgcttgtatattacttgcttgtcttgctataagaaactgtttttgcttgttaagtggtggctggaatattgggatgttagaattgaatgtgcttggcgctatcttggccctagggtaaaccctagggacgagaagcttgcttttgctttactcttttctttcttttacccttctttgtttcgctgtgtatatttgcctttagctgttatatgtgggtccttttccatgatcaacaaacgcgttcaaaagatctcgtatcttcgacgcgccgaggtcctggttcgggtcctttctatctgcagggatgtttatggtagcaaatgggttcagtggtgtatcttatggctagcttctcattttggtactttcctttataaccaccttaccccaatagacacttgggctgcacctggcctgagagaccttatgcactctttggagcctataactctctcacctatggagaggaaagttgtcaaatggggcgcgaggaaggttgcttgtggcgacattcttcgtggcctgcccgtgtccgcgaggcttggtaaagaaatttgccttggtcccgctgataaacttacgagcaaaggctggaggcttttgtcaccaattaccgcgaccgtcactaagacacgcggcattccttcagccattgtttgttgccttacgggtagggacaagtatccccatagaggtcattgttatatcttaacctctctgaccaaaactttcatgggcaccgtctgtaaaggtgtgctgtggtcggtccatcatggtggtggtactgctactcttgcttctgataaatcatctttgcttcaagtgttgtgttcccccggtgacgatctggtggcatggcctgcccctgctgggtccaaatctttccagccgtgcacttgcggttcggctgatgtgtttttggttacccggacggggcaggtcgttccagccagaaagacctccgagaaggacgcttcacttatttctcctttgcctatttcatcgctgaaaggcagttcaggtggcccggtcctgtgtaaggatggggatcttgtaggtatcttttgttctgcttctgtcactaggggggtggctaagcgcatacacttcgcagacatgcgaacgcggagcgtctctagttgtcctcccaagtatactgatttggattctcctcctgctgttccttcatcttaccaggtttctttccttcatgctcccactggtagcggcaaatccaccaagatgccgctatcctatgtggagcttgggtaccatgtcttagttctcaacccttcagttgcttcaactctcagttttgggccctacatggacaagacctatggggaatgtcctaacatcaggacaggcgcaagctgtaagactacaggctccaaacttacttattccacctacggtaagttcttagctgacgggggtgtttctgccggcgcttatgacattataatatgtgatgagtgccatagcactgattctacctccgtgctaggaataggctctgtccttgatggagcagagtctaagggtgtaaagcttgttgtacttgctaccgctacgccgcccgggtctcagactgttcctcaccctaacattgatgaggaggcccttactcagagcggggacatacccttttacgggaagatgttgaaatcgtccttactccttagtgggagacatcttatcttctgccattcgaagaagaagtgtgaagagatcgcgcttcttctgaggaaggcgggagctaacgctgtgacctactaccggggtttggatgtttccgtcataccgaatgagggtaatgtcgtcgtcgttgccacggatgccctgatgacagggtattctggaaacttcgacagcgttactgattgtaacaccgctgtggaactggacattgagttttcccttgatccaactttttctatggttaccactcctaaaccatcagatgctgtttgtagaacacagcgcagggggaggactggtaggggccgcaggggcacttactactatgttaatagtggtgagcgtccttctggggtcctatcgtcttccgtcctgtgcgagtgctatgacagcggcttagcttggttcggcctgagcccagcgcaggtgactgtgctgcttcaggcttaccttaagcaacctggccttcccaccggacttgaccatactgaattttgggagagcgtcttcattggcttgcctacagtcgacgctttctttttgtctcagcttaaacaacaaggtgtaactttcccctatctgactgccatccaagcaactgtatgccttaatgctcaagctaaggcacccagcaaagacgaacgctggaaggtgctgtcacggtatattaccacaaaccgtactccaacgcccttgttatacaggcttgaggatacccacgacgatcttacttttactcacccaataaccaaatacattcaggcctgcatggaggctgaaattgatacccagaccaacgcctgggttattgcgggtgggtgtgttgcagctctggttgctgtcgctgcccttacgggtagtgttgcgatcatagctgaagtacacgtcaatgagaaggtcgtggtggtccctcacaagggcgttctctacgctgattttgacgagttggaagaatgttttgaccatcatcaatacatccagcagggatacgagtgggcgtcgcgtgctgcgcagaaaatccgcgaggttgcggcgtccatcgaccctccagctggccaaacacaaccgttgctatcggctgtagagaaattttggaatcagcacatgtggaacatcttatcaggtgtgcagtacttggcaggtcttacaacactcccttataatccatctgtggcttgtctaatgggttttgtctcaggtctcaccacgggattgccaagaccagctatggcattcctcaccattctaggtggttgggcggcaagcatggtggcccccccccaggctgcgtccacttttgtcggcgctgggctggctggcatcgctatcggtgcagtcggtttcactgacgtcattgtgggcttgcttgcggggtacggggccggcgtggctggcgcccttactgccttcaaaattttgagtggtgtcaccccaagtggtgaagacctgattaatttattgccatctttgctcaaccctggcgcacttgctgtaggtgtgggcgccgcctttatcctgaagaggtatacaggcggtagtgaaggacttgtcgcatgggtcaaccggctcatagcgttctgttctagggggaaccacgtctcccccgatcactatgtccagcaacagcaggtggttagggatgtcattgcttgccttgaatctttaaccctaactagattagtgaaaactattcataactttgttacaagtgaaaatgatcagaattgtgactttactgccatctatttcttcatacaatggttaatgaaaacactttatgactgtttcacctgggcaaagggaatcattcttcctcaccttccaggtttcccgatcatatcgtgtgattcgggctactctggccgatgggctggagagggccttgtttccactcggtgtggttgtggtaacatgatcactggaaatgtaaggaatgaaagaattaggatcacaggctcgcggaagtgccgcaacgtctggctcaataccttcccaattaactcttctactactggtgggcctcggcctaatccatacgatgtgtggaaaacagccgttttgagagtaacttccacagagtatgttgagttcaagagggaggggactgctgtcagagttattggcgcgaccgctgacaaattacgtatcccttgccaggttcctgaacctgatttgatgacattcatagacggagtccgcatacataggctggctccagatccaaaaccaatgttgcgcgatgaggtagtggtcttaattggcaatcacacttacccggtcggcgcgacacttccatgcactccagaaccagatgttgacactgtatcttccctgctaactgatccaggacatgtcactgcagaaactgcggctagaaggctcagacgcggtcgcaccgttgatgttgagtcctcgagcggctccgagctatcggcggtttcgcgaggcgcagcttctcgtgtgtcagaagaacatgagatggctggtggccctgtgagaccgctgaccggcgaagacgagctcgcttggattcgatcattttacggccgatctgtgacgattgaggttgatgacaaggttatcaactttgattcatggaccatcaattctgggtctgagggggagcactctcgagaatcagtcgttgcgccagacaacgaccaggtggtggtggctgcaccgcctccgccgcccggtccagcctggatgcgcaaagactatgtcccagctctcgtttccgggtgtcccatcaagcccggaagtgctacgcctgagcccagtgagccctcagctacagaatctgccccagttgaggaaaaggaggaacctgtggtcgacgataaaggcgaggaaacagaccctgatatgcctccgctcgaaggtgaagaacctgaggatgcggatgacagtcagtgggagaccacgtctgaaaaggcggagtcatgttccctgtcttattcgtggaccggtgccctcgtcacggcaacccgccgcgaggagcgacgtcatcctataggtcccctttccaatacactcattaccaaacacaacctcgtgtatcagacaacaacagcgtctgctagcgcgaggatggccaaagtgacaattgatagagaacaagtcttagacaaattctactttgacactgtcacccaagtcaaaaagaaggcttcagaggtggaagcaggcttgctctcgtgggatgaggttgctcgccttactccaaaaaacacggctcgtgcgaaatcaggactttccggttctgatgttcgtcaacttactcgagctgcgaggcgggaacttcagtctacatggcaggatctcctttcatcctctgatgagcctatccccactacaattatggcaaagaatgaggttttcgtttcgtcgccaacctctcgcaagccagccaggttgatcgtctatcctgatttgcctgtgcgggcatgtgagaagagggcgatgtatgacctcttccaaaaattaccctatgctgtcatggagaaagcctatggatttcaatacactccccgccaacgtgttgatagactcttagacatgtggcgacattttaagaatcctatgggcttctcctacgacaccaaatgctttgactccaccgtcactcctcacgatatagacactgagagggatatctttctaagtgccaacctccctgatgaggccaagacagtcattaagaacttgacgtccaggctttacagagggtccccaatgtacaattcacgtggggatcttgttggcaaaagagagtgccgtgcttctggggttttccccacaagcatgggcaacacccttacaaactttatcaaagcttccgcagctgccaaggctgctggatttgctgaccctcagttcttaatctgtggggatgaccttgtttgtgtcacctccagcaaaggcgttgaggaagatgaacaggcgttacgtgagttcacaaacgccatgacaaagtactccgccatccctggagatttcccaaagccatattatgacttggagcaaataacatcttgttcctccaatgtgactgttgctcaagacagaaatgggcgaccttattacttccttactcgagacccaacaacaccgctggctcgtgcgagttgggagacaattagtcacagtcctgttaacagttggttaggaaacataattgcttttgctcctacggtgtgggttagacttgttttcctcactcatttcttcggtctactgctccagcaagacgctgtggatcggaattatgagtttgagatgtacgggtcaacctactccgtaaatccacttgatttacctgcaataatttataagcttcatggccctgaggcctttgaccttacaaactattctccttacgaagtccaaagagtggctgcggcactccagaaactggggtcacctccccttcgagcttggaagcgcagggctaagcttgtgcgctctaagcttaaggtaaggggtggccgctattctgttgttgcggactatcttttcggcttcgcttctgcttacagaccaaagaggcctgcgcctcccggtgtcaattcgattgatgtgtctggatggttctccattggagatgactccattggggacatttacaggcaactaccgaatgtcgctgggaagtggatccctcttcttcttttgcttcctttactggctgcaattctttacttcaacaaaaaaaaaaaaaaaaaaaaaa

SEQ ID NO: 9 - DNA, 9185 bp, Unknown
  Description of Unknown Non-primate hepacivirus H10-094-GBX2 polynucleotide isolated from horses
ggcacggtgcgttgtcagcgttttgcgcttgcatgcgctacacgcgtcgtccaacgcggagggattttcacattaccatgtgtcactccccctatggagggttccaccccgcctacacggaaatgggttaaccatacccatagtacgggtgagcgggtcctcctagggcccccccggcaggtcgagggagctgaaattcgtgaatccgtgagtacacggaaatcgcggcttgaacgtcatacgtgaccttcggagccgaaatttgggcgtgccccacgaaggaaggcgggggcggtgttgggccgccgccccctttatcccacggtctgataggatgcttgcgagggcacctgccggtctcgtagaccataggacatgggcaacaaatctaaaaaccaaaaaccaaaaccccaaagaggaccacggaatagggtgaagggccagtcgcggtctggtcccgtagtattcccgtccggcgctgttctcgtgggggggagatacatcccccccccaaagaaggccatccgcgggcctcgtagaggtttggtgcaggctcctaagtcatcggagcgaactgccccccggcataggcgccaacccccgccacaaaccgacagctcgtggcgaaagtatttcccaaaattctggggggataagggatacccatggccttacgttgactccgttcttcagtggggggcgtgggggtcctctcctggtgcctatcgtaccaggtggggccctcgtgacccccgacacagatcgcgcaacttgggtcgggtcattgacactctcacttgtggggttgccgacctcgctgggtatgtcccagtgctaggcgctcccgcgggtgctttgtgccgtggcgcggctcatcttgtgcggtttgttgaggacggagccaactttatcactggcaacattccaggcatgggcttctctatctttctcttagctctcttctcagccgtatcttttggtgaagcatccgttgtgcggaacggaggccacgttgtcagcaacgattgtaatcagtcgcagattctttgggctgcgtctgactgggctatccacgaagtgggatgcgtcccctgcgtggacagtgcttgttgggtgccgctcacttctagcatctctgttagaaatgaatctgtgattgttcgtggtctcgggtcgcatatagatgttgtagccgctatggcttctgtttgttctactcttgggattggcgaggcttgtggtactgcaactttaacctacatcactttcttgtctcgtttctttataaggcttaacttgacttatgattgtgagtgctttctttaccccggcgccatttccacttttgagtttactatgagagctttacaatccatgatgcccaacttgtctgggttcgtttctatgttcagcggattaccaaatacgctgttcactatcttcacgaacgggcattggggcgtcattcttgctctttgtctttatggcacaactaataattactttaaattatgcctactactcttggcttattctggcttagtctcttgttctgaagattacatcaatgtctccttatcatgtaactttaccattaagcagatgtggggttggacgtttttcccgaagtgggcattgctgaatggccagaggcttaattgtaccccgggttcaccatacaacccgaagtgcaaaggacctatggacttcaacatcacgaccgacccggttgtaacctacagcgggacgcgttcacacccgccctgcccctatcatgtatctaggccttgctcagtccttaatgcctccagagtctgtggcaaacccacctgtttcggccctgcgccgattgaggtgggcgtcactgacagggatggtaatctggcttcttggaatgataccggtcaatttttctttgatcttcggtccccccaccgaccacctcgtggacggtggtacgggtgtgtttggcttaattctaccggttgggtcaagcagtgtggtgctccgccttgcgtcatgaacctcgtgtccaataagagcaggacatttatctgcccgtccgattgttttaggcaaaaccccaaggccacataccaactctgtggccagggaccgtggatcacacataattgtttgattgattatactgacagatacctccattttccatgcactgaaaatttcacagtctaccctgtccgcatgattctcggggacggcgctagggatgtccgtgtggcgtgtaattttaatagatctgtcagctgcaggactgaggataggttgagagcgagtatcgtctccctactctacagtgtgactaccgcagctgtcccaccttgtcacttttcgccactccctgccttcactactggtttaattcacttagatcgtaatctgagtgatgttcagtatgtttgggcaatgactcccagcgccgtcaacgttttcttgaggcttgagtgggctgtctttttcttacttttgctcatggatgctaaggtctgtgcaattctctggttctgtttatgcttggccctgcaagctgaagcttatctttctgacactatgagacttatagctctgtcttacattgctgatgattccctgctttgggctttggttttttactgtgtaatttactttaccccaagcagacttcctcctttttgtgtctttgtatattactggaaatttgctttagctttcatggttttggctttaccacatcgtgcttgggcttttgataatgctagtgccgttactgccgcctttagcatggctttattctgtttgtatgttacttgcttgtcttgctataaaaggctttttatgctagtaaagtggtggttggaatattgggatgttagggtcgaatgcgcttggcgctatctcggccctagggtgaatccgagagatgaaaaacttgcttttgcgctaatttttgctttccttcatcctccccttttccgctgtatatatttacctttagcagtggtgtgtggatccttttccatgatcaacaagcgtgtccagaagataagttatcttcgacgcgctgaggtcttggttaaggttttggctttctgcagggacctttatggtagcaagtgggtgcagtggtgtgttctgtgggtagcttcttattttggtactttcctttataatcatcttacccctattgacacttgggccgcccctgggttgagagaccttatgcattccttggagcccattactctctctcctatggagaggaaggtagttaaatggggcgcgaggaaagttgcttgtggcgacatcctccatggcttgcctgtttccgctcggctcggcagggaaatctgcctgggccccgccgacaaacttagtagtaagggatggagacttttatcaccaattaccgcaaccgtcacaaagacgcgcggcatcccctctgctatagtctgttgtctcacaggcagggacaagtatccccatagaggccactgttacatcttgacctctttaactaaaactttcatgggcaccgtctgcaaaggtgtgctttggtccgtccatcacggtggcggcactgctactcttgcttctgataaatcatctctgttgcaggtgttgtgttctcccggtgacgatctggtggcttggcctgccccggctggttctaaatcatttcagccttgcacttgcggctcggctgacgtgtttttggtcaccaggacgggtcaggtcgttcctgccaggaagacttccgagaaggacgcttcactcatttcccctttacccatttcatcacttaaaggtagttcaggtggcccggtcctttgtaaggatggggatcttgtaggtattttctgttctgcttctgtcacccgtggggttgccaagcgcatacatttcgcagacatgcggactcgtagcgtttcgtcttgccccccgaagtacacagacttagacactcctcctgctgttccctcttcttatcaggtttcttttcttcatgcacccacaggtagtggcaagtccaccaaaatgccgttatcatatgtggagttgggctatcatgtactagttctcaatccatctgtagcatcaacactcagtttcgggccttatatggacaagacttacggtgagtgccccaacatcaggacgggtgctagttgcaagacgacaggctcaaagttaacttattccacctatggaaaattcctggcagatggcggcgtctctgcaggtgcttatgacattatcatttgtgatgagtgccatagcacagattccacctctgtcttaggaataggttctgtccttgatggtgcagaatctaaaggtgttaagcttgttgttcttgccactgccactccgcctggctctcaaaccgttcctcatcctaacatcgatgaggaggcccttacacagagtggggacatccccttctatgggaagatgttgaagtcgtccatgcttcttagtgggaggcaccttatcttctgccattcaaagaagaaatgcgaagaagtcgcgttgcttttacgaaaggcgggagctaatgctgtcacctactatcgaggcttggatgtttcagtcatacccaatgaggggaatgtcgttgtggtcgccacggatgccttgatgacagggtattctggcaatttcgacaccgtcactgactgcaacactgctgtagagctagacatagagttctctctagaccctactttcactatggttactactcctaaaccatctgatgccgtttgtcgcacgcaacgcagggggaggactggcaggggccggaggggtacttactattatgttaatagtggtgagcgcccttctggagtcctgtcttcctccgtcctgtgtgagtgttacgatagcggcttggcttggtttggcttgagcccagctcaggtcactgttttgctgcaagcttaccttaagcaacctggacttcccactggacttgaccacactgaattttgggagagcgtcttcatagggttacctactgttgacgctttcttcctgtctcaattgaaacaacaaggagttacattcccttatctgactgccatccaagcaactgtatgcttgaacgcccaggctaaagctcccagcaaagatgaacgttggaaggtgctgtctcggtatattacaaccaaccgaactcccacacctttgctgtataggcttgaggatacccatgacgatctcactttcactcacccggtgactaaatacatccaagcctgcatggaggctgagattgacacccaaaccaacgcttgggttattgcgggtggctgtatcgcagctttggttgctgttgctgccttgaccggcagcgtcgctatcatagctgaggtacatgtaaatgagaaagtcgtggttgttcctcataaaggtgtcctatatgccgattttgacgagctggaggaatgttttgaccaccatcaatacattcagcaaggttatgaatgggcgtcgcgagctgcgcaaaaaatccgcgaagtcgcgtcatccattgatcctcctactggtcaagcacaaccaatgctatcggctatagagaagttttggaatcagcacatgtggaacattttgtcaggtgttcagtatttagcagggcttacaacactcccttacaatccatctgtagcttgtcttatgggatttgtttctggtctcactacaggattgcctagaccagctatggctttcctcaccattctaggcgggtgggctgcgagcatggtggccccgcctcaggcagcatccacatttgtcggtgccggattggctggcatcgctatcggtgccgtcggtttcactgacgtaatcgtgggtctgcttgcaggttacggggctggcgttgccggagcccttaccgctttcaagatcttgagtggtgttactccaagtggtgaagacctcatcaatcttttaccatctttgctcaaccctggtgctcttgctgttggtgtgggcgccgccttcatcctgaaaaggtatactggcggcagtgaagggcttgtcgcgtgggtcaacagactcattgcgttttgctctcgggggaatcatgtttccccagatcactacgtccagcagcagcaggtcgtcagggacgttattgcctgtcttgaatctttaactcttaccagattagtcaaaactattcataattttgttacaagtgaaaatgatcaaaattgtgattttactgccatttacttttttattcaatggctaatgaaagccctttatgattgcttcacttgggcaaagggaattatccttcctcacctcccgggactcccgatcatatcgtgtgattatggctactctggccgttgggccggagatggtattatcacaacgcggtgtggttgtgggaatttgatcaccgggaacgtgaggaatgaaaaaattagaatcacaggctcgcggaggtgccgtaatctttggctcaatactttcccaattaattcgaccactactggtggaccccggccgaatccttatgacacctggaaaacagccgtcttgagaatcacttccaccgagtatgtcgagtttaagagggagagaaatgctgtcagagttatcggcgctaccgcagacaaattacgcattccctgccaagtacctgaacctgatttaatgacatttatagatggggtcagaatccatcgactggctccgtctccaaaacctatgttgcgcgacgaggtggtggtcttaattggcaatcacacctacccagtcggcgcgacgctcccttgcactccagaaccagacgtagacactgtatcttctttactcactgatccaggacatgtcactgcagagactgctgcccgacggctcaggagaggacgcaccgttgacgtcgagtcctcgagcggctctgagctatcagcagtttcgcggggtgctgcctctcgtgtgtcagaagagcacgagatggccggcggccctgtgagaccgttgactggcgaagacgagctggcgtggattagatcgttttacggacgttctgttacaattgaagtcgatgacaaagttatcaacttcgattcatggaccatcaattctgggtcagagggagagcactctcgagaatcagtcggcgctccagacgacgaccgggtggtggtggctgaaccacctccacctcccggtccagcatggatgcggaaagactacgttccagctctcgtttccgggtgtcccatcaagcccggtagtgctactcctgaacccagtgagccttcggccacggaatctgcccccgtggaggagacagaggagcctaaggaagacgagaagggtgaggagacagaccctgacatgcctcctctcgaaggtgaagaacctgaggaggatgacagcagtcagtgggagactacgtctgacaaggcggagtcgtgctccttgtcctactcgtggactggcgcactcgtcacggcaacacgccgcgaggagaggcgccaccctataggccctctgtccaacactctaatcaccaggcacaatctcgtctatcagaccacaacagcgtcagctagtgcgagaatggccaaagtgacaattgatagggaacaagtcttagacaaatactactttgacactgttacccaaatcaaaaacagggccagagaggtgacggctcacctgctatcctgggatgaggtggcccgtctaacaccaaagaacacagctcgggctaagtcgggtctatccggatccgatgtgcgaaagctgactcgagcagctaggcgggaactgaattccacgtggcaggatttgctgtcatcctctgatgagcccatccctacaacagtcatggctaagaatgaggtgttcgtatcgtcgccaaccgctcggaagccggccaggttgatcgtttaccccgatttgtctgtgcgggcttgcgaaaagagagcgatgtatgacctcttccaaaagttaccttatgctattatggggaaggcctatgggttccaatacactccccgtcagcgtgtctccagattgctggacatgtggcggactttcaagaaccctatgggcttttcctatgacaccaaatgctttgactccacggtcactcctcatgatatagatactgagagagacattttccttagtgccaaccttcctgatgaggcccagactgtcatcaagaacttaacatctaggctttacagagggtcccctatgtacaactctcgtggggatctcgttggaaaaagagaatgccgtgcttctggggttttccccacgagcatgggcaacactctcactaattacatcaaagcaacagccgctgccaaggcagctggctttgccgaccctcagtttcttatctgtggagatgatcttgtttgtgtcacctccagtaaaggcgttgaggaagatgagcaggcgttacgtgagttcactaatgccatgacgaagtactccgccatacctggagatctccccaaaccatactatgacttggagcagataacatcttgttcctctaatgtgactgttgcccaagacaggaatgggcgaccctactatttccttactcgcgatccgacaacgccacttgctcgggcgagttgggaaacaatcagtcacagtcctgtaaacagctggttagggaacataattgcctttgctcctactttatgggttaggctcgttttcctcactcatttctttggcctgctgcttcagcaagacgcggtggatcagaattatgagtttgagatgtacgggtctacctattctgtcaatcctcttgatttgccggcaataatttataagctccatggccctgaggcctttgaccttactaattattctccttacgaggtccagagagtggccgctgcgcttcaaaagttggggtcaccacctcttcgagcttggaaacgtagggctaagctcgttcgttctaagcttaaagtacgtggtggtcgctactctgttgtcgcggactatctctttggcttcgcttctgcttaccgaccgaagaggcctgcgcctcccggcgtaaattcgattgatgtttccggttggttctccattggagatgactcccttggggatatctaccggcaactaccgatcgttgctgggaggtggattcctcttcttctcttacttcctttattggctgc

SEQ ID NO: 10 - DNA, 9228 bp, Unknown
  Description of Unknown Non-primate hepacivirus H3-H3-GBX-1 polynucleotide isolated from horses
cctccgtgctaggcacggtgcgttgtcagcgtattgcgcttgcatgcgctccacgcgtcgtccaacgcggagggaatttcacattaccatgtgtcactccccctgtggaggaacccacccgtttacacggaaatgggttaaccatacccaaagtacgggtactcgggtcctcccagggcccccccggcaggtcgagggagctggaattcgtgaatccgtgagtacacggaaatcgcggcttgaacgtcttttaatgaccttcggagccgaaatttgggcgtgccccacgaaggaaggcgggggcggtgttgggccgccgccccctttatcccacggtctgataggatgcttgcgagggcacctgccggtctcgtagaccataggacatgggcaacaactctaaaaaccaaaaaccaaaaccccaaagaggaccacggaatagggtcaggggccagtcgcggtctggtcctgtagtattcccgtccggcgctgttctcgttggaggaaggtatattcctccaccaaagaaggccatccgcgggcctcgtagaggtttggtgcaggctcctaagtcatcggagcgcggttccccccggaagaagcgccaacctccgccgcaaactgatagctcgtggcgaaagtactttcctaaattctggggggatagaggatacccttggccatatgtcgatcctgttcttcagtggagtgcatggggtacctcgcctggagcataccgcaccaggtggggtccccgtgaccctcgacacaagtcgcgcaatcttgggcgcgtcattgacactctcacctgcggggttgccgatctcgccggatacgttccggtgctcggtgcccccgctggtgcgttatgccgtggcgctgctcaccttgtacggtttgtggaggacggggccaatttcatcactggcaacatacctggcatgggtttctctatttttttgttggctttgctctcagctgtgtctttcggcgaggcttctgttgtgcgtaacgggggacatgttgtcagtaacgattgtaattcttcacagatactgtgggctgcctccgactgggctatccacgaagtagggtgtgtcccttgcgtggacagcacttgttgggtgcctctcacgtcaagtatttctgttaggaatgaatctgttattgtacgtggccttggttcgcatatagatgttctcgctgcaatggcatctgtctgttccactctaggtattggtgaggcttgtggtactgcaactctaacctatattacttttttgtctaggtttttcatgtctttgaatttaacaaatgactgtgaatgttttctctaccctggcgccatttccacctttgagtttactttaagagctttgcaatctatgatgccaaatttgtccggttttgtttccatgtttagtggggtaccaaataccctatttaccatctttacaaacgggcattggggcgtcattcttgccttatgtctttatggcacaactaataattactttaagctttgcctattgcttttggcatattctggcttagtttcttgtgattctgattatatcaatgtttctttgtcctgtaatttcactgtcaaacagatgtggggctggacgttyttcccaaagtgggcactactgaatggtcagaggcttaattgcacagagggatcaccctataacccgaaatgcaaaggacctatggactttaacataacaactgatccggttgtggcttacagcgggacacgttcacacccaccctgtccttaccatgtttctagaccttgttctgtacttaatgcgtccagagtctgtggcaaaccgacctgcttcggccctgcgccgatcgaggtcggtgttactgaccgggatggaaatctagcttcttggaatgatactggccaattttattttgatcttcggtcccctcaccgaccccctcgagggcgttggtacgggtgtgtttggcttaatagcacagggtgggttaagcaatgtggggctcctccgtgtaacatgaagctcatgtctaacacgagcaaaacgtttgtttgcccgtccgactgttttaggcaaaaccccaaagccacttaccaactttgtggccaaggaccgtggatcagccataattgcttgattgattacactgatagatatcttcatttcccatgcacagagaatttcaccgtctacccggtccggatgattcttggtgacggcgctcgggatgtgagggtggcttgtaagttcaatagatcgattagctgcaggactgaagacaggttgagagcgagcatcgtctccttgctctacagtgtgactactgcagctgttccaccttgccacttctcccccttacccgctttcactaccgggctcattcacttagaccgaaatctgagtgatgtgcagtatgtgtgggcgatgacacccagcgccgttaacatttttctgagacttgagtgggcggtattcttcttgcttttgctcatggatgctaaagtttgtgctattctctggttctgtttgtgcttggccttgcaagccgaggcttacctttcagatactatgaggcttattgctctatcttatattgcagatgattccttgctttatgctctgttgttttattgtataatttactttactccaagcagagtacctcctttctgcgtttttgtgtattattggaagttttccttggctttcatggttttggctttaccacatcgagcctgggcttttgacaataccagtgcggtaactgctgcttttagcatagctctgttttgtttatacattacttgcctatcatgctataagaaacttttcctttttgttaaatggtggcttgaatattgggatgttaggattgaatgtgcttggcgctatctcggccctagggttaacccaaaggacgagaaacttgcttttgctttatgcttctcctttttctatccatccttgtttcgtgctgtttacttacctctagcagtggtgtgtggatctttctccatgatcaacaagcgcgtacaaaaggtggcgtaccttaggcgcgctgaagtcctggtgagggtcctcactatctgtagggatgtctatggtagtaagtgggtacagtggtgtgtcttgtggattgcttctcactttggcactttcctttatgatcacttaactcctattgacacttgggccgctccagggttgagggacctcatgcactctttagaacccataactctctctcctatggagaggaaagtggtcaaatggggtgcacggaaggtcgcttgtggcgacattctgcatggtctgcccgtctcggcgaggttgggtagagaaatctgcctaggccctgctgatagacttacaagcaaaggctggagattgttgtcacctattaccgccactgtcactaagacacgtggcattccttcagcaattgtttgttgtcttacgggtagagataaatatccccatagaggccattgttacatcttgacctcgttgaccaaaactttcatgggtactgtttgtaagggtgtgttgtggtccgtccaccacggtggcggcactgctactcttgcttctgataagtcttccctgctgcaagtcttgtgctctcctggcgatgaccttgtagcgtggcctgccccggccgggtccaaatctttccagccttgcacatgtggctcggctgatgtgtttttggtcacccgaactggtcaggttgtacctgcgaggaaaacgtcagagaaagacgcttctctcatttctccactgccaatttcctcacttaaaggcagctcaggaggcccggtcctttgtaaggatggggaccttgtgggcatcttttgctcagcttctgttactaggggagtggcaaagcgaatacatttcgcagacatgcgaacacgcagcgtgtcttcttgccccccgaagtatacagatttggactctccacctgcagttccttcttcataccaggtgtcttttctccatgcccctacaggcagtggcaaatccaccaagatgccgctgtcgtatgtagagttgggttaccatgtcctagttcttaatccatcggtcgcctccactctaagctttgggccatacatggacaagacctatggtgaatgtcctaacatcaggacaggtgccagttgcaaaactacaggctctaagctcacctattccacctatgggaaattcctagcggacggcggggtatcggctggcgcttatgacataatcatttgtgatgagtgccatagcacggactccacatccgttttgggaattggatctgtccttgacggggcagagtctaaaggtgtaaagcttgttgtacttgctactgccaccccaccaggttctcaaactgtccctcatcccaacatcgatgaggaagcccttacacaaagtggggacatccccttctatgggaagatgctaaagtcgtccttactccttagtggaaggcatcttatcttctgccattctaagaagaagtgcgaggaggtcgctctccttctgagaaaggctggagctaatgctgtcacctactatcggggcctggatgtctctgtcatcccgaacgaggggaacgttgtcgtcgtggccacggatgctcttatgacagggtactctggcaactttgatagcgtcaccgattgcaacactgctgtagaactggatattgaattttctcttgacccaactttctccatggttaccactcctaaaccatctgacgccgtctgtcgtacacagcgcagggggaggactggcagaggtcgtaggggtacttactactatgtgaatagcggtgagcgcccttctggtgtgctgtcgtcttctgtcctgtgtgagtgctatgatagtggcctggcatggttcggcctaagccctgcgcaggttactgtgctgctacaggcctatttgaaacaacctggactccctaccggactggaccacactgagttttgggagagcgtcttcataggcttgcctactgttgatgctttctttttatctcagctaaaacaacaaggtgttacattcccatatttgactgccatacaagctactgtttgcctcaatgcccaggctaaagcccccagcaaagatgaacgctggaaggtgctgtcccggtatatcacaactaaccggaccccaacgcccttactgtatagacttgaggatacacacgatgacttgacgtttacacatcctgtgaccaagtatattcaggcctgtatggaggccgagattgacactcagacaaatgcttgggtcatagctggtggatgcgtcgcggcattggtcgctgttgctgccctcacaggcagcgtcgcgattattgccgaggtgcatgtcaacgaaaaggttgtcgttgttcctcataagggcgttctctacgctgattttgacgagttggaggaatgttttgatcatcatcaatacatccaacaaggttatgaatgggcatcgcgtgctgcgcaaaagatccgcgaggtcgcgacgtccattgaacctcccactggtcaggcacaacctatgctgtcagcaatggagaaattttggaaccagcacatgtggaacattttgtcaggagtacagtacctggcagggttgactacactcccctacaatccatctgtagcttgtttaatggggtttgtctcaggtctcaccacaggactgccacgtccagcaatggctttcctcaccattctaggcgggtgggctgctagcatggtggctccacctcaagcagcttccacgttcgtcggtgccggactcgctggtattgctatcggcgcagtagggtttaccgacgttattgtgggactccttgcaggttacggggctggcgttgccggcgctctaaccgcctttaaaatcttgagtggtgttactccaactggtgaagacctgatcaatttgcttccatcattgctcaatcctggcgctcttgctgtgggcgtgggagctgcctttatcctgaaaaggtatacaggtggcagtgagggtcttgttgcctgggtcaatcggctcatagccttttgctcccggggcaatcatgtctctcccgaccactacgtccaacaacaacaggtcgtcagggatgtcattgcttgccttgaatccttaaccctaaccaggctagtgaaaactatccataattttgttacaagtgaaaatgatcagaattgtgattttactgccatttacttcttcattcagtggctaatgaaaaccctttatgattgtttcacctgggcaaagggaatcattcttccccacctcccgggtttcccgatcatatcgtgtgacactggctactctggccgttgggctggagacggtcttgtgactactcgatgtggttgtggcaacatgatcactgggaacgtgaggaatgaaaagattaggataacaggctcgcggaagtgccgtaatgtctggctcaatgctttcccgatcaattcgacgactaccggtggacctcgacctaatccttatgacacctggaaaacggctgttttgaggattacttctaccgagtacgttgagtttgagaggcgaggcactgctgtcagagttactggtgctactgcagacaaattaagaattccctgccaggttcctgaacctgatttaatgaccttcatagatggggttcggatccacaggctggctccgactccaaagccaatgttgcgtgacgaggtggtggtcttaattggcaatcacacttaccccgtgggcgcgacacttccatgcactcctgaaccagatgtagacactgtatcttcccttttgacagatccaggacatgtcactgcagagactgcagcaagacggcttagacgcgggcgtaccgtggacgtcgagtcctctagcggctccgagctgtcggccgtctcgcggggcgccgcgtctcgtgtttcagaagaacacgagatgcagggtggccctgtgagaccgctaactggtgaagacgagctggcttggattcgttcgttttacggacgctccgtgacgattgaggttgacgacaaagttatcaacttcgattcgtggaccatcaattctgggtctgagggggagcactctcgagaatcggtcgtcgccccagacaacgaccaggtcgtggtggcagaacctccgccacctcctggtcccgcctggatgcgcaaagactatgtcccagctctcgtttctgggtgtcccatcaagcccggaagtgctactcctgagcccagtgagccctctgctacggaatctgctcctacagaggagaaagaggaacctagggtcgacgagactggcaatgagacagaccctgacatgcctcctctcgaaggcgaagagcctactgaggaggatgatgacggggaatgggagactacgtctgagaaggcggagtcatgctcgttgtcatactcgtggactggcgcccttgtcacggctacccgccgtgaagagaggcgacatcccataggtcctctctccaacacactcattaccaaacataacctcgtttaccagacgacaactgcgtctgctagtgcgaggatgaccaaagtgacaattgacagagaacaaattttggacaaattttattttgacactgtcaccgcagtcaagaaaaaggcctcggaggtggcagcggacctgcttacgtgggatgaggttgcccgcttgacgcccaagaacacagccagagcgaaatctggactcagtggttcagatgttcgccagcttactagggcggccaggcgggagctaaattccatgtggcaggacctcctgtcagactctgatgaccctatcccaacaacagtcatggcaaagaatgaggtcttcgtgtcatctccgaccgctcgcaagccagccaggctgatcgtttaccccgacctgcctgtgcgtgcctgtgagaagagggccatgtatgaccttttccaaaaactaccttatgctattatggggaaggcctatgggtttcagtacactcctcgccaacgtgtcgataggctactcgacatgtggcggcatttcaaaaaccctatgggcttttcctatgataccaagtgctttgactccactgtcactccccatgatatagatactgagagggacattttccttagtgcaacattacctgatgaagcccggacagtcatcaaaaatctgacatcaaggctttacagaggatcccccatgtacaattcccgtggggatcttgttggaaagagggaatgtcgtgcttcgggtgtcttcccgactagcatgggcaatacccttaccaacttcattaaggctaccgcggccgccaaggctgctggtttaagtgaccctcagttcctaatttgtggggatgaccttgtatgcataacctccagtaagggcgttgaggaagatgaacaagcgctacgagagttcactagcgcaatgaccaagtactccgccattcctggggaccttcccaaaccctactatgaccttgaacagataacatcctgctcatctaatgtgaccgtcgcccaagacagaaatggtcgaccttattatttcctcactcgtgaccccacaacaccacttgcgagggcgagttgggaaacgattagtcacagtcctgtaaatagttggttagggaatataattgcttttgcacctactgtatgggttcgactcgttttcctcactcattttttcggcctgctgctccaacaggatgcagtggatcggaactatgagtttgagatgtacgggtcaacctactcagtaaatccacttgatttgccagcaataatttataaacttcatggccctgaggcctttgatcttacaaattattctccttacgaagtccagagagtggctgcagcgctccagaagttgggatcacctccgcttcgcgcttggaaacgtcgggctaagctcgttcgttcaaagctcaaggtgcgagggggtcgctacgctgtcgtcgctgactatctttttggcttcgcttctgcttatcgtccaaagaggccagcacctcctggcgtaaattcgatcgatgtgtccggttggttctccattggagacgactccattggggatatctaccggcaacttccggtcgttgctgggcggtggatacctcttttgcttttgctacctcttttggctgcaattctttactttaataaataaaataaat

SEQ ID NO: 11 - protein, 2942 aa, Unknown
  Description of Unknown Non-primate hepacivirus NZP-1-GBX2 polypeptide isolated from horses
MSNKSKNQKPKPQRGPRGRVRGQSRSGPVVFPSGAVLVGGRYIPPPKKAIRGPRRGLVQAPKSSERISPRKKRQPPPQTDSSWRKYFSKFWGDRGYPWPYVDPVLQWGAWGSSPGAYRTRWGPRDPRHKSRNLGRVIDTLTCGVADLAGYVPVLGAPAGALCRGAAHLVRFVEDGANFITGNIPGMGFSIFLLALFSAVSFGEASVVRNGGHVVSNDCNSSQILWASSDWAIHEVGCIPCVGGVCWVPLTSSISVSNESVIVRGLGSHIDVLSAMASVCSTLGIGEACGAATLTYITFLSRFFMPLNLTNDCECFLYPGAISTFEFTMRALQSMMPNLSGFLSMFSGLPNTLFTIFTNGHWGVILALCLYGTTNNYFKLCLLLLAYSGLVSCDNYLNVSLSCNFTVKEMWGWTFFPKWALLNGQRLNCTEGSPYNPKCKGPFDFNVTTDPYIAYSGTRSHPPCPYHVSRPCSVLDASRVCGKPTCFGPAPIEVGVTDRDGKLASWNDSGQFFFDLRSPHRPPRGRWYGCVWLNSTGWVKQCGAPPCNMRLMSNKSKPFVCPTDCFRQNPKATYQLCGQGPWITHSCLIDYTDRYLHFPCTENFTVYPVRMILGDGARDVRVACKFNRSVSCRTEDRLRASIVSLLYSVTTAAVPPCHFSPLPAFTTGLIHLDRNLSDVQYVWAMTPSAVNIFLRLEWAVFFLLLLMDAKVCAILWFCLCLALQAEAYLSDTMRLIALSYIADDSLLWALVFYCVIYFTPSRVPPFCVFVYYWKFALAFMVLALPHRAWAFDNASAVTAAFSIALFCLYITCLSCYKKLFMLVKWWLEYWDVRIECAWRYLGPRVNPRDEKLAFALLFSFFHPSLFRCIYLPLAVICGSFSMINKRVQKISYLRRAEVLVRVLSICRDVYGSKWVQWCILWLASHLGTFLYDHLTPIDTWAAPGLRDLMHSLEPITLSPMERKVVKWGARKIACGDILRGLPVSARLGREICLGPADKLTSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMLKSSLLLSGRHLIFCHSKKKCEEVALLLRKAGANAVTYYRGLDVSVIPNEGNVVVVATDALMTGYSGNFDTVTDCNTAVELDIEFSLDPTFSMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHVNEKVVVVPHKGVLYADFDEMEECFDHHQYIQQGYEWASRAAQKIREVAASIDPPTGQAQPLLSAVEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPSGEDLINLLPSLLNPGALAVGVGAAFILKRYTGGSEGLVAWVNRLIAFCSRGNHVSPDHYVQQQQVVRDVIACLESLTLTRLVKTIHNFVTSENDQNCDFTAIYFFIQWLMKILYDCFTWAKGIILPHLPGFPIISCDSGYSGRWAGDGLVSTRCGCGNLITGNVRNERIRITGSRKCRNVWLNTFPINSTTTGGPRPNPYDVWKTAVLRITSTEYVEFKREGTAVRVTGATADKLRIPCQVPEPDLMTFIDGVRIHRLAPNPKPMLRDEVVVLIGNHTYPVGATLPCTPEPDVDTVSSLLTDPGHVTAETAARRLRRGRTVDVESSSGSELSAVSRGAASRVSEEHEMAGGPVRPLTGEDELAWIRSFYGRSVTIEVDDKVINFDSWTINSGSEGEHSRESVHAPDDDRVVVAAPPPPPGPAWMRKDYVPALVSGCPIKPGSATPEPSEPSATESAPVEEKEEPKVDDKGEETDPDMPPLEGEEPEEGDDSQWETTSEKGESCSLSYSWTGALVTATRREERRHPIGPLSNTLITKHNLVYQTTTASASARMAKVTIDREQVLDKFYFDTVTQVKKKASEVTADLLTWDEVARLTPKNTARAKSGLSGSDIRQLTRAARRELQSTWQDLLSTSDEPIPTTIMAKNEVFVSSPTSRKPARLIVYPDLPVRACEKRAMYDLFQKLPYAVMGKAYGFQYTPRQRVDRLLDMWRHFRNPMGFSYDTKCFDSTVTPHDIDTERDIFLSANLPDEAKTVIKNLTSRLYRGSPMYNSRGDLVGRRECRASGVFPTSMGNTLTNFIKASAAAKAAGFADPQFLICGDDLVCVTSSKGVEEDEQALREFTNAMTKYSAIPGDFPKPYYDLEQITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYSVVADYLFGFASAYKPKRPAPPGVNTIDVSGWFSIGDDSIGDIYRQLPLVTGRWIPLLLLLPLLAAILYFNK

SEQ ID NO: 12 - protein, 2945 aa, Unknown
  Description of Unknown Non-primate hepacivirus G1-073-GBX2 polypeptide isolated from horses
MGNNSKNQKPKPQRGPRNRVRGQSRSGPVVFPSGAVLVGGRYIPPPKKAIRGPRRGLVQTPKSSERGSPRKKRQPPPQTDSSWRKYFPKFWGDRGYPWPYVDPVLQWSAWGTSPGAYRTRWGPRDPRHKSRNLGRVIDTITCGVADLAGYVPVVGAPAGALCRGAAHLVRFVEDGANFITGNIPGMGFSIFILALLSALSFGEASVVRNGGHVVSNDCNHSQILWAASDWAIHEVGCVPCVNSSCWVPLTSSISVRNESVIVRGLGSHIDVLAAMASVCSTLGIGEACGTATLTYITFLSRFFMSLNLTNDCECFLYPGPISTFQFTMRALQSMMPNLSGFVSMFSGVPNTLLTIFTNGHWGVILALCLYGTTNNYFKLCLLLLAYSGLVSCDQTEYLNVSLSCNFTVKQMWGWTFFPKWALLNDQRLNCTEGSPYNPKCKGPMDFNITDDPFIAYTGTRSHPPCPYHVSRPCSVLNASRVCGKPTCFGPAPIEVGVTDRDGKLASWNDTGQFFFDLRSPHRPPRGRWYGCVWLNSTGWVKQCGAPPCNMRLVSNRSRTFVCPSDCFRQNPKATYQLCGQGPWISYNCLIDYTDRYLHFPCTENFTVYPVRMVLGDGARDVRVACKYNRSVSCSTEDRLRASIVSLLYSVTTAAVPPCHFSPLPAFTTGLIHLDRNLSDVQYVWAMTPSAVNIFLRLEWAVFFLLLLMDAKVCAILWFCLCLALQAEAYLSDTMRLIALSYIADDSLLYALLFYCVIYFTPSRVPPFCVFMYYWKFSLAFMVLALPHRAWAFDNTSAVTAAFSIALFCLYVTCLSCYKKLFMLVKWWLEYWDVRVESAWRYLGPRVNPKSDEKLAFALVFTFLYPSLFRAVYLPLAVVCGSFSMINKRIQKVQYLRRAEVLVRVLTICRDIYGSKWVQWCVLWVASHFGTFLYDHLTPIDTWAAPGLRDLLHSLEPITLSPMERKVVKWGARKVACGDILHGLPVSARLGREICLGPADRLTSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVYLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQNGDIPFYGKMLKSSLLLSGRHLIFCHSKKKCEEVALLLRKAGANAVTYYRGLDVSVIPNEGNVVVVATDALMTGYSGNFDTVTDCNTAVELDIEFSLDPTFSMVTTPKPSDAVCRTQRRGRTGRGRRGTYYYVNSGERPSGVLSSSTLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHVNEKVVVVPHKGVLYADFDELEECFDHHQYIQQGYEWASRAAQKIREVATSIEPPTGQAQPMLSAIEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPSGEDLINLLPSLLNPGALAVGVGAAFILKRYTGGSEGLVAWVNRLIAFCSRGNHVSPDHYVQQQQVVKDVIACLESLTLTRLVKTIHNFVTSENDQNCDFTAIYFFIQWLMKALYDCFTWAKGIILPHLPGFPIISCDTGYSGRWAGEGLVTTRCGCGNMITGNVRNEKIRITGSRKCRNLWLNAFPINSTTTGGPRPNPYDTWKTAVLRITSTEYVEFERKGTAVKVIGATADKLRIPCQVPEPDLMTYIDGVRIHRLAPTPKPMLRDEVVVLIGNHTYPVGATLPCTPEPDVDTVSSLLTDPGHVTAETAARRLRRGRTVDVESSSGSELSAVSRGAASRVSEEHEMQGGPVRPLTGEDELAWIRSFYGRSVTIEVDDKVINFDSWTINSGSEGEHSRESVVAPDNDQVVVAQPPPPPGPAWMRKDYVPALVSGCPIKPGSATPEPSEPSATESAPVEEKEELRVDEADVETDPEMPPLEGEEPEGDDDGEWETTSEKAESCSLSYSWTGALVTATRREERRHPIGPLSNTLITKHNLVYQTTTASASARMAKVTIDREQIFDKHYFDTVTAVKKRASEVTADLLTWDEVARLTPKNTAKAKSGLSGSDVRKLTRAARRELNSMWQDLLSGFEEPIPTTVMAKNEVFVSSPTARKPARLIVYPDLPVRACEKRAMYDLFQKLPYAIMGKAYGFQYTPRQRVERLLDMWRHFKNPMGFSYDTKCFDSTVTPHDIDTERDIFLSANLPDEAKTVIKNLTSRLYRGSPMYNSRGDLVGKRECRASGVFPTSMGNTLTNYIKATAAAKAAGLSDPQFLICGDDLVCITSSKGVEEDEQALRNFTSAMTKYSAIPGDLPKPYYDLEEITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYAVVADYLFGFASAYRPKRPAPPGVNSIDVSGWFSIGDDSIGDIYRRLPVVAGRWIPLLLLLPLLAAILYFNK

SEQ ID NO: 13 - protein, 2942 aa, Unknown
  Description of Unknown Non-primate hepacivirus A6-006-GBX2 polypeptide isolated from horses
MSNKSKNQKPKPQRGPRSRVRGQSRSGPVVFPSGAVLVGGRYIPPPKKAIRGPRRGLVQAPKSSERTSPRKKRQPPPQTDSSWRKYFPKFWGDRGYPWPYVDPVLQWGAWGSSPGAYRTRWGPRDPRHKSRNLGRVIDTLTCGVADLAGYVPVLGAPAGALCRGAAHLVRFVEDGANFITGNIPGMGFSIFLLALFSAVSFGEASVVRNGGHVVSNDCNSSQILWASSDWAIHEVGCVPCVDSACWVPLTSSISVRNESVIVRGLGSHIDVLSAMASVCSTLGIGEACGTATLTYITFLSRFFMSLNLTNDCECFLYPGAISTFEFTLRALQSMMPNLSGFLSMFSGLPNTLFTIFTNGHWGVILALCLYGTTNNYFKLCLLLLAYSGLVSCDDYINVSLSCNFTVKQMWGWTFFPKWALLNGQRLNCTEGSPYNPKCKGPFDFNITADPFIAYTGTRSHPPCPYHVSRPCSVISASKVCGKPTCFGPAPIEVGITDRNGKLASWNESGKFYFDLRSPHRPPRGRWYGCVWLNSTGWVKQCGAPPCNMPFMSNRSRTFVCPTDCFRQNPKATYQLCGQGPWITHSCLIDYTDRYLHFPCTENFTVYPVRMILGDGARDVRVACKYNRSVSCTTEKRLRASIVSLLYSVTTAAVPPCHFSPLPAFTTGLIHLDRNLSDVQYVWAMTPSAVNIFLRLEWAVFFLLLLMDAKICAILWFCLCLALQAEAYLSDTMRLIALSYIADDSLLWALAFYCVIYFTPSRVPPFCVFVYYWKFALAFMVLALPHRAWAFDNTSAVTAAFSIALFCLYITCLSCYKKLFLLVKWWLEYWDVRIECAWRYLGPRVNPRDEKLAFALLFSFFYPSLFRCIYLPLAVICGSFSMINKRVQKISYLRRAEVLVRVLCICRDIYGSKWVQWCILWLASHFGTFLYDHLTPIDTWAAPGLRDLMHSLEPITLSPMERKVVKWGARKVACGDILRGLPVSARLGKEICLGPADKLTSKGWRLLSPITATVTKTRGIPSAIVCCLTGRDKYPHRGHCYILTSLTKTFMGTVCKGVLWSVHHGGGTATLASDKTSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMLKSSLLLSGRHLIFCHSKKKCEEVALLLRKAGANAVTYYRGLDVSVIPNEGNVVVVATDALMTGYSGNFDTVTDCNTAVELDIEFSLDPTFSMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHINEKVVVVPHKGVLYADFDELEECFDHHQYIQQGYEWASRAAQKIREVAASIDPPAGQAQPLLSAVEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPSGEDLINLLPSLLNPGALAVGVGAAFILKRYTGGSEGLVAWVNRLIAFCSRGNHVSPDHYVQQQQVVRDVIACLESLTLTRLVKTIHNFVTSENNQDCDFTAIYFFIQWLIKTLYDCFTWAKGIILPHLPGFPIISCDSGYSGRWAGDGLVSTRCGCGNIITGNVRNERIRITGSRKCRNVWLNTFPINSSTTGGPRPNPYDVWKTAVLRITSTEYVEFKREGTAVRVTGATADKLRIPCQVPEPDLMTFIDGVRIHRLAPDPKPMLRDEVVVLIGNHTYPVGATLPCTPEPDVDTVSSLLTDPGHVTAETAARRLRRGRTVDVESSSGSELSAVSRGAASRVSEEHEMAGGPVRPLTGEDELAWIRSFYGRSVTIEVDDKVINFDSWTINSGSEGEHSRESVVAPDNDQVVVAAPPPPPGPAWMRKDYVPALVSGCPIKPGSATPEPSEPSATESATVEEKEEPKPDEKGEETDPDMPPLEGEEPEEEDDSQWETTSEKAESCSLSYSWTGALVTATRREERRHPIGPLSNTLITKHNLVYQTTTASASARMAKVTIDREQVLDKFYFDTVTQVKKKASEVTADLLAWDEVARLTPKNTARAKSGLSGSDIRQLTRAARRELQSTWQDLLSSSDEPIPTTIMAKNEVFVSSPTARKPARLIVYPDLPVRACEKRAMYDLFQKLPYVVMGKAYGFQYTPRQRVDRLLDMWRHFKNPMGFSYDTKCFDSTVTPHDIDTERDVFLSANLPDEAKTVIKNLTSRLYRGSPMYNSRGDLVGRRECRASGVFPTSMGNTLTNYIKASAAAKAAGFADPQFLICGDDLVCVTSSKGVEEDEQALREFTNAMTKYSAIPGDLPKPYYDLEQITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYSVVADYLFGFASAYRPKRPAPPGVNSIDVSGWFSIGDDSIGDIYRQLPLVTGRWIPLLLLLPLLAAVLYFNK

SEQ ID NO: 14 - protein, 2946 aa, Unknown
  Description of Unknown Non-primate hepacivirus B10-022-GBX2 polypeptide isolated from horses
MSNKSKNQKPKPQRGPRNRVKGQSRSGPVVFPSGAVLVGGRYIPPPKKAIRGPRRGLVQAPKSSERTAPRKRRQPPPQTDTSWRKYFPKFWGDKGYPWPYIDPVLQWGAWGSSPGAYRTRWGPRDPRHKSRNLGRVIDTLTCGVADLAGYVPVLGAPAGALCRGAAHLVRFVEDGANFITGNIPGMGFSIFLLALFSAVSLGEASVVRNGGHVVSNDCNSSQILWAASDWAIHEVGCVPCVESSCWVPLTSSISVRNESVIVHGLGSHIDVLAAMASVCSTLGIGEACGAATVTYITFLSRFFMNLDLTNDCECFLYPGAISTFEFTMRALQSMMPNLSGFVSMLSGVPNTLFTIFTNGHWGVILALCLYGTTNNYFKLCLLLLAYSGLVSCEPEKSEYINVSLACNFTVKELWGWTFFPKWALLNGRRLNCTEGSPYNPKCKGPMDFNTTTDPVIAHTGMRSHPPCPYHVSRPCSVINASRVCGKPTCFGPAPIEVGVTDRDGNLASWNDTGQFFFDLRSPHRPPRGRWYGCVWLNSTGWVKQCGAPPCNMQLESKPDKTFVCPSDCFRQNPKATYQLCGQGPWITHSCLIDYTDRYLHFPCTENFTVYPVRMILGNGARDVRVACKYNRSVSCRTEDRLRASIVSLLYSVTTAAVPPCHFSPLPAFTTGLIHLDRNLSDVQYVWATTPSAVNIFLRLEWAVFFLLLLMDAKVCAILWFCLCLALQAEAYLSDTMRLIALSYIADDSLLWALVFYCVIYFTPXRIPPFCVFVYYWKFALAFMVLALPHRAWAFDNTSAVTAAFSIALFCLYITCLSCYKRLFLLVRWWLEYWDVRVECAWRYLGPRVNPKDEKLAFALLFAFFYPSLFRCVYLPLAVVCGSFSMINKRVQKITYLRRAEVLVRVLTLCRDLYGSKWVQWCVLWVASYFGTFLYNHLTPIDTWAAPGLRDLMHSLEPITLSPMERKIVKWGARKVACGDILQGLPVSARLGREICLGPADRLSSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDTPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMXKSSTLLSGRHLIFCHSKKKCEEVAXLLRKAGANAVTYYRGLDVSVIPNEGNVVVVATDALMTGXSGNFDTVTDCNTAVELDIEFSLDPTFTMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHVNEKVVVVPHKGVLYADFDELEECFDHHQYIQQGYEWAARAAQKIREVASSIDPPTGQAQPMLSAIEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVSPSGEDLVNLLPALLNPGALAVGVGAAFILKRYTGGSEGLVAWVNRLIAFCSRGNHVSPDHYVQQQQVVRDVIACLESLTLTRLVKTIHNFVTSENDQNCDFTTIYFFIQWLMKALHDCFTWAKGIILPHLPGLPIISCDYGYSGRWAGDGIITTRCGCGNMISGTVRNERIRITGSRKCRNVWLNTFPINSSTTGGPRPNPYDTWKTAVLRITSTEYVEFKREGTAVRVIGATADKLRIPCQVPEPDFMTFIDGVRIHRLAPSPKPMLRDEVVVLIGNHTYPVGATLPCTPEPDVDTVSSLLTDPGHVTAETAARRLRRGRTVDIESSSGSELSAVSRGAASRVSEEHEMAGGPVRPLTGEDELAWIRSFYGRSVTIEVDDKVINFDSWTINSGSEGEHSRESVDAPDDDRVVVAEPPPPPGPAWMRKDYVPALVSGCPIKPGSATPEPSEPSATESAPVEETEEPMVDEKGEETDPDMPPLEGEEPEEGDDSQWETTSEKAESCSLSYSWTGALVTATRREERRHPIGPLSNTLITRHNLVYQTTTASASARMAKVTIDREQILDKYYFDTVTQVKNRAKEVTADLLSWDEVARLTPKNTARARSGLSGSDVRKLTRSARRELNSMWQDLLSSSEEPIPTTVMAKNEVFVSSPTARKPARLIVYPDLPVRACEKRAMYDLFQKLPYAVMGKAYGFQYTPRQRVSRLLDMWRTFKNPMGFSYDTKCFDSTVTPHDIETERDIFLSANLPDEARTVIKNLTSRLYRGSPMYNSRGDLVGKRECRASGVFPTSMGNTLTNYIKATAAAKAAGLADPQFLICGDDLVCVTSSKGVEEDEQALRDFTNAMTKYSAIPGDLPKPYYDLEQITSCSSNVTVAQDRNGRPYYFLTRDPTTPLARASWETISHSPVNSWLGNIIAFAPTLWVRLVFLTHFFGLLLQQDAVDQNYEFEMYGSTYSVNPLDLPAIIYKLHGPEAFDLTNYSPYEVQRVAAALQKLGSPPLRAWKRRAKLVRSKLKVRGGRYSVVADYLFGFASAYRPKRPAPPGVNSIDVSGWFSIGDDSIGDIYRQLPIVAGRWIPLLLLLPLLAAILYFNK

SEQ ID NO: 15 - protein, 2946 aa, Unknown
  Description of Unknown Non-primate hepacivirus F8-068-GBX2 polypeptide isolated from horses
MGNNSKNQKPKPQRGPRNRVRGQSRSGPVVFPSGAVLVGGRYIPPPKKAIRGPRKGLVQAPKSSERGSPRKKRQPPPQTDSSWRKYFPKFWGDKGYPWPYVDPVLQWSAWGTSPGAYRTRWGPRDPRHKSRNLGRVIDTLTCGVADLAGYVPVLGAPAGALCRGAAHLVRFVEDGANFITGNIPGMGFSIFILALLSVLSFGEASVVRAGGHIVSNDCNSSQILWAASDWAIHEVGCVPCVESTCWVPLTSSISVKNESVIVRGLGSHIDVLAAMASVCSTLGIGEACGAATLGYITFLSRFFMSLNLTNDCECFLYPGAISTFEFTMRALQSMMPNLSGFVSMFSGVPNTLFTIFTNGHWGVILALCLYGTTNNYFKLCLLLLAYSGLVSCESDYLNVSLSCNFTVKQMWGWTFFPKWAILNDQRLNCTEGSPYNPKCKGPMDFNITADPVIGYSGTRSHPPCPYHVSRPCSILDASRVCGKPTCFGPAPIEVGVTDQDGNLVSWNDSGKFFFDLRSPHRPPRGRWYGCVWLNSTGWVKQCGAPPCNMALMSGKGKTFVCPSDCFRQNPKATYQLCGQGPWISYNCLIDYTDRYLHFPCTENFTVYPVRMVLGDGARDVRVACKYNRSERCRTEDRLRASIVSLLYSVTTAAVPPCHFSPLPAFTTGLIHLDRNLSDVQYVWAMTPSAVNVFLRLEWAVFFLLLLMDAKVCAILWFCLCLALQAEAYLSHTMRLIALSYIADDSLLYALLFYCVIYYTESRVPPFCVFMYYWKFSLAFLVLALPHRAWAFDNASAVTAAFSIALFCLYVTCLSCYKKLFMLVKWWLEYWDVRVECAWRHLGPGVSPHSERFAFILVFAFLYPPLFRAVYLPLAVVCGSFSMINKRVQKIQYLRRAEVLVRVLTICRDVYGSKWVQWCVLWVASHFGTFLYDHLTPIDTWAAPGLRDLMHSLEPITLSPMERMVVKWGARKIACGDILHGLPVSARLGREICLGPADRLTSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVYLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQNGDIPFYGKMLKSSLLLSGRHLIFCHSKKKCEEVALLLRKAGANAVTYYRGLEVSVIPNEGNVVVVATDALMTGYSGNFDTVTDCNTAVELDIEFSLDPTFSMVTTPKPSDAVCRTQRRGRTGRGRRGTYYYVNSGERPSGVLSSSVMCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHVNEKVVVVPHKGVLYADFDELEECFDHHQYIQQGYEWASRAAQKIREVATSIEPPTGQTQPMLSAIEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPNGEDLINLLPSLLNPGALAVGVGAAFILKRYTGGSEGLVAWVNRLIAFCSRGNHVSPDHYVQQQQVVKDVIACLESLTLTRLVKTIHNFVTSENDQNCDFTAIYFFIQWLMKALYDCFTWAKGIILPHLPGFPLISCDTGYSGRWAGEGLVTTRCGCGNMITGNVRNERIRITGSRKCRNLWLNAFPINSTTTGGPRPNPYDTWKTAVLRITSTEYVEFERKGTAVRVIGATADKLRIPCQVPEPDLMTYIDGVRIHRLAPTPKPMLRDEVVVLIGNHTYPVGATLPCTPEPDVDTVSSLLTDPGHVTAETAARRLRRGRTVDVESSSGSELSAVSRGAVSRVSEEHEMQGGPVRPLTGEDELAWIRSFYGRSVTIEVDDKVINFDSWTINSGSEGGHSRESVVAPDNDQVVVAEPPPPPGPAWMRKDYVPALVSGCPIKPGSATPEPSEPSATESARVEEKEEPKVDEDGAETDPEMPPLEGEEPEGDDDGEWETDPSGSEAPAVDANCSLSYSWTGALVTATRREERRHPIGPLSNTLITKHNLVYQTTTASASARMAKVTIDREQIFDKHYFDTVTAVKKRASEVAADLLTWDEVARLTPKNTAKAKSGLSGSDVRKLTRAARRELNSMWQDLLSDSEEPIPTTVMAKNEVFVSSPTARKPARLIVYPDLPVRACEKRAMYDLFQKLPYAIMGKAYGFQYTPRQRVERLLDMWRHFKNPMGFSYDTKCFDSTVTPHDIDTERDIFLSANLPDEAKIVIKNLTSRLYRGSPMYNSRGDLVGKRECRASGVFPTSMGNTLTNYIKAAAAAKAAGLSDPQFLICGDDLVCITSSKGVEEDEQALRDFTSAMTKYSAIPGDLPKPYYDLEQITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYAVVADYLFGFASAYRPKRPAPPGVNSIDVSGWFSIGDDSIGDIYRQLPVVAGKWIPLLLLLPLLAAILY

SEQ ID NO: 16 - protein, 2950 aa, Unknown
  Description of Unknown Non-primate hepacivirus G5-077-GBX2 polypeptide isolated from horses
MSNKSKNQKPKPQRGPRNRVRGQSRSGPVVFPSGAVLVGGRYIPPPKKAIRGPRRGLVQAPKSSERTSPRKKRQPPPQTDSSWRKYFPKFWGDKGYPWPYVDPVLQWGAWGSSPGAYRTRWGPRDPRHKSRNLGRVIDTLTCGVADLAGYVPVVGAPAGALCRGAAHLVRFVEDGANFITGNIPGMGFSIFLLALFSAVSFGEASVVRNGGHVVSNDCNSSQILWASSDWAIHEVGCVPCVDSACWVPLTSSISVRNESVIVRGLGSHIDVLSAMASVCSTLGIGEACGTATLTYITFLSKFFMSLNLTNDCECFLYPGAISTFEFTLRALQSMMPNLSGFVSMFSGLPNTLFTIFTNGHWGVILALCLYGTTNNYFKLCLLLLAYSALVSCDDSTYLNVSLACNFTVKQMWGWTFFPKWALLNGQRLNCTEGSPYNPKCKEPFDFNITTDPVIAYSGTRSHPPCPYHVSRPCSVLNASRVCGKPTCFGPAPIEVGVTDRDGNLASWNDSGQFYFDLRSPHRPPRGRWYGCVWLNSTGWVKQCGAPPCNMKLMSNRSKTFVCPTDCFRQNPKATYQLCGQGPWITHSCLIDYTDRYLHFPCTENFTVYPVRMILGDGARDVRVACKFNRSRSCRTEDRLRASIVSLLYSVTTAAVPPCHFSPLPAFTTGLIHLDRNLSDVQYVWAMTPSAVNIFLRLEWAVFFLLLLMDAKVCAILWFCLCLALQAEAYLSDTMRLIALSYVADDSLLWALVFYCVIYFTPSRVPPFCVFVYYWKFALAFMVLALPHRAWAFDNASAVTAAFSIALFCLYITCLSCYKKLFLLVKWWLEYWDVRIECAWRYLGPRVNPRDEKLAFALLFSFFYPSLFRCVYLPLAVICGSFSMINKRVQKISYLRRAEVLVRVLSICRDVYGSKWVQWCILWLASHFGTFLYNHLTPIDTWAAPGLRDLMHSLEPITLSPMERKVVKWGARKVACGDILRGLPVSARLGKEICLGPADKLTSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMLKSSLLLSGRHLIFCHSKKKCEEIALLLRKAGANAVTYYRGLDVSVIPNEGNVVVVATDALMTGYSGNFDSVTDCNTAVELDIEFSLDPTFSMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPITKYIQACMEAEIDTQTNAWVIAGGCVAALVAVAALTGSVAIIAEVHVNEKVVVVPHKGVLYADFDELEECFDHHQYIQQGYEWASRAAQKIREVAASIDPPAGQTQPLLSAVEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPSGEDLINLLPSLLNPGALAVGVGAAFILKRYTGGSEGLVAWVNRLIAFCSRGNHVSPDHYVQQQQVVRDVIACLESLTLTRLVKTIHNFVTSENDQNCDFTAIYFFIQWLMKTLYDCFTWAKGIILPHLPGFPIISCDSGYSGRWAGEGLVSTRCGCGNMITGNVRNERIRITGSRKCRNVWLNTFPINSSTTGGPRPNPYDVWKTAVLRVTSTEYVEFKREGTAVRVIGATADKLRIPCQVPEPDLMTFIDGVRIHRLAPDPKPMLRDEVVVLIGNHTYPVGATLPCTPEPDVDTVSSLLTDPGHVTAETAARRLRRGRTVDVESSSGSELSAVSRGAASRVSEEHEMAGGPVRPLTGEDELAWIRSFYGRSVTIEVDDKVINFDSWTINSGSEGEHSRESVVAPDNDQVVVAAPPPPPGPAWMRKDYVPALVSGCPIKPGSATPEPSEPSATESAPVEEKEEPVVDDKGEETDPDMPPLEGEEPEDADDSQWETTSEKAESCSLSYSWTGALVTATRREERRHPIGPLSNTLITKHNLVYQTTTASASARMAKVTIDREQVLDKFYFDTVTQVKKKASEVEAGLLSWDEVARLTPKNTARAKSGLSGSDVRQLTRAARRELQSTWQDLLSSSDEPIPTTIMAKNEVFVSSPTSRKPARLIVYPDLPVRACEKRAMYDLFQKLPYAVMEKAYGFQYTPRQRVDRLLDMWRHFKNPMGFSYDTKCFDSTVTPHDIDTERDIFLSANLPDEAKTVIKNLTSRLYRGSPMYNSRGDLVGKRECRASGVFPTSMGNTLTNFIKASAAAKAAGFADPQFLICGDDLVCVTSSKGVEEDEQALREFTNAMTKYSAIPGDFPKPYYDLEQITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYSVVADYLFGFASAYRPKRPAPPGVNSIDVSGWFSIGDDSIGDIYRQLPNVAGKWIPLLLLLPLLAAILYFNKKKKKKK

SEQ ID NO: 17 - protein, 2936 aa, Unknown
  Description of Unknown Non-primate hepacivirus H10-094-GBX2 polypeptide isolated from horses
MGNKSKNQKPKPQRGPRNRVKGQSRSGPVVFPSGAVLVGGRYIPPPKKAIRGPRRGLVQAPKSSERTAPRHRRQPPPQTDSSWRKYFPKFWGDKGYPWPYVDSVLQWGAWGSSPGAYRTRWGPRDPRHRSRNLGRVIDTLTCGVADLAGYVPVLGAPAGALCRGAAHLVRFVEDGANFITGNIPGMGFSIFLLALFSAVSFGEASVVRNGGHVVSNDCNQSQILWAASDWAIHEVGCVPCVDSACWVPLTSSISVRNESVIVRGLGSHIDVVAAMASVCSTLGIGEACGTATLTYITFLSRFFIRLNLTYDCECFLYPGAISTFEFTMRALQSMMPNLSGFVSMFSGLPNTLFTIFTNGHWGVILALCLYGTTNNYFKLCLLLLAYSGLVSCSEDYINVSLSCNFTIKQMWGWTFFPKWALLNGQRLNCTPGSPYNPKCKGPMDFNITTDPVVTYSGTRSHPPCPYHVSRPCSVLNASRVCGKPTCFGPAPIEVGVTDRDGNLASWNDTGQFFFDLRSPHRPPRGRWYGCVWLNSTGWVKQCGAPPCVMNLVSNKSRTFICPSDCFRQNPKATYQLCGQGPWITHNCLIDYTDRYLHFPCTENFTVYPVRMILGDGARDVRVACNFNRSVSCRTEDRLRASIVSLLYSVTTAAVPPCHFSPLPAFTTGLIHLDRNLSDVQYVWAMTPSAVNVFLRLEWAVFFLLLLMDAKVCAILWFCLCLALQAEAYLSDTMRLIALSYIADDSLLWALVFYCVIYFTPSRLPPFCVFVYYWKFALAFMVLALPHRAWAFDNASAVTAAFSMALFCLYVTCLSCYKRLFMLVKWWLEYWDVRVECAWRYLGPRVNPRDEKLAFALIFAFLHPPLFRCIYLPLAVVCGSFSMINKRVQKISYLRRAEVLVKVLAFCRDLYGSKWVQWCVLWVASYFGTFLYNHLTPIDTWAAPGLRDLMHSLEPITLSPMERKVVKWGARKVACGDILHGLPVSARLGREICLGPADKLSSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDTPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMLKSSMLLSGRHLIFCHSKKKCEEVALLLRKAGANAVTYYRGLDVSVIPNEGNVVVVATDALMTGYSGNFDTVTDCNTAVELDIEFSLDPTFTMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCIAALVAVAALTGSVAIIAEVHVNEKVVVVPHKGVLYADFDELEECFDHHQYIQQGYEWASRAAQKIREVASSIDPPTGQAQPMLSAIEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPSGEDLINLLPSLLNPGALAVGVGAAFILKRYTGGSEGLVAWVNRLIAFCSRGNHVSPDHYVQQQQVVRDVIACLESLTLTRLVKTIHNFVTSENDQNCDFTAIYFFIQWLMKALYDCFTWAKGIILPHLPGLPIISCDYGYSGRWAGDGIITTRCGCGNLITGNVRNEKIRITGSRRCRNLWLNTFPINSTTTGGPRPNPYDTWKTAVLRITSTEYVEFKRERNAVRVIGATADKLRIPCQVPEPDLMTFIDGVRIHRLAPSPKPMLRDEVVVLIGNHTYPVGATLPCTPEPDVDTVSSLLTDPGHVTAETAARRLRRGRTVDVESSSGSELSAVSRGAASRVSEEHEMAGGPVRPLTGEDELAWIRSFYGRSVTIEVDDKVINFDSWTINSGSEGEHSRESVGAPDDDRVVVAEPPPPPGPAWMRKDYVPALVSGCPIKPGSATPEPSEPSATESAPVEETEEPKEDEKGEETDPDMPPLEGEEPEEDDSSQWETTSDKAESCSLSYSWTGALVTATRREERRHPIGPLSNTLITRHNLVYQTTTASASARMAKVTIDREQVLDKYYFDTVTQIKNRAREVTAHLLSWDEVARLTPKNTARAKSGLSGSDVRKLTRAARRELNSTWQDLLSSSDEPIPTTVMAKNEVFVSSPTARKPARLIVYPDLSVRACEKRAMYDLFQKLPYAIMGKAYGFQYTPRQRVSRLLDMWRTFKNPMGFSYDTKCFDSTVTPHDIDTERDIFLSANLPDEAQTVIKNLTSRLYRGSPMYNSRGDLVGKRECRASGVFPTSMGNTLTNYIKATAAAKAAGFADPQFLICGDDLVCVTSSKGVEEDEQALREFTNAMTKYSAIPGDLPKPYYDLEQITSCSSNVTVAQDRNGRPYYFLTRDPTTPLARASWETISHSPVNSWLGNIIAFAPTLWVRLVFLTHFFGLLLQQDAVDQNYEFEMYGSTYSVNPLDLPAIIYKLHGPEAFDLTNYSPYEVQRVAAALQKLGSPPLRAWKRRAKLVRSKLKVRGGRYSVVADYLFGFASAYRPKRPAPPGVNSIDVSGWFSIGDDSLGDIYRQLPIVAGRWIPLLLLLPLLA

SEQ ID NO: 18 - protein, 2944 aa, Unknown
  Description of Unknown Non-primate hepacivirus H3-H3-GBX-1 isolated from horses
MGNNSKNQKPKPQRGPRNRVRGQSRSGPVVFPSGAVLVGGRYIPPPKKAIRGPRRGLVQAPKSSERGSPRKKRQPPPQTDSSWRKYFPKFWGDRGYPWPYVDPVLQWSAWGTSPGAYRTRWGPRDPRHKSRNLGRVIDTLTCGVADLAGYVPVLGAPAGALCRGAAHLVRFVEDGANFITGNIPGMGFSIFLLALLSAVSFGEASVVRNGGHVVSNDCNSSQILWAASDWAIHEVGCVPCVDSTCWVPLTSSISVRNESVIVRGLGSHIDVLAAMASVCSTLGIGEACGTATLTYITFLSRFFMSLNLTNDCECFLYPGAISTFEFTLRALQSMMPNLSGFVSMFSGVPNTLFTIFTNGHWGVILALCLYGTTNNYFKLCLLLLAYSGLVSCDSDYINVSLSCNFTVKQMWGWTXFPKWALLNGQRLNCTEGSPYNPKCKGPMDFNITTDPVVAYSGTRSHPPCPYHVSRPCSVLNASRVCGKPTCFGPAPIEVGVTDRDGNLASWNDTGQFYFDLRSPHRPPRGRWYGCVWLNSTGWVKQCGAPPCNMKLMSNTSKTFVCPSDCFRQNPKATYQLCGQGPWISHNCLIDYTDRYLHFPCTENFTVYPVRMILGDGARDVRVACKFNRSISCRTEDRLRASIVSLLYSVTTAAVPPCHFSPLPAFTTGLIHLDRNLSDVQYVWAMTPSAVNIFLRLEWAVFFLLLLMDAKVCAILWFCLCLALQAEAYLSDTMRLIALSYIADDSLLYALLFYCIIYFTPSRVPPFCVFVYYWKFSLAFMVLALPHRAWAFDNTSAVTAAFSIALFCLYITCLSCYKKLFLFVKWWLEYWDVRIECAWRYLGPRVNPKDEKLAFALCFSFFYPSLFRAVYLPLAVVCGSFSMINKRVQKVAYLRRAEVLVRVLTICRDVYGSKWVQWCVLWIASHFGTFLYDHLTPIDTWAAPGLRDLMHSLEPITLSPMERKVVKWGARKVACGDILHGLPVSARLGREICLGPADRLTSKGWRLLSPITATVTKTRGIPSAIVCCLTGRDKYPHRGHCYILTSLTKTFMGTVCKGVLWSVHHGGGTATLASDKSSLLQVLCSPGDDLVAWPAPAGSKSFQPCTCGSADVFLVTRTGQVVPARKTSEKDASLISPLPISSLKGSSGGPVLCKDGDLVGIFCSASVTRGVAKRIHFADMRTRSVSSCPPKYTDLDSPPAVPSSYQVSFLHAPTGSGKSTKMPLSYVELGYHVLVLNPSVASTLSFGPYMDKTYGECPNIRTGASCKTTGSKLTYSTYGKFLADGGVSAGAYDIIICDECHSTDSTSVLGIGSVLDGAESKGVKLVVLATATPPGSQTVPHPNIDEEALTQSGDIPFYGKMLKSSLLLSGRHLIFCHSKKKCEEVALLLRKAGANAVTYYRGLDVSVIPNEGNVVVVATDALMTGYSGNFDSVTDCNTAVELDIEFSLDPTFSMVTTPKPSDAVCRTQRRGRTGRGRRGTYYYVNSGERPSGVLSSSVLCECYDSGLAWFGLSPAQVTVLLQAYLKQPGLPTGLDHTEFWESVFIGLPTVDAFFLSQLKQQGVTFPYLTAIQATVCLNAQAKAPSKDERWKVLSRYITTNRTPTPLLYRLEDTHDDLTFTHPVTKYIQACMEAEIDTQTNAWVIAGGCVAALVAVAALTGSVAIIAEVHVNEKVVVVPHKGVLYADFDELEECFDHHQYIQQGYEWASRAAQKIREVATSIEPPTGQAQPMLSAMEKFWNQHMWNILSGVQYLAGLTTLPYNPSVACLMGFVSGLTTGLPRPAMAFLTILGGWAASMVAPPQAASTFVGAGLAGIAIGAVGFTDVIVGLLAGYGAGVAGALTAFKILSGVTPTGEDLINLLPSLLNPGALAVGVGAAFILKRYTGGSEGLVAWVNRLIAFCSRGNHVSPDHYVQQQQVVRDVIACLESLTLTRLVKTIHNFVTSENDQNCDFTAIYFFIQWLMKTLYDCFTWAKGIILPHLPGFPIISCDTGYSGRWAGDGLVTTRCGCGNMITGNVRNEKIRITGSRKCRNVWLNAFPINSTTTGGPRPNPYDTWKTAVLRITSTEYVEFERRGTAVRVTGATADKLRIPCQVPEPDLMTFIDGVRIHRLAPTPKPMLRDEVVVLIGNHTYPVGATLPCTPEPDVDTVSSLLTDPGHVTAETAARRLRRGRTVDVESSSGSELSAVSRGAASRVSEEHEMQGGPVRPLTGEDELAWIRSFYGRSVTIEVDDKVINFDSWTINSGSEGEHSRESVVAPDNDQVVVAEPPPPPGPAWMRKDYVPALVSGCPIKPGSATPEPSEPSATESAPTEEKEEPRVDETGNETDPDMPPLEGEEPTEEDDDGEWETTSEKAESCSLSYSWTGALVTATRREERRHPIGPLSNTLITKHNLVYQTTTASASARMTKVTIDREQILDKFYFDTVTAVKKKASEVAADLLTWDEVARLTPKNTARAKSGLSGSDVRQLTRAARRELNSMWQDLLSDSDDPIPTTVMAKNEVFVSSPTARKPARLIVYPDLPVRACEKRAMYDLFQKLPYAIMGKAYGFQYTPRQRVDRLLDMWRHFKNPMGFSYDTKCFDSTVTPHDIDTERDIFLSATLPDEARTVIKNLTSRLYRGSPMYNSRGDLVGKRECRASGVFPTSMGNTLTNFIKATAAAKAAGLSDPQFLICGDDLVCITSSKGVEEDEQALREFTSAMTKYSAIPGDLPKPYYDLEQITSCSSNVTVAQDRNGRPYYFLTRDPTTPLARASWETISHSPVNSWLGNIIAFAPTVWVRLVFLTHFFGLLLQQDAVDRNYEFEMYGSTYSVNPLDLPAIIYKLHGPEAFDLTNYSPYEVQRVAAALQKLGSPPLRAWKRRAKLVRSKLKVRGGRYAVVADYLFGFASAYRPKRPAPPGVNSIDVSGWFSIGDDSIGDIYRQLPVVAGRWIPLLLLLPLLAAILYFNK

SEQ ID NO: 19 - protein, 12 aa, Hepatitis C virus
TVPASAYQVRNS

SEQ ID NO: 20 - protein, 12 aa, Hepatitis C virus
TTPVSAAEVKNI

SEQ ID NO: 21 - protein, 12 aa, Hepatitis C virus
INPAASLEWRNT

SEQ ID NO: 22 - protein, 12 aa, Hepatitis C virus
TVPASAVNYRNV

SEQ ID NO: 23 - protein, 12 aa, Hepatitis C virus
TVPASAVPYRNA

SEQ ID NO: 24 - protein, 12 aa, Hepatitis C virus
TTPASALTYGNS

SEQ ID NO: 25 - protein, 12 aa, Hepatitis C virus
TVPASAYEVRNS

SEQ ID NO: 26 - DNA, 378 bp, Artificial Sequence
  Description of Artificial Sequence Synthetic polynucleotide
cugcguugucagcguuuugcgcuugcaugcgcuacacgcgucguccaacgcggagggaacuucacaucaccaugugucacucccccuauggaggguuccaccccgcuuacacggaaauggguuaaccauacccaaaguacggguaugcggguccuccuagggcccccccggcaggucgagggagcuggaauucgugaauucgugaguacacgaaaaucgcggcuugaacgucuuugaccuucggagccgaaauuugggcgugccccacgaaggaaggcgggggcgguguugggccgccgcccccuuuaucccacggucugauaggaugcuugcgagggcaccugccggucucguagaccataggacatgagtaataaa

SEQ ID NO: 27 - protein, 511 aa, Hepatitis GB virus B
RVTDPDTNTTILTNCCQRNQVIYCSPSTCLHEPGCVICADECWVPANPYISHPSNWTGTDSFLADHIDFVMGALVTCDALDIGELCGACVLVGDWLVRHWLIHIDLNETGTCYLEVPTGIDPGFLGFIGWMAGKVEAVIFLTKLASQVPYAIATMFSSVHYLAVGALIYYASRGKWYQLLLALMLYIEATSGNPIRVPTGCSIAEFCSPLMIPCPCHSYLSENVSEVICYSPKWTRPITLEYNNSISWYPYTIPGARGCMVKFKNNTWGCCRIRNVPSYCTHGTDAVWNDTRNTYEACGVTPWLTTAWHNGSALKLAILQYPGSKEMFKPHNWMSGHLYFEGSDTPIVYFYDPVNSTLLPPERWARLPGTPPVVRGSWLQVPQGFYSDVKDLATGLITKDKAWKNYQVLYSATGALSLTGVTTKAVVLILLGLCGSKYLILAYLCYLSLCFGRASGYPLRPVLPSQSYLQAGWDVLSKAQVAPFALIFFICCYLRCRLRYAALLGFVPMAA

SEQ ID NO: 28 - protein, 522 aa, Canine hepacivirus
SVVRNGGHVVSNDCNSSQILWASSDWAIHEVGCIPCVDGVCWVPLTSSISVRNESVIVRGLGSHIDVLSAMASVCSTLGIGEACGAATLTYITFLSRFFMPLNLTNDCECFLYPGAISTFEFTMRALQSMMPNLSGFLSMFSGLPNTLFTIFTNGHWGVILALCLYGTTNNYFKLCLLLLAYSGLVSCDDYLNVSLSCNFTVKEMWGWTFFPKWALLNGQRLNCTEGSSYNPKCKGPFDFNVTTDPYIAYSGTRSHPPCPYHVSRPCSVLDASRVCGKPTCFGPAPIEVGVTDRDGKLASWNDSGQFFFDLRSPHRPPRGRWYGCVWLNSTGWVKQCGAPPCNMRLMSNKSKPFVCPTDCFRQNPKATYQLCGQGPWITHSCLIDYTDRYLHFPCTENFTVYPVRMILGDGARDVRVACKFNRSVSCRTEDRLRASIVSLLYSVTTAAVPPCHFSPLPAFTTGLIHLDRNLSDVQYVWAMTPSAVNIFLRLEWAVFFLLLLMDAKVCAILWFCLCLALQAEA

SEQ ID NO: 29 - protein, 555 aa, Hepatitis C virus
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 30 - protein, 555 aa, Hepatitis C virus
YEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 31 - protein, 559 aa, Hepatitis C virus
AEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTMTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 32 - protein, 559 aa, Hepatitis C virus
AQVKNTSSSYMVTNDCSNDSITWQLEAAVLHVPGCVPCERVGNTSRCWVPVSPNMAVRQPGALTQGLRTHIDMVVMSATFCSALYVGDLCGGVMLAAQVFIVSPQYHWFVQECNCSIYPGTITGHRMAWDMMMNWSPTATMILAYVMRVPEVIIDIVSGAHWGVMFGLAYFSMQGAWAKVIVILLLAAGVDAGTTTVGGAVARSTNVIAGVFSHGPQQNIQLINTNGSWHINRTALNCNDSLNTGFLAALFYTNRFNSSGCPGRLSACRNIEAFRIGWGTLQYEDNVTNPEDMRPYCWHYPPKPCGVVPARSVCGPVYCFTPSPVVVGTTDRRGVPTYTWGENETDVFLLNSTRPPQGSWFGCTWMNSTGFTKTCGAPPCRTRADFNASTDLLCPTDCFRKHPDATYIKCGSGPWLTPKCLVHYPYRLWHYPCTVNFTIFKIRMYVGGVEHRLTAACNFTRGDRCDLEDRDRSQLSPLLHSTTEWAILPCTYSDLPALSTGLLHLHQNIVDVQYMYGLSPAITKYVVRWEWVVLLFLLLADARVCACLWMLILLGQAEA

SEQ ID NO: 33 - protein, 561 aa, Hepatitis C virus
LEWRNTSGLYVLTNDCSNSSIVYEADDVILHTPGCVPCVQDDNTSTCWTPVTPTVAVRYVGATTASIRSHVDLLVGAATLCSALYVGDMCGAVFLVGQAFTFRPRRHQTVQTCNCSLYPGHVSGHRMAWDMMMNWSPAVGMVVAHILRLPQTLFDILAGAHWGILAGLAYYSMQGNWAKVAIVMIMFSGVDAETYVTGGSVAHSARGLTSLFSMGAKQKLQLVNTNGSWHINSTALNCNESINTGFIAGLFYYHKFNSTGCPQRLSSCKPIISFRQGWGPLTDANITGPSDDRPYCWHYAPRPCSVVPASSVCGPVYCFTPSPVVVGTTDIKGKPTYNWGENETDVFLLESLRPPSGRWFGCAWMNSTGFLKTCGAPPCNIYGGEGDPENETDLFCPTDCFRKHPEATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSDMVGWALKWEFVILVFLLLADARVCVALWLMLMVSQAEA

SEQ ID NO: 34 - protein, 555 aa, Hepatitis C virus
VNYRNVSGIYHVTNDCPNSSIVYEADHHIMHLPGCVPCVREGNQSRCWVALTPTVAAPYIGAPLESLRSHVDLMVGAATVCSGLYIGDLCGGLFLVGQMFSFRPRRHWTTQDCNCSIYTGHITGHRMAWDMMMNWSPTTTLVLAQVMRIPTTLVDLLSGGHWGVLVGVAYFSMQANWAKVILVLFLFAGVDAETHVSGAAVGRSTAGLANLFSSGSKQNLQLINSNGSWMINRTALNCNDSLNTGFLASLFYTHKFNSSGCSERLACCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSAYLWMMFMVSQVEA

SEQ ID NO: 35 - protein, 556 aa, Hepatitis C virus
VPYRNASGVYHVTNDCPNSSIVYEAEDLILHAPGCVPCVRQGNVSRCWVQITPTLSAPSLGAVTAPLRRAVDYLAGGAALCSALYVGDACGAVFLVGQMFTYSPRRHNVVQDCNCSIYSGHITGHRMAWDMMMNWSPTTALVMAQLLRIPQVVIDIIAGAHWGVLFAAAYYASAANWAKVVLVLFLFAGVDANTRTVGGSAAQGARGLASLFTPGPQQNLQLINTNGSWHINRTALNCNDSLQTGFVAGLLYYHKFNSTGCPQRMASCRPLAAFDQGWGTISYAAVSGPSDDKPYCWHYPPRPCGIVPARGVCGPVYCFTPSPVVVGTTDRKGNPTYSWGENETDIFLLNNTRPPTGNWFGCTWMNSTGFVKTCGAPPCNLGPTGNNSLKCPTDCFRKHPDATYTKCGSGPWLTPRCLVHYPYRLWHYPCTLNYTIFKVRMYIGGLEHRLEVACNWTRGERCDLEDRDRAELSPLLHTTTQWAILPCSFTPTPALSTGLIHLHQNIVDTQYLYGLSSSIVSWAVKWEYIVLAFLLLADARICTCLWIMLLVCQAEA

SEQ ID NO: 36 - protein, 561 aa, Hepatitis C virus
LTYGNSSGLYHLTNDCPNSSIVLEADAMILHLPGCLPCVRVNNNQSICWHAVSPTLAIPNASTPATGFRRHVDLLAGAAVVCSSLYIGDLCGSLFLAGQLFTFQPRRHWTVQDCNCSIYTGHVTGHRMAWDMMMNWSPTTTLVLSSILRVPEICASVIFGGHWGILLAVAYFGMAGNWLKVLAVLFLFAGVEATTTIGHQVGRTTGGLASLFSIGPRQNLQLINTNGSWHINRTALNCNDSLQTGFITSLFYAKNVNSSGCPERMAACKPLADFRQGWGQITYKVNISGPSDDRPYCWHYAPRPCDVVSARTVCGPVYCFTPSPVVVGTTDKLGIPTYNWGENETDVFMLESLRPPTGGWFGCTWMNSTGFTKTCGAPPCQIVPGDYNSSANELLCPTDCFRKHPEATYQRCGSGPWITPRCLVDYPYRLWHYPCTVNFTLHKVRMFVGGIEHRFDAACNWTRGERCDLHDRDRIEMSPLLFSTTQLAILPCSFSTMPALSTGLIHLHQNIVDVQYLYGVSSSVTSWVVKWEYIVLVFLVLADARICTCLWLMLLITNVEA

SEQ ID NO: 37 - protein, 559 aa, Hepatitis C virus
LTYGNSSGLYHLTNDCSNSSIVLEADAMILHLPGCLPCVRVGNQSTCWHAVSPTLATPNASTPATGFRRHHDLLAGAAVVCSSLYIGDLCGSLFLAGQLFAFQPRRHWTVQDCNCSIYTGHVTGHKMAWDMMMNWSPTTTLVLSSILRVPEICASVIFGGHWGILLAVAYFGMAGNWLKVLAVLFLFAGVEAQTMIAHGVSQTTSGFASLLTPGAKQNIQLINTNGSWHINRTALNCNDSLQTGFLASLFYTHKFNSSGCPERMAACKPLAEFRQGWGQITHKNVSGPSDDRPYCWHYAPRPCEVVPARSVCGPVYCFTPSPVVVGTTDKRGNPTYTWGENETDVFHLESLRPPTGGWFGCTWMNSTGFTKTCGAPPCQIVPGNYNSSANELLCPTDCFRKHPEATYQRCGSGPWVTPRCLVDYAYRLWHYPCTVNFTLHKVRMFVGGTEHRFDVACNWTRGERCELHDRNRIEMSPLLFSTTQLSILPCSFSTMPALSTGLIHLHQNIVDVQYLYGVSTNVTSWVVKWEYIVLMFLLLADARICTCLWLMLLISTVEA

SEQ ID NO: 38 - protein, 500 aa, Artificial Sequence
  Description of Artificial Sequence Synthetic consensus sequence
XXXXNSGXXYHVTNDCXNSSIVYEAXXXILHXPGCVPCVRGNNXXSRCWVVPTXXXXXXXXXXXXASTLRXXHDLLVGXXXACSALYVGDLCGXXFLGXXXXXQFPRRHXXXVQDCNCSIYPPTGHTGHRMAWDMMMNXWSPTVARXXXXXXXXXXXXXXXXPDGHWGVLGXLAYFXXMGNWAKXXXVVLLFAGVDATXXXXXXXXXXXGTGLSXLFGXXXXXXQQLINTNGSWWINRTALNCNDSLTGXXXFALFYXHKFNSSGCPERXXXXXXXXACFQGWGPYNGPSXXXXXXXDRPYCWHYXPRPCGVVPAXXVCGPVYCFTPSPVVVGTTDRXXGPTYXWGENETDVFLLNSTRPPXXGWFGCTWMNSTGFTKTCGAPPCGLCPXXXXXXXXXXXXXXTDCFRKHPXATYXXCGSGPWLTPRCLVDYPYRLWHYPCTVNFTXFKVRMVGGXXEHRLXAACNWTRGERCXLEDRDRXELSPLLSTTXXXAILPCSF

SEQ ID NO: 39 - RNA, 542 nt, Canine hepacivirus
gaaacauaauugcuuuugcuccuacgguguggguuagacucguuuuccucacucauuucuucggucugcugcuccaacaggaugcuguggaucgaaauuaugaguuugagauguacggaucaaccuacuccguaaacccacuugacuuaccagcaauaauuuauaagcuccauggcccugaggccuuugaucuuacaaacuauucuccuuacgagguccagaggguggccgcggcgcuccagaaguuggggucaccuccgcuucgggcuuggaagcgcagagcuaagcucgugcgcucuaagcucaaggugcggggaggccgcuacuccguugucgcggacuaccuuuucggcuucgcuucuccuuacaaacaaagaggccugcaccucccggugucaauacgauugacgugucuggaugguuuuccauuggagaugacuccauuggggacauuuacaggcaguuaccgcuugucacugggagguggaucccucuucuccuuugcuuccuuuauuggcugcaauccuuuacuucaaaaaaaa

SEQ ID NO: 40 - DNA, 394 bp, Artificial Sequence
  Description of Artificial Sequence Synthetic polynucleotide
ccuccgugucaggcacggugcguuggcagcguuuugcgcuugcaugcgcuccacgcgucguccaacgcggagggaacuucacaucaccaugugucacucccccuauggaggguuccacccgcuuacacggaaauggguuaaccauacccaaaguacggguaugcggguccuccuagggcccccccggcaggucgagggagcuggaauucgugaauucgugaguacacgaaaaucgcggcuugaacgucuuugaccuucggagccgaaauuugggcgugccccacgaaggaaggcgggggcgguguugggccgccgcccccuuuaucccacggucugauaggaugcuugcgagggcaccugccggucucguagaccataggacatgagtaataaa

SEQ ID NO: 41 - DNA, 23 bp, Artificial Sequence
  Description of Artificial Sequence Synthetic primer
tccacctatggtaagttcttagc

SEQ ID NO: 42 - DNA, 19 bp, Artificial Sequence
  Description of Artificial Sequence Synthetic primer
accctgtcataagggcgtc

SEQ ID NO: 43 - DNA, 23 bp, Artificial Sequence
  Description of Artificial Sequence Synthetic primer
cctatggtaagttcttagctgac

SEQ ID NO: 44 - DNA, 20 bp, Artificial Sequence
  Description of Artificial Sequence Synthetic primer
cctgtcataagggcgtccgt

SEQ ID NO: 45 - DNA, 19 bp, Artificial Sequence
  Description of Artificial Sequence Synthetic primer
gccatagcacagactccac

SEQ ID NO: 46 - DNA, 21 bp, Artificial Sequence
  Description of Artificial Sequence Synthetic primer
gacggaaacatccaaaccccg

SEQ ID NO: 47 - DNA, 27 bp, Artificial Sequence
  Description of Artificial Sequence Synthetic primer
gagggatccatggctggtaaacagccc

SEQ ID NO: 48 - DNA, 30 bp, Artificial Sequence
  Description of Artificial Sequence Synthetic primer
gagctcgagtcaagggcctgtgttaggtgc

SEQ ID NO: 49 - DNA, 27 bp, Artificial Sequence
  Description of Artificial Sequence Synthetic primer
gagggatccaacaccactacagggtca

SEQ ID NO: 50 - DNA, 30 bp, Artificial Sequence
  Description of Artificial Sequence Synthetic primer
gagctcgagtcaatccagtggggtcaatct

SEQ ID NO: 51 - DNA, 23 bp, Artificial Sequence
  Description of Artificial Sequence Synthetic primer
tccacctatggtaagttcttagc

SEQ ID NO: 52 - DNA, 19 bp, Artificial Sequence
  Description of Artificial Sequence Synthetic primer
accctgtcataagggcgtc

SEQ ID NO: 53 - DNA, 23 bp, Artificial Sequence
  Description of Artificial Sequence Synthetic primer
cctatggtaagttcttagctgac

SEQ ID NO: 54 - DNA, 20 bp, Artificial Sequence
  Description of Artificial Sequence Synthetic primer
cctgtcataagggcgtccgt

SEQ ID NO: 55 - DNA, 19 bp, Artificial Sequence
  Description of Artificial Sequence Synthetic primer
gccatagcacagactccac

SEQ ID NO: 56 - DNA, 21 bp, Artificial Sequence
  Description of Artificial Sequence Synthetic primer
gacggaaacatccaaaccccg

SEQ ID NO: 57 - protein, 559 aa, Hepatitis C virus
YEVRNSSGVYHLTNDCPNASIVYETDNAILHEPGCVPCVREGNTSRCWEPVAPTLAVRYRGALTDDLRTHIDLVVASATLCSALYVGDICGAIFIASQAVLWKPGGGRIVQDCNCSIYPGHVTGHRMAWDMMQNWAPALSMVAAYAVRVPGVIITTVAGGHWGVLFGLAYFGMAGNWAKVILIMLLMSGVDAETMAVGARAAHTTGALVSLLNPGPSQRLQLINTNGSWHINRTALNCNDSLQTGFIAALFYTHKRNSSGCPERMASCKPLSDFDQGWGPLWYNSTERPSDQRPYCWHYAPAPCGIVPAKDVCGPVYCFTPSPVVVGTTDRRGVPTYTWGENESDVFLLNSTRPPQGSWFGCSWMNTTGFTKTCGGPPCKIRPQGAQSNTSLTCPTDCFRKHPRATYSACGSGPWLTPRCMVHYPYRLWHYPCTVNFTIHKVRLYIGGVEHRLDAACNWTRGERCDLEDRDRVDMSPLLHSTTELAILPCSFVPLPALSTGLIHLHQNIVDAQYLYGLSPAIISWAIRWEWVVLVFLLLADARICACLWMMMLMAQAEA

SEQ ID NO: 58 - protein, 12 aa, Hepatitis C virus
FAGVDAETEVTG

SEQ ID NO: 59 - protein, 11 aa, Hepatitis C virus
SQAEAALENIV

SEQ ID NO: 60 - protein, 12 aa, Hepatitis C virus
PQRAYALDTEVA

SEQ ID NO: 61 - protein, 12 aa, Hepatitis C virus
KGWRLLAPITAY

SEQ ID NO: 62 - protein, 11 aa, Hepatitis C virus
DLEVVTSTWVL

SEQ ID NO: 63 - protein, 12 aa, Hepatitis C virus
DEMEECSQHLPY

SEQ ID NO: 64 - protein, 12 aa, Hepatitis C virus
ECTTPCSGSWLR

SEQ ID NO: 65 - protein, 10 aa, Hepatitis C virus
VVCCSMSYSW

SEQ ID NO: 66 - protein, 12 aa, Hepatitis C virus
AAGVDAQTETVG

SEQ ID NO: 67 - protein, 11 aa, Hepatitis C virus
GQAEAALEKIV

SEQ ID NO: 68 - protein, 12 aa, Hepatitis C virus
PQQAYAYDASVH

SEQ ID NO: 69 - protein, 12 aa, Hepatitis C virus
KGWSLLAPITAY

SEQ ID NO: 70 - protein, 11 aa, Hepatitis C virus
DLEVMTSTWVL

SEQ ID NO: 71 - protein, 12 aa, Hepatitis C virus
DEMEECASRAAL

SEQ ID NO: 72 - protein, 12 aa, Hepatitis C virus
DCPIPCSGSWLR

SEQ ID NO: 73 - protein, 12 aa, Hepatitis C virus
DSVVCCSMSYSW

SEQ ID NO: 74 - protein, 12 aa, Hepatitis C virus
FSGVDAHYETTG

SEQ ID NO: 75 - protein, 11 aa, Hepatitis C virus
SQTEAALENIV

SEQ ID NO: 76 - protein, 12 aa, Hepatitis C virus
PQRAYAWSGEDS

SEQ ID NO: 77 - protein, 12 aa, Hepatitis C virus
MGWRLLAPITAY

SEQ ID NO: 78 - protein, 11 aa, Hepatitis C virus
DLEVTTSTWVL

SEQ ID NO: 79 - protein, 12 aa, Hepatitis C virus
DEMEECSQAAPY

SEQ ID NO: 80 - protein, 12 aa, Hepatitis C virus
DYPSPCSDDWLR

SEQ ID NO: 81 - protein, 12 aa, Hepatitis C virus
QSVVCCSMSYSW

SEQ ID NO: 82 - protein, 12 aa, Hepatitis C virus
FAGVDAETEVSG

SEQ ID NO: 83 - protein, 11 aa, Hepatitis C virus
SQVEAALSNII

SEQ ID NO: 84 - protein, 12 aa, Hepatitis C virus
PERAYAYDQEVA

SEQ ID NO: 85 - protein, 12 aa, Hepatitis C virus
KGWRLLAPITAY

SEQ ID NO: 86 - protein, 11 aa, Hepatitis C virus
DLEVVTSTWVL

SEQ ID NO: 87 - protein, 12 aa, Hepatitis C virus
DEMEECSKHLPL

SEQ ID NO: 88 - protein, 12 aa, Hepatitis C virus
DCSTPCAESWLW

SEQ ID NO: 89 - protein, 10 aa, Hepatitis C virus
VVCCSMSYSW

SEQ ID NO: 90 - protein, 12 aa, Hepatitis C virus
FAGVDGRTETVG

SEQ ID NO: 91 - protein, 11 aa, Hepatitis C virus
CQAEATCKNVI

SEQ ID NO: 92 - protein, 12 aa, Hepatitis C virus
PHRALALDSSDG

SEQ ID NO: 93 - protein, 12 aa, Hepatitis C virus
SGWRLLAPITAY

SEQ ID NO: 94 - protein, 11 aa, Hepatitis C virus
DLEVITSTWVL

SEQ ID NO: 95 - protein, 12 aa, Hepatitis C virus
DEMEECSASLPY

SEQ ID NO: 96 - protein, 12 aa, Hepatitis C virus
DYSTPCDGTWLR

SEQ ID NO: 97 - protein, 11 aa, Hepatitis C virus
NVVCCSMSYTW

SEQ ID NO: 98 - protein, 12 aa, Hepatitis C virus
FAGVEAQTNIAH

SEQ ID NO: 99 - protein, 11 aa, Hepatitis C virus
STVEAAVERIV

SEQ ID NO: 100 - protein, 12 aa, Hepatitis C virus
PPRAYAMDNEQA

SEQ ID NO: 101 - protein, 12 aa, Hepatitis C virus
GGWKLLAPITAY

SEQ ID NO: 102 - protein, 11 aa, Hepatitis C virus
DLEVITSTWVL

SEQ ID NO: 103 - protein, 12 aa, Hepatitis C virus
DEMEECSRHIPY

SEQ ID NO: 104 - protein, 12 aa, Hepatitis C virus
DTATPCATSWLR

SEQ ID NO: 105 - protein, 11 aa, Hepatitis C virus
DVVCCSMSYSW

SEQ ID NO: 106 - protein, 12 aa, Hepatitis C virus
MSGVDAETNAVG

SEQ ID NO: 107 - protein, 11 aa, Hepatitis C virus
AQAEAALENII

SEQ ID NO: 108 - protein, 12 aa, Hepatitis C virus
PPRAYALDREVS

SEQ ID NO: 109 - protein, 12 aa, Hepatitis C virus
MGWQLLAPISAY

SEQ ID NO: 110 - protein, 11 aa, Hepatitis C virus
DLEVTTSTWVI

SEQ ID NO: 111 - protein, 12 aa, Hepatitis C virus
DEMEECSKAPEL

SEQ ID NO: 112 - protein, 12 aa, Hepatitis C virus
DYSAPCAGSWLR

SEQ ID NO: 113 - protein, 10 aa, Hepatitis C virus
VICCSMSYSW

SEQ ID NO: 114 - protein, 12 aa, Canine hepacivirus
VSFGEASVVRNG

SEQ ID NO: 115 - protein, 12 aa, Canine hepacivirus
CLLLLAYSGLVD

SEQ ID NO: 116 - protein, 12 aa, Canine hepacivirus
ALQAEAYLSDTM

SEQ ID NO: 117 - protein, 12 aa, Canine hepacivirus
PHRAWAFDNASA

SEQ ID NO: 118 - protein, 12 aa, Canine hepacivirus
KGWRLLSPITAT

SEQ ID NO: 119 - protein, 11 aa, Canine hepacivirus
EIDTQTNAWVI

SEQ ID NO: 120 - protein, 12 aa, Canine hepacivirus
DEMEECFDHHQY

SEQ ID NO: 121 - protein, 12 aa, Canine hepacivirus
ENDQNCDFTAIY

SEQ ID NO: 122 - protein, 8 aa, Canine hepacivirus
SCSLSYSW

SEQ ID NO: 123 - protein, 12 aa, Hepatitis GB virus B
CPCSGARVTDPD

SEQ ID NO: 124 - protein, 12 aa, Hepatitis GB virus B
MLYIEATSGNPI

SEQ ID NO: 125 - protein, 12 aa, Hepatitis GB virus B
FVPMAAGLPLTF

SEQ ID NO: 126 - protein, 12 aa, Hepatitis GB virus B
VTPASAFDTEII

SEQ ID NO: 127 - protein, 12 aa, Hepatitis GB virus B
DGWAITAPFTLQ

SEQ ID NO: 128 - protein, 12 aa, Hepatitis GB virus B
FTEVNTSGTAAL

SEQ ID NO: 129 - protein, 12 aa, Hepatitis GB virus B
EIVEECASFIPL

SEQ ID NO: 130 - protein, 12 aa, Hepatitis GB virus B
PTEDDCGLIAWG

SEQ ID NO: 131 - protein, 9 aa, Hepatitis GB virus B
FSCSMSYTW

SEQ ID NO: 132 - RNA, 23 nt, Unknown
  Description of Unknown miRNA-122 oligonucleotide
uggagugugacaaugguguuugu

SEQ ID NO: 133 - DNA, 9289 bp, Hepatitis C virus
cagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtatccttggcccctctatggcaatgagggctgcgggtgggcgggatggctcctgtctccccgtggctctcggcctagttggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaactcctcggggctttaccatgtcaccaatgattgccctaactcgagtattgtgtacgaggcggctgatgccatcctgcacactccggggtgtgtcccttgcgttcgcgagggtaacgcctcgaggtgttgggtgccggtaacccccacggtggccaccagggacggcaaactccccacaacgcagctacgacgtcacatcgatctgcttgtcgggggtgccaccctctgttcggccctctacgtgggggacctgtgcgggtctgtcttccttgtcggtcagctgttcaccttctctcccaggctccactggacgacgcaagactgcaattgttctatctatcccggccatataacgggtcaccgcatggcatgggatatgatgatgaactggtcccccacgacggcgttggtagtagctcagctgctccggatcccacaagccatcttggacatgatcgctggtgctcactggggagtcctggcgggcatagcgtatttttccatggtggggaactgggcgaaggtcctggtagtgctgctgctatttgccggcgtcgacgcagaaacccacgtcaccgggggaagcgccgccaaggctgcgtttggacttactagtttcttcagacgaggtgccaggcagaacatccagctgatcaacgccagcggcagttggcacatcaataggacggccttgaactgcaatgagagcctcgacaccggctgggtagcagggcttatctattaccacaaattcaactcttcaggctgtcccgagaggttggccagctgccgacccatcacccattttgaccagggctggggccctatcagccatgccaacgtaagcagccccgaccaacgcccctactgctggcactaccccccaaaaccttgtggtattgtgcccgcaaggacagtgtgtggcccggtgtattgcttcactcccagccctgtggtggtgggaacgaccgacaggtcgggcgcgcctacttacaactggggtgcaaatgaaacggacgttttcgtccttaacaacaccaggccaccgctgggcaattggttcggttgcacctggatgaactcgtccggattcaccaaagtgtgcggagcgcccccctgtgccatcggaggggggggcaacaacaccttgcattgccccactgattgtttccgcaaacatccggaagctacatactctcggtgcggctccggtccctggattacacccaggtgcctggtcgactacccatataggctttggcattacccttgtaccatcaactacaccatatttaaagtcaggatgtacgtgggaggggtcgagcacaggctggacgctgcttgcaactggacgcggggcgaacgttgtgatctggaagacagggacaggtccgagctcagcccgttgctgctgtccaccacacagtggcaggtcctcccgtgttcctttacgaccctgccagccttgtccactggcctcatccacctccaccagaacattgtggacgtgcagtacctgtacggggtggggtcaagcatcgtgtcctgggccattaagtgggaatacgtcgttctcctgttccttctgcttgcagacgcgcgcgtctgctcctgcttgtggatgatgttactcatatcccaagtggaggcggctttggagaacctcgtaatactcaatgcagcatccctggccgggacgcatggccttgtgtccttcctcgtgttcttctgctttgcgtggtatctgaagggtaagtgggtgcccggagcggtttacgccctctatgggatgtggcctctcttcctgctcctgttggcgttgccccagcgggcatacgcgctggacacggaggtggccgcgtcgtgtggcggcgttgttcttgtcgggttaatggcgctgactctgtcaccatattacaagcgctatatcagctggtgcttatggtggcttcagtattttctgaccagagtagaagcgcaactgcacgtgtgggttccccccctcaacgtccgaggggggcgcgacgctgtcatcttactcatgtgtgtagtacacccgactctgacatttgacatcaccaaacagctgctggccgtcttcggacccctttggattcttcaagccagtctgctcaaagtgccctactttgtgcgcgttcaaggcctcctccggatctgcgcgctagcgcggaagatggtcggaggccattacgtgcaaatggccatcatcaagttgggggcgcttactggcacctatgtttacaaccatctcacccctcttcgggactgggcgcacaacggcctgcgagatctggccgtggctgtagagccagtcgtcttctcccgaatggagaccaagctcatcacgtggggggcagacaccgctgcgtgcggtgatatcatcaacggcttgcccgtctccgcccgtaggggccgggagatactgctcggaccagccgacggaatggtctccaagggttggaggttgctggcgcccatcacggcgtacgcccagcagacaaggggcctcctagggtgtataatcaccagcctgactggccgggataaaaaccaagtggagggtgaggtccagattgtgtcaactgctgcccaaactttcctggcaacgtgcatcaatggggtatgctggactgtctaccacggagctggaacgaggaccatcgcatcacccaagggtcctgtcatccaaatgtacaccaatgtagataaagacctcgtgggctggcccgctcctcagggcagccgctcactgacaccctgcacctgcggctcctcggacctttacctggtcacgaggcacgccgatgtcattcccgtgcgccggcggggtgatagcaggggcagcctgctctcgccccggcccatctcttacttgaagggctcctcgggaggtccgctgttgtgccccgcgggacacgccgtaggcatattcagggctgcggtgtgcacccgtggtgtggctaaggcggtggactttatccctgtggagaacctagagacaaccatgaggtccccggtgttcacagacaattcctccccaccagcagtgccacagagcttccaggtggcccacctgcatgctcccacaggcagcggcaagagcaccaaggtcccggctgcatatgcagcccagggctacaaggtgctagtgctcaacccctctgttgctgcaacgctgggctttggtgcttacatgtccaaggcccatggggtcgatcctaacatcaggaccggggtaagaacaattaccactggcagccccattacgtattccacctacggcaagttccttgccgatggcgggtgctcagggggtgcttatgacataataatctgtgacgagtgccactccacggatgccacatccatcttgggcatcggcactgtccttgaccaagcagagactgcgggggcgagactggttgtgctcgccaccgctacccctccgggctccgtcactgtgccccatcctaacatcgaggaggtcgctctgtccaccaccggagagatccctttttacggcaaggctattcccctcgaggtgatcaagggggggagacatctcatcttctgccactcaaagaagaagtgcgacgagctcgccgcaaagctggtcgcactgggcatcaatgccgtggcctactaccgcggtcttgacgtgtccgtcattccgaccagcggcgatgttgtcgtcgtggcaactgatgctctcatgaccggctataccggcgacttcgactcggtgatagactgcaacacgtgtgtcacccagacagtcgatttcagccttgaccctaccttcaccattgagacaactacgcttccccaagatgctgtctcccgcactcaacgtcggggcaggactggcagggggaagccaggcatctacagatttgtggcaccgggggaacgcccctccggcatgttcgactcgtctgtcctctgcgagtgctatgacgcgggctgtgcttggtatgagctcacgcccgccgagactacagttaggctacgagcgtacatgaacaccccggggcttcccgtgtgccaggaccatcttgaattttgggagggcgtctttacgggtctcacccatatagatgcccacttcttatctcagacaaagcagagcggggaaaactttccttacctggtagcgtaccaagccaccgtgtgcgctagggctcaagcccctcccccatcgtgggaccagatgtggaagtgcttaacccgcctgaagcccaccctccatgggccaacacccctgctgtacagactgggcgccgttcagaatgaagtcaccctgacgcacccaatcaccaaatacatcatgacatgtatgtcggccgacctggaggtcgtcacgagcacctgggtgctcgttggcggcgtcctggctgctttggctgcgtattgcctgtcaacaggctgcgtggtcatagtgggcaggattgtcttgtccgggaagccggcgatcatacctgacagggaagtactctaccgggagttcgatgagatggaagagtgctctcagcacttaccgtacatcgagcaagggatgatgctcgccgagcagttcaagcagaaggccctcggcctcctgcagaccgcgtcccgccaggcagaggttatcacccctgttgtccagaccaactggcaaaaactcgaggccttctgggcgaagcatatgtggaacttcatcagtgggatacaatacttggcgggcttgtcaacgttgcctggcaaccccgccattgcttcattgatggcttttacagcagctgtcaccagcccactaaccactagccaaaccctcctctttaacatattgggggggtgggtggctgcccagctcgccgcccccggtgccgctaccgcctttgtgggcgctggcttagccggcgccgccatcggcagtgttggactggggaaggtcctcatagacatccttgcagggtatggcgcgggcgtggcgggagctcttgtagcattcaagatcatgagcggtgaggtcccctctacggaggacctggtcaacctactacccgccatcctctcgcctggagccctcgtagtcggtgtggtctgtgcagcaatactgcgccggcacgttggcccgggcgagggggcagtgcaatggatgaaccggctcatagccttcgcctcccgggggaaccatgtttcccccacgcactacgtgccggagagcgatgcagctgcccgcgtcactgccatactcagcagcctcactgtaacccagctcctgaggcgactgcaccagtggataagctcggagtgtaccactccgtgctccggttcctggctgagggacatctgggactggatatgcgaggtgctgagcgactttaagacctggctaaaagccaagctcatgccacaactgcctgggattcccttcgtgtcctgccagcgcgggtataaaggggcctggcggggggacggcatcatgcacactcgctgccactgtggagctgagatcactggacatgtcaaaaacgggacgatgaggatcgtcggtcccaagacctgcaggaacatgtggagtggaaccttccccattaacgcctacacaacgggcccctgcaccccccttcctgcgccgaactatacgttcgcgctgtggagggtatctgcagaggaatacgtggagataaggcaagtgggggacttccactacgtgacgggtatgactactgacaatcttaaatgcccgtgccaggtcccatcgcccgaatttttcacagagttggacggggtgcgcctacacaggtttgcgcccccctgcaagcccttgctgcgggaggaggtatcatttagagtaggactccacgagtacccggtagggtcgcaattaccttgcgagcctgaaccggacgtggccgtgttgacgtccatgctcactgatccctcccatataacagcagaggcggccgggagaaggttagcgagggggtcacccccctctgtggccagctcctcggctagccagttgtccgctccatctctcaaggcaacttgcaccgccaatcatgactcccctgacgccgagctcatagaggctaacctcctgtggaggcaggagatgggcggcaacatcaccagggtcgagtcagagaacaaagtggtgattctggactccttcgatccgcttgtggcggaggaggatgagcgggagatctccgtacccgcagaaatcctgcggaagtctcggagattcgccccggccctgcccatctgggcgcggccggactacaaccccccgctattggagacgtggaaaaagcctgactacgaaccacctgtggtccatggctgcccgcttccacctccacagtctcctcctgtgcctccacctcggaagaagcggacggtggtcctcaccgaatcaaccgtatctactgccttggccgagcttgccaccaaaagttttggcagctcctcaacttccggtattgcggctgacaatacgacaatatcctctgagcccgcctcttctggttgccccccggactccgacgctgagtcctattcttccatgccccccctggagggggagcctggggatccggatctcagcgacgggtcgtggtcgacggtcagtagtgaggccggcacagaggatgtcgtgtgctgctcaatgtcctattcctggacaggcgcactcatcaccccgtgcgccgcggaagaacagaaactgcccatcaacgcactgagcaactcgttgctacgtcaccacaatctggtgtattccaccacttcacgcagtgcttgccaaaggcagaagaaagtcacatttgacagactgcaagtcctggacagccattaccaggacgtgcttaaggaggttaaagcagcggcgtcaaaagtgaaggccaatttgctatccgtagaggaagcttgtagcctgacgcccccacactcagccaaatccaagtttggctatggggcaaaagacgtccgttgccatgccagaaaggccgtaaaccacatcaactccgtgtggaaagaccttctggaagacagtgtaacaccaatagacaccaccatcatggctaagaacgaggttttctgcgttcagcctgagaaggggggtcgtaagccagctcgtctcatcgtgttccccgacttgggtgtgcgcgtgtgcgagaagatggccctgtacgacgtggttagcaaactccccctggccgtgatgggaagctcctacggattccaatactctccaggacagcgggttgaattcctcgtgaaagcgtggaagtccaagaagaccccaatggggttttcgtatgatacccgctgctttgactccacagtcactgagagcgatatccgtacggaagaggcaatctaccaatgttgtgacctggacccccaagcccgcgtggccatcaagtccctcactgagaggctttatgttgggggccctcttaccaattcaaggggggaaaactgcggctatcgcaggtgccgcgcgagcggcgtactgacaactagctgtggtaacaccctcacttgctacatcaaggcccgggcagcctgtcgagccgcagggctccaggactgcaccatgctcgtgtgtggcgacgacctagtcgttatctgtgaaagtgcgggagtccaggaggacgcggcgagcctgagagccttcacggaggctatgaccaggtactccgccccccccggggaccccccacaaccagaatacgacttggagctcataacatcatgctcctccaacgtgtcagtcgcccacgacggcgctggaaaaagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcaacttgaacaggcccttgattgcgagatctacggcgcctgctactccatagaaccattggatctacctccaatcattcaaagactccatggccttagcgcattttcactccatagttactctccaggtgaaatcaatagggtggccgcatgcctcaggaaacttggggtcccgcccttgcgagcttggagacaccgggcccggagcgtccgcgctaggcttctgtccagaggaggcagggctgccatatgtggcaagtacctcttcaactgggcagttagaacaaagctcaaactcactccaatagcggccgctggtcagctggacttgtccggctggtttacggctggctacagcgggggagacatttatcacagcgtgtctcatgcccggccccgctggttctggttttgcctactcctgcttgctgcaggggtaggcatctacctcctccccaaccgatgaaggttggggtaaacactcc

SEQ ID NO: 134 - DNA, 9188 bp, Canine hepacivirus; identical to SEQ ID NO: 1

SEQ ID NO: 135 - protein, 9 aa, Unknown
  Description of Unknown Non-primate hepacivirus G1-073-GBX2 peptide isolated from horses
TKKKKKKKK

SEQ ID NO: 136 - protein, 5 aa, Unknown
  Description of Unknown Non-primate hepacivirus B10-022-GBX2 peptide isolated from horses
ERKEK

(19) United States
(12) Patent Application Publication
(10) Pub. No.: US 2014/0128447 A1
(43) Pub. Date: May 8, 2014

(54) NOVEL NON-PRIMATE HEPACIVIRUS

(71) Applicant: The Trustees Of Columbia University In The City Of New York, New York, NY (US)

(72) Inventors: Amit KAPOOR, New York, NY (US); W. Ian LIPKIN, New York, NY (US)

(73) Assignee: The Trustees Of Columbia University In The City Of New York, New York, NY (US)

(21) Appl. No.: 13/905,065

(22) Filed: May 29, 2013

Related U.S. Application Data
(63) Continuation-in-part of application No. PCT/US11/62575, filed on Nov. 30, 2011.
(60) Provisional application No. 61/418,249, filed on Nov. 30, 2010.

(51) Int. Cl. C07K 14/005; C07K 16/10

(52) U.S. Cl. CPC C07K 14/005; C07K 16/10
USPC 514/44 A; 536/23.72; 530/350; 530/387.9; 435/5; 536/24.5

(57) ABSTRACT

The invention is directed to immunogenic compositions and methods for inducing an immune response against Non-Primate Hepacivirus in an animal. In another aspect, the invention relates to antibodies that bind Non-Primate Hepacivirus polypeptides. In yet another aspect, the invention relates to methods for preventing, or reducing NPHV infection in an animal.

This application is a continuation-in-part of International Application No. PCT/US2011/62575, filed Nov. 30, 2011, which claims priority to U.S. Provisional Patent Application No. 61/418,249 filed Nov. 30, 2010, the contents of each of which are hereby incorporated by reference in their entireties.

This invention was made with government support under AI090196, AI081132, AI079231, AI57158, AI070411, AI090055, and AI072613 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### BACKGROUND

The Non-Primate Hepacivirus (NPHV) virus described herein has not been previously isolated. There is a need for immunogenic compositions and treating NPHV infection in animals. This invention addresses these needs.

### SUMMARY OF THE INVENTION

The invention relates to Non-Primate Hepacivirus (NPHV), a novel and highly diverted species of hepacivirus, and isolated nucleic acids sequences and peptides thereof. The invention is also related to antibodies against antigens derived from NPHV sequences and method for generating such antibodies. The invention is also related to immunogenic compositions for inducing an immune response against NPHV in an animal.

In one aspect, the invention provides an isolated nucleic acid having a sequence of any of SEQ ID NO: 1 or SEQ ID NO: 3-10.

In another aspect, the invention provides an isolated nucleic acid comprising 10 consecutive nucleotides having a sequence of any of SEQ ID NO: 1 or SEQ ID NO: 3-10.

In still a further aspect, the invention provides an isolated nucleic acid which is a variant of any of SEQ ID NO: 1 or SEQ ID NO: 3-10 and has at least about 60% identity to any of SEQ ID NO: 1 or SEQ ID NO: 3-10. In one embodiment, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10. In one embodiment, the identity is determined by analysis with a sequence comparison algorithm. Protein and/or nucleic acid sequence identities may be evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993). In one embodiment, the sequence comparison algorithm is FASTA version 3.0t78 using default parameters.

In another aspect, the invention provides an isolated nucleic acid complementary to a sequence of any of SEQ ID NO: 1 or SEQ ID NO: 3-10.

In still another aspect, the invention provides an isolated nucleic acid comprising 10 consecutive nucleotides complementary to a sequence of any of SEQ ID NO: 1 or SEQ ID NO: 3-10

In still a further aspect, the invention provides an isolated nucleic acid which is a complementary to a variant of any of SEQ ID NO: 1 or SEQ ID NO: 3-10 and wherein the variant has at least about 60% identity to any of SEQ ID NO: 1 or SEQ ID NO: 3-10. In one embodiment, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10. In one embodiment, the identity is determined by analysis with a sequence comparison algorithm. In one embodiment, the sequence comparison algorithm is FASTA version 3.0t78 using default parameters.

In yet another aspect, the invention provides an isolated polypeptide having a sequence of any of SEQ ID NO: 2 or SEQ ID NO: 11-18.

In still a further aspect, the invention provides an isolated polypeptide comprising 8 consecutive amino acids having a sequence of any of SEQ ID NO: 2 or SEQ ID NO: 11-18.

In yet another aspect, the invention provides an isolated polypeptide which is a variant of any of SEQ ID NO: 2 or SEQ ID NO: 11-18 and has at least about 70% identity to any of SEQ ID NO: 2 or SEQ ID NO: 11-18. In one embodiment, the variant has at least about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 2 or SEQ ID NO: 11-18. In another embodiment, the identity is determined by analysis with a sequence comparison algorithm. In still a further embodiment, the sequence comparison algorithm is FASTA version 3.0t78 using default parameters.

In yet another aspect, the invention provides an isolated diagnostic antibody that specifically binds to a polypeptide encoded by the nucleotide sequence shown in of any of SEQ ID NO: 1 or SEQ ID NO: 3-10.

In still another aspect, the invention provides an isolated diagnostic antibody that specifically binds to a polypeptide having the sequence of any of SEQ ID NO: 2 or SEQ ID NO: 11-18. In one embodiment, the diagnostic antibody is a polyclonal antibody. In another embodiment, the diagnostic antibody is a monoclonal antibody.

In yet another aspect, the invention provides an oligonucleotide probe comprising from about 10 nucleotides to about 50 nucleotides, wherein at least about 10 contiguous nucleotides are at least 95% complementary to a nucleic acid target region within a nucleic acid sequence of any of SEQ ID NO: 1 or SEQ ID NO: 3-10. In one embodiment, the probe is at least about 95.5%, about 96%, about 96.5%, about 97%, about 97.5%, about 98%, about 98.5%, about 99%, about 99.5% or about 99.9% complementary to any of SEQ ID NO: 1 or SEQ ID NO: 3-10. In still a further embodiment, the oligonucleotide probe consists essentially of from about 10 to about 50 nucleotides.

In another aspect, the invention provides a synthetic nucleic acid which has a sequence consisting of from about 10 to about 30 consecutive nucleotides from a nucleic acid sequence selected from the group consisting of: any of SEQ ID NO: 1 or SEQ ID NO: 3-10.

In still a further aspect, the invention provides a method for determining the presence or absence of NPHV in a biological sample, the method comprising: a) contacting nucleic acid from a biological sample with at least one primer which is a synthetic nucleic acid which has a sequence consisting of from about 10 to about 30 consecutive nucleotides from a nucleic acid sequence selected from the group consisting of: any of SEQ ID NO: 1 or SEQ ID NO: 3-10, b) subjecting the nucleic acid and the primer to amplification conditions, and c) determining the presence or absence of amplification product, wherein the presence of amplification product indicates the presence of RNA associated with NPHV in the sample.

In still a further aspect, the invention provides a synthetic nucleic acid which has a sequence consisting of from about 10 to about 30 consecutive nucleotides from a nucleic acid sequence which is complementary to a nucleic acid sequence selected from the group of sequences consisting of any of SEQ ID NO: 1 or SEQ ID NO: 3-10.

In still a further aspect, the invention provides a method for determining the presence or absence of NPHV in a biological sample, the method comprising: a) contacting nucleic acid from a biological sample with at least one primer which is a synthetic nucleic acid which has a sequence consisting of from about 10 to about 30 consecutive nucleotides from a nucleic acid sequence selected from the group consisting of: any of SEQ ID NO: 1 or SEQ ID NO: 3-10, b) subjecting the nucleic acid and the primer to amplification conditions, and c) determining the presence or absence of amplification product, wherein the presence of amplification product indicates the presence of RNA associated with NPHV in the sample.

In still a further aspect, the invention provides a primer set for determining the presence or absence of NPHV in a biological sample, wherein the primer set comprises at least one synthetic nucleic acid sequence of a) a synthetic nucleic acid which has a sequence consisting of from about 10 to about 30 consecutive nucleotides from a nucleic acids sequence selected from the group of sequences consisting of any of SEQ ID NO: 1 or SEQ ID NO: 3-10, and b) a synthetic nucleic acid which has a sequence consisting of from about 10 to about 30 consecutive nucleotides from a nucleic acids sequence which is complementary to a nucleic acid sequence selected from the group of sequences consisting of any of SEQ ID NO: 1 or SEQ ID NO: 3-10.

In still another aspect, the invention provides a method for determining whether or not a sample contains NPHV, the method comprising: a) contacting a biological sample with an antibody that specifically binds a polypeptide encoded by the nucleic sequence acid of any of SEQ ID NO: 1 or SEQ ID NO: 3-10, and b) determining whether or not the antibody binds to an antigen in the biological sample, wherein binding indicates that the biological sample contains NPHV. In one embodiment, the determining comprises use of a lateral flow assay or ELISA.

In still another aspect, the invention provides a method for determining whether or not a biological sample has been infected by NPHV, the method comprising: a) determining whether or not a biological sample contains antibody that specifically binds a polypeptide encoded by the nucleic sequence acid of any of SEQ ID NO: 1 or SEQ ID NO: 3-10.

In a further aspect, the invention provides an interfering RNA (iRNA) comprising a sense strand having at least 15 contiguous nucleotides complementary to the anti-sense strand of a gene from a virus comprising a nucleic acid sequence selected from the group of sequences consisting of any of SEQ ID NO: 1 or SEQ ID NO: 3-10.

In another aspect, the invention provides an interfering RNA (iRNA) comprising an anti-sense strand having at least 15 contiguous nucleotides complementary to the sense strand of gene from a virus comprising a nucleic acid sequence selected from the group of sequences consisting of any of SEQ ID NO: 1 or SEQ ID NO: 3-10.

In still another aspect, the invention provides a method for reducing the levels of a viral protein, viral mRNA or viral titer in a cell in an animal comprising: administering an iRNA agent to an animal, wherein the iRNA agent comprises a sense strand having at least 15 contiguous nucleotides complementary to gene from a NPHV comprising a nucleic acid sequence selected from the group of sequences consisting of any of SEQ ID NO: 1 or SEQ ID NO: 3-10 and an antisense strand having at least 15 contiguous nucleotides complementary to the sense strand. In one embodiment, the method further comprises co-administering a second iRNA agent to the animal, wherein the second iRNA agent comprises a sense strand having at least 15 or more contiguous nucleotides complementary to second gene from the NPHV comprising a nucleic acid sequence selected from the group of sequences consisting of any of SEQ ID NO: 1 or SEQ ID NO: 3-10 and an antisense strand having at least 15 or more contiguous nucleotides complementary to the sense strand.

In another aspect, the invention provides a method of reducing the levels of a viral protein from at least one gene of a NPHV in a cell in an animal, the method comprising administering an iRNA agent to an animal, wherein the iRNA agent comprises a sense strand having at least 15 or more contiguous nucleotides selected from the group of sequences consisting of any of SEQ ID NO: 1 or SEQ ID NO: 3-10 complementary to a gene from a NPHV and an antisense strand having at least 15 or more contiguous nucleotides complementary to the sense strand of a nucleic acid sequence selected from the group of sequences consisting of any of SEQ ID NO: 1 or SEQ ID NO: 3-10.

In one embodiment, the sample used in conjunction with any of the methods described herein is from a canine.

In one embodiment, the sample used in conjunction with any of the methods described herein is from a canine.

In yet another aspect, the invention provides an isolated virus comprising of the nucleic acid sequences of any of SEQ ID NO: 1 or SEQ ID NO: 3-10.

In still another aspect, the invention provides an isolated virus comprising a polypeptide encoded by the nucleic sequence acid of any of SEQ ID NO: 1 or SEQ ID NO: 3-10.

In one aspect, the invention provides a NPHV immunogenic composition comprising a NPHV nucleic acid. In one embodiment, the NPHV nucleic acid is a nucleic acid sequence of any of SEQ ID NO: 1 or SEQ ID NO: 3-10. In another embodiment, the NPHV nucleic acid comprises least 24 consecutive nucleic acids of any of SEQ ID NO: 1 or SEQ ID NO: 3-10. In still another embodiment, the NPHV nucleic acid is substantially identical to the nucleic acid sequence of any of SEQ ID NO: 1 or SEQ ID NO: 3-10. In still a further embodiment, the NPHV nucleic acid is a variant of any of SEQ ID NO: 1 or SEQ ID NO: 3-10 having at least about 60% identity to any of SEQ ID NO: 1 or SEQ ID NO: 3-10. In one embodiment, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10.

In yet another aspect, the invention provides a NPHV immunogenic composition comprising a NPHV polypeptide. In one embodiment, the NPHV polypeptide is a polypeptide encoded by any of SEQ ID NO: 1 or SEQ ID NO: 3-10. In yet another embodiment, the NPHV polypeptide is a polypeptide encoded by a nucleic acid comprising least 24 consecutive nucleic acids of any of SEQ ID NO: 1 or SEQ ID NO: 3-10. In still a further embodiment, the NPHV polypeptide is a polypeptide encoded by a nucleic acid that is substantially identical to the nucleic acid sequence of any of SEQ ID NO: 1 or SEQ ID NO: 3-10. In still a further embodiment, the NPHV polypeptide is a polypeptide encoded by a nucleic acid that is a variant of any of SEQ ID NO: 1 or SEQ ID NO: 3-10 having at least about 60% identity to any of SEQ ID NO: 1 or SEQ ID NO: 3-10. In still a further embodiment, the variant has at least about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10. In yet another embodiment, the NPHV polypeptide is a polypeptide comprising the amino acid sequence of any of SEQ ID NO: 2 or SEQ ID NO: 11-18. In yet another embodiment, the NPHV polypeptide is a polypeptide comprising least 8 consecutive amino acids of any of SEQ ID NO: 2 or SEQ ID NO: 11-18. In still a further embodiment, the NPHV polypeptide is substantially identical to the amino acid sequence of any of SEQ ID NO: 2 or SEQ ID NO: 11-18. In still another embodiment, the NPHV polypeptide is a variant of any of SEQ ID NO: 2 or SEQ ID NO: 11-18 and having at least about 70% identity to any of SEQ ID NO: 2 or SEQ ID NO: 11-18. In still a further embodiment, the variant has at least about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 2 or SEQ ID NO: 11-18.

In another aspect, the invention provides an antibody that binds a NPHV or a NPHV polypeptide and inhibits, neutralizes or reduces the function or activity of the NPHV or NPHV polypeptide. In one embodiment, the antibody is a polyclonal antibody. In another embodiment, the antibody is a monoclonal antibody. In still another embodiment, the antibody is an IgM antibody. In yet another embodiment, the antibody is a chimeric antibody.

In another aspect, the invention provides an immunogenic composition comprising a killed virus comprising a NPHV polypeptide. In still another aspect, the invention provides an immunogenic composition comprising an attenuated virus comprising a NPHV polypeptide. In one embodiment, any of the immunogenic compositions described herein further comprise at least one excipient, additive or adjuvant. In one embodiment, any of the immunogenic compositions described herein further comprise at least one polypeptide, or fragment thereof, from an additional virus.

In another aspect, the invention provides an immunogenic composition comprising a fusion polypeptide, wherein the fusion polypeptide comprises a NPHV polypeptide, a fragment, of a variant thereof and at least one polypeptide, or fragment thereof, from an additional virus.

In another aspect, the invention provides a method of inducing an immune response in an animal, the method comprising administering any NPHV immunogenic composition described herein.

In another aspect, the invention provides a method for preventing, or reducing NPHV infection in an animal, the method comprising administering any NPHV immunogenic composition described herein.

In another aspect, the invention provides a method for preventing, or reducing NPHV infection in an animal, the method comprising administering to the animal any antibody described herein.

In one embodiment, the method of any administration in the methods described herein is oral administration, immersion administration or injection administration.

In yet another aspect, the invention provides for use of any of the immunogenic compositions described herein for the treatment of condition NPHV infection in an animal.

In yet another aspect, the invention provides for use of any of the immunogenic compositions described herein for preventing or reducing a condition NPHV infection in an animal.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 shows in situ hybridization of NPHV RNA in canine liver. FIG. 1A: Uninfected liver. FIGS. 1B and 1C: Infected liver. Top, Middle, and Bottom represent fluorescent, bright-field, and superimposed images, respectively (bright red dots indicate probe bound to NPHV genomic RNA; blue is hematoxylin counterstain).

FIG. 2 shows a structural and functional map of the NPHV genome. FIG. 2A: Structural protein cleavage is mediated by cellular signal peptidase (black triangle); NS2-NS3 cleavage is mediated by the NS2-NS3 autoprotease (white triangle); and cleavage of other nonstructural proteins is mediated by NS3-NS4A protease complex (gray triangles). FIG. 2B Amino acid sequence divergence scan of NPHV polyprotein, HCV genotypes, and GBV-B. FIG. 2C: Amino acid sequence of different viruses adjacent to predicted protease cleavage sites (10 aa on each side are shown). FIG. 2D Reported functional role of different proteins in the virus life cycle. Show are the sequences of HCV1a (SEQ ID NO: 19), HCV2a (SEQ ID NO: 20), HCV3a (SEQ ID NO: 21), HCV4a (SEQ ID NO: 22), HCV5a (SEQ ID NO: 23), HCV6a (SEQ ID NO: 24), and HCV7a (SEQ ID NO: 25).

FIG. 3 shows the sequence and secondary structure of NPHV 5′ UTR (SEQ ID NO: 26). Bases conserved among different hepaciviruses are shown with different colored circles. The miR-122 binding sites and different internal ribosome entry site stems are labeled according to previously reported hepacivirus 5′ UTR structures (Honda et al., 1996).

FIG. 4 A-F shows sequence alignment of envelope proteins E1 and E2 of NPHV (SEQ ID NO: 28), GB virus B (GBV-B) (SEQ ID NO: 27), HCV genotypes 1a through 7a (SEQ ID NOs: 29-37) and a consensus sequence (SEQ ID NO: 38). Cysteine and asparagine residues are highlighted in yellow and green, respectively. Cysteines experimentally determined to form disulfide bridges in HCV E2 are shown in blue boxes, and blue numbers indicate disulfide connectivity (Krey et al., 2010). Predicted N-glycosylation sites in E1 and experimentally determined sites in E2 are shown in red boxes (Whidby et al., 2009).

FIG. 5 shows RNA folding prediction with the thermodynamic folding energy minimization algorithm (MFOLD) of the terminal 540 nt of the NPHV coding sequence (SEQ ID NO: 39). Base positions are numbered according to the HCV H77 numbering reference sequence.

FIG. 6 shows phylogenetic analysis of conserved regions in the helicase (motifs I-VI) (FIG. 6A) and RdRp (FIG. 6B) genes of NPHV aligned with representative members of the Hepacivirus, Pegivirus (GBV viruses A, C, and D), Pestivirus, and Flavivirus genera. Translated amino acid sequences were aligned with the program ClustalW. Trees were constructed by neighbor joining of pairwise amino acid distances with the program MEGA5 (according to the distance scale provided). Bootstrap resampling was used to determine robustness of branches; values of ≧70% (from 1,000 replicates) are shown. Regions compared corresponded to positions 3697-4477 (helicase domain of NS3) and 7705-8550 (RdRp in NSSB; numbered according to the AF011751 HCV genotype 1a reference sequence).

FIG. 7 shows evolutionary analysis. Bayesian MCMC estimation of the TMRCA for the HCV strains, GBV-B, and NPHV. Maximum Glade credibility phylogeny of representative members of HCV (HCV 1: NC_{—}004102; HCV 2: NC_{—}009823; HCV 3: NC_{—}009824; HCV 4: NC_{—}009825; HCV 5: NC_{—}009826; and HCV 6: NC_{—}009827), hepatitis GBV-B (NC_{—}001655), and NPHV-01. TMRCAs were calculated by calibration with evolutionary rates estimated for NS5B based on HCV subtypes 1a and 1b (4) (FIG. 7A) and HCV subtype 6 (5) (FIG. 7B). The mean TMRCAs with associated 95% highest probability densities for each node are shown to the left of the node, and the Bayesian posterior probabilities are given to the right. The scale bars are in units of years before present (ybp). A listing of virus abbreviations and original accession numbers for each sequence are provided in Table 2.

FIG. 8 shows results of LIPS assay used to identify the natural host of NPHV. The left panel shows anti-NPHV helicase IgG antibody titers in serum samples of different animal species. The right panel shows heat-map of serum samples reactivity against NPHV and HCV helicase proteins.

FIG. 9 shows phylogenetic analysis, genetic composition and genome wide divergence scanning of the eight NPHV genomes. FIG. 9A shows neighbor-joining trees of nucleotide sequences from different genome regions. Trees were constructed from maximum composite likelihood pairwise distances calculated using the program MEGA version 5 (Tamura et al., 2011); datasets were bootstrap re-sampled 500 times to indicate robustness of branching (values≧70% shown on branches). The HCV genotype 1a sequence, M62321, was used as an outgroup (not shown). FIG. 9B shows Mean nucleotide pairwise distances (uncorrected, y-axis) and ratios of synonymous to non-synonymous Jukes-Cantor distances (dN/dS) between horse derived hepaciviruses in different genome regions (red bars). These values were compared with equivalent calculations for human Pegivirus (Stapleton et al., 2010) (HPgV) (blue bars) and HCV (green bars). FIG. 9C shows amino acid sequence divergence across the genome of horse-derived NPHV sequences (top) and comparison with HCV and HPgV (middle and bottom)) using 300 base fragments incrementing by 9 bases across each virus alignment (mid-point plotted on y-axis). Genome diagrams above each graph show gene boundaries using the same x-axis scale as the divergence graph.

FIG. 10 shows RNA structure analysis of NPHV 5′UTR and complete genomes (SEQ ID NO: 40). FIG. 10A shows predicted RNA structure for the 5′UTR of xHV based on minimum free energy predictions and comparison with homologous sequences of HCV and GBV-B (Kapoor et al., 2011a). Stem-loops numbered as in reference −28 (Honda et al., 1996). Sequences homologous to targets of miRNA-122 (Jopling et al., 2005) are indicated by heavy line. FIG. 10B-C shows secondary structure prediction for NPHV genome sequences using mean MFED differences (y-axis) of 200 and 250 base fragments (30 base increment; mid-point plotted on x-axis) for NPHV and the 8 horse-derived hepacivirus sequences (FIG. 10B) and Analysis of the 8 NPHV sequences be ALIFOLD by default parameters (see Wobus et al., 2006) for explanation of color coding). Due to restriction in the server, this figure was built as a composite of 6 separate overlapping 2000 base fragments incrementing by 1500 bases (FIG. 10C).

FIG. 11 shows graphical phylogenetic representation of NPHV and Hepacivirus relatedness. Phylogenetic analysis confirms the identity of the Non-Primate Hepacivirus (NPHV) as a novel member of family Flaviviridae. NPHV sequences described herein are genetically similar to human hepatitis C virus.

FIG. 12A-C Neighbor-joining trees of nucleotide sequences from different genome regions. Trees were constructed from maximum composite likelihood pairwise distances calculated using the program MEGA version 5 (6); datasets were bootstrap re-sampled 500 times to indicate robustness of branching (values≧70% shown on branches). The HCV genotype 1a sequence, M62321, was used as an outgroup.

FIG. 13 shows phylogenetic analysis of Novel Hepaciviruses. Until recently, hepatitis C virus (HCV, purple) and GBV-B (light blue) were the only members of the hepacivirus genus. NPHV (blue) and the newly discovered rodent hepacivirus (RHV (Kapoor et al. (2013) mBio 4:e00216-13), yellow/orange/red) are new genus members.

FIG. 14 shows NPHV genome organization and untranslated regions (UTRs). The NPHV genome of ˜9538 nt contains one long ORF encoding a polyprotein of 2942aa that is predicted to be cleaved into structural (core, E1, E2), p7 [red] and non-structural (NS2-5B [blue]) proteins. Cleavage sites for cellular proteases (black), the NS2-3 autoprotease (white) and the NS3-4A protease (grey) are indicated with triangles. The NPHV 5′untranslated region (UTR) of 384 nt is slightly longer than for HCV (˜341 nt) and has a similar fold and IRES structure. Only the second miR-122 seed site is conserved. Since miR-122 is conserved in the horse, this could indicate hepatotropism. The NPHV 3′UTR of 328 nt has several poly-nucleo1de tracts and is much longer than for HCV. The terminal ˜100 nt polyU-tract and 3′× region resemble those of HCV, but with considerable sequence difference in the 3′× region. While HCV only has a short variable region upstream of the polyU---tract, NPHV has a polyA---tract, a variable region, a polyU/C-tract and an intermediate conserved region.

FIG. 15 shows prevalence of NPHV and in horses. The prevalence of NPHV (blue) in horse serum was assessed by RT-PCR using conserved primers in the NS3 helicase or 5′UTR regions. The Burbelo 2012 and Lyons 2012 NPHV herds were previously published (Burbelo et al. (2012) J Virol. 86:6171-6178, Lyons et al. (2012) Emerg Infect Dis 18:1976-82). The AU herd was from Alabama; the LA herd was from Louisiana.

FIG. 16 shows persistence of NPHV in horses. Persistence of NPHV (blue) and EPgV (purple) in individual horses over time. Dark colors indicate the presence of virus, light color the absence and intermediate color the time during which seroconversion occurred. Hatched areas indicate time points with no data for the particular horse. It remains to be determined what influences clearance of EPgV. Spontaneous clearance of NPHV was not observed.

FIG. 17 shows NPHV is a hepatotropic virus. NPHV RNA quantities were measured by quantitative RT-PCR in serum, liver and lymph node biopsies, PBMCs and tracheal wash samples from a horse with high NPHV serum titers. High quantities of NPHV RNA was found only in serum and liver. The presence of negative-strand RNA in the liver was confirmed by 5′RACE on (−)RNA, indicating replication of NPHV in horse liver.

FIG. 18 shows NPHV horse serum titers. A quantitative RT-PCR was established for NPHV to determine viral RNA levels in serum. NPHV RNA titers in serum from individual horses (blue) or commercial serum pools (red). GE, genome equivalents. These results indicate a widespread contamination of commercial cell culture horse serum with NPHV.

FIG. 19A-E shows a Non-Primate Hepacivirus (NPHV) nucleic acid sequence isolated from dogs (SEQ ID NO: 1).

FIG. 20A-B shows a Non-Primate Hepacivirus (NPHV) amino acid sequence isolated from dogs (SEQ ID NO: 2).

FIG. 21A-P shows CLUSTALW W alignment the genomes of the novel non-primate hepacivirus virus (NPHV) and published hepatitis C virus genome showing that NPHV is a novel highly divergent hepacivirus.

FIG. 22 shows the nucleic acid sequence of NZP-1-GBX2 (FIG. 22 A-E), a NPHV isolated from horses (SEQ ID NO: 3).

FIG. 23 shows the nucleic acid sequence of G1-073-GBX2 (FIG. 23 A-E), a NPHV isolated from horses (SEQ ID NO: 4).

FIG. 24 shows the nucleic acid sequence of A6-006-GBX2 (FIG. 24 A-E), a NPHV isolated from horses (SEQ ID NO: 5).

FIG. 25 shows the nucleic acid sequence of B10-022-GBX2 (FIG. 25 A-E), a NPHV isolated from horses (SEQ ID NO: 6).

FIG. 26 shows the nucleic acid sequence of F8-068-GBX2 (FIG. 26 A-E), a NPHV isolated from horses (SEQ ID NO: 7).

FIG. 27 shows the nucleic acid sequence of G5-077-GBX2 (FIG. 27 A-E), a NPHV isolated from horses (SEQ ID NO: 8).

FIG. 28 shows the nucleic acid sequence of H10-094-GBX2 (FIG. 28 A-E), a NPHV isolated from horses (SEQ ID NO: 9).

FIG. 29 shows the nucleic acid sequence of H3-H3-GBX-1 (FIG. 29 A-E), a NPHV isolated from horses (any of SEQ ID NO: 1 or SEQ ID NO: 3-100).

FIG. 30 shows the amino acid sequence of NZP-1-GBX2 (FIG. 30 A-B), a NPHV isolated from horses (any of SEQ ID NO: 1 or SEQ ID NO: 3-101).

FIG. 31 shows the amino acid sequence of G1-073-GBX2 (FIG. 31 A-B), a NPHV isolated from horses (any of SEQ ID NO: 1 or SEQ ID NO: 3-102).

FIG. 32 shows the amino acid sequence of A6-006-GBX2 (FIG. 32 A-B), a NPHV isolated from horses (any of SEQ ID NO: 1 or SEQ ID NO: 3-103).

FIG. 33 shows the amino acid sequence of B10-022-GBX2 (FIG. 33 A-B), a NPHV isolated from horses (any of SEQ ID NO: 1 or SEQ ID NO: 3-104).

FIG. 34 shows the amino acid sequence of F8-068-GBX2 (FIG. 34 A-B), a NPHV isolated from horses (any of SEQ ID NO: 1 or SEQ ID NO: 3-105).

FIG. 35 shows the amino acid sequence of G5-077-GBX2 (FIG. 35 A-B), a NPHV isolated from horses (any of SEQ ID NO: 1 or SEQ ID NO: 3-106).

FIG. 36 shows the amino acid sequence of H10-094-GBX2 (FIG. 36 A-B), a NPHV isolated from horses (any of SEQ ID NO: 1 or SEQ ID NO: 3-107).

FIG. 37 shows the amino acid sequence of H3-H3-GBX-1 (FIG. 37 A-B) (any of SEQ ID NO: 1 or SEQ ID NO: 3-108).

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, “NPHV” refers to isolates of the Non-Primate Hepacivirus described herein. Like many other animal viruses, NPHV are capable of cross species infectivity and/or replication in different animal species, including primates. As used herein the name “Non-Primate Hepacivirus” is not meant to imply or indicate that NPHV does not infect primates or that HPHV cannot grow, replicate or be cultivated in primate cells or in an expression system comprising primate proteins.

As used herein the term CHV refers to a canine hepacivirus. Because CHV is a NPHV, the term CHV encompasses NPHV.

As used herein, the term “NPHV” polypeptide includes a NPHV polypeptide, a NPHV polypeptide fragment or a NPHV polypeptide variant, or a polypeptide substantially identical to a NPHV polypeptide.

As used herein, the term “animal” refers to a vertebrate, including, but not limited to a canine (e.g. a dog), an equine (e.g. a horse), a non-primate or a primate (e.g. a human).

As used herein, the term “antibody” refers to an antibody that binds to a NPHV polypeptide, a NPHV polypeptide fragment or a NPHV polypeptide variant, or a polypeptide substantially identical to a NPHV polypeptide and inhibit, neutralize or reduce the activity or function of a NPHV polypeptide or a NPHV. The term antibody specifically excludes diagnostic antibodies which bind a NPHV polypeptide, a NPHV polypeptide fragment or a NPHV polypeptide variant, or a polypeptide substantially identical to a NPHV polypeptide and which do not inhibit, neutralize or reduce the activity or function of the polypeptide or the NPHV.

In one aspect, the invention is directed to expression constructs, for example plasmids and vectors, and isolated nucleic acids which comprise NPHV nucleic acid sequences of any of SEQ ID NO: 1 or SEQ ID NO: 3-10, fragments, complementary sequences, and/or variants thereof.

The NPHV polypeptides and amino acid sequences described herein may be useful for, inter alia, expression of NPHV-encoded proteins or fragments, variants, or derivatives thereof, and generating immunogenic compositions against NPHV.

The nucleic acid sequences and polypeptides described herein may be useful for multiple applications, including, but not limited to, generation of diagnostic antibodies and diagnostic nucleic acids.

In another aspect, the invention is directed to a polypeptide comprising the amino acid sequence of any of SEQ ID NO: 2 or SEQ ID NO: 11-18.

In one aspect, the invention provides an isolated NPHV nucleic acid having the sequence of any of SEQ ID NO: 1 or SEQ ID NO: 3-10, or a fragment thereof.

In another aspect, the invention provides an isolated NPHV nucleic acid which comprises consecutive nucleotides having a sequence of any of SEQ ID NO: 1 or SEQ ID NO: 3-10, or a fragment thereof.

In another aspect, the invention provides an isolated NPHV nucleic acid which comprises consecutive nucleotides having a sequence selected from a variant of any of SEQ ID NO: 1 or SEQ ID NO: 3-10 or a fragment thereof. In one embodiment, the variant has at least about 60% identity to any of SEQ ID NO: 1 or SEQ ID NO: 3-10, or a fragment thereof. In one embodiment of the above aspect of the invention, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10, or a fragment thereof.

In one aspect, the invention provides an isolated NPHV nucleic acid complementary to a NPHV nucleic acid sequence of any of SEQ ID NO: 1 or SEQ ID NO: 3-10, or a fragment thereof.

In another aspect, the invention provides an isolated NPHV nucleic acid which comprises consecutive nucleotides complementary to a NPHV nucleic acid sequence of any of SEQ ID NO: 1 or SEQ ID NO: 3-10, or a fragment thereof.

In other aspects, the invention is directed to expression constructs, for example plasmids and vectors, and isolated nucleic acids which comprise NPHV nucleic acid sequences of any of SEQ ID NO: 1 or SEQ ID NO: 3-10, fragments, complementary sequences, and/or variants thereof.

The nucleic acid sequences and polypeptides described herein may be useful for multiple applications, including, but not limited to, generation of antibodies and generation of immunogenic compositions. For example, in one aspect, the invention is directed to an immunogenic composition comprising a polypeptide encoded by a NPHV nucleic sequence acid of any of SEQ ID NO: 1 or SEQ ID NO: 3-10.

In another aspect, the invention is directed to an immunogenic composition comprising a polypeptide comprising the amino acid sequence of any of SEQ ID NO: 2 or SEQ ID NO: 11-18.

In one aspect, the invention provides an isolated NPHV nucleic acid having the sequence of any of SEQ ID NO: 1 or SEQ ID NO: 3-10, or a fragment thereof.

In another aspect, the invention provides an isolated NPHV nucleic acid which comprises consecutive nucleotides having a sequence of any of SEQ ID NO: 1 or SEQ ID NO: 3-10, or a fragment thereof.

In another aspect, the invention provides an isolated NPHV nucleic acid which comprises consecutive nucleotides having a sequence selected from a variant of any of SEQ ID NO: 1 or SEQ ID NO: 3-10 or a fragment thereof. In one embodiment, the variant has at least about 60% identity to any of SEQ ID NO: 1 or SEQ ID NO: 3-10, or a fragment thereof. In one embodiment of the above aspect of the invention, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10, or a fragment thereof.

In one aspect, the invention provides an isolated NPHV nucleic acid complementary to a NPHV nucleic acid sequence of any of SEQ ID NO: 1 or SEQ ID NO: 3-10, or a fragment thereof.

In another aspect, the invention provides an isolated NPHV nucleic acid which comprises consecutive nucleotides complementary to a NPHV nucleic acid sequence of any of SEQ ID NO: 1 or SEQ ID NO: 3-10, or a fragment thereof.

In another aspect, the invention provides an isolated NPHV nucleic acid which comprises consecutive nucleotides complementary to a NPHV nucleic acid sequence of any of SEQ ID NO: 1 or SEQ ID NO: 3-10, or a fragment thereof. In one embodiment, the variant has at least about 60% identity to any of SEQ ID NO: 1 or SEQ ID NO: 3-10, or a fragment thereof. In one embodiment of the above aspect of the invention, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10, or a fragment thereof.

In another aspect, the invention provides an isolated NPHV nucleic acid having a sequence substantially identical to a NPHV nucleic acid sequence of any of SEQ ID NO: 1 or SEQ ID NO: 3-10, or a fragment thereof.

In another aspect, the invention provides an isolated NPHV nucleic acid having a sequence substantially identical to a sequence complementary to a NPHV nucleic acid sequence of any of SEQ ID NO: 1 or SEQ ID NO: 3-10, or a fragment thereof.

In one aspect, the invention provides an isolated NPHV nucleic acid complementary to a NPHV nucleic acid sequence of any of SEQ ID NO: 1 or SEQ ID NO: 3-10, or a fragment thereof.

In another aspect, the invention provides an isolated NPHV nucleic acid which comprises consecutive nucleotides complementary to a NPHV nucleic acid sequence of any of SEQ ID NO: 1 or SEQ ID NO: 3-10, or a fragment thereof.

In another aspect, the invention provides an isolated NPHV nucleic acid which comprises consecutive nucleotides complementary to a NPHV nucleic acid sequence of any of SEQ ID NO: 1 or SEQ ID NO: 3-10, or a fragment thereof. In one embodiment, the variant has at least about 60% identity to any of SEQ ID NO: 1 or SEQ ID NO: 3-10, or a fragment thereof. In one embodiment of the above aspect of the invention, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10, or a fragment thereof.

In another aspect, the invention provides an isolated NPHV nucleic acid having a sequence substantially identical to a NPHV nucleic acid sequence of any of SEQ ID NO: 1 or SEQ ID NO: 3-10, or a fragment thereof.

In another aspect, the invention provides an isolated NPHV nucleic acid having a sequence substantially identical to a sequence complementary to a NPHV nucleic acid sequence of any of SEQ ID NO: 1 or SEQ ID NO: 3-10, or a fragment thereof.

In another aspect, the invention provides an oligonucleotide probe which comprises from about 10 nucleotides to about 50 nucleotides, wherein at least about 10 contiguous nucleotides are at least 95% complementary to a nucleic acid target region within a NPHV nucleic acid sequence of any of SEQ ID NO: 1 or SEQ ID NO: 3-10, wherein the oligonucleotide probe hybridizes to the nucleic acid target region under moderate to highly stringent conditions to form a detectable nucleic acid target:oligonucleotide probe duplex. In one embodiment, the oligonucleotide probe is at least about 95.5%, about 96%, about 96.5%, about 97%, about 97.5%, about 98%, about 98.5%, about 99%, about 99.5% or about 99.9% complementary to any of SEQ ID NO: 1 or SEQ ID NO: 3-10. In another embodiment the oligonucleotide probe consists essentially of from about 10 to about 50 nucleotides.

Polynucleotides homologous to the sequences illustrated in the SEQ ID NOs 1-10 can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations.

In certain aspects, the invention is directed to primer sets comprising isolated nucleic acids as described herein, which primer set are suitable for amplification of nucleic acids from samples which comprises Non-Primate Hepacivirus represented by any of SEQ ID NO: 1 or SEQ ID NO: 3-10, or variants thereof. Primer sets can comprise any suitable combination of primers which would allow amplification of a target nucleic acid sequences in a sample which comprises Non-Primate Hepacivirus represented by any of SEQ ID NO: 1 or SEQ ID NO: 3-10, or variants thereof. Amplification can be performed by any suitable method known in the art, for example but not limited to PCR, RT-PCR, transcription mediated amplification (TMA).

Hybridization conditions: As used herein, the phrase “stringent hybridization conditions” refers to conditions under which a probe, primer or oligonucleotide will hybridize to its target sequence, and can hybridize, for example but not limited to, variants of the disclosed polynucleotide sequences, including allelic or splice variants, or sequences that encode orthologs or paralogs of presently disclosed polypeptides. The precise conditions for stringent hybridization are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having 60% identity, or about 70% identity, or about 80% or greater identity with disclosed nucleic acid sequences.

Stringent conditions are known to those skilled in the art and can be found in Current Protocols In Molecular Biology, John Wiley & Sons, N.Y. (1989), 6.3.1-10.3.6. In certain embodiments, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× sodium chloride/sodium citrate (SSC), 50 mM Tris-HCl (pH 7.5), 1 nM EDTA, 0.02% PVP, 0.02% Ficoll, 0.02% BSA, and 500 mg/ml denatured canine sperm DNA at 65° C. This hybridization is followed by one or more washes in 0.2×SSC, 0.01% BSA at 50° C. Another non-limiting example of stringent hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equation: DNA-DNA: Tm(° C.)=81.5+16.6(log [Na+])+0.41(% G+C)−0.62(% formamide)−500/L (1) DNA-RNA: Tm(° C.)=79.8+18.5(log [Na+])+0.58(% G+C)+0.12(% G+C)²−0.5(% formamide)−820/L (2) RNA-RNA: Tm(C)=79.8+18.5(log [Na+])+0.58(% G+C)+0.12(% G+C)²−0.35(% formamide)−820/L (3), where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated canine sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments. The stringency can be adjusted either during the hybridization step or in the post-hybridization washes. Salt concentration, formamide concentration, hybridization temperature and probe lengths are variables that can be used to alter stringency. As a general guidelines high stringency is typically performed at Tm-5° C. to Tm-20° C., moderate stringency at Tm-20° C. to Tm-35° C. and low stringency at Tm-35° SC to Tm-50° C. for duplex>150 base pairs. Hybridization may be performed at low to moderate stringency (25-50° C. below Tm), followed by post-hybridization washes at increasing stringencies. Maximum rates of hybridization in solution are determined empirically to occur at Tm-25° C. for DNA-DNA duplex and Tm-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. In certain embodiments, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide. In certain embodiments, stringent temperature conditions will ordinarily include temperatures of at least about 30° C., and in other embodiment at least about 37° C., and in other embodiments at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed. In a certain embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide. In another embodiment, hybridization will occur at 42 C in 250 mM NaCl, 25 mM trisodium citrate, 1% SDS, 50% formamide. Useful variations on these conditions will be readily apparent to those skilled in the art.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 0.1×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C. For identification of less closely related homolog, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art.

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid. It may be desirable to select conditions for a particular assay such that a higher signal to noise ratio, that is, about 15× or more, is obtained. Accordingly, an animal nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

Encompassed by the invention are polynucleotide sequences that are capable of hybridizing to the claimed polynucleotide sequences, including any of the nucleic acid sequences disclosed herein, and fragments thereof under various conditions of stringency (See, for example, Wahl and Berger (1987) Methods Enzymol. 152: 399-407; and Kimmel (1987) Methods Enzymol. 152: 507-511). With regard to hybridization, conditions that are highly stringent, and means for achieving them, are well known in the art. See, for example, Sambrook et al. (1989) “Molecular Cloning: A Laboratory Manual” (2nd ed., Cold Spring Harbor Laboratory); Berger and Kimmel, eds., (1987) “Guide to Molecular Cloning Techniques”, In Methods in Enzymology:152: 467-469; and Anderson and Young (1985) “Quantitative Filter Hybridisation.” In: Hames and Higgins, ed., Nucleic Acid Hybridisation, A Practical Approach. Oxford, IRL Press, 73-111.

### Primers and Probes

The isolated nucleic acid of the invention which can be used as primers and/or probes can comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 consecutive nucleotides from of any of SEQ ID NO: 1 or SEQ ID NO: 3-10, or sequences complementary to of any of SEQ ID NO: 1 or SEQ ID NO: 3-10. The isolated nucleic acid of the invention which can be used as primers and/or probes can comprise from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 and 100 consecutive nucleotides from of any of SEQ ID NO: 1 or SEQ ID NO: 3-10, or sequences complementary to of any of SEQ ID NO: 1 or SEQ ID NO: 3-10. The invention is also directed to primer and/or probes which can be labeled by any suitable molecule and/or label known in the art, for example but not limited to fluorescent tags suitable for use in Real Time PCR amplification, for example TaqMan, cybergreen, TAMRA and/or FAM probes; radiolabels, and so forth. In certain embodiments, the oligonucleotide primers and/or probe further comprises a detectable non-isotopic label selected from the group consisting of: a fluorescent molecule, a chemiluminescent molecule, an enzyme, a cofactor, an enzyme substrate, and a hapten.

In yet a further aspect, the invention provides a synthetic nucleic acid which has a sequence consisting of from about 10 to about 30 consecutive nucleotides from a nucleic acid sequence selected from the group of sequences consisting of any of SEQ ID NO: 1 or SEQ ID NO: 3-10.

In yet a further aspect, the invention provides a synthetic nucleic acid which has a sequence consisting of from about 10 to about 30 consecutive nucleotides from a nucleic acid consisting of consecutive nucleotides having a sequence which is a variant of SEQ ID NOS 1-10 having at least about 95% identity to any of SEQ ID NO: 1 or SEQ ID NO: 3-10. In one embodiment, the variant has at least about 95.5%, about 96%, about 96.5%, about 97%, about 97.5%, about 98%, about 98.5%, about 99%, about 99.5% or about 99.9% identity to any of SEQ ID NO: 1 or SEQ ID NO: 3-10.

In another aspect, the invention provides a composition comprising one or more nucleic acids having a sequence consisting of from about 10 to about 30 consecutive nucleotides from a nucleic acids sequence selected from the group of sequences consisting of any of SEQ ID NO: 1 or SEQ ID NO: 3-10.

In another aspect, the invention provides a composition comprising one or more nucleic acids having a sequence consisting of from about 10 to about 30 consecutive nucleotides from a nucleic acid consisting of consecutive nucleotides having a sequence which is a variant of SEQ ID NOS 1-10 having at least about 95% identity to any of SEQ ID NO: 1 or SEQ ID NO: 3-10. In one embodiment, the variant has at least about 95.5%, about 96%, about 96.5%, about 97%, about 97.5%, about 98%, about 98.5%, about 99%, about 99.5% or about 99.9% identity to any of SEQ ID NO: 1 or SEQ ID NO: 3-10.

In yet another aspect, the invention provides a synthetic nucleic acid which has a sequence consisting of from about 10 to about 30 consecutive nucleotides from a nucleic acid sequence which is complementary to a nucleic acid sequence selected from the group of sequences consisting of any of SEQ ID NO: 1 or SEQ ID NO: 3-10.

In yet another aspect, the invention provides a synthetic nucleic acid which has a sequence consisting of from about 10 to about 30 consecutive nucleotides complementary to a nucleic acid consisting of consecutive nucleotides having a sequence which is a variant of SEQ ID NOS 1-10 having at least about 95% identity to any of SEQ ID NO: 1 or SEQ ID NO: 3-10. In one embodiment, the variant has at least about 95.5%, about 96%, about 96.5%, about 97%, about 97.5%, about 98%, about 98.5%, about 99%, about 99.5% or about 99.9% identity to any of SEQ ID NO: 1 or SEQ ID NO: 3-10.

In yet another aspect, the invention a composition comprising one or more synthetic nucleic acids which have a sequence consisting of from about 10 to about 30 consecutive nucleotides from a nucleic acid sequence which is complementary to a nucleic acid sequence selected from the group of sequences consisting of any of SEQ ID NO: 1 or SEQ ID NO: 3-10.

In yet another aspect, the invention provides a composition comprising one or more synthetic nucleic acids which have a sequence consisting of from about 10 to about 30 consecutive nucleotides complementary to a nucleic acid consisting of consecutive nucleotides having a sequence which is a variant of SEQ ID NOS 1-10 having at least about 95% identity to any of SEQ ID NO: 1 or SEQ ID NO: 3-10. In one embodiment, the variant has at least about 95.5%, about 96%, about 96.5%, about 97%, about 97.5%, about 98%, about 98.5%, about 99%, about 99.5% or about 99.9% identity to any of SEQ ID NO: 1 or SEQ ID NO: 3-10.

In other aspects the invention is directed to isolated nucleic acid sequences such as primers and probes, comprising nucleic acid sequences derived from of any of SEQ ID NO: 1 or SEQ ID NO: 3-10. Such primers and/or probes may be useful for detecting the presence of the NPHV of the invention, for example in samples of bodily fluids such as blood, saliva, or urine from an animal, and thus may be useful in the diagnosis of NPHV infection. Such probes can detect polynucleotides of any of SEQ ID NO: 1 or SEQ ID NO: 3-10 in samples which comprise NPHV represented by any of SEQ ID NO: 1 or SEQ ID NO: 3-10. The isolated nucleic acids which can be used as primer and/probes are of sufficient length to allow hybridization with, i.e. formation of duplex with a corresponding target nucleic acid sequence, a nucleic acid sequences of any of SEQ ID NO: 1 or SEQ ID NO: 3-10, or a variant thereof.

In one embodiment, the invention is directed to an isolated nucleic acid sequence comprising from about 10 to about 50 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 100 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 200 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 300 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 400 consecutive nucleotides from any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 500 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 600 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 700 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 800 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 900 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 1000 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 1500 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 2000 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 2500 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 3000 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 3500 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 3600 consecutive nucleotides from of any of SEQ ID NO: 1 or SEQ ID NO: 3-10 or a sequence complementary to of any of SEQ ID NO: 1 or SEQ ID NO: 3-10. In one embodiment, the invention is directed to an isolated nucleic acid sequence comprising from about 10 to about 3621 consecutive nucleotides from of any of SEQ ID NO: 1 or SEQ ID NO: 3-10 or a sequence complementary to of any of SEQ ID NO: 1 or SEQ ID NO: 3-10.

In a further aspect, the invention provides a primer set for determining the presence or absence of the NPHV in a biological sample, wherein the primer set comprises at least one synthetic nucleic acid sequence of a synthetic nucleic acid which has a sequence consisting of from about 10 to about 30 consecutive nucleotides from a nucleic acids sequence selected from the group of sequences consisting of any of SEQ ID NO: 1 or SEQ ID NO: 3-10, a synthetic nucleic acid which has a sequence consisting of from about 10 to about 30 consecutive nucleotides from a nucleic acids sequence which is complementary to a nucleic acid sequence selected from the group of sequences consisting of any of SEQ ID NO: 1 or SEQ ID NO: 3-10. In one embodiment, the biological sample is derived from an animal suspected of having the NPHV.

In an further aspect, the invention provides a method for determining the presence or absence of a NPHV in a biological sample, the method comprising: a) contacting nucleic acid from a biological sample with at least one primer which is a nucleic acid sequence consisting of from about 10 to about 30 consecutive nucleotides from a nucleic acids sequence which is complementary to a nucleic acid sequence selected from the group of sequences consisting of any of SEQ ID NO: 1 or SEQ ID NO: 3-10, b) subjecting the nucleic acid and the primer to amplification conditions, and c) determining the presence or absence of amplification product, wherein the presence of amplification product indicates the presence of RNA associated with NPHV in the sample. In one embodiment, the biological sample is derived from an animal suspected of having a NPHV.

In another aspect, the invention provides a method for determining the presence or absence of the NPHV in a biological sample, the method comprising: a) contacting nucleic acid from a biological sample with at least one primer which is a synthetic nucleic acid which has a sequence consisting of from about 10 to about 30 consecutive nucleotides from a nucleic acids sequence selected from the group of sequences consisting of any of SEQ ID NO: 1 or SEQ ID NO: 3-10, b) subjecting the nucleic acid and the primer to amplification conditions, and c) determining the presence or absence of amplification product, wherein the presence of amplification product indicates the presence of RNA associated with NPHV in the sample.

In still a further aspect, the invention provides for an interfering RNA (iRNA) comprising a sense strand having at least 15 contiguous nucleotides complementary to a nucleic acid sequence of any of SEQ ID NO: 1 or SEQ ID NO: 3-10.

In still another aspect, the invention provides a method of reducing the levels of a viral protein, viral mRNA or viral titer in a cell in an animal comprising: administering at least one iRNA agent to an animal, wherein the iRNA agent comprising a sense strand having at least 15 contiguous nucleotides complementary to gene from a NPHV comprising any of any of SEQ ID NO: 1 or SEQ ID NO: 3-10 and an antisense strand having at least 15 contiguous nucleotides complementary to the sense strand. In one embodiment, the iRNA agent is administered to an animal. In another embodiment, the iRNA agent is administered via nebulization to an animal. In yet another embodiment, the method further comprises co-administering a second iRNA agent to the animal, wherein the second iRNA agent comprising a sense strand having at least 15 or more contiguous nucleotides complementary to second gene from the NPHV, and an antisense strand having at least 15 or more contiguous nucleotides complementary to the sense strand.

In another aspect, the invention provides a method of reducing the levels of a viral protein in a cell in an animal comprising the step of administering an iRNA agent to an animal, wherein the iRNA agent comprises a sense strand having at least 15 or more contiguous nucleotides complementary to a gene from a NPHV comprising any of SEQ ID NO: 1 or SEQ ID NO: 3-10 and an antisense strand having at least 15 or more contiguous nucleotides complementary to the sense strand.

In certain aspects, the invention is directed to iRNA molecules which target nucleic acids from NPHV, for example but not limited to any of SEQ ID NO: 1 or SEQ ID NO: 3-10, and variants thereof, and silence a target gene.

An “iRNA agent” (abbreviation for “interfering RNA agent”) as used herein, is an RNA agent, which can down-regulate the expression of a target gene, e.g. a NPHV gene. An iRNA agent may act by one or more of a number of mechanisms, including post-transcriptional cleavage of a target mRNA sometimes referred to in the art as RNAi, or pre-transcriptional or pre-translational mechanisms. An iRNA agent can be a double stranded (ds) iRNA agent.

A “ds iRNA agent” (abbreviation for “double stranded iRNA agent”), as used herein, is an iRNA agent which includes more than one, and in certain embodiments two, strands in which interchain hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected, e.g. by a linker, e.g. a polyethyleneglycol linker, to form but one molecule. At least one strand can include a region which is sufficiently complementary to a target RNA. Such strand is termed the “antisense strand”. A second strand comprised in the dsRNA agent which comprises a region complementary to the antisense strand is termed the “sense strand”. However, a ds iRNA agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule.

iRNA agents as described herein, including ds iRNA agents and siRNA agents, can mediate silencing of a gene, e.g., by RNA degradation. For convenience, such RNA is also referred to herein as the RNA to be silenced. Such a gene is also referred to as a target gene. In certain embodiments, the RNA to be silenced is a gene product of a NPHV gene.

As used herein, the phrase “mediates RNAi” refers to the ability of an agent to silence, in a sequence specific manner, a target gene. “Silencing a target gene” means the process whereby a cell containing and/or secreting a certain product of the target gene when not in contact with the agent, will contain and/or secret at least 10%, 20%, 30%, 40%, 50%, 60%, 70%, 80%, or 90% less of such gene product when contacted with the agent, as compared to a similar cell which has not been contacted with the agent. Such product of the target gene can, for example, be a messenger RNA (mRNA), a protein, or a regulatory element.

In the anti viral uses of the present invention, silencing of a target gene can result in a reduction in “viral titer” in the cell or in the animal, wherein “reduction in viral titer” refers to a decrease in the number of viable virus produced by a cell or found in an organism undergoing the silencing of a viral target gene. Reduction in the cellular amount of virus produced can lead to a decrease in the amount of measurable virus produced in the tissues of an animal undergoing treatment and a reduction in the severity of the symptoms of the viral infection. iRNA agents of the present invention are also referred to as “antiviral iRNA agents”.

As used herein, a “NPHV gene” refers to of the genes identified in the NPHV genome.

In other aspects, the invention provides methods for reducing viral titer in an animal, by administering to an animal, at least one iRNA which inhibits the expression of a NPHV gene.

In other aspects, the invention provides methods for identifying and/or generating anti-viral drugs. For example, in one aspect the invention provides methods for identifying drugs that bind to and/or inhibit the function of the NPHV-encoded proteins of the invention, or that inhibit the replication or pathogenicity of the NPHV of the invention. Methods of identifying drugs that affect or inhibit a particular drug target, such as high throughput drug screening methods, are well known in the art and can readily be applied to the proteins and viruses of the present invention.

In one aspect, the invention provides an isolated NPHV polypeptide encoded by a NPHV nucleic acid sequence of any of SEQ ID NO: 1 or SEQ ID NO: 3-10, or a fragment thereof.

The NPHV polypeptides and amino acid sequences described herein may be useful for, inter alia, expression of NPHV-encoded proteins or fragments, variants, or derivatives thereof, generation of diagnostic antibodies against NPHV proteins, generation of primers and probes for detecting NPHV and/or for diagnosing NPHV infection, and screening for drugs effective against Non-Primate Hepaciviruses described herein.

In one embodiment, the NPHV polypeptide fragment can be a polypeptide comprising about 8 consecutive amino acids of a NPHV polypeptide described herein. In another embodiment, the fragment can be a polypeptide comprising about 10 consecutive amino acids of a NPHV polypeptide described herein. In another embodiment, the fragment can be a polypeptide comprising about 14 consecutive amino acids of a NPHV polypeptide described herein. In another embodiment, the fragment can be a polypeptide comprising about 16 consecutive amino acids of a NPHV polypeptide described herein. In another embodiment, the fragment can be a polypeptide comprising about 18 consecutive amino acids of a NPHV polypeptide described herein. In another embodiment, the fragment can be a polypeptide comprising about 20 consecutive amino acids of a NPHV polypeptide described herein. In another embodiment, the fragment can be a polypeptide comprising about 21 or more consecutive amino acids of a NPHV polypeptide described herein.

In yet another embodiment, the NPHV polypeptide fragment can be a polypeptide comprising from about 8 to about 50, about 8 to about 100, about 8 to about 200, about 8 to about 300, about 8 to about 400, about 8 to about 500, about 8 to about 600, about 8 to about 700, about 8 to about 800, about 8 to about 900 or more consecutive amino acids from a NPHV polypeptide.

In another aspect, the invention provides an isolated NPHV polypeptide encoded by a nucleic acid which comprises consecutive nucleotides having a sequence selected from a NPHV nucleic acid sequence of any of SEQ ID NO: 1 or SEQ ID NO: 3-10, or a fragment thereof.

In another aspect, the invention provides an isolated NPHV polypeptide encoded by a nucleic acid which comprises consecutive nucleotides having a sequence selected from a variant of a NPHV nucleic acid sequence of any of SEQ ID NO: 1 or SEQ ID NO: 3-10 or a fragment thereof. In one embodiment, the variant has at least about 60% identity to any of SEQ ID NO: 1 or SEQ ID NO: 3-10, or a fragment thereof. In one embodiment of the above aspect of the invention, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10, or a fragment thereof.

In one aspect, the invention provides an isolated NPHV polypeptide encoded by a nucleic acid complementary a NPHV nucleic acid sequence of any of SEQ ID NO: 1 or SEQ ID NO: 3-10, or a fragment thereof.

In another aspect, the invention provides an isolated NPHV polypeptide encoded by a nucleic acid which comprises consecutive nucleotides a NPHV nucleic acid sequence of any of SEQ ID NO: 1 or SEQ ID NO: 3-10, or a fragment thereof.

In another aspect, the invention provides an isolated NPHV polypeptide encoded by a nucleic acid having a sequence substantially identical to a NPHV nucleic acid sequence of any of SEQ ID NO: 1 or SEQ ID NO: 3-10, or a fragment thereof.

In another aspect, the invention provides an isolated NPHV polypeptide encoded by a nucleic acid having a sequence substantially identical to a sequence complementary to a NPHV nucleic acid sequence of any of SEQ ID NO: 1 or SEQ ID NO: 3-10, or a fragment thereof.

In one aspect, the invention provides an isolated NPHV polypeptide having the sequence of any of SEQ ID NO: 2 or SEQ ID NO: 11-18, or a fragment thereof.

In another aspect, the invention provides an isolated NPHV polypeptide which comprises consecutive amino acids having a sequence of any of SEQ ID NO: 2 or SEQ ID NO: 11-18, or a fragment thereof.

In another aspect, the invention provides an isolated NPHV polypeptide which comprises consecutive amino acids having a sequence selected from a variant of any of SEQ ID NO: 2 or SEQ ID NO: 11-18, or a fragment thereof. In one embodiment, the variant has at least about 70% identity to any of SEQ ID NO: 2 or SEQ ID NO: 11-18, or a fragment thereof. In one embodiment of the above aspect of the invention, the variant has at least about 60%, about 65%, about 70%, about 75%, about 80%, about 85%, about 90%, about 95.5%, about 96%, about 96.5%, about 97%, about 97.5%, about 98%, about 98.5%, about 99%, about 99.5% or about 99.9% identity to that of any of SEQ ID NO: 1 or SEQ ID NO: 3-10, or a fragment thereof.

In another aspect, the invention provides an isolated NPHV polypeptide having a sequence substantially identical to a NPHV amino acid sequence of any of SEQ ID NO: 2 or SEQ ID NO: 11-18, or a fragment thereof.

In one aspect, the invention provides an isolated NPHV polypeptide encoded by a NPHV nucleic acid sequence of any of SEQ ID NO: 1 or SEQ ID NO: 3-10, or a fragment thereof.

In one embodiment, the isolated NPHV polypeptide fragment can be a polypeptide comprising about 8 consecutive amino acids of a NPHV amino acid sequence of any of SEQ ID NO: 2 or SEQ ID NO: 11-18. In another embodiment, the fragment can be a polypeptide comprising about 10 consecutive amino acids of a NPHV amino acid sequence of any of SEQ ID NO: 2 or SEQ ID NO: 11-18. In another embodiment, the fragment can be a polypeptide comprising about 14 consecutive amino acids of a NPHV amino acid sequence of any of SEQ ID NO: 2 or SEQ ID NO: 11-18. In another embodiment, the fragment can be a polypeptide comprising about 16 consecutive amino acids of a NPHV amino acid sequence of any of SEQ ID NO: 2 or SEQ ID NO: 11-18. In another embodiment, the fragment can be a polypeptide comprising about 18 consecutive amino acids of a NPHV amino acid sequence of any of SEQ ID NO: 2 or SEQ ID NO: 11-18. In another embodiment, the fragment can be a polypeptide comprising about 20 consecutive amino acids of a NPHV amino acid sequence of any of SEQ ID NO: 2 or SEQ ID NO: 11-18. In another embodiment, the fragment can be a polypeptide comprising about 21 or more consecutive amino acids of a NPHV amino acid sequence of any of SEQ ID NO: 2 or SEQ ID NO: 11-18.

In yet another embodiment, the isolated NPHV polypeptide fragment can be a polypeptide comprising from about 8 to about 50, about 8 to about 100, about 8 to about 200, about 8 to about 300, about 8 to about 400, about 8 to about 500, about 8 to about 600, about 8 to about 700, about 8 to about 800, about 8 to about 900 or more consecutive amino acids of a NPHV amino acid sequence of any of SEQ ID NO: 2 or SEQ ID NO: 11-18.

In another aspect, the invention provides an isolated NPHV polypeptide which comprises consecutive amino acids having a sequence selected from a NPHV amino acid sequence of any of SEQ ID NO: 2 or SEQ ID NO: 11-18.

In another aspect, the invention provides an isolated NPHV polypeptide which comprises consecutive nucleotides having a sequence selected from a variant a NPHV amino acid sequence of any of SEQ ID NO: 2 or SEQ ID NO: 11-18, or a fragment thereof. In one embodiment, the variant has at least about 70% identity to any of SEQ ID NO: 2 or SEQ ID NO: 11-18, or a fragment thereof. In one embodiment of the above aspect of the invention, the variant has at least about 70%, about 75%, about 80%, about 85%, about 90%, about 95.5%, about 96%, about 96.5%, about 97%, about 97.5%, about 98%, about 98.5%, about 99%, about 99.5% or about 99.9% identity to any of SEQ ID NO: 2 or SEQ ID NO: 11-18, or a fragment thereof.

In another aspect, the invention provides an isolated NPHV polypeptide substantially identical to variant a NPHV amino acid sequence of any of SEQ ID NO: 2 or SEQ ID NO: 11-18, or a fragment thereof.

The NPHV nucleic acid sequences described herein may be useful for, inter alia, expression of NPHV-encoded proteins or fragments, variants, or derivatives thereof, generation of antibodies against NPHV proteins, generating immunogenic compositions against NPHV, and screening for drugs effective against the NPHVs or NPHV amino acids described herein.

In one aspect, the invention provides an isolated NPHV polypeptide encoded by a NPHV nucleic acid sequence of any of SEQ ID NO: 1 or SEQ ID NO: 3-10, or a fragment thereof.

In one embodiment, the NPHV polypeptide fragment can be a polypeptide comprising about 8 consecutive amino acids of a NPHV polypeptide described herein. In another embodiment, the fragment can be a polypeptide comprising about 10 consecutive amino acids of a NPHV polypeptide described herein. In another embodiment, the fragment can be a polypeptide comprising about 14 consecutive amino acids of a NPHV polypeptide described herein. In another embodiment, the fragment can be a polypeptide comprising about 16 consecutive amino acids of a NPHV polypeptide described herein. In another embodiment, the fragment can be a polypeptide comprising about 18 consecutive amino acids of a NPHV polypeptide described herein. In another embodiment, the fragment can be a polypeptide comprising about 20 consecutive amino acids of a NPHV polypeptide described herein. In another embodiment, the fragment can be a polypeptide comprising about 21 or more consecutive amino acids of a NPHV polypeptide described herein.

In one aspect, the invention provides an isolated NPHV polypeptide encoded by a NPHV nucleic acid sequence of any of SEQ ID NO: 1 or SEQ ID NO: 3-10, or a fragment thereof.

In one embodiment, the isolated NPHV polypeptide fragment can be a polypeptide comprising about 8 consecutive amino acids of a NPHV amino acid sequence of any of SEQ ID NO: 2 or SEQ ID NO: 11-18. In another embodiment, the fragment can be a polypeptide comprising about 10 consecutive amino acids of a NPHV amino acid sequence of any of SEQ ID NO: 2 or SEQ ID NO: 11-18. In another embodiment, the fragment can be a polypeptide comprising about 14 consecutive amino acids of a NPHV amino acid sequence of any of SEQ ID NO: 2 or SEQ ID NO: 11-18. In another embodiment, the fragment can be a polypeptide comprising about 16 consecutive amino acids of a NPHV amino acid sequence of any of SEQ ID NO: 2 or SEQ ID NO: 11-18. In another embodiment, the fragment can be a polypeptide comprising about 18 consecutive amino acids of a NPHV amino acid sequence of any of SEQ ID NO: 2 or SEQ ID NO: 11-18. In another embodiment, the fragment can be a polypeptide comprising about 20 consecutive amino acids of a NPHV amino acid sequence of any of SEQ ID NO: 2 or SEQ ID NO: 11-18. In another embodiment, the fragment can be a polypeptide comprising about 21 or more consecutive amino acids of a NPHV amino acid sequence of any of SEQ ID NO: 2 or SEQ ID NO: 11-18.

In yet another embodiment, the isolated NPHV polypeptide fragment can be a polypeptide comprising from about 8 to about 50, about 8 to about 100, about 8 to about 200, about 8 to about 300, about 8 to about 400, about 8 to about 500, about 8 to about 600, about 8 to about 700, about 8 to about 800, about 8 to about 900 or more consecutive amino acids of a NPHV amino acid sequence of any of SEQ ID NO: 2 or SEQ ID NO: 11-18.

In another aspect, the invention provides an isolated NPHV polypeptide which comprises consecutive amino acids having a sequence selected from a NPHV amino acid sequence of any of SEQ ID NO: 2 or SEQ ID NO: 11-18.

In another aspect, the invention provides an isolated NPHV polypeptide which comprises consecutive nucleotides having a sequence selected from a variant a NPHV amino acid sequence of any of SEQ ID NO: 2 or SEQ ID NO: 11-18, or a fragment thereof. In one embodiment, the variant has at least about 70% identity to any of SEQ ID NO: 2 or SEQ ID NO: 11-18, or a fragment thereof. In one embodiment of the above aspect of the invention, the variant has at least about 70%, about 75%, about 80%, about 85%, about 90%, about 95.5%, about 96%, about 96.5%, about 97%, about 97.5%, about 98%, about 98.5%, about 99%, about 99.5% or about 99.9% identity to any of SEQ ID NO: 2 or SEQ ID NO: 11-18, or a fragment thereof.

In another aspect, the invention provides an isolated NPHV polypeptide substantially identical to variant a NPHV amino acid sequence of any of SEQ ID NO: 2 or SEQ ID NO: 11-18, or a fragment thereof.

“Substantially identical,” in the context of two nucleic acids or polypeptides, refers to two or more sequences or subsequences that have at least of at least 98%, at least 99% or higher nucleotide or amino acid residue identity, when compared and aligned for maximum correspondence, as measured using one of the following sequence comparison algorithms or by visual inspection. Thus, in certain embodiments, polypeptides that a substantially identical to the NPHV polypeptides described herein can also be used to generate antibodies that bind to the NPHV polypeptides described herein.

“Percent identity” in the context of two or more nucleic acids or polypeptide sequences, refers to the percentage of nucleotides or amino acids that two or more sequences or subsequences contain which are the same. A specified percentage of amino acid residues or nucleotides can have a specified identity over a specified region, when compared and aligned for maximum correspondence over a comparison window, or designated region as measured using one of the following sequence comparison algorithms or by manual alignment and visual inspection. In one aspect, the invention provides a NPHV polypeptide which is a variant of a NPHV polypeptide and has at least about 70%, about 75%, about 80%, about 85%, about 90%, about 95.5%, about 96%, about 96.5%, about 97%, about 97.5%, about 98%, about 98.5%, about 99%, about 99.5% or about 99.9% identity to a NPHV polypeptide shown in any of SEQ ID NO: 2 or SEQ ID NO: 11-18.

It will be understood that, for the particular NPHV polypeptides described here, natural variations can exist between individual NPHV strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979) Amino acid replacements between related amino 15 acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science, 227, 1435-1441, 1985) and determining the functional similarity between homologous proteins. Such amino acid substitutions of the exemplary embodiments of this invention, as well as variations having deletions and/or insertions are within the scope of the invention as long as the resulting proteins retain their immune reactivity. It is know that polypeptide sequences having one or more amino acid sequence variations as compared to a reference polypeptide may still be useful for generating antibodies that bind the reference polypeptide. Thus in certain embodiments, the NPHV polypeptides and the antibodies and antibody generation methods related thereto encompass NPHV polypeptides isolated from different virus isolates that have sequence identity levels of at least about 90%, while still representing the same NPHV protein with the same immunological characteristics.

The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

An example of an algorithm that is suitable for determining percent sequence identity and sequence similarity is the FASTA algorithm, which is described in Pearson, W. R. & Lipman, D. J., Proc. Natl. Acad. Sci. U.S.A. 85: 2444, 1988. See also W. R. Pearson, Methods Enzymol. 266: 227-258, 1996. Exemplary parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another example of algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www ncbi.nlm.nih.gov/). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of an algorithm that is suitable for multiple DNA and amino acid sequence alignments is the CLUSTALW program (Thompson, J. D. et al., Nucl. Acids. Res. 22:4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89:10915-10919, 1992).

In yet a further aspect, the invention provides a computer readable medium having stored thereon (i) a nucleic acid sequence of a NPHV nucleic acid sequence of any of SEQ ID NO: 1 or SEQ ID NO: 3-10, a sequence substantially identical to a NPHV nucleic acid sequence of any of SEQ ID NO: 1 or SEQ ID NO: 3-10; a sequence variant of a NPHV nucleic acid sequence of any of SEQ ID NO: 1 or SEQ ID NO: 3-10; or (ii) an amino acid sequence encoded by a nucleic acid sequence of a NPHV nucleic acid sequence of any of SEQ ID NO: 1 or SEQ ID NO: 3-10, an amino acid sequence encoded by a sequence substantially identical to a NPHV nucleic acid sequence of any of SEQ ID NO: 1 or SEQ ID NO: 3-10; an amino acid sequence encoded by a sequence variant of a NPHV nucleic acid sequence of any of SEQ ID NO: 1 or SEQ ID NO: 3-10.

The NPHV nucleic acid sequences described herein may be useful for, inter alia, expression of NPHV-encoded proteins or fragments, variants, or derivatives thereof, generation of diagnostic antibodies against NPHV proteins (e.g. for determining whether an animal has been infected with NPHV), generation of primers and probes for detecting NPHV and/or for diagnosing NPHV infection, and screening for drugs effective against Non-Primate Hepaciviruses described herein.

The polypeptides described herein can be used for raising antibodies (e.g. for immunization purposes). In one aspect, the invention provides antibody that binds a NPHV polypeptide, a NPHV polypeptide fragment or a NPHV polypeptide variant, or a polypeptide substantially identical to a NPHV polypeptide and wherein the antibody is a vaccine antibody that inhibits, neutralizes or reduces the activity or function of the polypeptide or a NPHV. In some embodiments, the antibody is a polyclonal antibody, a monoclonal antibody, or a chimeric antibody.

In certain embodiments, the polypeptides of the present invention can be suitable for use as antigens to detect antibodies against NPHV represented by any of SEQ ID NO: 1 or SEQ ID NO: 3-10, and variants thereof. In other embodiments, the polypeptides of the present invention which comprise antigenic determinants can be used in various immunoassays to identify animals exposed to and/or samples which comprise NPHV represented by any of SEQ ID NO: 1 or SEQ ID NO: 3-10, and variants thereof.

In one aspect, the invention provides a diagnostic NPHV antibody that binds a NPHV, a NPHV polypeptide, a NPHV polypeptide fragment or a NPHV polypeptide variant, or a polypeptide substantially identical to a NPHV polypeptide and wherein the antibody is an antibody that binds a NPHV or a NPHV polypeptide but does not inhibit, neutralize or reduce the activity or function of the polypeptide or the NPHV. In some embodiments, the diagnostic antibody is a polyclonal antibody, a monoclonal antibody, or a chimeric antibody.

In another aspect, the invention provides a method for determining whether or not a sample contains a NPHV, the method comprising: (a) providing an immunoassay comprising a diagnostic antibody against a NPHV derived antigen, (b) contacting the diagnostic antibody with a biological sample, (c) detecting binding between antigens in the test sample and the diagnostic antibody. In one embodiment, the immunoassay is a lateral flow assay or ELISA. In one embodiment, the biological sample is derived from an animal suspected of having a NPHV.

In still a further aspect, the invention provides a method for determining whether or not a sample contains antibodies against NPHV, the method comprising: (a) providing an immunoassay comprising an antigen from a NPHV, (b) contacting the antigen with a biological sample, (c) detecting binding between antibodies in the test sample and the antigen.

The diagnostic antibodies of the invention can also be used to purify polypeptides of any polypeptide encoded by the nucleic sequence acid of any of SEQ ID NO: 1 or SEQ ID NO: 3-10, polypeptides comprising the sequence of any of SEQ ID NO: 2 or SEQ ID NO: 11-18, or variants or fragments thereof.

In other embodiments, the diagnostic antibodies of the invention can be used to identify expression and localization of a NPHV polypeptide or variants or fragments thereof. Analysis of expression and localization of NPHV polypeptides, or variants or fragments thereof, can be useful in diagnosing a NPHV infection or for determining potential role of a NPHV polypeptide.

In other embodiments, the antibodies of the present invention can be used in various immunoassays to identify animals exposed to and/or samples which comprise antigens from NPHV.

Any suitable immunoassay which can lead to formation of antigen-antibody complex can also be used. Variations and different formats of immunoassays, for example but not limited to ELISA, lateral flow assays for detection of analytes in samples, immunoprecipitation, are known in the art. In various embodiments, the antigen and/or the antibody can be labeled by any suitable label or method known in the art. For example enzymatic immunoassays may use solid supports, or immunoprecipitation. Immunoassays which amplify the signal from the antigen-antibody immune complex can also be used with the methods described herein.

In certain aspects the invention provides methods for assaying a sample to determine the presence or absence of a NPHV polypeptide, or a fragment or a variant thereof. In certain embodiments, methods for assaying a sample, include, but are not limited to, methods which can detect the presence of nucleic acids, methods which can detect the presence of NPHV polypeptides, methods which can detect the presence of antibodies against NPHV polypeptides, or any polypeptide encoded by a NPHV nucleic acid.

In still a further aspect, the invention provides a NPHV diagnostic kit comprising a NPHV nucleic acid, a NPHV nucleic acid fragment or a NPHV nucleic acid variant, a nucleic acid substantially identical to a NPHV nucleic acid, or a NPHV diagnostic antibody.

One of skill in the art will recognize that when diagnostic antibodies or nucleic acid are used for diagnostic purposes, it is not necessary to use the entire nucleic acid or diagnostic antibody to detect a NPHV or a NPHV polypeptide in an animal or in a sample. In certain aspects, the invention is directed to methods for generating diagnostic antibodies that bind to the NPHV polypeptides described herein by generating antibodies that bind to a fragment of a polypeptide described herein. Thus, in one aspect, the invention relates to diagnostic kits for detecting NPHV infection or the presence of NPHV in a sample, that comprise a NPHV nucleic acid or a NPHV diagnostic antibody.

In still a further aspect, the invention provides a NPHV immunogenic composition comprising a NPHV polypeptide, a NPHV polypeptide fragment or a NPHV polypeptide variant, or a polypeptide substantially identical to a NPHV polypeptide.

As used herein, the term “immunogenic polypeptide” refers to a NPHV polypeptide, or a fragment of a NPHV polypeptide capable of inducing an immune response in a vertebrate host. The term “immunogenic polypeptide” also refers to a NPHV polypeptide, or a fragment of a NPHV polypeptide that can be used to generate an antibody against the NPHV polypeptide, or a fragment of a NPHV polypeptide using other antibody generation techniques known in the art, including, but not limited to, hybridoma, phage display and ribosome display technologies.

In still a further aspect, the invention provides a NPHV immunogenic composition comprising a NPHV nucleic acid, a NPHV nucleic acid fragment or a NPHV nucleic acid variant, a nucleic acid substantially identical to a NPHV nucleic acid, a NPHV polypeptide, a NPHV polypeptide fragment or a NPHV polypeptide variant, or a polypeptide substantially identical to a NPHV polypeptide.

One of skill in the art will recognize that when polypeptides are used for raising antibodies, it is not necessary to use the entire polypeptide to generate an antibody capable of recognizing the full length polypeptide. In certain aspects, the invention is directed to methods for generating antibodies that bind to the NPHV polypeptides described herein by generating antibodies that bind to a fragment of a polypeptide described herein. Thus, in one aspect, the invention relates to immunogenic compositions for combating NPHV infection, that comprise a protein or immunogenic fragments of a NPHV polypeptide. Still another embodiment of the present invention relates to the NPHV proteins described herein or immunogenic fragments thereof. In still another embodiment, the invention relates to the use of the NPHV proteins described herein or immunogenic fragments thereof for combating NPHV infections.

In one embodiment, the NPHV immunogenic compositions described herein are capable of ameliorating the symptoms of a NPHV infection and/or of reducing the duration of a NPHV infection. In another embodiment, the immunogenic compositions are capable of inducing protective immunity against NPHV infection. The immunogenic compositions of the invention can be effective against the NPHV disclosed herein, and may also be cross-reactive with, and effective against, multiple different clades and strains of NPHV, and against other hepaciviruses.

In other aspect, the invention provides a nucleic acid vectors comprising a NPHV nucleic acid sequence, a NPHV nucleic acid fragment or a NPHV nucleic acid variant, or a nucleic acid substantially identical to a NPHV nucleic acid.

In another aspect, the invention provides a nucleic acid vector encoding a NPHV polypeptide, a NPHV polypeptide fragment or a NPHV polypeptide variant, or a polypeptide substantially identical to a NPHV polypeptide. Non-limiting examples of vectors include, but are not limited to retroviral, adenoviral, adeno-associated viral, and lentiviral vectors.

In yet another aspect, the invention provides a host organism comprising a nucleic acid vector encoding a NPHV polypeptide, a NPHV polypeptide fragment or a NPHV polypeptide variant, or a polypeptide substantially identical to a NPHV polypeptide. In one embodiment, the host organism is a prokaryote, a eukaryote, or a fungus. In another embodiment the organism is a canine (e.g. a dog). In another embodiment the organism is equine (e.g. a horse). In another embodiment the organism is a non-primate. In another embodiment the organism is a primate (e.g. a human).

In another aspect, the invention provides a method of inducing an immune response in an animal, the method comprising administering a NPHV nucleic acid, a NPHV polypeptide or a NPHV immunogenic composition to the animal. Methods for administering polypeptides to animals, and methods for generating immune responses in animals by administering immunogenic peptides in immunogenically effective amounts are known in the art.

The polypeptides described herein can be used in the form of a NPHV immunogenic composition to immunize an animal according to any method known in the art. An immunogenic composition can also include attenuated live virus, inactivated (killed) viral vaccines, and subunits. In certain embodiments, NPHVs may be attenuated by removal or disruption of viral sequences whose products cause or contribute to the disease and symptoms associated with NPHV infection, and leaving intact those sequences required for viral replication. In this way an attenuated NPHV can be produced that replicates in animals, and induces an immune response in animals, but which does not induce the deleterious disease and symptoms usually associated with NPHV infection. One of skill in the art can determine which NPHV sequences can or should be removed or disrupted, and which sequences should be left intact, in order to generate an attenuated NPHV. NPHV compositions may also comprise inactivated NPHV, such as by chemical treatment, to “kill” the viruses such that they are no longer capable of replicating or causing disease in animals, but still induce an immune response in an animal. There are many suitable viral inactivation methods known in the art and one of skill in the art can readily select a suitable method and produce an inactivated “killed” NPHV suitable for use as an immunogenic composition.

Methods of purification of polypeptides and of inactivated virus are known in the art and may include one or more of, for instance gradient centrifugation, ultracentrifugation, continuous-flow ultracentrifugation and chromatography, such as ion exchange chromatography, size exclusion chromatography, and liquid affinity chromatography. Additional method of purification include ultrafiltration and dialfiltration. See J P Gregersen “Herstellung von Virussimpfstoffen aus Zellkulturen” Chapter 4.2 in Pharmazeutische Biotechnology (eds. 0. Kayser and R H Mueller) Wissenschaftliche Verlagsgesellschaft, Stuttgart, 2000. See also, O'Neil et al., “Virus Harvesting and Affinity Based Liquid Chromatography. A Method for Virus Concentration and Purification”, Biotechnology (1993) 11:173-177; Prior et al., “Process Development for Manufacture of Inactivated HIV-1”, Pharmaceutical Technology (1995) 30-52; and Majhdi et al., “Isolation and Characterization of a Coronavirus from Elk Calves with diarrhea” Journal of Clinical Microbiology (1995) 35(11): 2937-2942.

Other examples of purification methods suitable for use in the invention include polyethylene glycol or ammonium sulfate precipitation (see Trepanier et al., “Concentration of human respiratory syncytial virus using ammonium sulfate, polyethylene glycol or hollow fiber ultrafiltration” Journal of Virological Methods (1981) 3(4):201-211; Hagen et al., “Optimization of Poly(ethylene glycol) Precipitation of Hepatitis Virus Used to prepare VAQTA, a Highly Purified Inactivated Vaccine” Biotechnology Progress (1996) 12:406-412; and Carlsson et al., “Purification of Infectious Pancreatic Necrosis Virus by Anion Exchange Chromatography Increases the Specific Infectivity” Journal of Virological Methods (1994) 47:27-36) as well as ultrafiltration and microfiltration (see Pay et al., Developments in Biological Standardization (1985) 60:171-174; Tsurumi et al., “Structure and filtration performances of improved cuprammonium regenerated cellulose hollow fiber (improved BMM hollow fiber) for virus removal” Polymer Journal (1990) 22(12):1085-1100; and Makino et al., “Concentration of live retrovirus with a regenerated cellulose hollow fiber, BMM”, Archives of Virology (1994) 139(1-2):87-96.).

Polypeptides and viruses can be purified using chromatography, such as ion exchange, chromatography. Chromatic purification allows for the production of large volumes of virus containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Anion exchange resins that may be used include DEAE, EMD TMAE. Cation exchange resins may comprise a sulfonic acid-modified surface. Viruses can be purified using ion exchange chromatography comprising a strong anion exchange resin (e.g. EMD TMAE) for the first step and EMD-SO₃ (cation exchange resin) for the second step. A metal-binding affinity chromatography step can optionally be included for further purification. (See, e.g., WO 97/06243).

A resin such as Fractogel EMD can also be used This synthetic methacrylate based resin has long, linear polymer chains covalently attached and allows for a large amount of sterically accessible ligands for the binding of biomolecules without any steric hindrance.

Column-based liquid affinity chromatography is another purification method that can be used invention. One example of a resin for use in purification method is Matrex Cellufine Sulfate (MCS). MCS consists of a rigid spherical (approx. 45-105 μm diameter) cellulose matrix of 3,000 Dalton exclusion limit (its pore structure excludes macromolecules), with a low concentration of sulfate ester functionality on the 6-position of cellulose. As the functional ligand (sulfate ester) is relatively highly dispersed, it presents insufficient cationic charge density to allow for most soluble proteins to adsorb onto the bead surface. Therefore the bulk of the protein found in typical virus pools (cell culture supernatants, e.g. pyrogens and most contaminating proteins, as well as nucleic acids and endotoxins) are washed from the column and a degree of purification of the bound virus is achieved.

Inactivated viruses may be further purified by gradient centrifugation, or density gradient centrifugation. For commercial scale operation a continuous flow sucrose gradient centrifugation would be an option. This method can be used to purify antiviral immunogenic compositions and is known to one skilled in the art.

Additional purification methods which may be used to purify viruses of the invention include the use of a nucleic acid degrading agent, a nucleic acid degrading enzyme, such as a nuclease having DNase and RNase activity, or an endonuclease, such as from Serratia marcescens, membrane adsorbers with anionic functional groups or additional chromatographic steps with anionic functional groups (e.g. DEAE or TMAE). An ultrafiltration/dialfiltration and final sterile filtration step could also be added to the purification method.

The purified immunogenic preparations described herein can be substantially free of contaminating proteins derived from the cells or cell culture and can comprise less than about 1000, 500, 250, 150, 100, or 50 pg cellular nucleic acid/μg virus antigen, and less than about 1000, 500, 250, 150, 100, or 50 pg cellular nucleic acid/dose.

In one aspect, immunization of animals may be performed by directly injecting the NPHV polypeptides, fragments or variants thereof into the animal to generate an immunogenic response. In certain embodiments, the NPHV polypeptides can be injected by themselves, or as immunogenic NPHV compositions comprising other components, including, for example, excipients, additives and adjuvants.

To produce the NPHV polypeptides and NPHV antibodies described herein, the NPHV nucleic acid sequences of the invention can be delivered to cultured cells, for example by transfecting cultured cells with plasmids or expression vectors containing NPHV nucleic acid sequences, or by infecting cultured cells with recombinant viruses containing NPHV nucleic acid sequences. NPHV polypeptides may then be expressed in a host cell or expression system and purified. A host cell may be a cell of bacterial origin, e.g. Escherichia coli, Bacillus subtilis and Lactobacillus species, in combination with bacteria-based plasmids as pBR322, or bacterial expression vectors as pGEX, or with bacteriophages. The host cell may also be of eukaryotic origin, e.g. yeast-cells in combination with yeast-specific vector molecules, or higher eukaryotic cells like insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), mammalian cells like Hela cells, Chinese Hamster Ovary cells (CHO) or Crandell Feline Kidney-cells, also with appropriate vectors or recombinant viruses. In vitro expression systems, such as in-vitro transcription and in-vitro translation systems can also be used to generate the NPHV polypeptides described herein. The purified proteins can then be incorporated into compositions suitable for administration to animals. Methods and techniques for expression and purification of recombinant proteins are well known in the art, and any such suitable methods may be used.

Immunization may also be performed by direct immunization with a DNA encoding a NPHV polypeptide. When using such compositions, the nucleic acid is administered to the animal, and the immunogenic polypeptide(s) encoded by the nucleic acid are expressed in the animal, such that an immune response against the proteins or peptides is generated in the animal. Subunit immunogenic compositions may also be proteinaceous immunogenic compositions, which contain the viral proteins or subunits themselves, or portions of those proteins or subunits.

Any suitable plasmid or expression vector capable of driving expression of a polypeptide may be used. Plasmids and expression vectors can include a promoter for directing transcription of the nucleic acid. The nucleic acid sequence encoding NPHV polypeptides may also be incorporated into a suitable recombinant virus for administration to the animal Examples of suitable viruses include, but are not limited to, vaccinia viruses, retroviruses, adenoviruses and adeno-associated viruses. One of skill in the art will be able to select a suitable plasmid, expression vector, or recombinant virus for delivery of the NPHV nucleic acid sequences of the invention. Direct immunization with DNA encoding proteins has been successful for many different proteins. (As reviewed in e.g. Donnelly et al. The Immunologist 2: 20-26 (1993)).

The NPHV antibodies described herein can also be generated using live recombinant carriers capable of expressing the polypeptides described herein. Live recombinant carriers are micro-organisms or viruses in which additional genetic information, e.g. a nucleic acid sequence encoding a NPHV polypeptide, or a fragment thereof has been cloned. Animals infected with such live recombinant carriers will produce an immunological response not only against the immunogens of the carrier, but also against the NPHV polypeptide or NPHV polypeptide fragment.

Alternatively, passive immunization can be performed by raising NPHV antibodies in a first animal species (e.g. a rabbit), from antibody-producing cell lines, or from in-vitro techniques before administering such antibodies (in purified or unpurified form) to second animal species (e.g. a canine). This type of passive immunization can be used when the second animal is already infected with a NPHV. In some cases, passive immunization can be useful where the infection in the second animal cannot, or has not had sufficient time to mount an immune response to the infection.

Many methods for the immunization of animals are known in the art. For example. Immunization with the NPHV nucleic acids and polypeptides described herein can be performed in animals by injection, immersion, dipping or through oral administration. The administration protocol can be optimized in accordance with standard immunization practice

For oral immunization of animals, the NPHV nucleic acids, polypeptides or immunogenic compositions described herein can be mixed with feed, coated on the feed or be administered in an encapsulated form. One skilled in the art will appreciate that these methods of administration may expose an antigen to potential breakdown or denaturation and thus the skill artisan will ensure that the method of immunization will be appropriate for a chosen antigen. In the case of oral immunization, the immunogenic compositions may also be mixed with one or more carriers. Carriers suitable for use in oral immunization include both metabolizable and non-metabolizable substances.

Another method for immunizing animals with the NPHV nucleic acids and polypeptides described herein is by injection immunization. In injection immunization, an immunogenic composition is injected into the abdominal cavity of an animal. In certain embodiments, the NPHV nucleic acids, polypeptides or immunogenic compositions can be delivered into the body cavity of the dog in an oil emulsion, or other adjuvants or additives that enhance and/or prolong immune responses.

The NPHV nucleic acids, polypeptides or immunogenic compositions described herein can also be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges may be formulated containing a powder mix of the compound and a suitable powder base such as lactose or starch.

The NPHV nucleic acids, polypeptides or immunogenic compositions described herein can be administered in any immunologically effective amount sufficient to trigger an immune response in an animal. In certain instances, this amount can be between about 0.01 and about 1000 micrograms of the NPHV nucleic acid, polypeptide or immunogenic composition per animal.

As used herein, the term “immunologically effective amount” refers to an amount capable of inducing, or enhancing the induction of, the desired immune response in an animal. The desired response may include, inter alia, inducing an antibody or cell-mediated immune response, or both. The desired response may also be induction of an immune response sufficient to ameliorate the symptoms of a NPHV infection, reduce the duration of a NPHV infection, and/or provide protective immunity in an animal against subsequent challenge with a NPHV. An immunologically effective amount may be an amount that induces actual “protection” against NPHV infection, meaning the prevention of any of the symptoms or conditions resulting from NPHV infection in animals. An immunologically effective amount may also be an amount sufficient to delay the onset of symptoms and conditions associated with infection, reduce the degree or rate of infection, reduce in the severity of any disease or symptom resulting from infection, and reduce the viral load of an infected animal.

One of skill in the art can readily determine what is an “immunologically effective amount” of the compositions of the invention without performing any undue experimentation. An effective amount can be determined by conventional means, starting with a low dose of and then increasing the dosage while monitoring the immunological effects. Numerous factors can be taken into consideration when determining an optimal amount to administer, including the size, age, and general condition of the animal, the presence of other drugs in the animal, the virulence of the particular NPHV against which the animal is being immunized, and the like. The actual dosage is can be chosen after consideration of the results from various animal studies.

The immunologically effective amount of the immunogenic composition may be administered in a single dose, in divided doses, or using a “prime-boost” regimen. The compositions may be administered by any suitable route, including, but not limited to oral, immersion, parenteral, intradermal, transdermal, subcutaneous, intramuscular, intravenous, intraperitoneal, intranasal, oral, or intraocular routes, or by a combination of routes. The skilled artisan will be able to formulate the immunogenic composition according to the route chosen.

In addition to immunization techniques, antibodies that bind NPHV polypeptides described herein can also be generated by any other method known in the art. Exemplary alternative in-vitro antibody generation technologies, transgenic animal technologies and hybridoma technologies. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, Antibodies, A Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

In-vitro technologies suitable for generating NPHV binding antibodies include, but are not limited to, ribosome display, yeast display, and bacterial display technologies. Ribosome display is a method of translating mRNAs into their cognate proteins while keeping the protein attached to the RNA. The nucleic acid coding sequence is recovered by RT-PCR (Mattheakis, L. C. et al. 1994. Proc Natl Acad Sci USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503). In comparison to hybridoma technology, phage and other antibody display methods afford the opportunity to manipulate selection against the antigen target in vitro and without the limitation of the possibility of host effects on the antigen or vice versa.

For example, antibodies that bind NPHV polypeptides may be obtained by selecting from libraries, e.g. a phage library. A phage library can be created by inserting a library of random oligonucleotides or a library of polynucleotides containing sequences of interest, such as from the B-cells of an immunized animal (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol Today 21(8) 371-10). The diversity of a phagemid library can be manipulated to increase and/or alter the immunospecificities of the monoclonal antibodies of the library to produce and subsequently identify additional antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more framework sequences and introducing collections of CDR cassettes derived from antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other antibody generation techniques suitable for generating antibodies against the NPHV polypeptide, or a fragment of a NPHV polypeptide described herein include, the PEPSCAN technique described in Geysen et al (Patent Application WO 84/03564, Patent Application WO 86/06487, U.S. Pat. No. 4,833,092, Proc. Natl. Acad. Sci. 81: 3998-4002 (1984), J. Imm. Meth. 102, 259-274 (1987).

Pepsin or papain digestion of whole antibodies that bind NPHV polypeptides can be used to generate antibody fragments that bind NPHV polypeptides. In particular, an Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An (Fab′)₂ fragment of an antibody can be obtained by treating a whole antibody molecule with the enzyme pepsin, without subsequent reduction. An Fab′ fragment of an antibody molecule can be obtained from (Fab′)₂ by reduction with a thiol reducing agent, which yields a molecule consisting of an intact light chain and a portion of a heavy chain. Two Fab′ fragments are obtained per antibody molecule treated in this manner.

Antibodies can be produced through chemical crosslinking of the selected molecules (which have been produced by synthetic means or by expression of nucleic acid that encode the polypeptides) or through recombinant DNA technology combined with in vitro, or cellular expression of the polypeptide, and subsequent oligomerization. Antibodies can be similarly produced through recombinant technology and expression, fusion of hybridomas that produce antibodies with different epitope specificities, or expression of multiple nucleic acid encoding antibody variable chains with different epitopic specificities in a single cell.

Antibodies may be either joined directly or indirectly through covalent or non-covalent binding, e.g. via a multimerization domain, to produce multimers. A “multimerization domain” mediates non-covalent protein-protein interactions. Specific examples include coiled-coil (e.g., leucine zipper structures) and alpha-helical protein sequences. Sequences that mediate protein-protein binding via Van der Waals' forces, hydrogen bonding or charge-charge bonds can also be used as multimerization domains. Additional examples include basic-helix-loop-helix domains and other protein sequences that mediate heteromeric or homomeric protein-protein interactions among nucleic acid binding proteins (e.g., DNA binding transcription factors, such as TAFs). One specific example of a multimerization domain is p53 residues 319 to 360 which mediate tetramer formation. Another example is human platelet factor 4, which self-assembles into tetramers. Yet another example is extracellular protein TSP4, a member of the thrombospondin family, which can form pentamers. Additional specific examples are the leucine zippers of jun, fos, and yeast protein GCN4.

Antibodies may be directly linked to each other via a chemical cross linking agent or can be connected via a linker sequence (e.g., a peptide sequence) to form multimers.

The antibodies described herein can be polyclonal or monoclonal. The antibodies can also be chimeric (i.e., a combination of sequences from more than one species, for example, a chimeric mouse-canine immunoglobulin). Species specific antibodies avoid certain of the problems associated with antibodies that possess variable and/or constant regions form other species. The presence of such protein sequences form other species can lead to the rapid clearance of the antibodies or can lead to the generation of an immune response against the antibody by an antibody.

The antibodies described herein can be antibodies that bind to other molecules (antigens) via heavy and light chain variable domains, V_{H} and V_{L}, respectively. The antibodies described herein include, but are not limited to IgY, IgY(ΔFc)), IgG, IgD, IgA, IgM, IgE, and IgL. The antibodies may be intact immunoglobulin molecules, two full length heavy chains linked by disulfide bonds to two full length light chains, as well as subsequences (i.e. fragments) of immunoglobulin molecules, with or without constant region, that bind to an epitope of an antigen, or subsequences thereof (i.e. fragments) of immunoglobulin molecules, with or without constant region, that bind to an epitope of an antigen. Antibodies may comprise full length heavy and light chain variable domains, V_{H} and V_{L}, individually or in any combination.

The basic immunoglobulin (antibody) structural unit can comprise a tetramer. Each tetramer can be composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V₁) and variable heavy chain (V_{H}) refer to these light and heavy chains respectively.

The antibodies described herein may exist as intact immunoglobulins or as a number of well characterized fragments produced by digestion with various peptidases. In particular, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially an Fab with part of the hinge region (see, Fundamental Immunology, W. E. Paul, ed., Raven Press, N.Y. (1993) for more antibody fragment terminology). While the Fab′ domain is defined in terms of the digestion of an intact antibody, one of skill will appreciate that such Fab′ fragments may be synthesized de novo either chemically or by utilizing recombinant DNA methodology. The Fab′ regions may be derived from antibodies of animal or human origin or may be chimeric (Morrison et al., Proc Natl. Acad. Sci. USA 81, 6851-10855 (1984) both incorporated by reference herein) (Jones et al., Nature 321, 522-525 (1986), and published UK patent application No. 8707252, both incorporated by reference herein).

The antibodies described herein can include or be derived from any mammal, such as but not limited to a mouse, a rabbit, a rat, a rodent, a primate, or any combination thereof and includes isolated dog, primate, rodent, mammalian, chimeric, humanized and/or CDR-grafted or CDR-adapted antibodies, immunoglobulins, cleavage products and other portions and variants thereof. In one embodiment the antibody is purified.

The antibodies described herein include full length antibodies, subsequences (e.g., single chain forms), dimers, trimers, tetramers, pentamers, hexamers or any other higher order oligomer that retains at least a part of antigen binding activity of monomer. Multimers can comprise heteromeric or homomeric combinations of full length antibody, subsequences, unmodified or modified as set forth herein and known in the art. Antibody multimers are useful for increasing antigen avidity in comparison to monomer due to the multimer having multiple antigen binding sites. Antibody multimers are also useful for producing oligomeric (e.g., dimer, trimer, tertamer, etc.) combinations of different antibodies thereby producing compositions of antibodies that are multifunctional (e.g., bifunctional, trifunctional, tetrafunctional, etc.).

Specific examples of antibody subsequences include, for example, Fab, Fab′, (Fab′)₂, Fv, or single chain antibody (SCA) fragment (e.g., scFv). Subsequences include portions which retain at least part of the function or activity of full length sequence. For example, an antibody subsequence will retain the ability to selectively bind to an antigen even though the binding affinity of the subsequence may be greater or less than the binding affinity of the full length antibody.

An Fv fragment is a fragment containing the variable region of a light chain V_{L} and the variable region of a heavy chain V_{H} expressed as two chains. The association may be non-covalent or may be covalent, such as a chemical cross-linking agent or an intermolecular disulfide bond (Inbar et al., (1972) Proc. Natl. Acad Sci. USA 69:2659; Sandhu (1992) Crit. Rev. Biotech. 12:437).

Other methods of producing antibody subsequences, such as separation of heavy chains to form monovalent light-heavy chain fragments, further cleavage of fragments, or other enzymatic, chemical, or genetic techniques may also be used, provided that the subsequences bind to the antigen to which the intact antibody binds.

A single chain antibody (“SCA”) is a genetically engineered or enzymatically digested antibody containing the variable region of a light chain V_{L} and the variable region of a heavy chain, optionally linked by a flexible linker, such as a polypeptide sequence, in either V_{L}-linker-V_{H} orientation or in V_{H}-linker-V_{L} orientation. Alternatively, a single chain Fv fragment can be produced by linking two variable domains via a disulfide linkage between two cysteine residues. Methods for producing scFv antibodies are described, for example, by Whitlow et al., (1991) In: Methods: A Companion to Methods in Enzymology 2:97; U.S. Pat. No. 4,946,778; and Pack et al., (1993) Bio/Technology 11:1271.

The NPHV nucleic acids, polypeptides and immunogenic compositions described herein can be used to generate antibodies that that inhibit, neutralize or reduce the activity or function of a polypeptide or a NPHV. In certain aspects, the invention is directed to a method for treating an animal, the method comprising administering to the animal NPHV nucleic acids, polypeptides and immunogenic compositions, or administering to the animal an antibody which specifically binds to a NPHV polypeptide such that the activity or function of a NPHV polypeptide or a NPHV is inhibited, neutralized or reduced.

In another aspect, the invention described herein relates to NPHV immunogenic compositions comprising NPHV polypeptides or NPHV nucleic acids. In some embodiments, the NPHV immunogenic compositions can further comprise carriers, adjuvants, excipients and the like. The NPHV immunogenic compositions described herein can be formulated readily by combining the active compounds with immunogenically acceptable carriers well known in the art. The NPHV immunogenic compositions described herein can be formulated in a conventional manner using one or more physiologically acceptable carriers comprising excipients and auxiliaries which facilitate processing of the active compounds into preparations which can be used to induce an immunogenic response. Such carriers can be used to formulate suitable tablets, pills, dragees, capsules, liquids, gels, syrups, slurries, suspensions and the like. In one embodiment, the immunogenic composition can be obtained by solid excipient, grinding a resulting mixture, and processing the mixture of granules, after adding suitable auxiliaries, if desired, to obtain tablets or dragee cores.

The immunogenic composition described herein can be manufactured in a manner that is itself known, e.g. by means of conventional mixing, dissolving, granulating, dragee-making, levigating, emulsifying, encapsulating, entrapping or lyophilizing processes. Proper formulation is dependent upon the route of administration chosen.

When a immunogenetically effective amount of a NPHV immunogenic composition is administered to an animal, the composition can be in the form of a pyrogen-free, parenterally acceptable aqueous solution. The preparation of such parenterally acceptable protein or other active ingredient solutions, having due regard to pH, isotonicity, stability, and the like, is within the skill in the art. For example, NPHV immunogenic compositions described herein can contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The immunogenic composition of the present invention may also contain stabilizers, preservatives, buffers, antioxidants, or other additives known to those of skill in the art. The NPHV immunogenic compositions can be formulated in aqueous solutions, physiologically compatible buffers such as Hanks's solution, Ringer's solution, or physiological saline buffer. For transmucosal administration, penetrants appropriate to the barrier to be permeated are used in the formulation. Such penetrants are generally known in the art.

When the NPHV immunogenic compositions is administered orally, protein or other active ingredient of the present invention can be in the form of a tablet, capsule, powder, solution or elixir. When administered in tablet form, the immunogenic composition of the invention may additionally contain a solid carrier such as a gelatin or an adjuvant.

The NPHV immunogenic compositions described herein can encode or contain any of the NPHV proteins or peptides described herein, or any portions, fragments, derivatives or mutants thereof, that are immunogenic in an animal. One of skill in the art can readily test the immunogenicity of the NPHV proteins and peptides described herein, and can select suitable proteins or peptides to use in subunit immunogenic compositions.

The NPHV immunogenic compositions described herein comprise at least one NPHV amino acid or polypeptide, such as those described herein. The compositions may also comprise one or more additives including, but not limited to, one or more pharmaceutically acceptable carriers, buffers, stabilizers, diluents, preservatives, solubilizers, liposomes or immunomodulatory agents. Suitable immunomodulatory agents include, but are not limited to, adjuvants, cytokines, polynucleotide encoding cytokines, and agents that facilitate cellular uptake of the NPHV-derived immunogenic component.

The NPHV immunogenic compositions described herein can also contain an immunostimulatory substance, a so-called adjuvant Adjuvants in general comprise substances that boost the immune response of the host in a non-specific manner. A number of different adjuvants are known in the art.

The NPHV immunogenic compositions described herein may also comprise a so-called “vehicle”. A vehicle is a compound to which the protein adheres, without being covalently bound to it. Such vehicles are e.g. biomicrocapsules, micro-alginates, liposomes and macrosols, all known in the art. In addition, the immunogenic compositions may comprise one or more suitable surface-active compounds or emulsifiers, e.g. Span or Tween. Certain organic solvents such as dimethylsulfoxide also may be employed.

The NPHV immunogenic compositions described herein can also be mixed with stabilizers, e.g. to protect degradation-prone proteins from being degraded, to enhance the shelf-life of the immunogenic composition, or to improve freeze-drying efficiency. Useful stabilizers are i.e. SPGA (Bovarnik et al; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates.

When administered in liquid form, a liquid carrier such as water, petroleum, oils of animal or plant origin such as peanut oil, mineral oil, soybean oil, or sesame oil, or synthetic oils may be added. The liquid form of the immunogenic composition may further contain physiological saline solution, dextrose or other saccharide solution, or glycols such as ethylene glycol, propylene glycol or polyethylene glycol. When administered in liquid form, the immunogenic composition contains from about 0.5 to 90% by weight of protein or other active ingredient of the present invention, and from about 1 to 50% protein or other active ingredient of the present invention.

The NPHV immunogenic compositions described herein include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The NPHV immunogenic compositions described herein can also be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

The NPHV immunogenic compositions described herein can also be in the form of a complex of the protein(s) or other active ingredient of present invention along with protein or peptide antigens.

The NPHV immunogenic compositions described herein can be made suitable for parenteral administration and can include aqueous solutions comprising NPHV nucleic acids or polypeptides in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient maybe in powder form for constitution with a suitable vehicle, e.g., sterile pyrogen-free water, before use.

The NPHV immunogenic compositions described herein can also be in the form of a liposome in which protein of the present invention is combined, in addition to other acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The NPHV immunogenic compositions described herein can also be formulated as long acting formulations administered by implantation (for example subcutaneously or intramuscularly) or by intramuscular injection. The compositions may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Carriers for use with the NPHV immunogenic compositions described herein can be a co-solvent systems comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:SW) consists of VPD diluted 1:1 with a 5% dextrose in water solution. This co-solvent system dissolves hydrophobic compounds well, and itself produces low toxicity upon systemic administration. The proportions of a co-solvent system may be varied considerably without destroying its solubility and toxicity characteristics. The identity of the co-solvent components can also be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

The immunogenic compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with immunogenically compatible counter ions. Such immunogenically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

Excipients suitable for use in the immunogenic compositions described herein include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The immunogenic compositions described herein can also be multivalent immunogenic compositions that further comprise additional polypeptides or nucleic acid sequences encoding additional polypeptides from other viruses.

The immunogenic compositions described herein can also be multivalent immunogenic compositions that further comprise additional polypeptide fragments or nucleic acid sequences encoding additional polypeptide fragments from other viruses.

The immunogenic compositions described herein can also be multivalent immunogenic compositions that further comprise additional viruses (e.g. viruses that are either attenuated, killed or otherwise deactivated) or nucleic acid sequences encoding additional viruses (e.g. viruses that are either attenuated, killed or otherwise deactivated).

The immunogenic compositions described herein can also comprise fusions proteins, or nucleic acids encoding fusion proteins comprising a NPHV polypeptide, or a fragment thereof, and a polypeptide, or a polypeptide fragment from another virus.

Other additives that are useful in immunogenic composition formulations are known and will be apparent to those of skill in the art.

The following examples illustrate the present invention, and are set forth to aid in the understanding of the invention, and should not be construed to limit in any way the scope of the invention as defined in the claims which follow thereafter.

### EXAMPLES

### Example 1

### Characterization of a Canine Homolog of Hepatitis C Virus

An estimated 3% of the world's population is chronically infected with hepatitis C virus (HCV). Although HCV was discovered more than 20 years ago, its origin remains obscure largely because no closely related animal virus homolog has been identified; furthermore, efforts to understand HCV pathogenesis have been hampered by the absence of animal models other than chimpanzees for human disease. Described herein is the identification in domestic dogs of a non-primate hepacivirus. Comparative phylogenetic analysis of the non-primate hepacivirus (NPHV) confirmed it to be the most genetically similar animal virus homolog of HCV. Bayesian Markov chains Monte Carlo and associated time to most recent common ancestor analyses suggest a mean recent divergence time of NPHV and HCV clades within the past 500-1,000 years, well after the domestication of canines. The discovery of NPHV may provide new insights into the origin and evolution of HCV and a tractable model system with which to probe the pathogenesis, prevention, and treatment of diseases caused by hepacivirus infection.

Viral zoonoses account for up to 70% of human emerging infectious diseases; nonetheless, biological and epidemiological barriers to interspecies transmission are high (Parrish and Kawaoka, 2005; Parrish et al, 2008), and the majority of viruses that infect wildlife and domestic animals do not infect humans. Sustained contact between humans and other species increases the likelihood of the emergence of a virus adapted to infect and replicate in humans either directly or through intermediate hosts (Parrish et al, 2008). Identification and characterization of animal virus homologs provide new insights into pathogenesis of human viruses, and, in some instances, models for investigating prevention and treatment of human disease must be pursued with related animal viruses (Hatziioannou et al., 2009; Tsai et al., 1995; Van Rompay 2010). Comparative genetic analysis of closely related viruses can also identify genomic regions (RNA structures, amino acid motifs, and residues) important for virus receptor binding, entry, replication, immunity, and other biological functions (Kapoor et al., 2009; Kapoor et al., 2010; Kapoor et al., 2008; Klatt et al., 2010; Rijnbrand et al., 2000).

Since its discovery about 20 y ago (Alter 1989; Choo et al., 1989), the origin of hepatitis C virus (HCV) remains obscure largely because a closely related animal virus homolog has not been identified (Simmonds 2004; Stapleton et al., 2011). Worldwide, 200 million people are chronically infected with HCV (Simmonds 2004; Ray Kim 2005) and are at risk for developing liver fibrosis, cirrhosis, and hepatocellular carcinoma. Given precedent with other blood-borne pathogens like HIV and hepatitis B virus, efforts to find homologs of HCV have focused on non-human primates. To date, these efforts have been unsuccessful (Stapleton et al, 2011).

Dogs were domesticated as early as 8,000 BCE (Protsch and Berger, 1973) and, as companion and working animals, occupy a unique niche at the human-animal interface. Several flavivirus-like sequences during an investigation to characterize respiratory viruses infecting domestic dogs. Phylogenetic analysis of −6,500 nt of continuous genomic sequence revealed the presence of a virus genetically most similar to HCV, tentatively named non-primate hepacivirus (NPHV).

HCV belongs to the genus Hepacivirus, one of the four genera in the family Flaviviridae (Stapleton wt al., 2011). These viruses are classified in three established genera (Flavivirus, Pestivirus, and Hepacivirus) and one proposed genus, Pegivirus (Stapleton et al., 2011). Studies to understand the pathogenesis of HCV have been hampered by its restricted replication in humans or chimpanzees and, until recently, its inability to replicate in cell culture (Lohmann et al., 1999; Lindenbach et al., 2005). One alternative model is the distantly related GB virus B (GBV-B) that infects tamarins (Saguinus sp.) (Nam et al., 2004; Bukh et al, 2001; Bukh et al, 1999). However, GBV-B is highly divergent from HCV. Moreover, its elusive origins and ongoing uncertainty over whether tamarins are the natural host for GBV-B further restrict its value as a model system to study HCV pathogenesis (Stapleton et al., 2011).

Described herein is the discovery and unique genomic features of a hepacivirus that infects domestic dogs and is genetically most related to HCV. NPHV was found in respiratory samples as well as in liver; NPHV may also be hepatotropic. The results described herein indicate that hepaciviruses are not restricted to primates and indicate that HCV may have been introduced in the human population through contact with canines or other non-primate species.

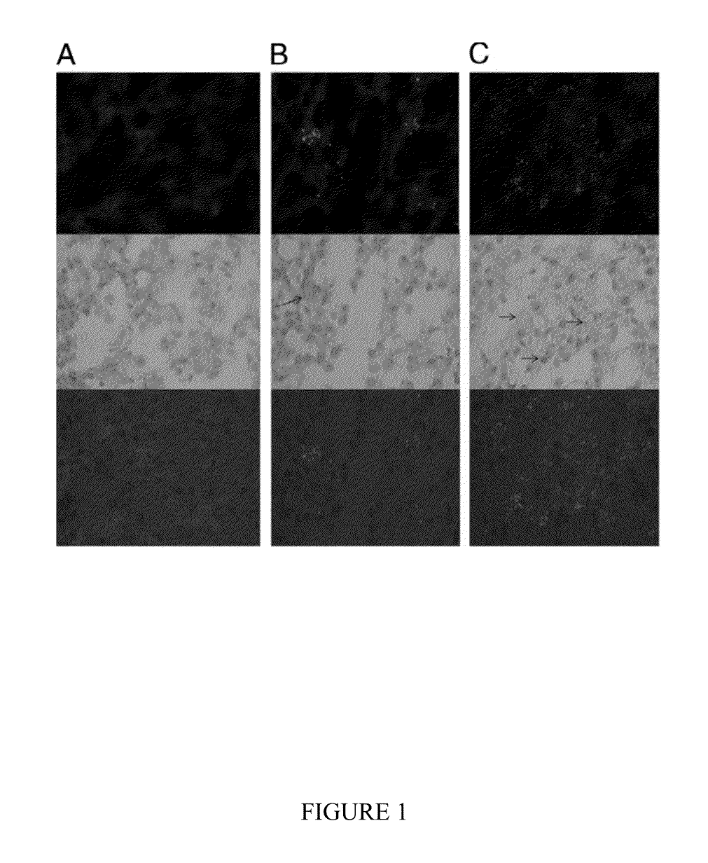

This study was undertaken to characterize the viral flora of companion animals. Respiratory samples of dogs associated with respiratory illness outbreaks were enriched for viral nucleic acids (Kapoor et al, 2008), randomly amplified, and subjected to unbiased high-throughput sequencing (Epstein et al., 2010). Bioinformatic analysis of sequences at the predicted amino acid level revealed the presence of several sequences substantially similar to flaviviruses. Sequence fragments were mapped to prototypic flavivirus genomes, and gaps in genomic sequences were filled by PCR using specific and degenerate primers. Preliminary phylogenetic analysis of ˜6,500 nt of continuous genomic sequence revealed the presence of a unique virus most closely related to HCV, tentatively named NPHV. Thereafter, specific primers targeting highly conserved helicase gene motifs in NPHV were used in RT-PCR to screen samples from 33 dogs representing five different outbreaks of respiratory disease. Six of 9 animals in one outbreak and 3 of 5 in another were positive for NPHV. Quantitative PCR assay yielded >107 copies of NPHV RNA per nasal swab of most infected animals. Partial NPHV sequences from the NS3 gene (399 nt) of viruses from different animals of the two outbreaks showed 99.2% sequence convergence (all substitutions occurred at synonymous sites). This high degree of genetic relatedness likely arose because both outbreaks occurred in same animal shelter facility within a period of 2 wk. To explore the epidemiology of NPHV infection in dogs, nasal swabs collected from 60 healthy pets were screened. No healthy animals were found infected with NPHV or its related variants. Also tested were 19 liver and five lung samples from 19 unrelated dogs that had died of unexplained gastrointestinal illness. In five dogs (three from Michigan and two from Montana), NPHV RNA was found in liver but not in lung tissue. Whereas respiratory samples contained >107 copies of NPHV genomic RNA per 2 ng of total RNA, levels in liver samples were <103 copies of NPHV genomic RNA in equivalent input. Blood, peripheral blood mononuclear cell, or other samples from these animals were not available for the study. The viral variants found in liver samples showed four synonymous and one non-synonymous mutation compared with NPHV (GenBank accession nos. JF744992 to JF744996). To test for the presence of NPHV in liver by using an independent method, an in situ hybridization assay was used to reveal focal as well as dispersed infection of canine liver and presence of viral RNA predominantly in the cytoplasm of hepatocytes (FIG. 1).

Attempts to culture NPHV in vitro using two continuous (Madin-Darby canine kidney and D17) and one primary (dog kidney primary) canine cell line have not been successful. Further studies can to determine the tissue tropism, pathogenic potential, and disease association of NPHV.

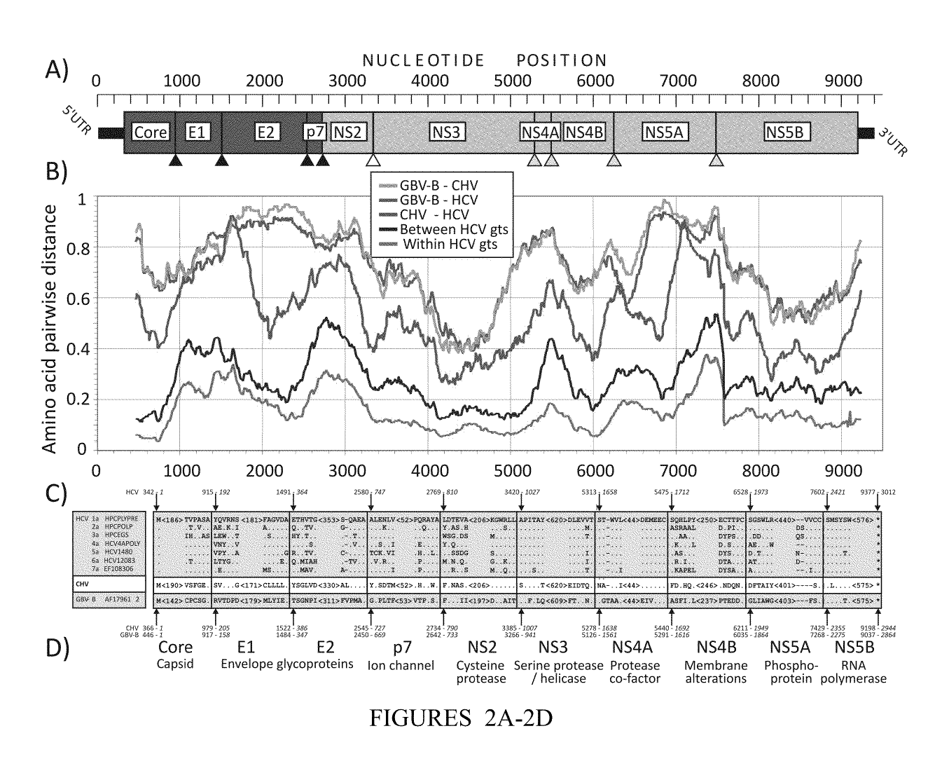

The genome sequence of NPHV was determined directly from a respiratory sample of one of the nine dogs with acute respiratory illness. The NPHV genome comprises at least 9,195 nt (GenBank accession no. JF744991) and encodes a 2,942-aa polyprotein and a short 5′ UTR (FIG. 2A). The complete 3′ UTR of NPHV was not cloned and sequenced, but note that the 3′ UTR of HCV was not cloned for several years after the initial identification of the virus (Tanaka et al., 2005; Kolykhalov et al., 1996). Based on analogy to HCV and GBV-B viruses, which contain a poly-U tract upstream of 3′-terminal RNA structures (3′ UTR), a poly-A primer was used to initiate reverse transcription in efforts to clone and characterize the 3′ UTR of NPHV. These efforts were unsuccessful after several attempts. However, cDNA construction using a poly-T primer resulted in early termination of NPHV genome, indicating the presence of a poly-A stretch near the 3′ terminus. This finding was confirmed by using an adapter ligation approach similar to the one described for cloning the 3′ terminus of HCV (Kolykhalov et al., 1996). The sequence is submitted in GenBank under accession number JF744997. These results are unusual and will require further study using high-titer NPHV samples.

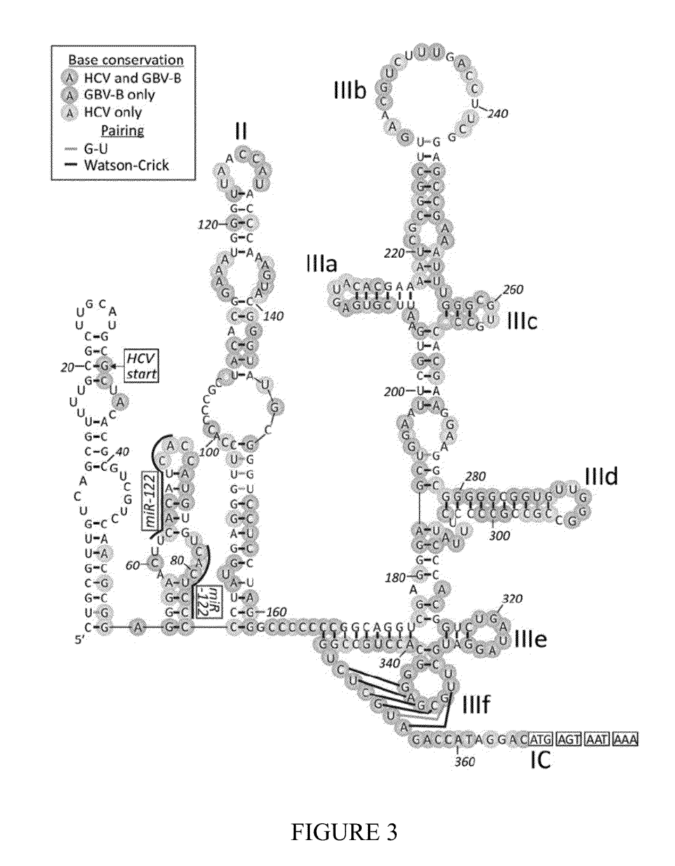

The G+C content of NPHV (50.7%) is similar to GBV-B (50.6%), lower than HCV (55.9-58.4%) or pegiviruses (55.9-60.6%), and higher than pestiviruses (45.5-46.7%) and the majority of classical flaviviruses (38.4-54.9%). Similar to HCV and GBV-B, the CpG (and UpA) dinucleotides were underrepresented (72% of expected value based on G+C content) compared with flaviviruses and pestiviruses. Using a sliding window analysis, the degree of amino acid sequence divergence of NPHV from other hepaciviruses was determined for the complete predicted polyprotein-coding region (FIG. 2). NPHV is more similar to HCV than to GBV-B throughout the genome (including the 5′ UTRs) (FIG. 3). Furthermore, regions of greater diversity between HCV genotypes are also more divergent between HCV and NPHV (E2 and 5′ end of nonstructural protein NSSA). Nonstructural proteins NS3 and NSSB of NPHV have maximum amino acid identity to HCV (>55-65%), whereas E1 (the N-terminal half of E2), NS2, and the C terminus of NSSA have the least amino acid identity (<35-45%) (FIG. 2).

In hepaciviruses, the structural and nonstructural proteins are typically generated by proteolytic cleavage by virus- and host encoded proteinases. The hypothetical cleavage map of the NPHV polyprotein was derived by alignment with representative sequences from the seven HCV genotypes (Simmonds 2004). Similar to HCV, cleavage of the NPHV polyprotein is predicted to create 10 viral proteins in a typical hepacivirus genomic organization (FIG. 2C).

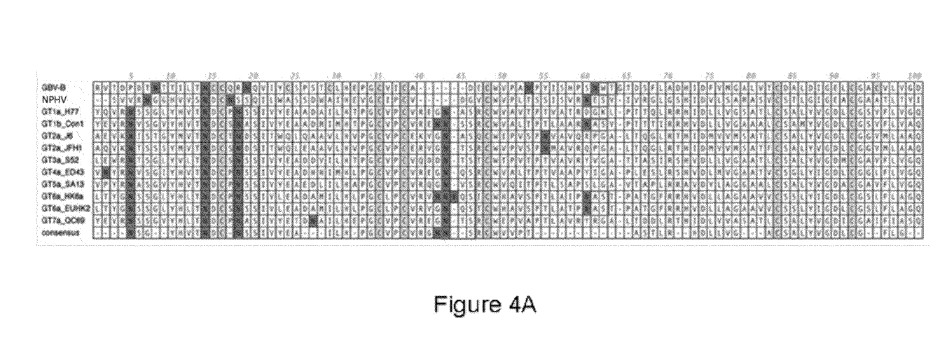

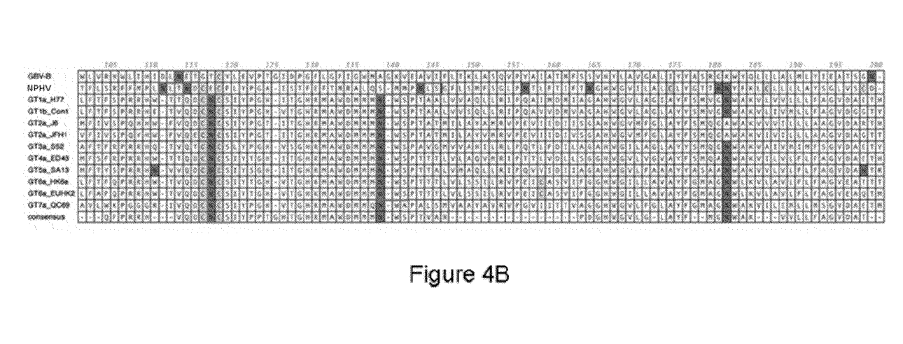

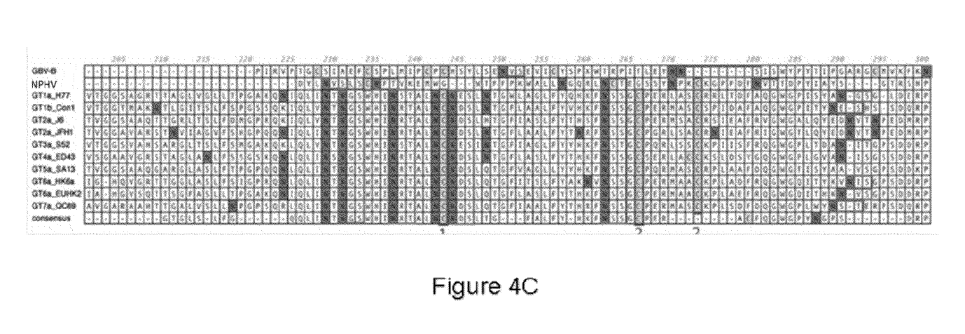

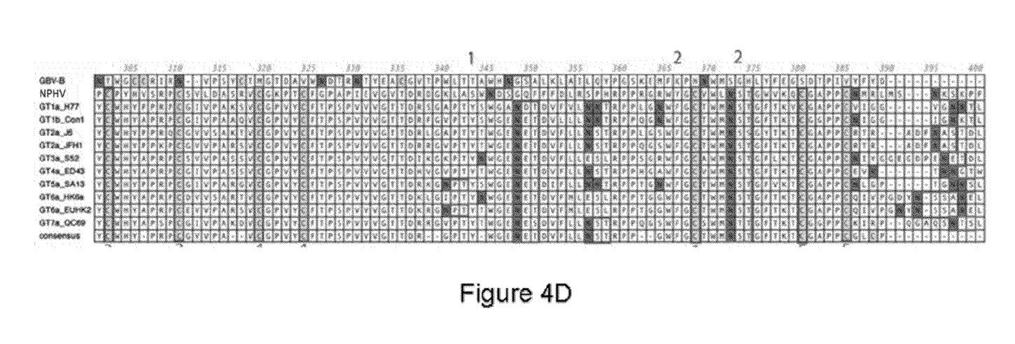

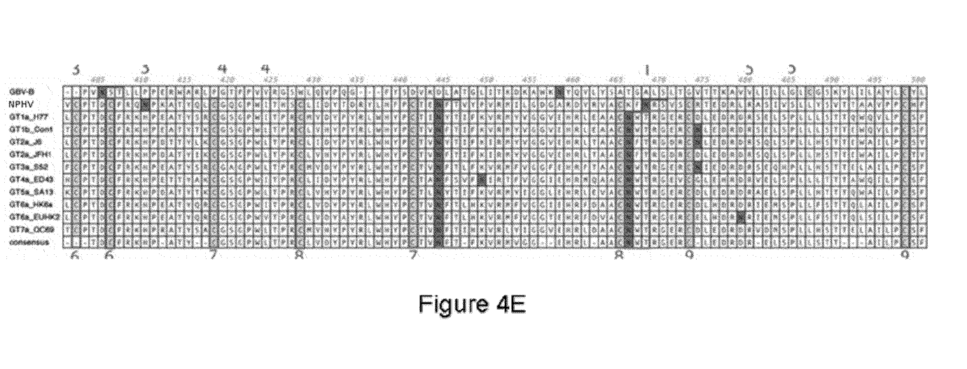

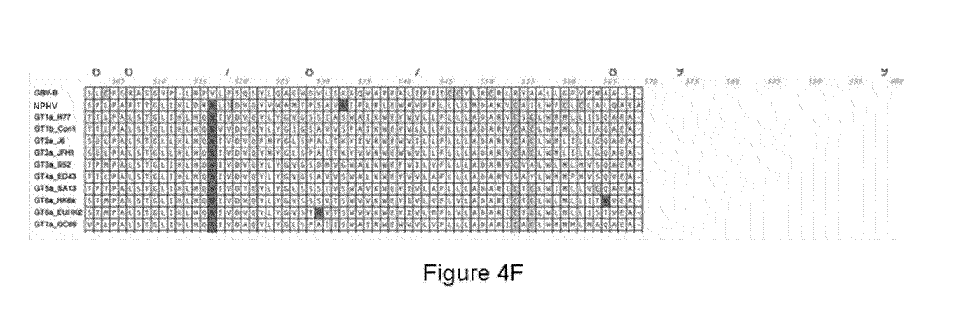

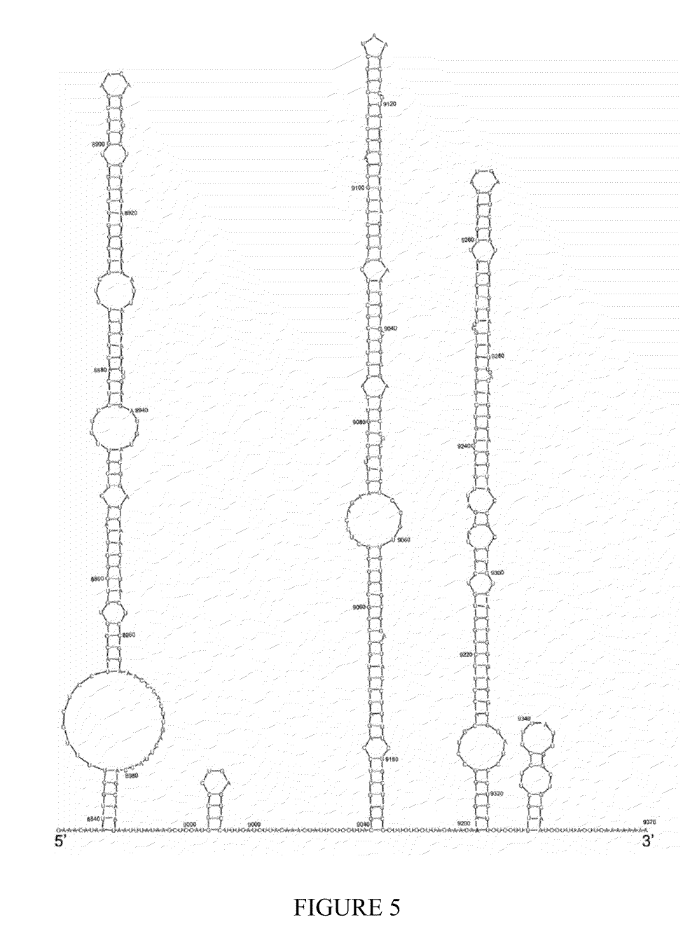

Signalase motifs (typically Ala-X-Ala preceding the cleavage site) are moderately conserved, as are the N52-N53 Zn-dependent cysteine protease and NS3-NS4A serine protease cleavage sites in the nonstructural polyprotein. Similar to HCV and GBV-B, the predicted NPHV core protein is highly basic (pI=11.4), consistent with an RNA binding/packaging function. Like HCV, the genome of NPHV contains a ribosomal slippery sequence (A5nnA5) in the core protein-coding region, possibly for generation of a frame-shift [F protein or alternative reading frame protein (ARFP)] product (Branch et al., 2005; Xu et al, 2001); however, the other two associated alternative reading frames in NPHV are interrupted by stop codons and have no significant homology to HCV F proteins. GBV-B also lacks an F protein ortholog, a finding that suggests that the F protein has a unique role in the life cycle of HCV. As between HCV and GBV-B, several long amino acid stretches in the E1E2 region with virtually no identifiable homology (>90% amino acid divergence) were observed. However, the E1E2 regions of NPHV were readily aligned with HCV, with marked similarity in the C-terminal half of E2 (FIG. 2B). The translated E1 and E2 sequences contained 4 and 10 N-linked glycosylation sites similar to sites predicted for HCV (5 and 11, respectively) (ref. Mohr and Stapleton 2009; FIG. 4) and greater than the number of sites in GBV-B (3 and 6, respectively) and members of the Pegivirus genus (e.g., 1 and 3 in GBV-A) (Stapleton et al., 2011). The ectodomain of E2 envelope protein of all HCV genotypes contain 18 highly conserved cysteine residues that form nine disulphide bonds crucial for proper 3D folding of the HCV structural protein (Krey et al, 2010). Interestingly, NPHV E2 contained 14 of the 18 highly conserved cysteine residues known to form disulfide bridges in HCV E2, whereas GBV-B lacked these cysteines (FIG. 4). The similar genome organization, presence of conserved protein motifs, processing sites, and the overall high sequence homology between NPHV and the other hepaciviruses (as determined with the National Center for Biotechnology Information Conserved Domains Database) can be use to predict the location and function of NPHV-encoded proteins (FIG. 2D) (Lindenbach et al., 2005; Lindenbach and Rice 2005).

Secondary RNA Structures in the NPHV Genome

The 5′ UTR of NPHV has 366 nt, which is more similar in length to HCV (341 nt) than to GBV-B (445 nt). Standard ClustalW-based sequence alignment demonstrated 66% and 57% nucleotide identity with HCV and GBV-B, respectively. The secondary structure of the NPHV 5′ UTR RNA was predicted by using a simple thermodynamic folding energy minimization algorithm (MFOLD) (FIG. 3) to reveal four stem-loops with moderate/high Pnum values (measure of the robustness of the predicted paired/unpaired state at each base position), two of which corresponded in position and shape to previously designated stem-loops II and III in the type IV internal ribosome entry sites of HCV, GBV-B, pestiviruses, and a few genera within the picornavirus family (Kieft 2008). Sequence conservation among NPHV, HCV, and GBV-B is most apparent in the pseudoknot region (the IIIf, IIIe, and IIIc stem-loops), whereas other regions (large parts of stem-loop II, the IIIb terminal loop, IIIa, and IIId) show conservation only in the predicted RNA secondary structure (multiple covariant sites or non-homologous base pairings). Substantial structural differences and lack of sequence homology are most evident at the 5′ end of the 5′ UTR between NPHV (and HCV) and the much longer equivalent region in GBV-B (Rijnbrand et al., 2000). Furthermore, the first stemloop predicted for NPHV is distinct from stem-loop I of HCV; indeed, based on the alignment, the 5′ base of the HCV genome falls within the 3′ half of the NPHV structure (FIG. 3). A second stem-loop of NPHV is formed by a stretch of 30 nt (positions 55-84) with evident homology to HCV (and, to a lesser extent, to GBV-B) (FIG. 3). However, in HCV, this region is predicted to be unpaired and contains binding sites for the human microRNA, miR-122 (Jangra et al., 2010; Jopling et al., 2005). Whether the stem-loop evident in NPHV precludes equivalent binding of the canine homolog of miR-122 (and indeed whether sequence variability in the first seed match is compatible with binding) remains to be determined (FIG. 3). A scan of the dog microRNA registry failed to identify any high-probability alternative seed matches in the NPHV 5′ UTR. Finally, the predicted NPHV 5′ UTR structure lacks the stem-loop around the polyprotein initiation codon found in HCV and GBV-B (but not in pestiviruses). The translational control mechanisms proposed for HCV mediated by this pairing (Honda et al., 1996) are thus unlikely to apply in NPHV.

RNA secondary structure within coding regions of the genome was characterized by extensive internal base pairing. For example, MFOLD analysis of the last 540 nt of the coding region of the NPHV genome (corresponding to the region containing a cis-replicating element in HCV) predicts three stable stem-loop structures (each spanning 120-145 nt) with long duplex regions (FIG. 5). RNA viruses containing genome-scale ordered RNA structure (GORS) with high mean folding energies (MFEs) are more likely to cause persistent infection (Simmonds et al., 2004). The NPHV genome sequence was analyzed for evidence of GORS by comparing folding energies of consecutive fragments of nucleotide sequence with random sequence-order controls (Simmonds et al., 2004). The MFE difference value of the NPHV genome at 13.8% is higher than that of HCV (7.8-9.5%), GBV-B (9.5%), or pegiviruses (10.3-14.4%). Based on the characteristics of other RNA viruses with GORS, these observations predict that NPHV infections may be persistent in its natural hosts.

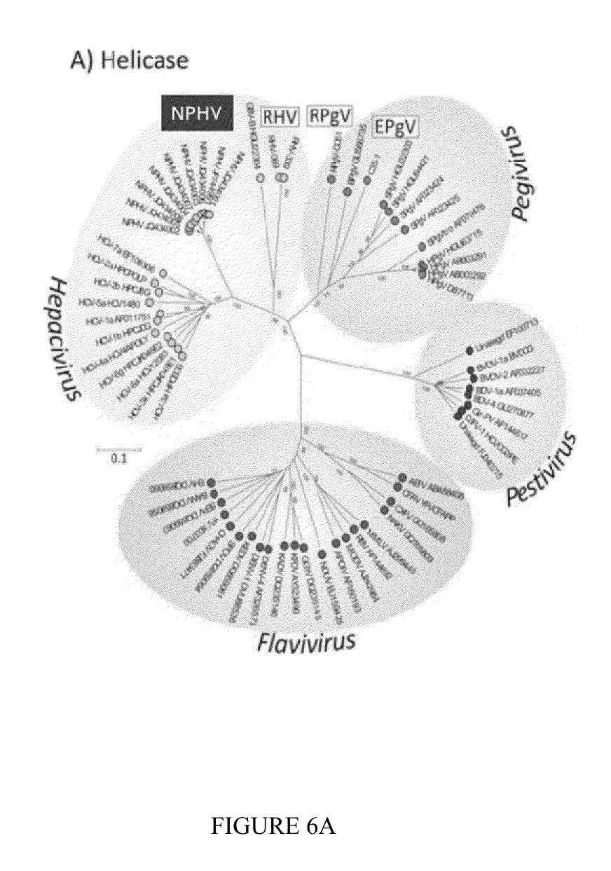

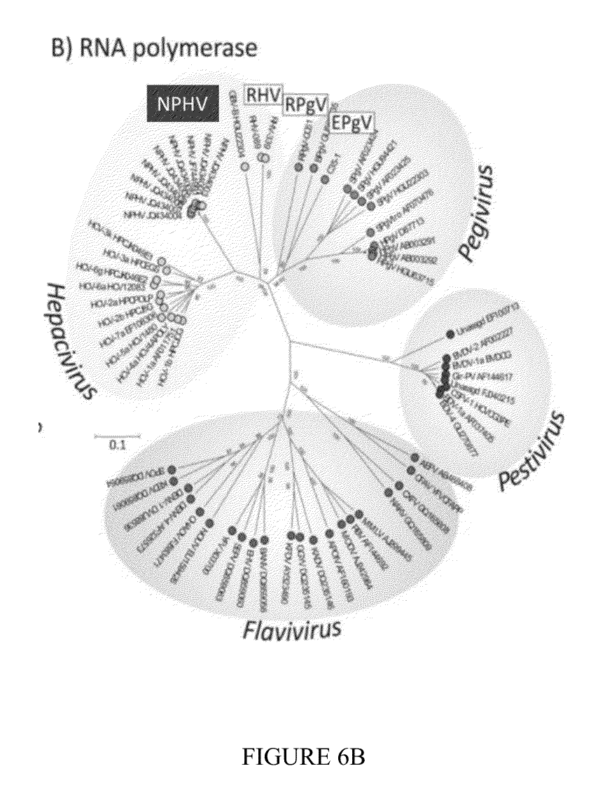

NPHV was phylogenetically classified by determining its genetic relatedness to representative viruses of different genera of Flaviviridae. These viruses are classified in three established genera (Flavivirus, Pestivirus, and Hepacivirus) and one proposed genus, Pegivirus (Stapleton et al., 2011). Comparative phylogenetic analysis of conserved regions in the predicted helicase (NS3) and RNA-dependent RNA polymerase (RdRp or NS5B) regions were congruent with NPHV consistently closest and equidistant from the seven HCV genotypes (FIG. 6). These clusters were consistent with the established taxonomic groups and supported by bootstrap values of above 90% (of 1,000 replicates) (Tamura et al., 2007). The phylogenetic position of NPHV relative to HCV and GBV-B is consistent with their pairwise distances (Table 1). A listing of virus abbreviations and original accession numbers for each sequence are provided in FIG. 6 are shown in Table 2.

TABLE 1
Pairwise distances between 5′ UTR, structural (S gene) and
nonstructural (NS gene) proteins of different hepadviruses

| Genome | | | | |
|---|---|---|---|---|
| Region | NPHV | HCV | GBV-B | PgV |
| 5′ UTR | | | | |
| NPHV | ND | | | |
| HCV | 66.0 | 95.2 | | |
| GBV-B | 56.7 | 62.8 | ND | |
| PgV | IH | IH | IH | 64.7 |
| S gene | | | | |
| NPHV | ND | | | |
| HCV | 44.1 (35.9) | 67.5 (71.6) | | |
| GBV-B | 29.6 (11.2) | 29.6 (12.1) | ND | |
| PgV | IH | IH | IH | 44.8 (35.2) |
| NS gene | | | | |
| NPHV | ND | | | |
| HCV | 52.3 (50.7) | 66.2 (72.5) | | |
| GBV-B | 41.1 (30.2) | 40.2 (30.6) | ND | |
| PgV | 37.8 (25.5) | 38.4 (25.6) | 36.9 (24.3) | 52.4 (49.9) |

Amino acid divergence is given in parentheses.
IH, Insufficient homology for valid comparison;
ND, not done (only one sequence available);
PgV, Pegivirus (GBV-A, -C, and -D).

TABLE 2
Sequences, accession nos., and virus abbreviations used in the
phylogenetic analysis described in FIG. 6

| | Accession | |
|---|---|---|
| Genus/Virus | No. | Description |
| Flavivirus | | |
| APOIV | AF160193 | Apoi virus polyprotein gene, complete cds\|Flavivirus\|Rio Bravo |
| | | virus group |
| BANV | DQ859056 | Banzi virus strain SAH 336 polyprotein gene, complete cds\| |
| | | Flavivirus\|Yellow fever virus group |
| CHAOV | FJ883471 | Chaoyang virus strain Deming polyprotein gene, complete cds\| |
| | | Flavivirus |
| DENV-4 | AF326573 | Dengue virus type 4 strain 814669, complete genome\|Flavivirus\| |
| | | Dengue virus group |
| EHV DQ | 859060 | Edge Hill virus strain YMP 48 polyprotein gene\|Flavivirus\| |
| | | Yellow fever virus group |
| GGYV | DQ235145 | Gadgets Gully virus from Australia polyprotein gene, complete cds\| |
| | | Flavivirus |
| KADV | DQ235146 | Kadam virus from Uganda polyprotein gene, complete cds\| |
| | | Flavivirus |
| KEDV | DQ859061 | Kedougou virus strain Dak AR D1470 polyprotein gene, complete |
| | | cds\|Flavivirus |
| MMLV | AJ299445 | Montana myotis leukoencephalitis virus complete genomic RNA\| |
| | | Flavivirus |
| MODV | AJ242984 | Modoc virus genomic RNA for polyprotein gene\|Flavivirus\| |
| | | Modoc virus group |
| NOUV | EU159426 | Nounane virus polyprotein mRNA, complete cds\|Flavivirus |
| RBV | AF144692 | Rio Bravo virus strain RiMAR polyprotein gene, complete cds\| |
| | | Flavivirus\|Rio Bravo virus group |
| SEPV | DQ859063 | Sepik virus strain 7148 polyprotein gene, complete cds\|Flavivirus\| |
| | | mosquito-borne viruses |
| SPOV | DQ859064 | Spondweni virus strain SM-6 V-1 polyprotein gene\|Flavivirus\| |
| | | Spondweni virus group |
| DENV-1 | DVU88536 | Dengue virus type 1 clone 45AZ5, complete genome\|Flavivirus\| |
| | | Dengue virus group |
| KFDV | AY323490 | Kyasanur forest disease virus polyprotein gene\|Flavivirus\|tick- |
| | | borne encephalitis virus group |
| YFV | X03700 | Yellow fever virus complete genome, 17D vaccine strain\| |
| | | Flavivirus\|Yellow fever virus group |
| AEFV | AB488408 | Aedes flavivirus genomic RNA, complete genome, strain Narita-21\| |
| | | Flavivirus |
| CFAV | YFVCFAPP | Flavivirus cell fusing agent polyprotein gene, complete cds\| |
| | | Flavivirus |
| CXFV | GQ165808 | Culex flavivirus strain Uganda08 polyprotein gene, partial cds\| |
| | | Flavivirus |
| NAKV | GQ165809 | Nakiwogo virus strain Uganda08 polyprotein gene, partial cds\| |
| | | Viruses\|Flaviviridae |
| Hepacivirus | | |
| HCV-1a | AF011751 | HCV strain H77 pCV-H77C polyprotein gene, complete cds |
| HCV-1b | HPCJCG | HCV ORF gene, complete cds\|Hepacivirus |
| HCV-2b | HPCJ8G | D10988 D01221 HCV genome |
| HCV-2a | HPCPOLP | HCV genomic RNA for polyprotein, complete cds\|Hepacivirus |
| HCV-3a | HPCEGS | HCV (isolate NZL1) genomic RNA, complete genome\| |
| | | Hepacivirus |
| HCV-3k | HPCJK049E1 | HCV (isolate JK049) genomic RNA, complete genome\| |
| | | Hepacivirus |
| HCV-4a | HCV4APOLY | Y11604 HCV type 4a RNA for HCV polyprotein |
| HCV-5a | HCV1480 | Y13184 HCV genotype 5a RNA for HCV polyprotein |
| HCV-6a | HCV12083 | Y12083 HCV genotype 6a RNA for HCV polyprotein |
| HCV-6g | HPCJK046E2 | HCV (isolate JK046) genomic RNA, complete genome\| |
| | | Hepacivirus |
| HCV-7a | EF108306 | HCV (isolate QC69) polyprotein gene, complete cds\|Hepacivirus |
| GBV-B | HGU22304 | U22304 hepatitis GBV-B polypeptide complete genome |
| Pegivirus | | |
| SPgV | AF023424 | Hepatitis GB virus A complete genome |
| SPgV | AF023425 | Hepatitis GB virus A complete genome |
| SPgV | HGU22303 | U22303 hepatitis GB virus A polyprotein, complete cds |
| SPgV | HGU94421 | U94421 hepatitis GB virus A strain Alab, complete genome |
| HPgV | AB003291 | Hepatitis GB virus C genomic RNA for polyprotein, isolate |
| | | CG12LC |
| HPgV | AB003292 | Hepatitis GB virus C genomic RNA for polyprotein, isolate G05BD |
| HPgV | D87713 | Hepatitis GB virus C genomic RNA, complete sequence, strain |
| | | K2141 |
| HPgV | HGU637155 | U63715 Hepatitis GB virus C polyprotein gene, complete cds |
| SPgVtro | AF070476 | GB virus C variant troglodytes, complete genome, |
| BPgV | GU566735 | GB virus D strain 93 polyprotein precursor, gene, partial cds |
| Pestivirus | | |
| BDV-1a | AF037405 | Border disease virus strain X818, complete genome\|Pestivirus |
| BVDV-1a | BVDCG | Bovine viral diarrhea virus 1-NADL, complete genome\|Pestivirus |
| BVDV-2 | AF002227 | Border disease virus strain C413, complete genome\|Pestivirus |
| CSFV-1 | HCVCG3PE | Classical swine fever virus, Brescia hog cholera virus protein |
| | | precursor\|Pestivirus |
| Gir-PV | AF144617 | Pestivirus giraffe-1 H138 complete genome\|Pestivirus |
| BDV-4 | GU270877 | Border disease virus strain H2121 (Chamois-1), complete genome\| |
| | | Pestivirus |
| | FJ040215 | Bovine viral diarrhea virus 3 Th/04 KhonKaen, complete genome\| |
| | | Pestivirus |
| Unassgd | EF100713 | Porcine pestivirus isolate Bungowannah polyprotein gene, partial |
| | | cds\|Pestivirus |

cds, coding sequence;
Unassgd, unassigned.

The dissimilarity in S region sequences between GBV-B and other hepaciviruses is reflected in the virtual absence of sequence similarity of amino acid sequences after alignment (11-12%) compared with 36% amino acid similarity between NPHV and HCV. The degree of sequence divergence between HCV and NPHV is almost the same as that observed between GBV-C (a human virus) and GBV-A (found in chimpanzees and New World primates) (FIG. 6). Considering the results of phylogenetic analyses, pairwise protein distances, and identification of NPHV in a different natural host, the results described herein indicate that NPHV should be classified as a prototype new virus species in the genus Hepacivirus.

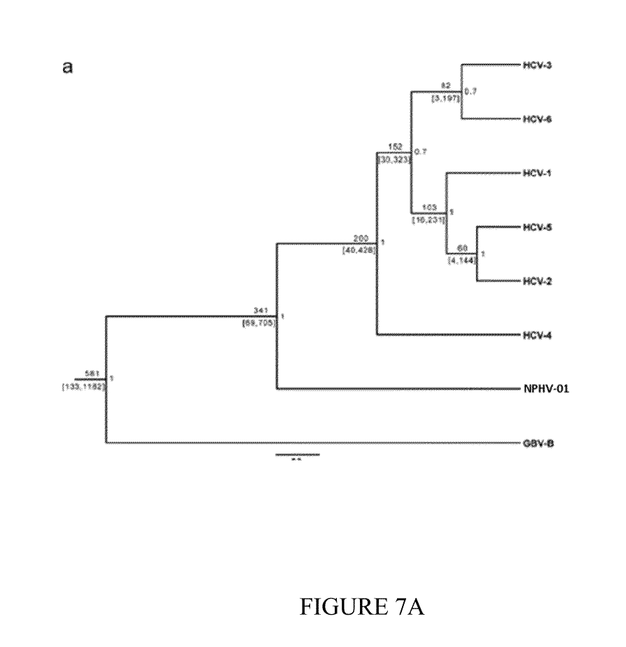

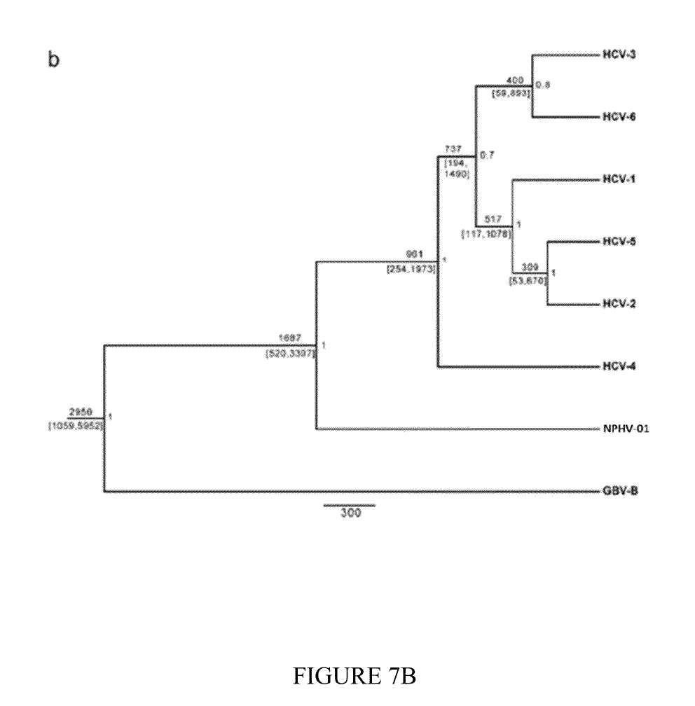

To determine the evolutionary relation between NPHV and other hepaciviruses, a Bayesian Markov chains Monte Carlo (MCMC) estimation of the time to most recent common ancestor (TMRCA) for the HCV genotypes, GBV-B, and NPHV was performed by using an external rate calibration based on the evolutionary rates estimated for (i) HCV subtypes 1a and 1b (Magiorkinis et al, 2009) and (ii) HCV subtype 6 (Pybus et al., 2009). The mean estimated TMRCA for the Hepacivirus Glade and NPHV is 341 y before present (ybp) [95% highest posterior density (HPD)=69-705 ybp] based on the HCV subtype 1 calibration and 1,680 ybp (521-3,291 ybp) based on the HCV subtype 6 calibration (FIG. 7). Thus, the shared common ancestor between NPHV and the HCV genotypes probably existed between 500 and 1,000 ybp. However, this should only be regarded as a minimum estimate given the difficulties associated with extrapolating short-term substitution rates to longer evolutionary periods (Keckesova et al., 2009, Worobey et al., 2010). The TMRCA between NPHV and HCV was estimated using a substitution rate previously used to infer the divergence times within HCV genotypes/subtypes (Magiorkinis et al, 2009; Pybus et al., 2009). However, whether these rates can be reliably applied to much more divergent NPHV and HCV sequences is unclear. Observation of time dependency in substitution rates in host genes (Ho et al, 2005) and recent evidence from endogenous viral elements for substantially slower long-term substitution rates in a variety of animal viruses argue against simple extrapolation of substitution rates measured over short observation periods (Kapoor et al., 2010; Belyi et al., 2010; Horie et al., 2010; Katzourakis et al., 2010).

Molecular characterization of NPHV indicates that it is the most genetically related homolog of HCV. Viral structural proteins typically contain major determinants of viral immunogenicity and host/cell tropism. The envelope protein E2 of HCV is among the most variable portions of its genome, yet it has remarkable sequence similarity with NPHV. Moreover, the number and position of cysteine residues in E2 protein of NPHV indicate that even the tertiary structure of NPHV is likely to be more similar to HCV than to other genetically related viruses (Krey et al, 2009). However, there are notable differences between NPHV and HCV that may have biological significance. Most strikingly, the potential occlusion of the binding site of miR-122 in the NPHV 5′ UTR and the absence of microRNA sequences in the dog genome capable of binding to the equivalent site in NPHV indicate that the interaction, which enhances the replication of HCV in human liver (Jangra et al., 2010; Jopling et al, 2005), may not be needed in NPHV infections. It remains to be determined whether the unique stem-loop I of NPHV allows it to replicate in a manner independent of miR-122 or influences tissue tropism. Although reverse-genetic experiments wherein genomic regions are swapped between HCV and NPHV can be used to further examine these questions, only low or undetectable levels of HCV RNA are typically detected in respiratory samples from HCV-infected humans even though NPHV is found at high levels in respiratory samples of infected animals (Ferreiro et al, 2005). A significant difference in life span of humans and canines can also affect the disease pattern caused by genetically related viruses. The availability of NPHV genome and its comparative genetic analysis with HCV genotypes can therefore advance the understanding of the role genetic elements and proteins play in the viral life cycle. Moreover, the sequence data presented here will help in designing reagents necessary to further explore the biology, pathogenesis, and tissue tropism of

The limited genetic diversity observed among NPHV variants is atypical for RNA viruses including HCV and is likely attributable to the study animals being in close contact (same disease outbreak) or the highly specific PCR assay used in this study. The use of broadly reactive reagents (e.g., degenerate primers) and samples from unrelated dogs from different geographies will result in identification of many diverse NPHV-related viruses. Without further information on the distribution of HCV-related viruses in other mammalian species, it is too early to draw conclusions on the evolutionary events underlying their distribution in humans and dogs and their apparent absence in non-human primates. Indeed, there is virtually no information on the existence of HCV-like viruses in other mammalian orders, which have probably remained untested because of a primate focus in screening paradigms. Hepaciviruses may be widely distributed among different mammalian species, perhaps highly host-specific, effectively transmitted by non-parenteral routes and largely nonpathogenic (as appears to be the case for pegiviruses in primates) (Simmonds et al., 2004). An alternative scenario is one where hepaciviruses are primarily canine viruses and HCV in humans arose zoonotically from contact from dogs or other related members of carnivore mammalian order that harbor these types of viruses. A zoonotic origin for HCV and lack of host adaptation can explain its high degree of pathogenicity in humans, inefficient transmission by non-parenteral routes, and apparent absence of HCV homolog in nonhuman primates. However, the miR-122 interaction appears to be human-specific and likely represents a virus/host co-adaptation, unlikely responsible for a recent zoonosis.

Although its hepatotropism and ability to establish persistence remain to be determined, the presence of NPHV in hepatocytes is reminiscent of HCV infections in humans. Furthermore, the presence of GORS in NPHV is consistent with persistence in other viral systems. Irrespective of its evolutionary origins, the discovery of NPHV provides the exciting prospect of a unique experimental model for HCV infections in humans and opens future avenues for research into the pathogenesis, prevention, and treatment of hepacivirus infections.

Samples and High-Throughput Sequencing.

Respiratory samples (nasal swabs) were collected in 3 mL of MEM from affected dogs in five respiratory disease outbreaks in four shelters (one each in Texas and Utah and three in Pennsylvania). Postmortem lung and liver samples were from euthanized dogs in clinics in Montana and Missouri. Centrifuged respiratory sample (140 μL) was filtered through a 0.45-μm filter to remove eukaryotic and bacterialsized particles. The filtrate was then treated with nucleases to digest nonparticle-protected nucleic acid. RNA from filtered (0.45-μm) respiratory samples or tissue homogenates was treated with DNase before random amplification and pyrosequencing (Kapoor et al., 2008; Victoria et al., 2009). After assembly (Newbler v2.3; 454 Life Sciences), sequence contigs and singletons were analyzed by using BlastX against a National Center for Biotechnology Information non-redundant protein database (Victoria et al., 2009). Genome Sequencing and Phylogenetic and Evolutionary Analyses.

RNA Structure and GORS Predictions.

Independent of phylogenetic information, the secondary structure of the NPHV 5′ UTR RNA was modeled with MFOLD. Labeling of the predicted structures in the 5′ UTR followed numbering used for reported homologous structures in HCV and GBV-B (Rijnbrand et al., 2000, Honda et al., 2006). The NPHV genome sequence was analyzed for evidence of GORS by comparing folding energies of consecutive fragments of nucleotide sequence with random sequence-order controls (Simmonds et al., 2004). MFEs of NPHV were calculated by using default setting in the program ZIPFOLD. MFE results were expressed as MFE differences, i.e., the percentage difference between the MFE of the native sequence from that of the mean value of the 50 sequence order-randomized controls.

In Situ Hybridization.

For in situ hybridization assay, multiple branched-chain probe amplifiers labeled with alkaline phosphatase were used against NPHV genomic RNA (nucleotides 840-2040 of NPHV genome corresponding to the coding region for partial core, envelope glycoprotein E1/E2, and partial NS1 protein). Liver sections (10 μm) were fixed with 4% formaldehyde at 4° C. overnight, dehydrated, permeabilized, and stained with Fast Red substrate for light and florescent microscopy.

Genome Sequencing and Phylogenetic Analysis.

Sequences with similarity to flaviviruses were assembled against prototype hepatitis C virus (HCV) strains. Gaps were filled by primer walking using specific and degenerate flavivirus primers. Both termini of the genome were acquired by using RACE (Kapoor et al., 2008). Thereafter, sequence validity was tested in 4× genome coverage by classical dideoxy Sanger sequencing. Nucleotide compositions of different flaviviruses and non-primate hepacivirus (NPHV) were determined by using EMBOSS compseq (http://emboss.bioinformatics.nl/cgi-bin/emboss/compseq). Translated amino acid sequences were aligned with ClustalW. Trees were constructed by neighbor joining of pairwise amino acid distances with the program MEGA5 (Kumar et al., 2008), using bootstrap resampling to determine robustness.

Screening and Quantitative PCR.

All respiratory and tissue samples were extracted with Qiagen viral RNA extraction kit and RNeasy tissue DNA/RNA extraction kit. RNA was converted to cDNA using random primers and then used in nested PCR with primers for the first round (NPHV-0F1: 5′-TCCACCTATGGTAAGTTCTTAGC-3′ (SEQ ID NO: 41) and NPHV-0R1: 5′-ACCCTGTCATAAGGGCGTC-3′ (SEQ ID NO: 42)) and the second round (NPHV-0F2: 5′-CCTATGGTAAGTTCTTAGCTGAC-3′ (SEQ ID NO: 43) and NPHV-0R2: 5′-CCTGTCATAAGGGCGTCCGT-3′ (SEQ ID NO: 44)). All PCR products were sequenced to confirm the presence of NPHV in samples. Quantitative PCR to determine the NPHV genome copy number in respiratory samples was performed by using SYBR green chemistry and a plasmid containing HCV helicase gene as a copy number standard. The primers used were 5′-GCCATAGCACAGACTCCAC-3′ (SEQ ID NO: 45) (NPHV-SG-F1) and 5′-GACGGAAACATCCAAACCCCG-3′ (SEQ ID NO: 46) (NPHV-SG-2R1) with ready-to-use PCR mix.

Evolutionary Analysis.

Bayesian Markov chains Monte Carlo (MCMC) phylogenies and associated time to most recent common ancestor (TMRCA) for representative members of the HCV strains, NPHV-01, and GHV-B were estimated by using a 555-nt segment of the NS5B gene in the program BEAST v1.6 (Drummond et al., 2007). TMRCA was estimated by using a relaxed molecular clock with an uncorrelated log-normal distribution on the rate that was calibrated by using external rate estimates based on the NS5B genes of (i) the global diversity of HCV subtypes 1a and 1b (Magiorkinis et al., 2009) and (ii) HCV subtype 6 diversity in Asia (Pybus et al., 2009). Normal and lognormal distributions were determined by the mean and 95% highest posterior densities (HPDs) of the reported substitution rates for all three codon positions as well as only the first and second codon positions to limit the effect of potential site saturation at the third position. A general time reversible of nucleotide substitution was used, with rate heterogeneity among sites modeled by a discrete gamma distribution with four rate categories, as determined by ModelTest (Posada et al., 1998). All analyses were performed with several tree priors, including a speciation model (Yule) and two unconstrained coalescent models, the Bayesian Skyline (Drummond et al., 2005) and Bayesian Skyride (Minin et al., 2008) demographic models. MCMC sampling was performed for 5×107 generations, sampling every 5,000 generations. Convergence and mixing were assessed with the program Tracer v1.5 (http://tree.bio.ed.ac.uk). Maximum clade credibility trees were generated with TreeAnnotator (Drummond et al., 2007).

For the data sets calibrated with both HCV subtypes 1 a/b and subtype 6, the Yule speciation model had the best fit to the data, as assessed by comparing the posterior tree likelihoods (FIG. 7). Analyses that included third-codon positions resulted in wider 95% HPDs around the mean TMRCA, likely because of an increased number of substitutions at that site. However, all modelprior combinations for each of the rate calibrations resulted in 95% HPDs that were overlapping between the analyses, indicating that estimates are robust to the choice of tree prior and inclusion of third-codon positions.

### Example 2

### Characterization of NPHV

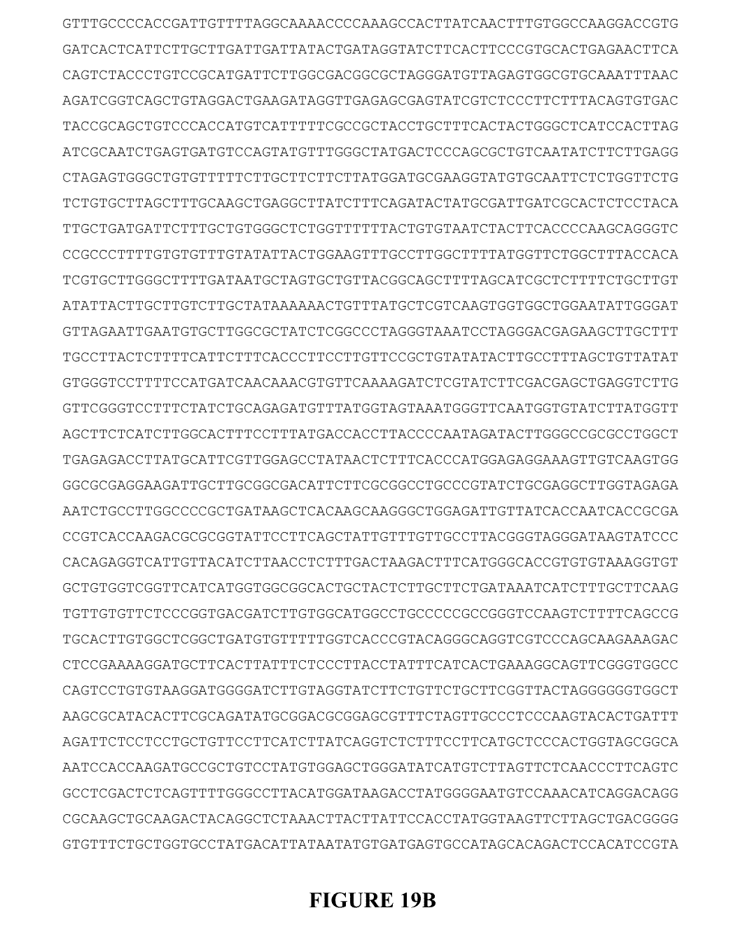

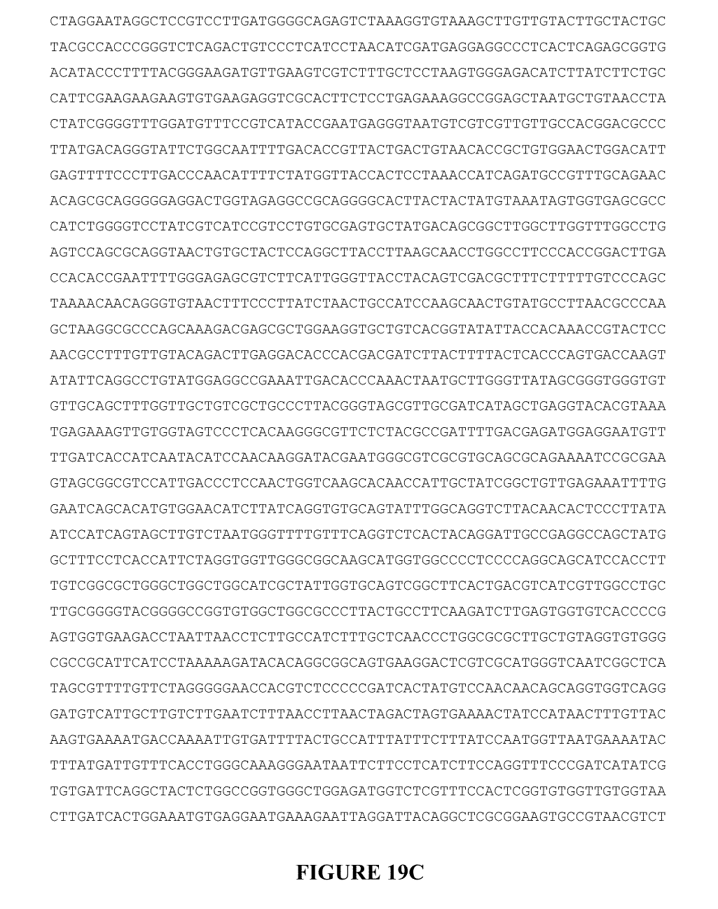

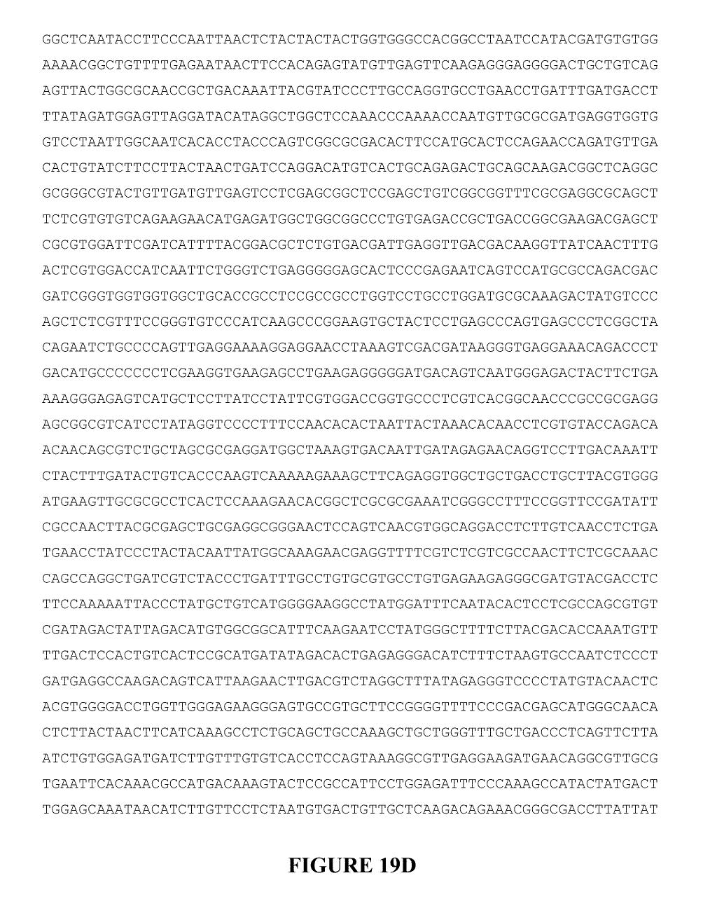

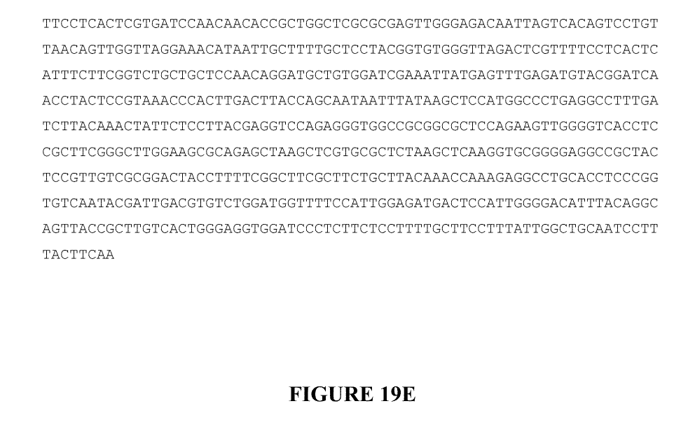

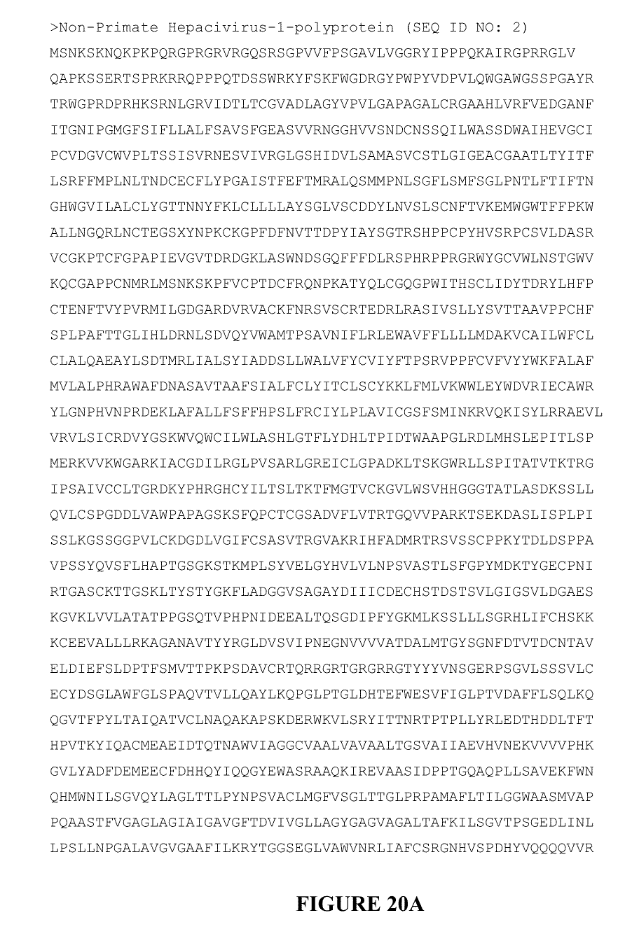

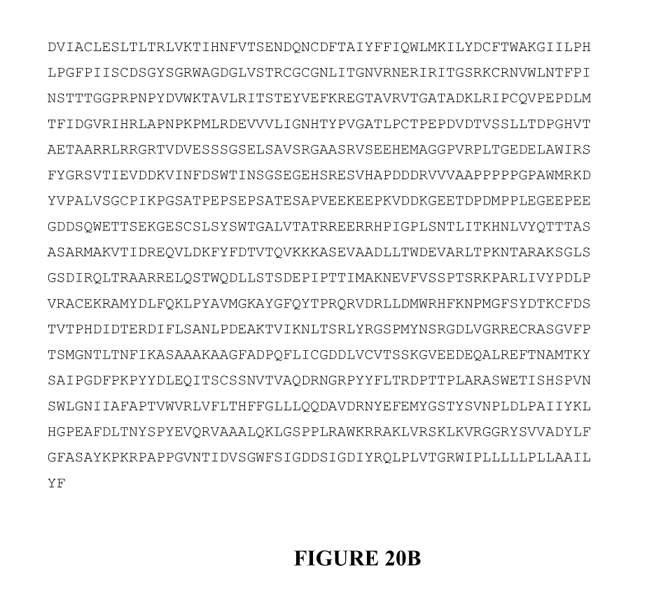

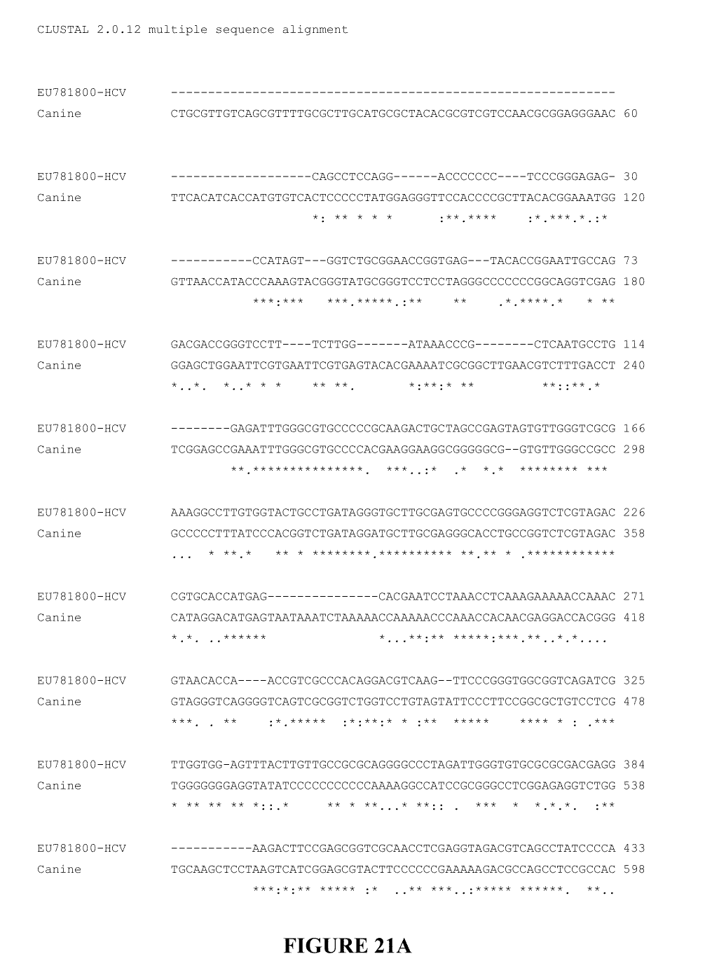

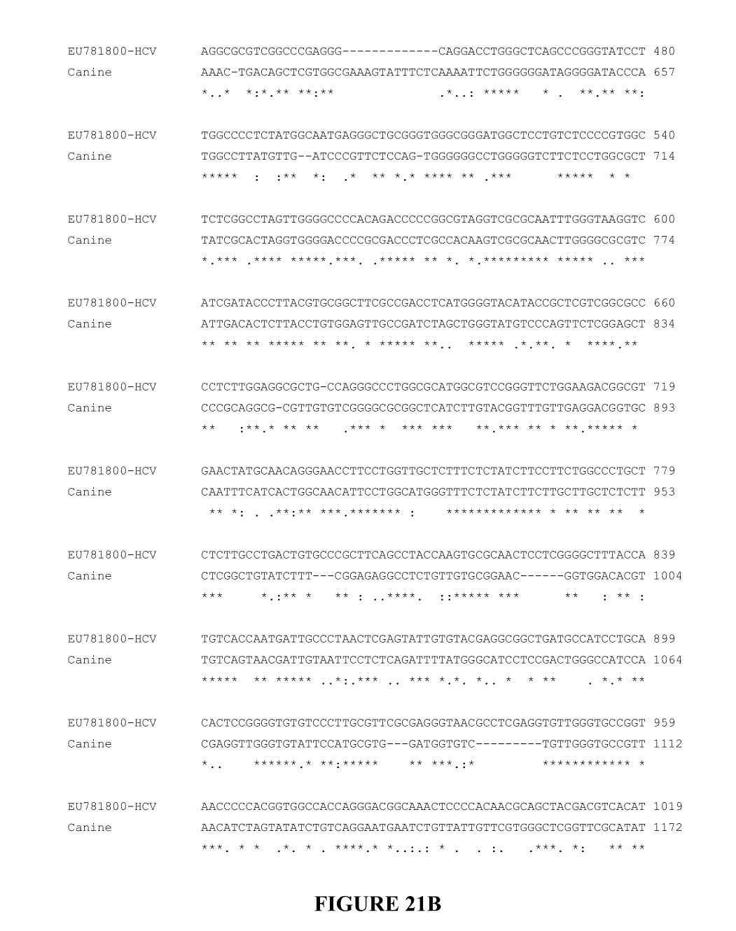

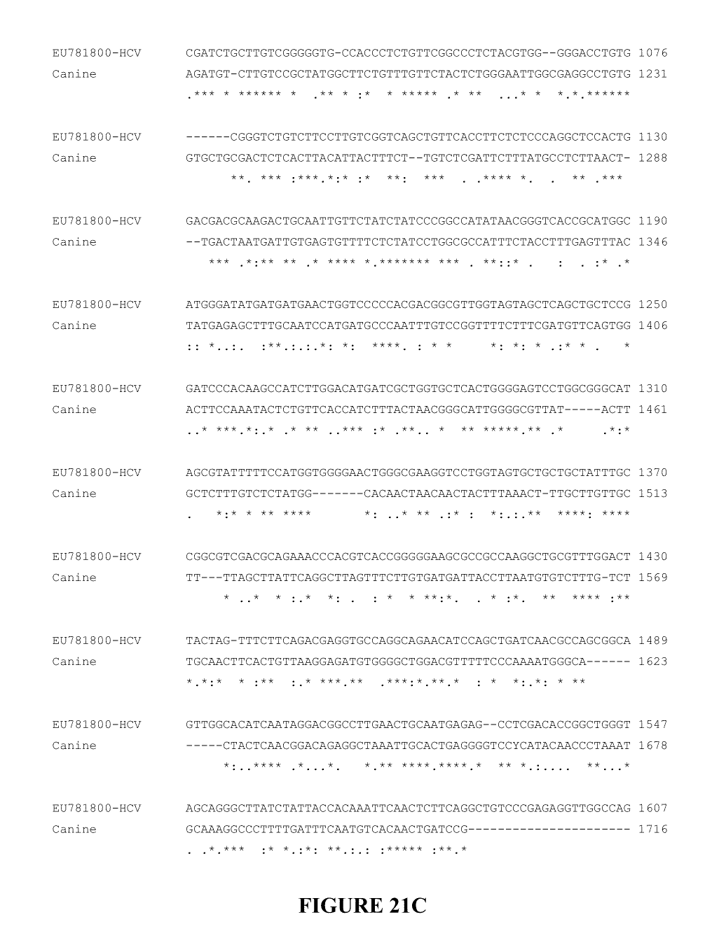

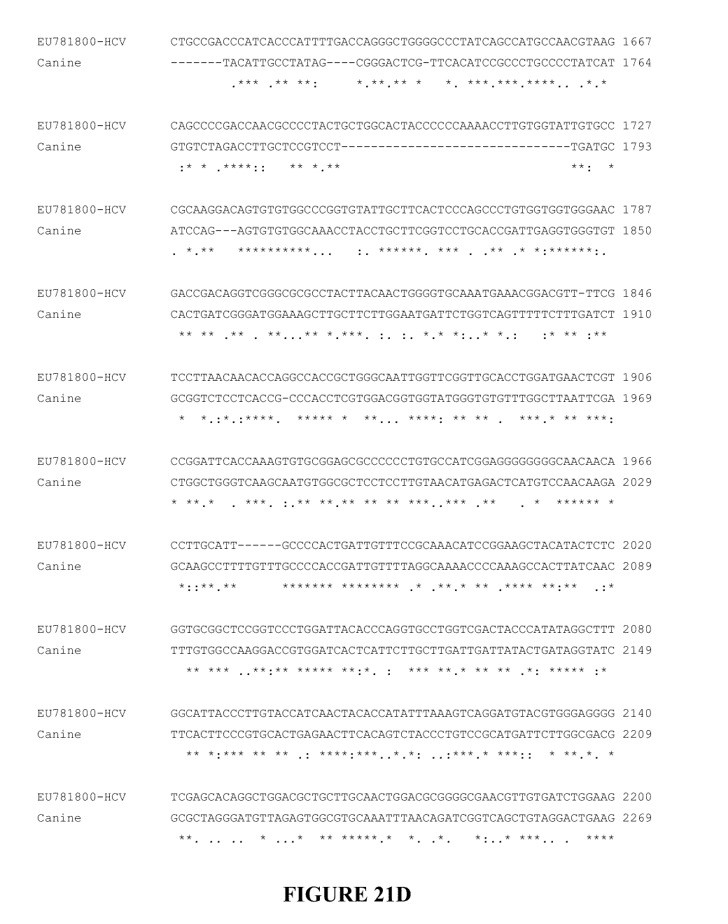

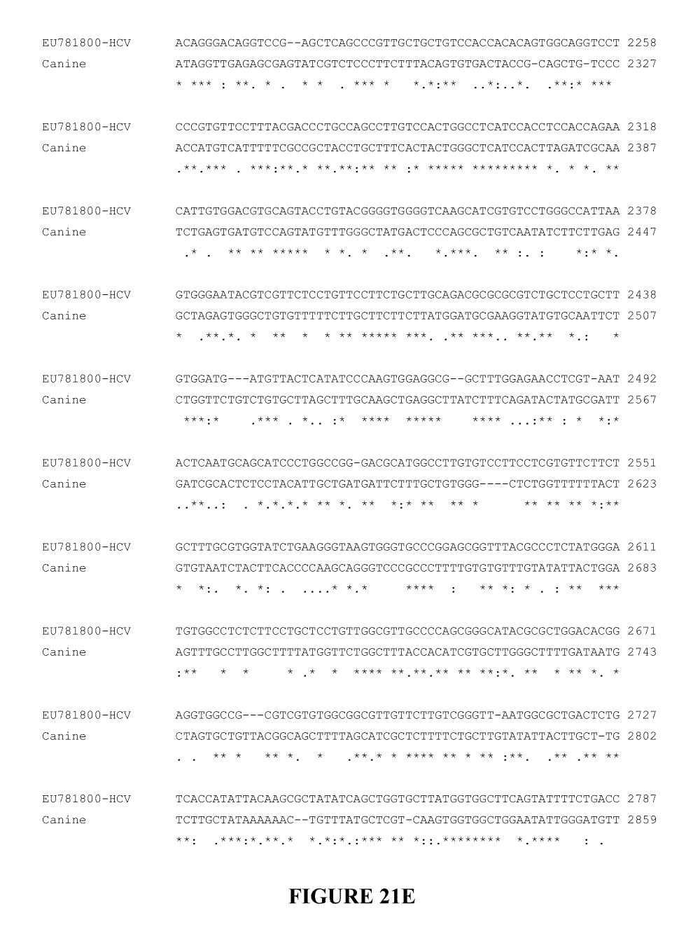

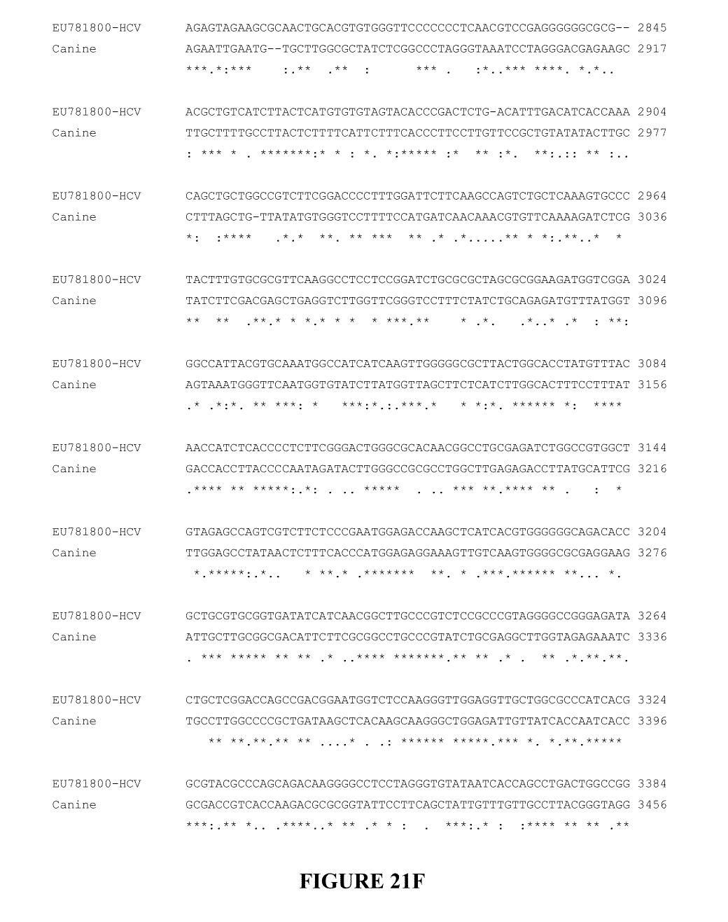

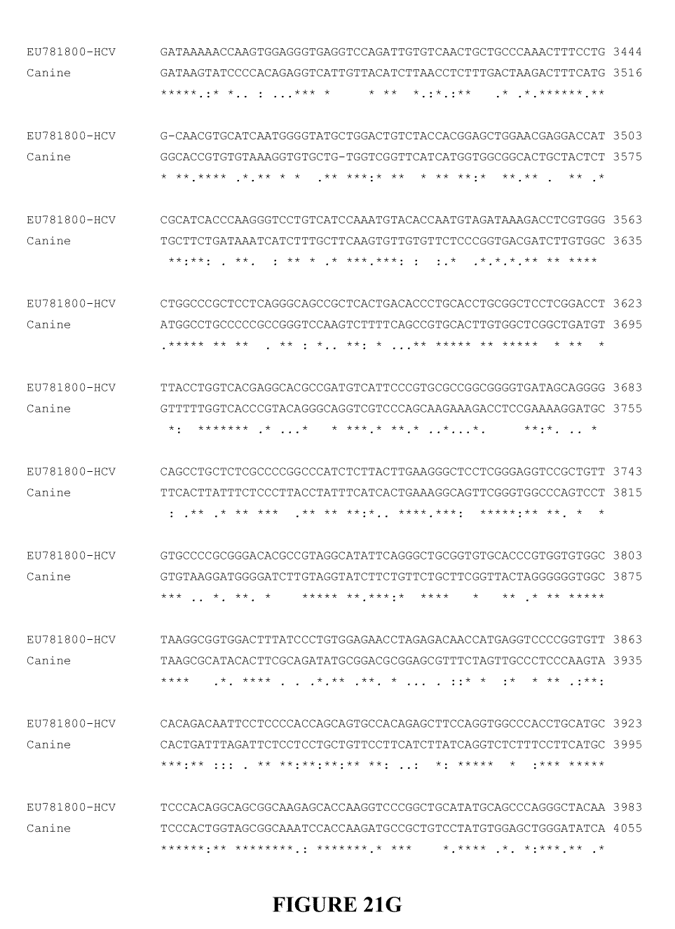

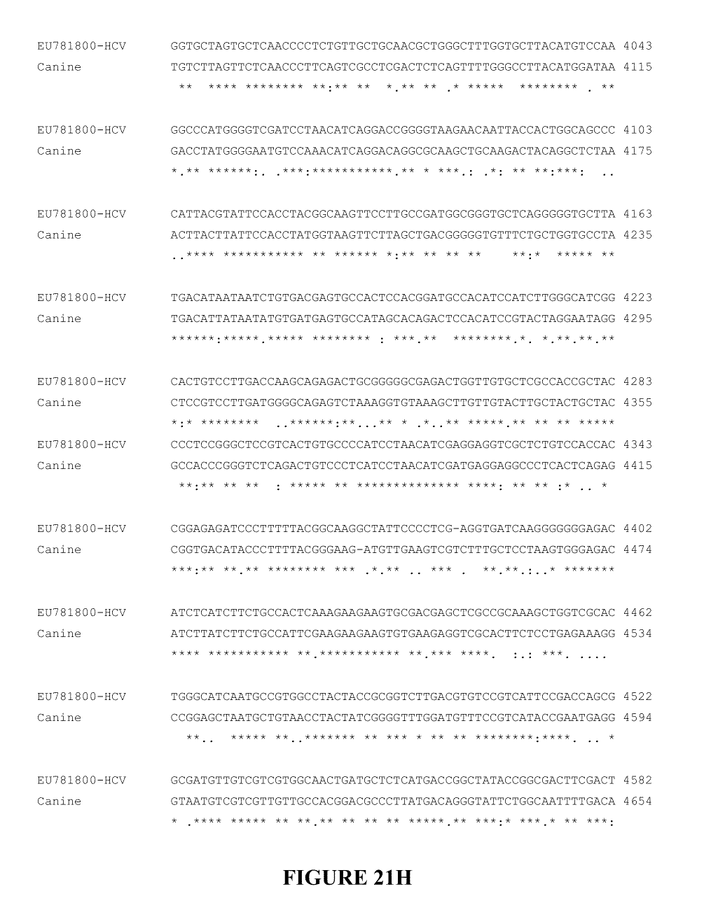

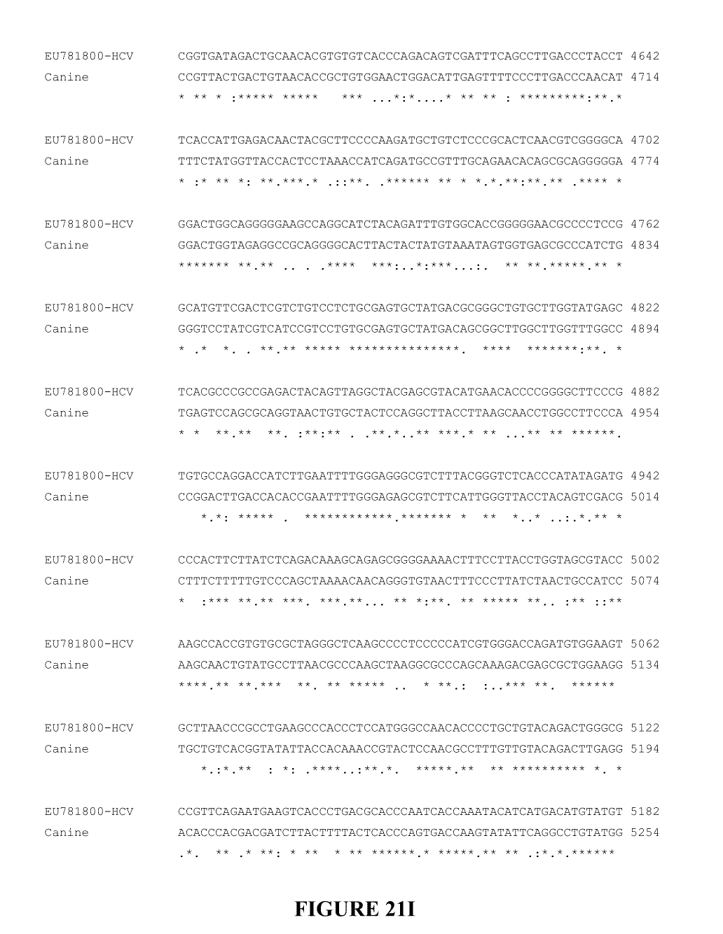

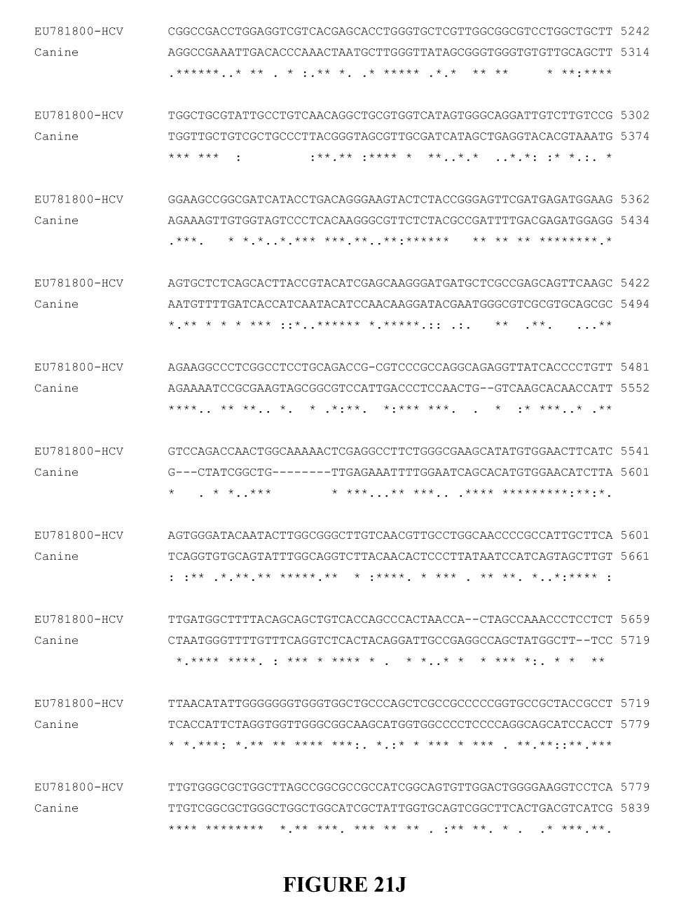

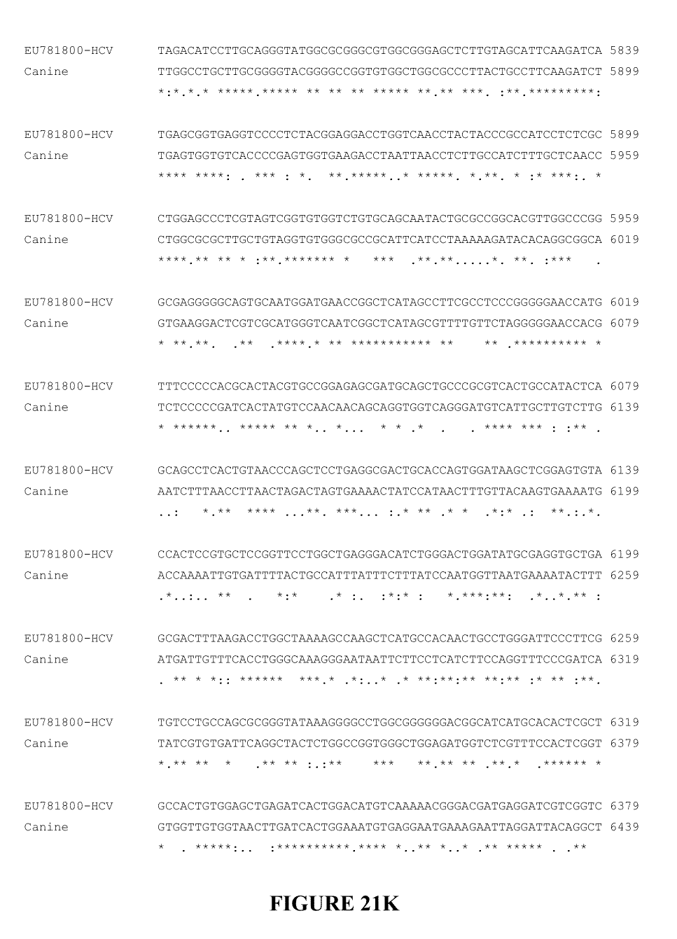

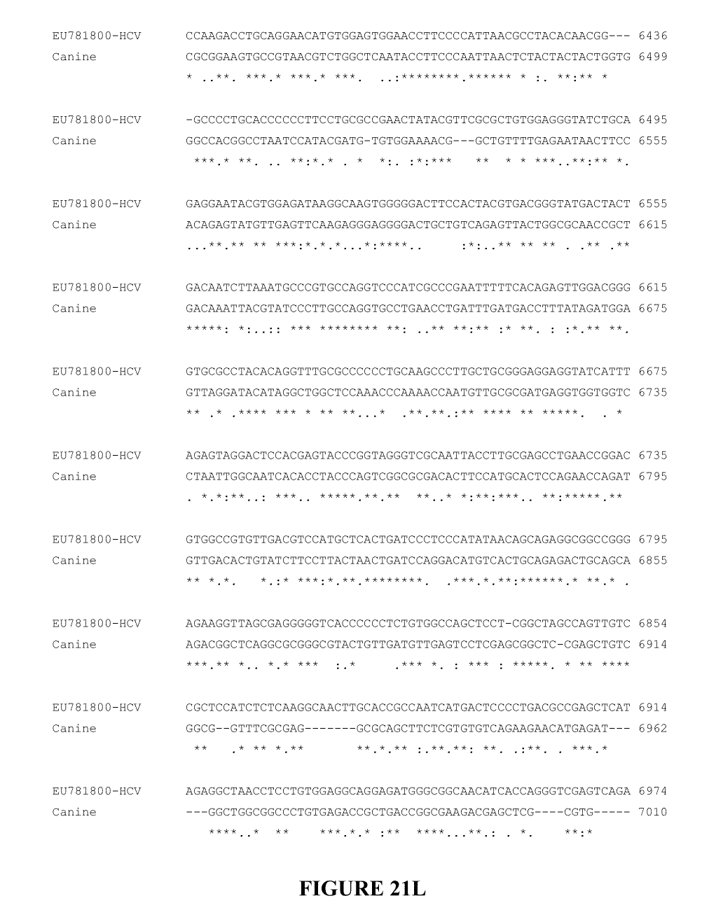

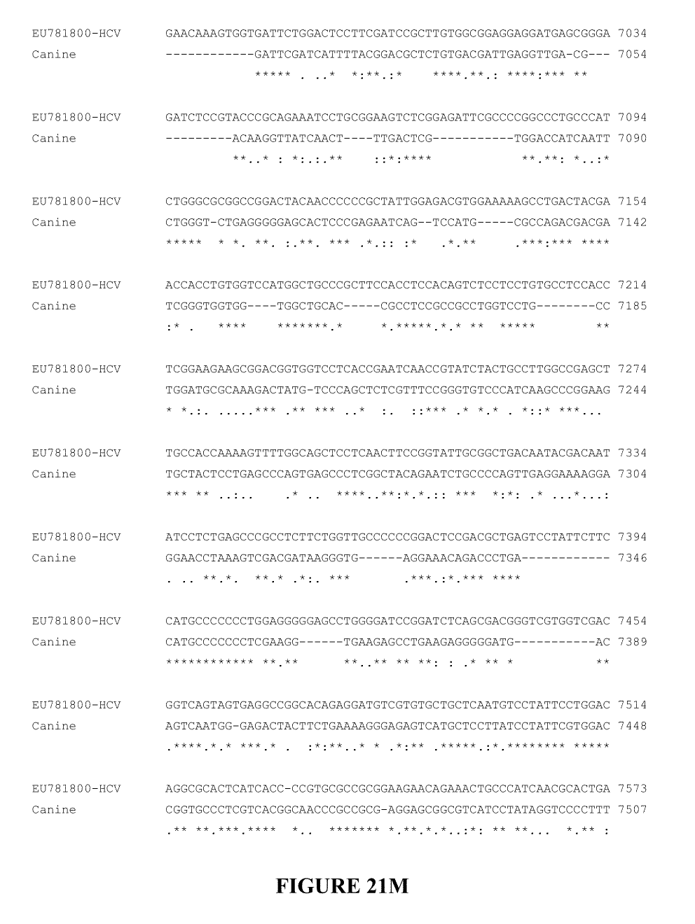

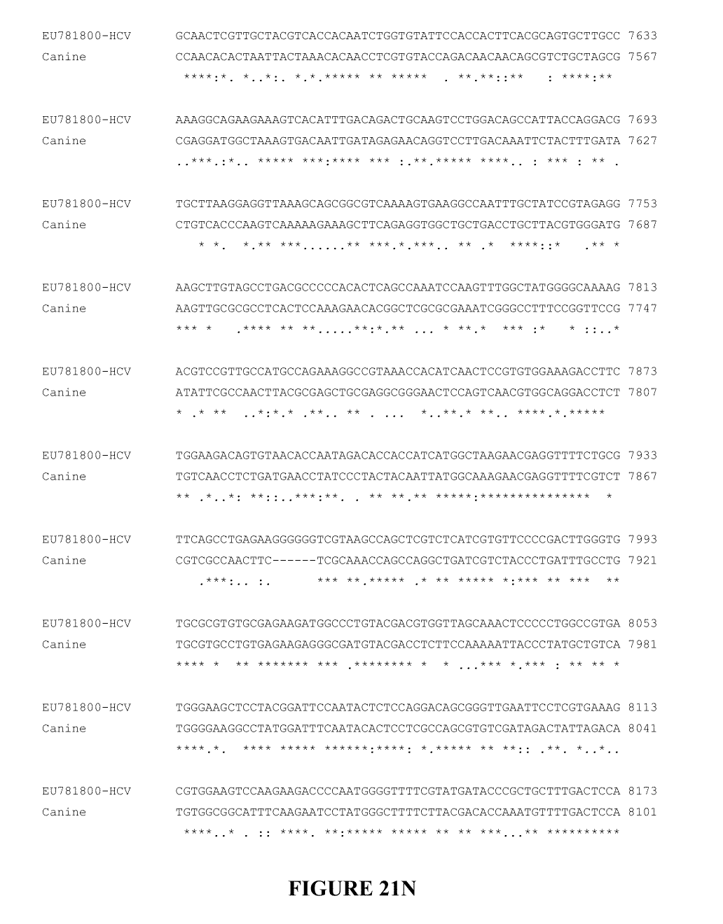

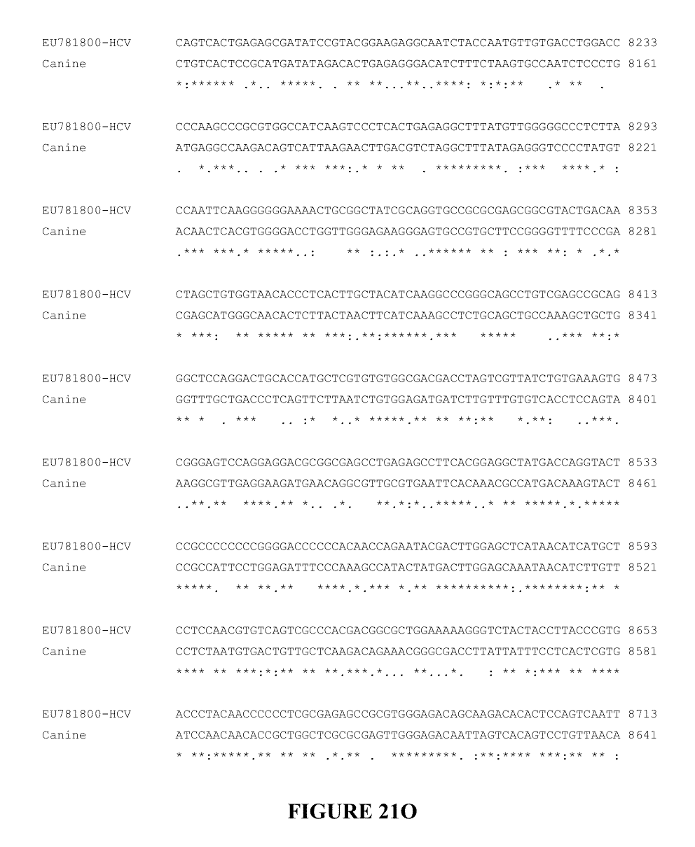

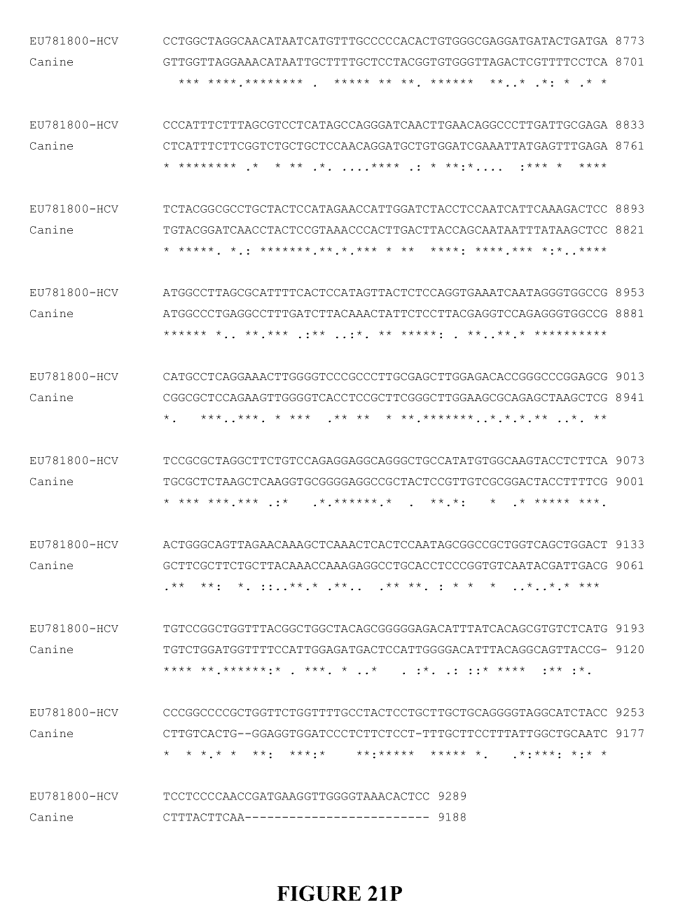

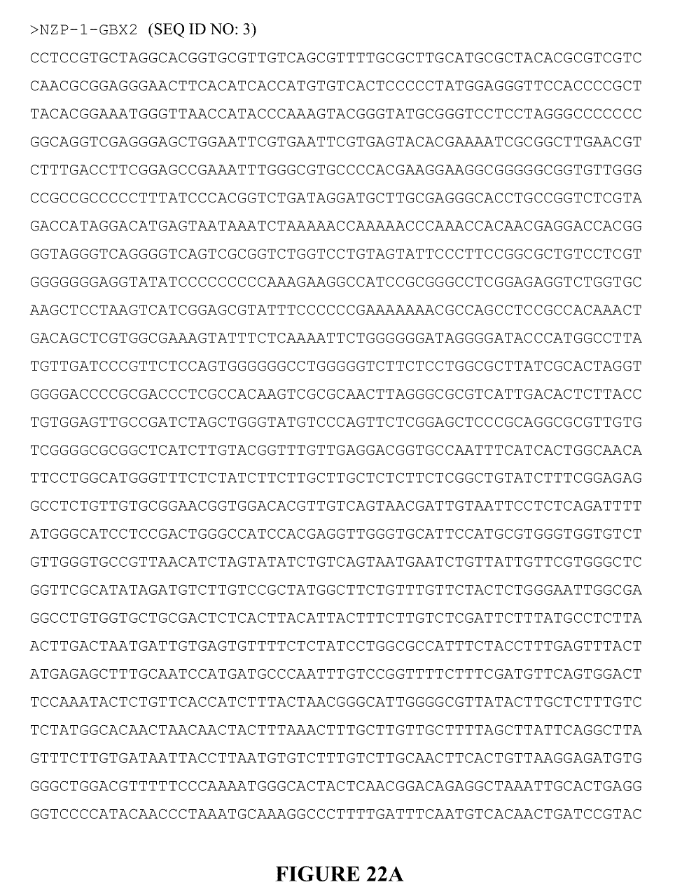

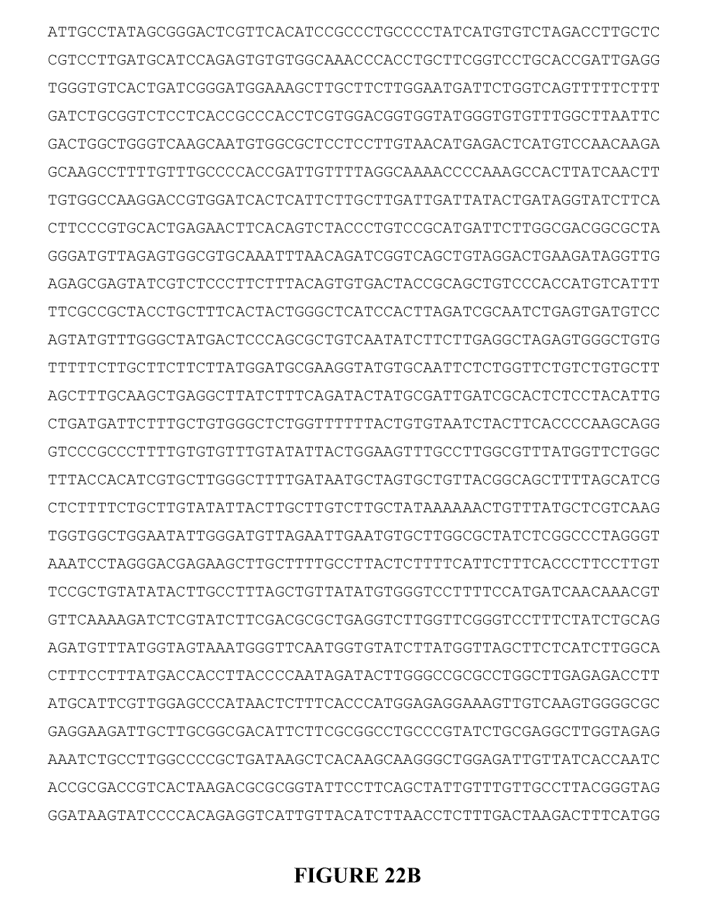

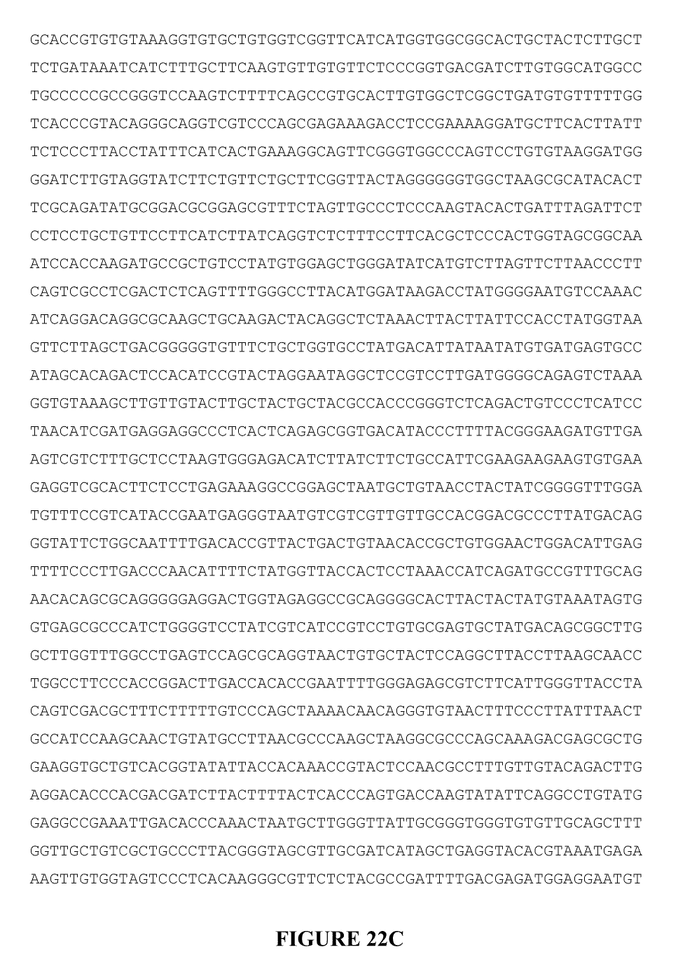

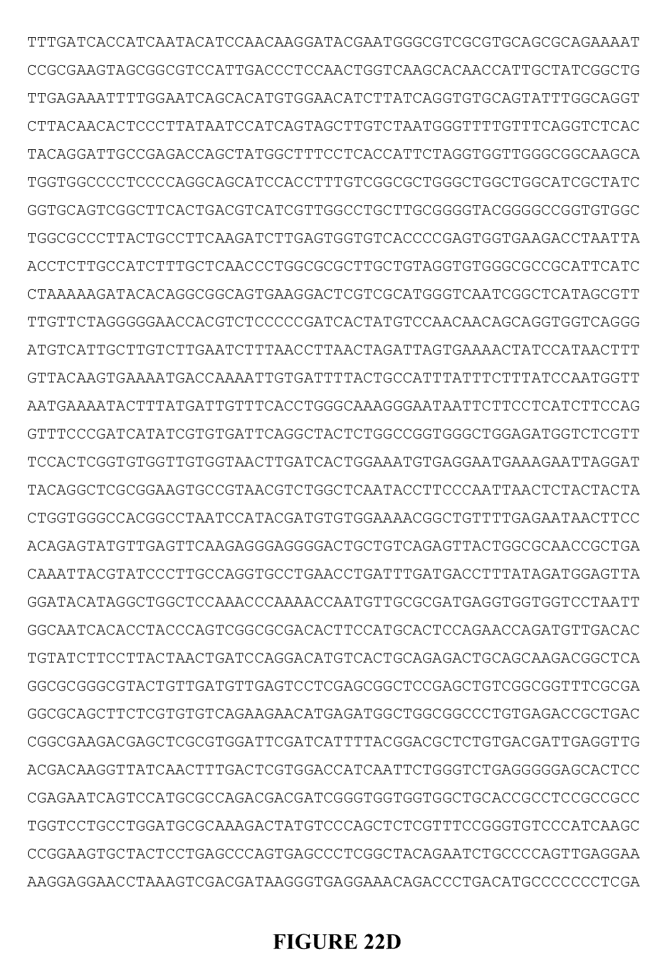

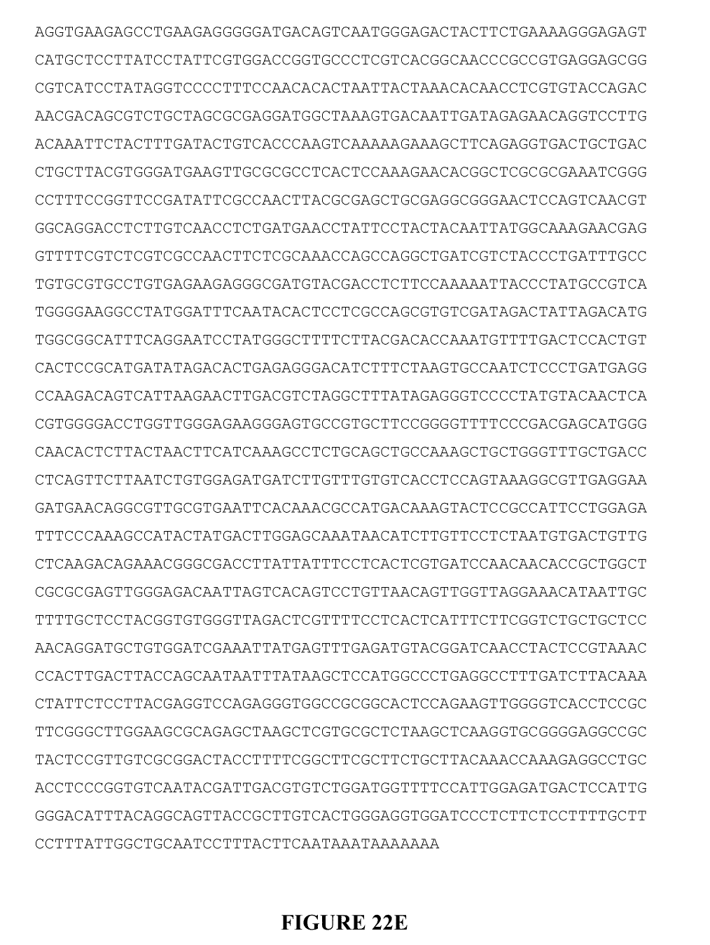

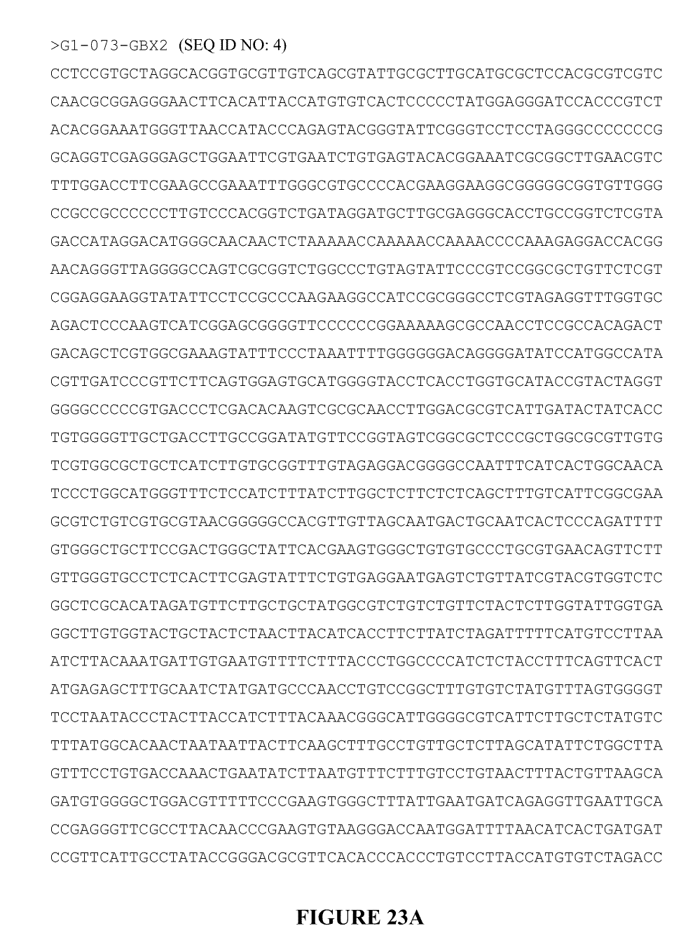

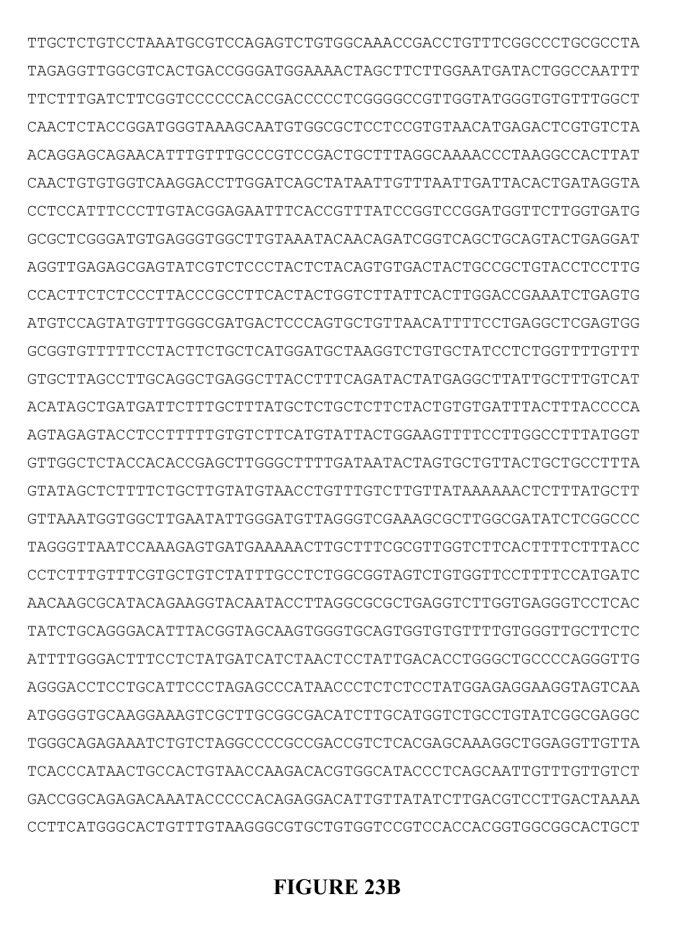

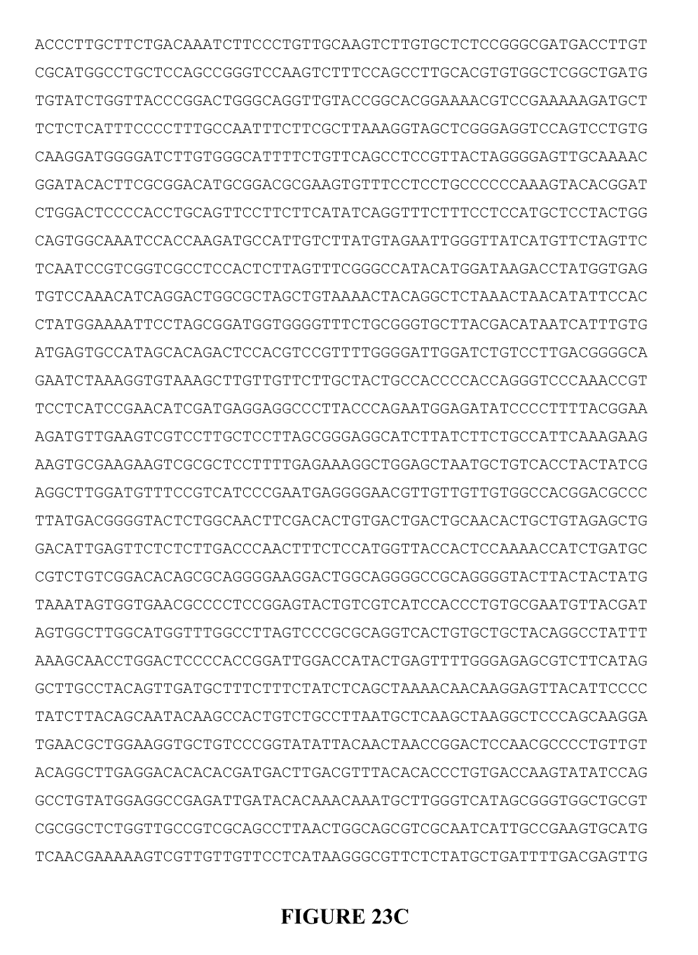

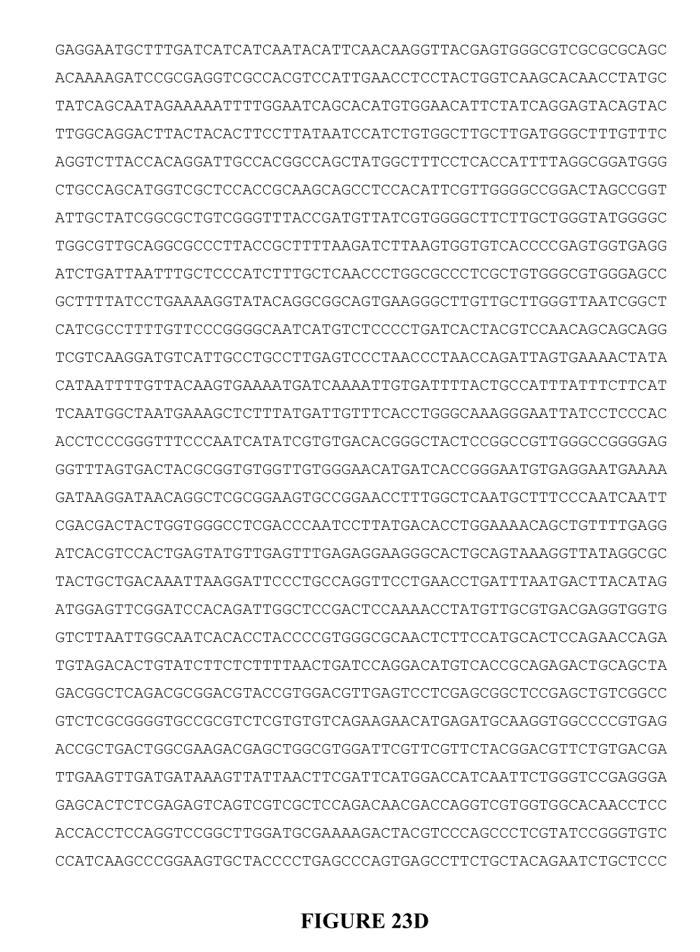

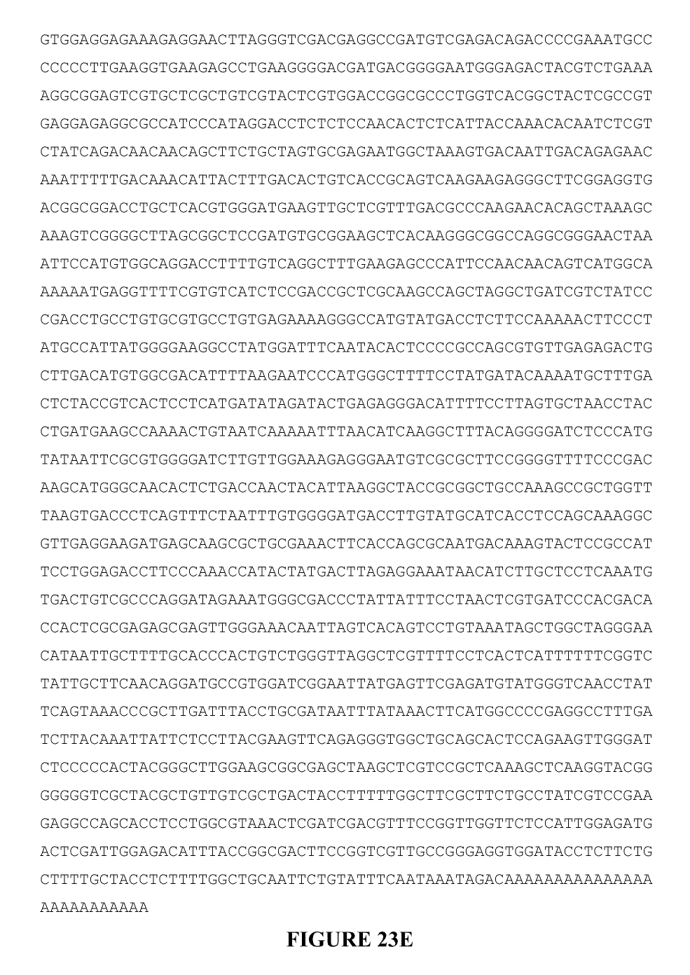

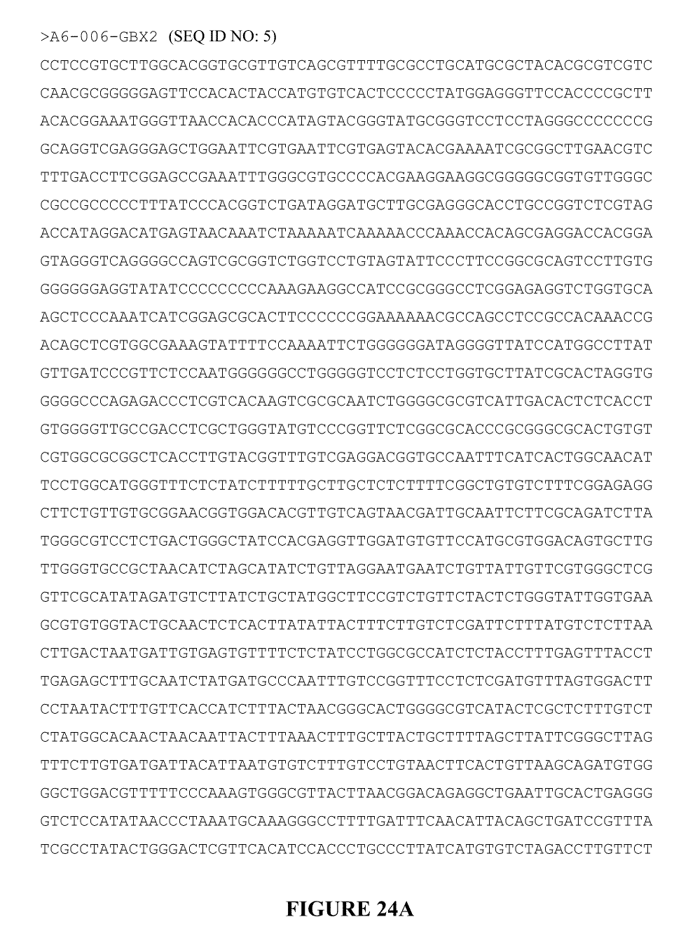

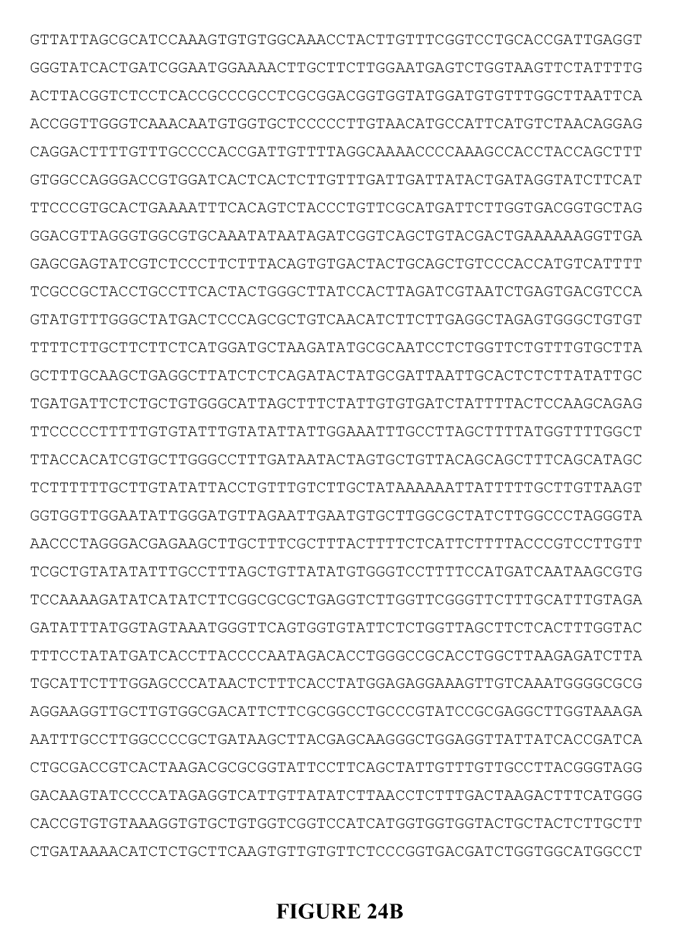

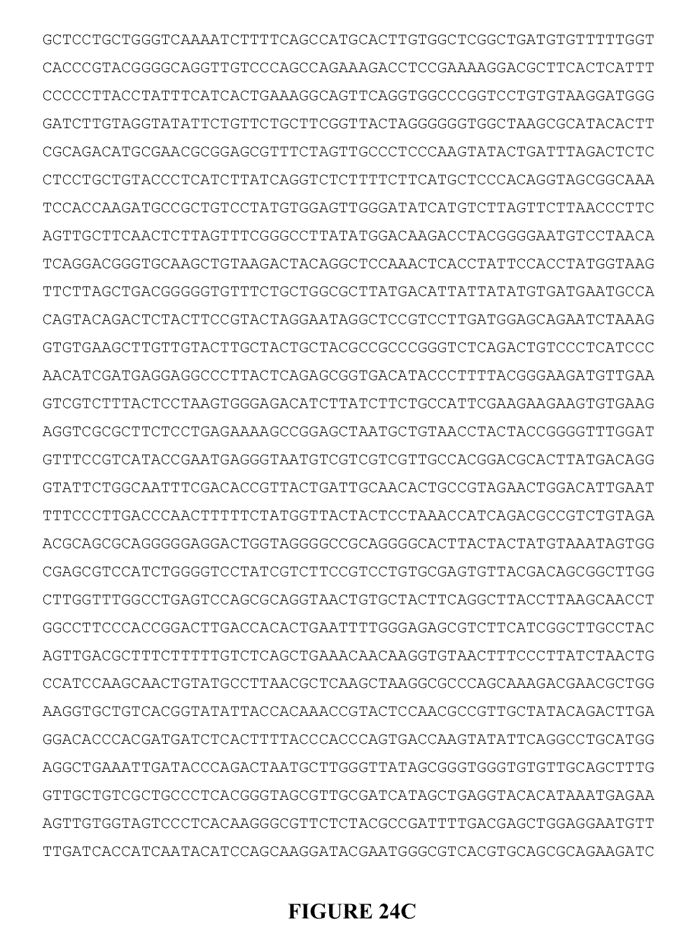

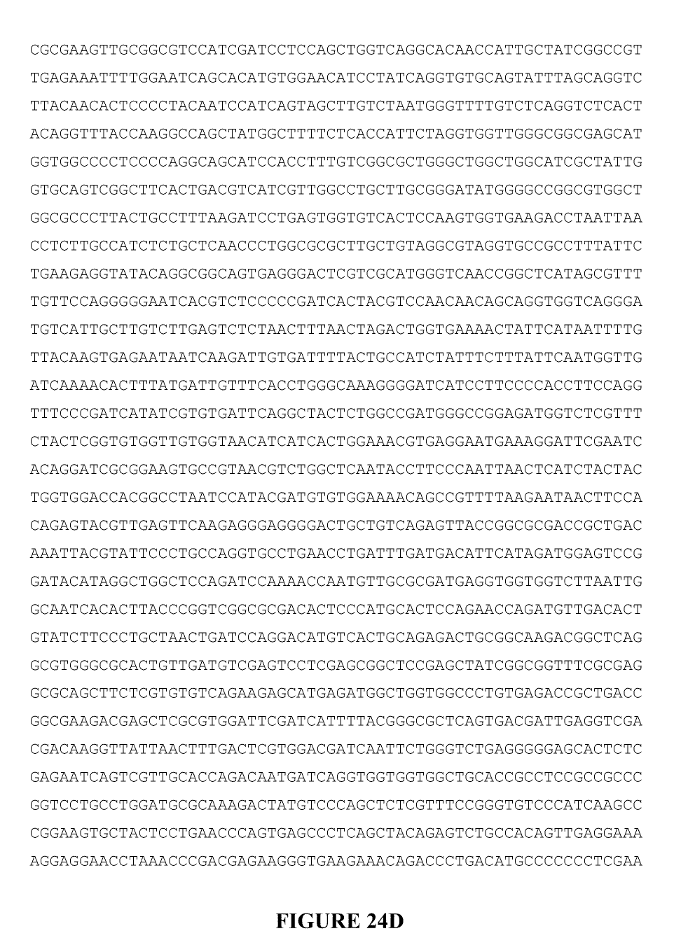

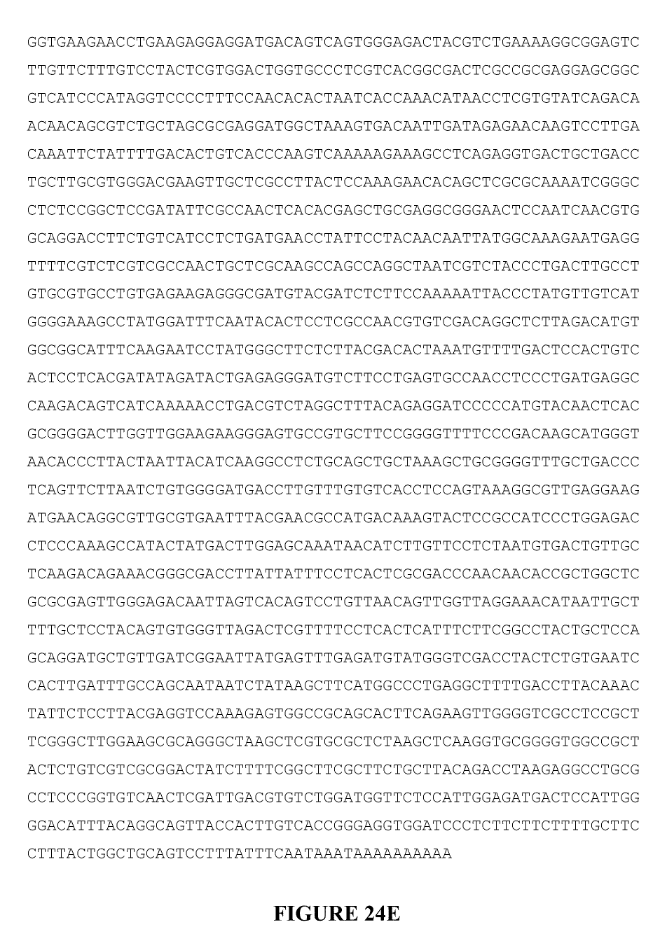

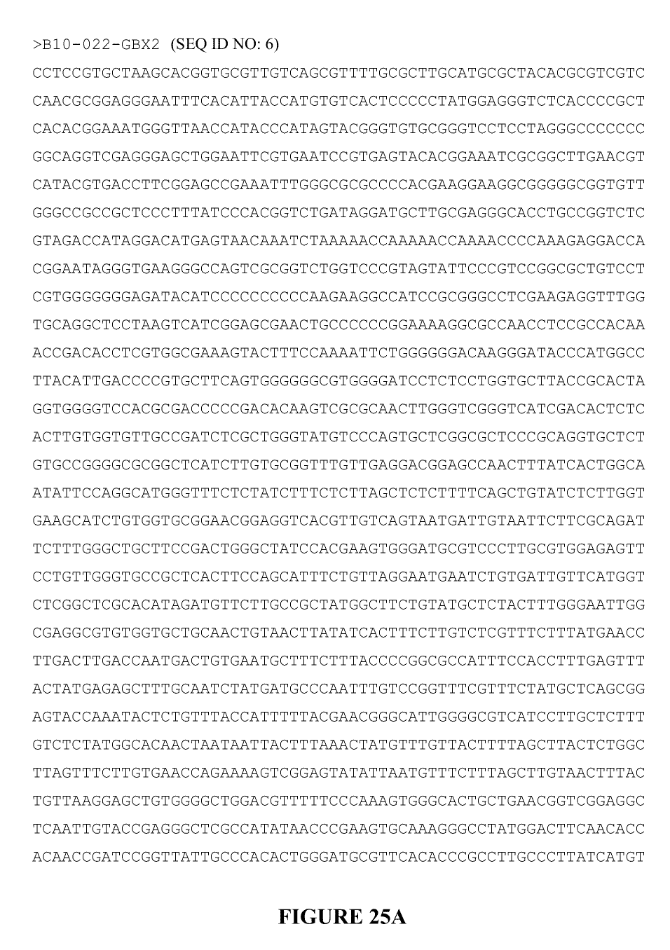

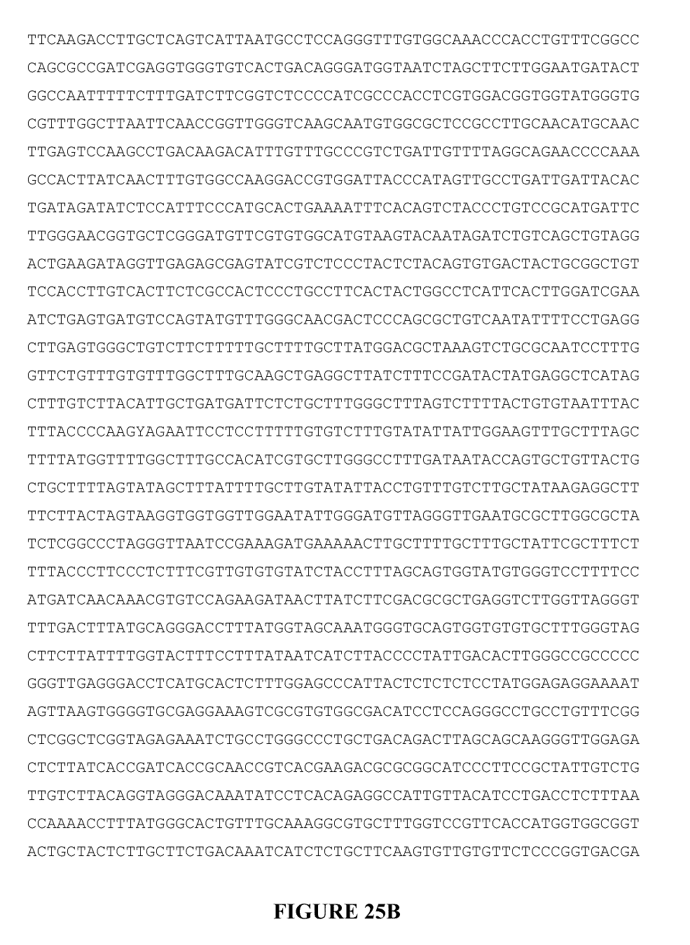

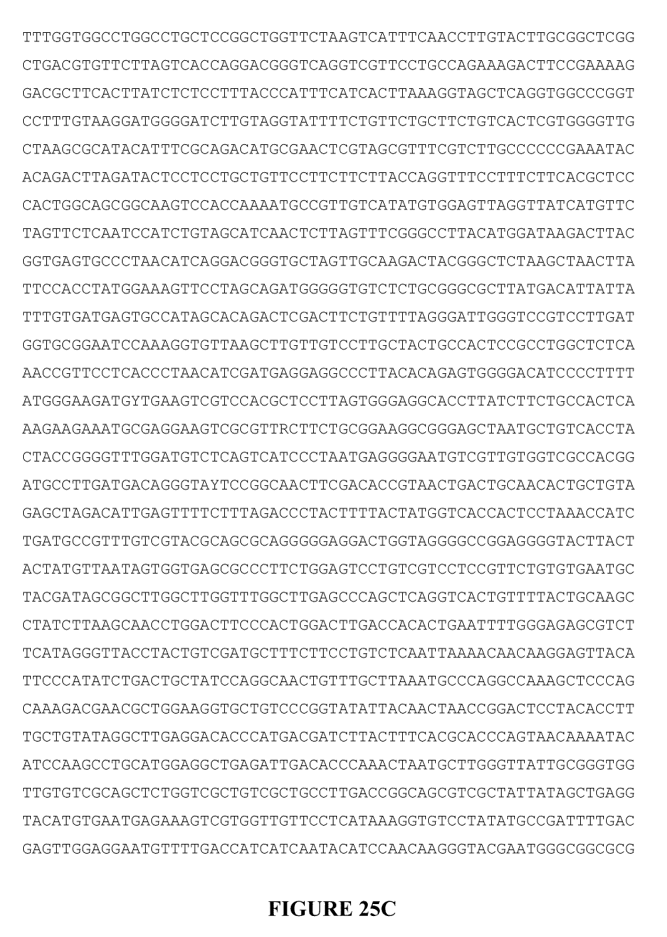

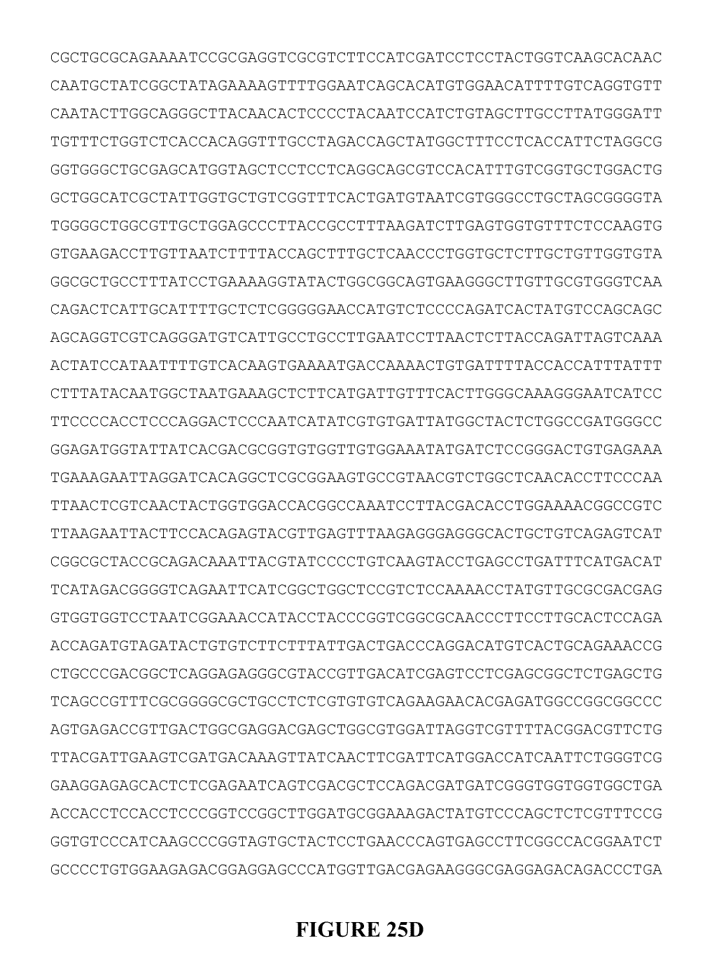

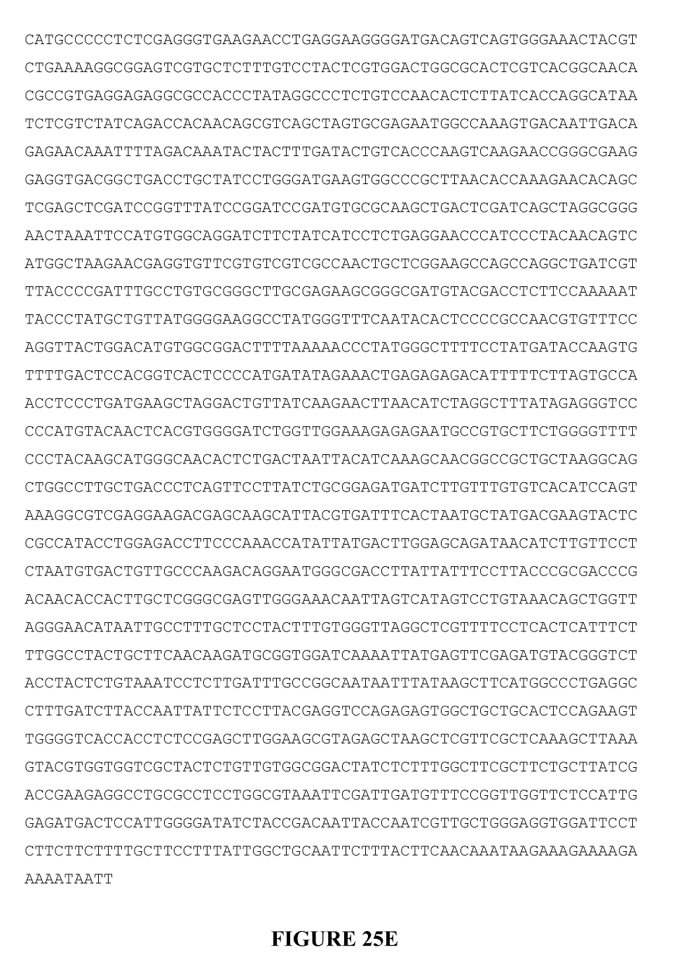

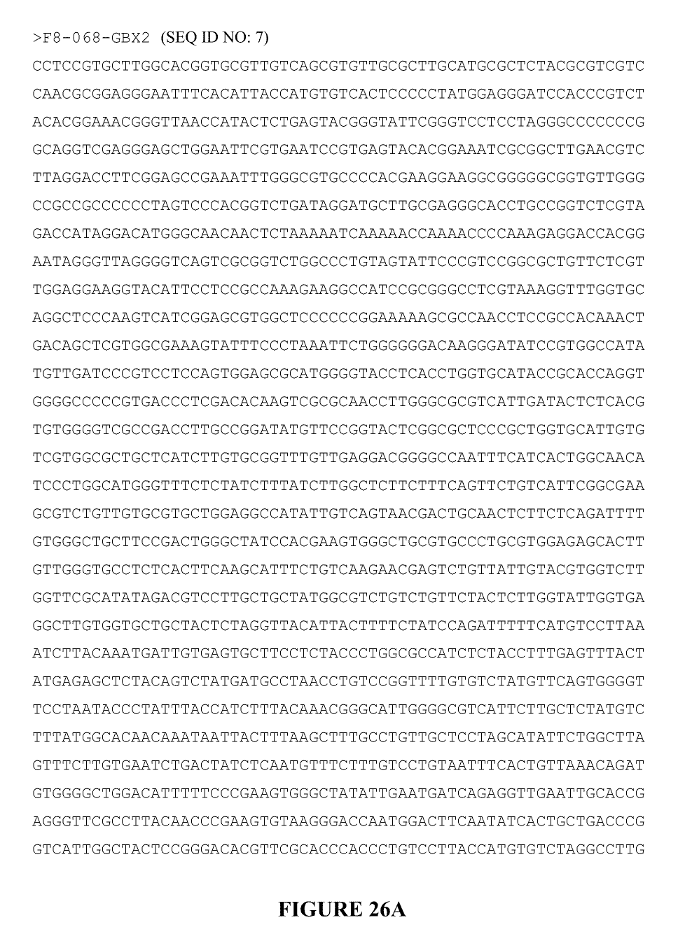

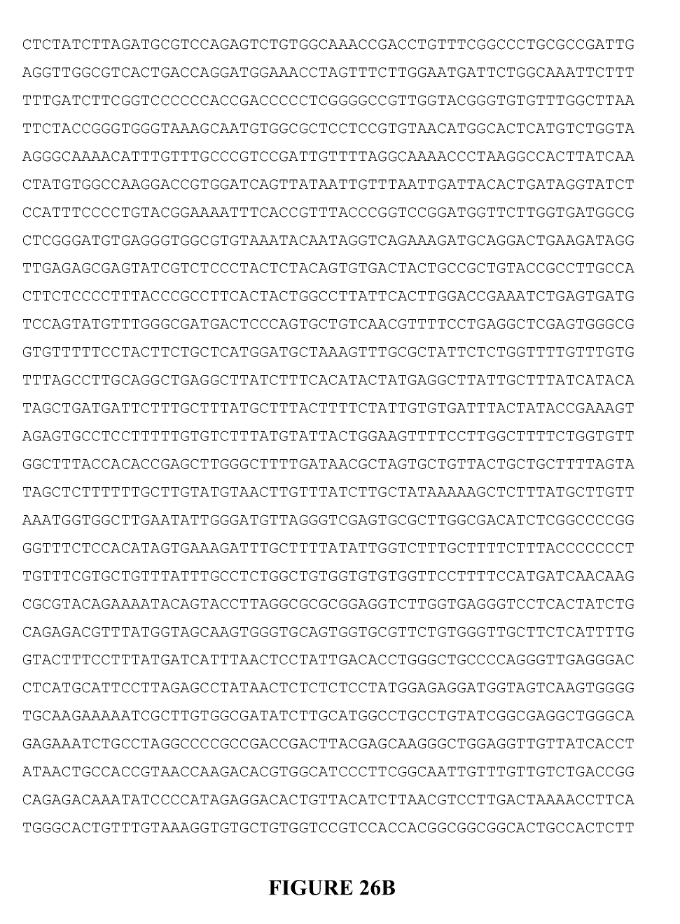

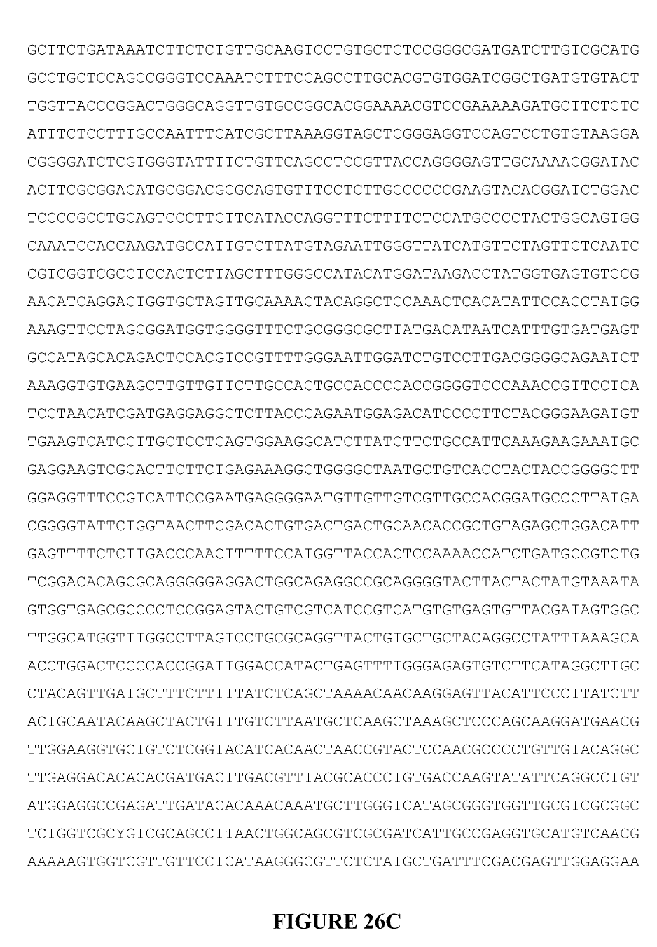

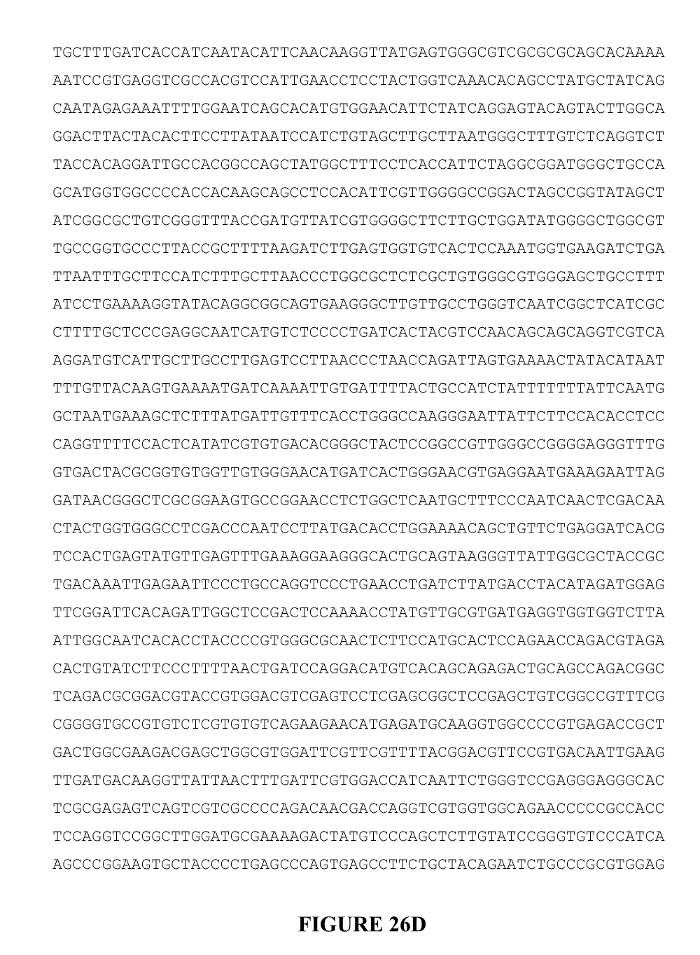

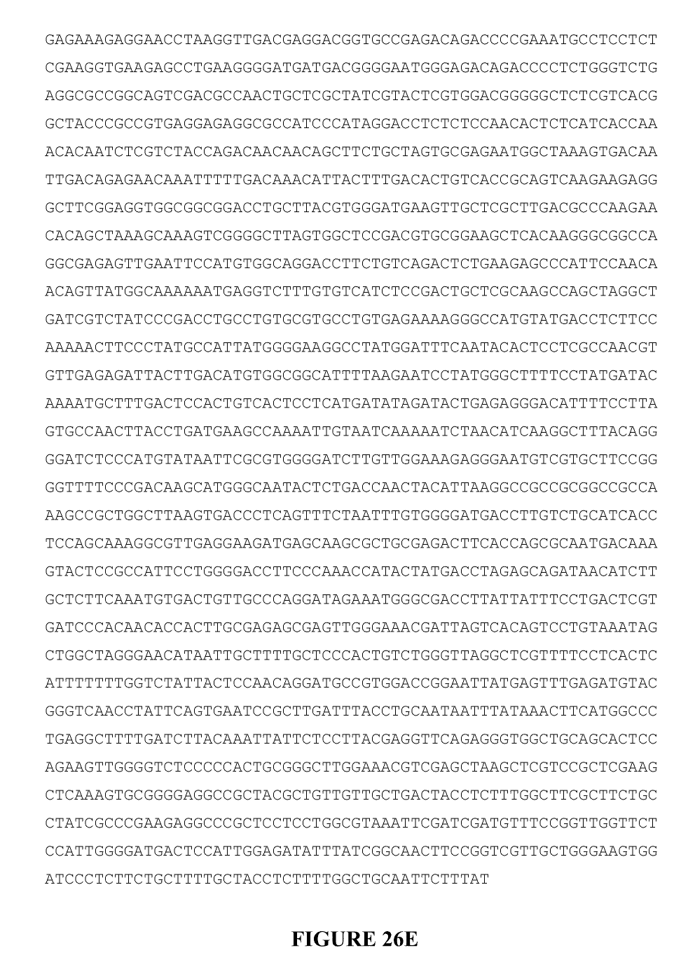

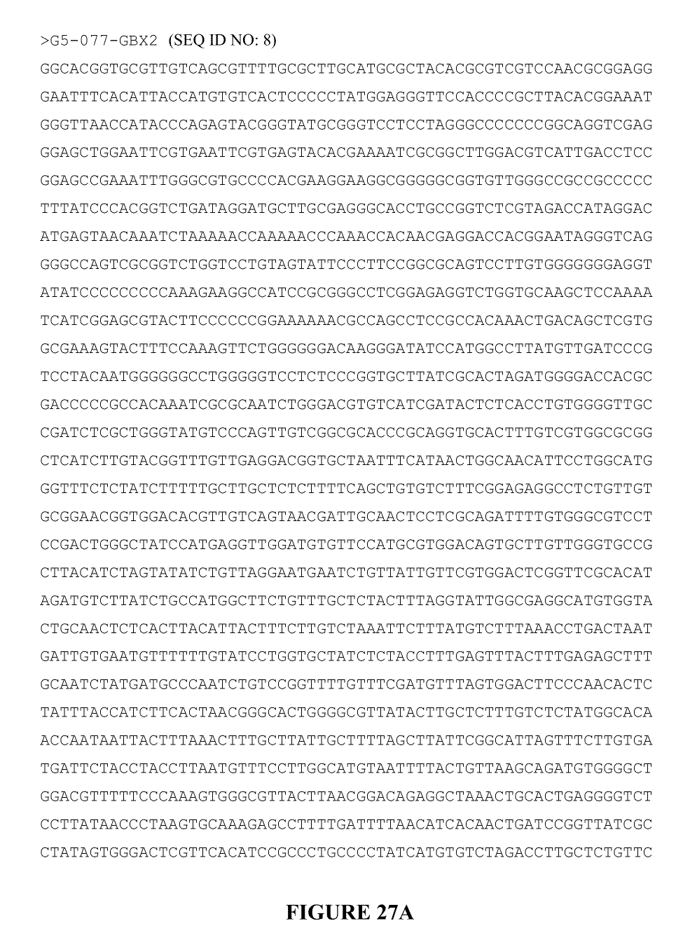

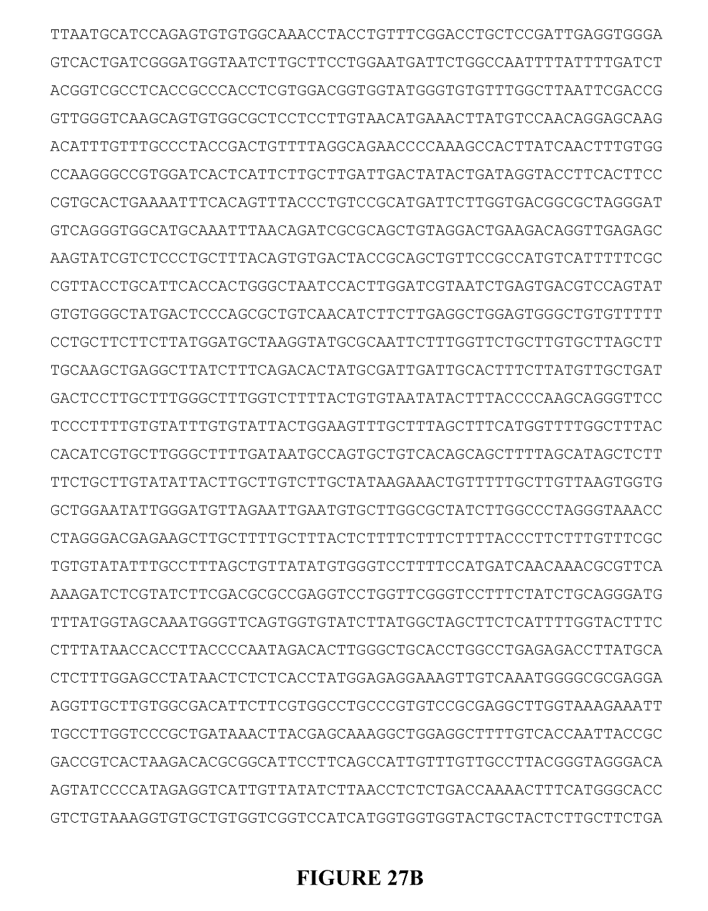

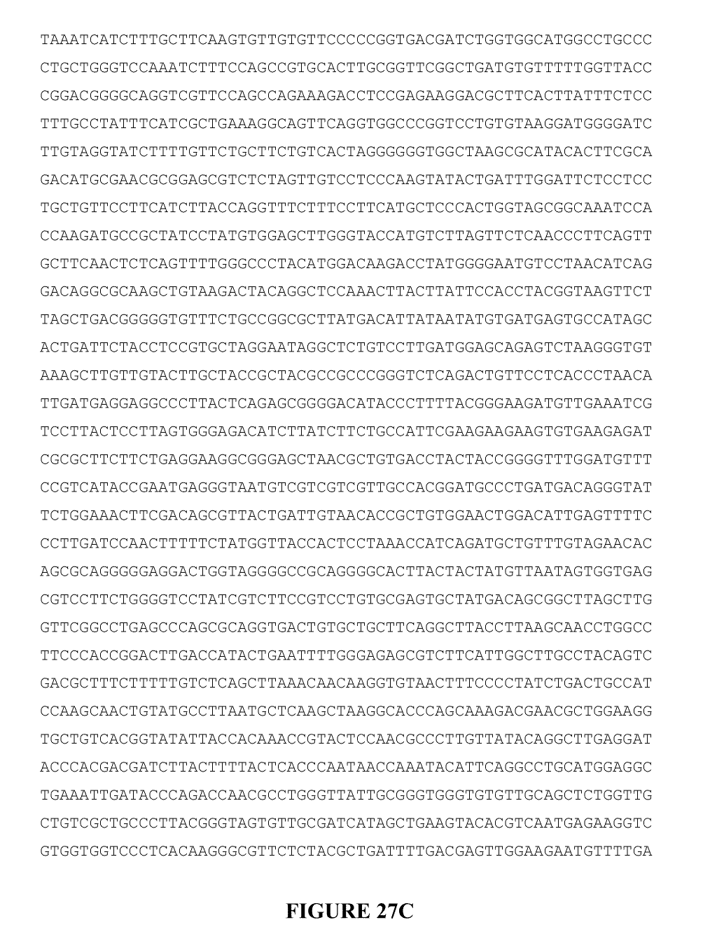

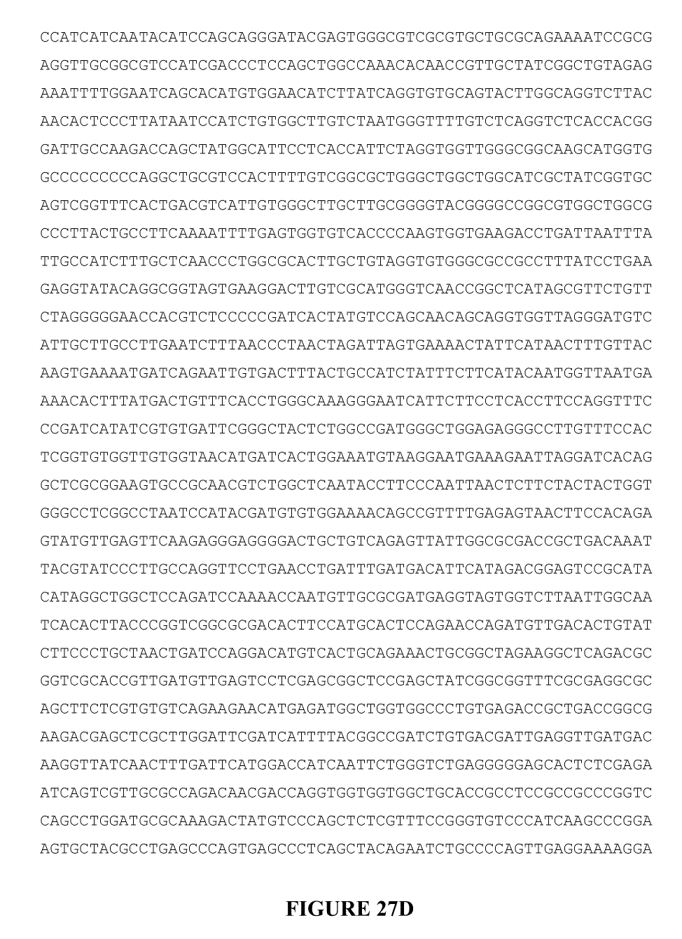

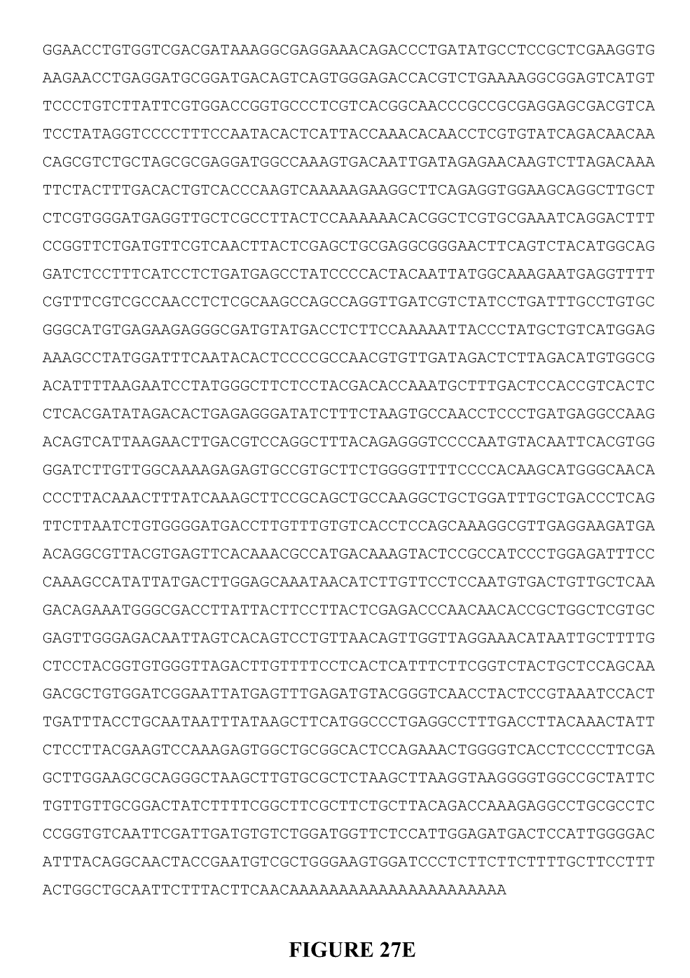

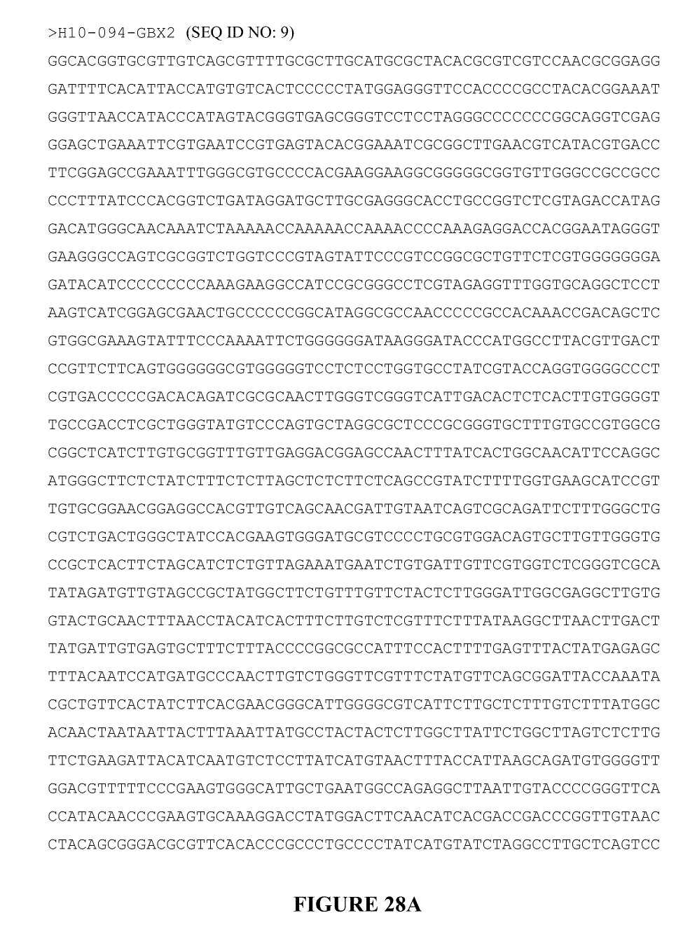

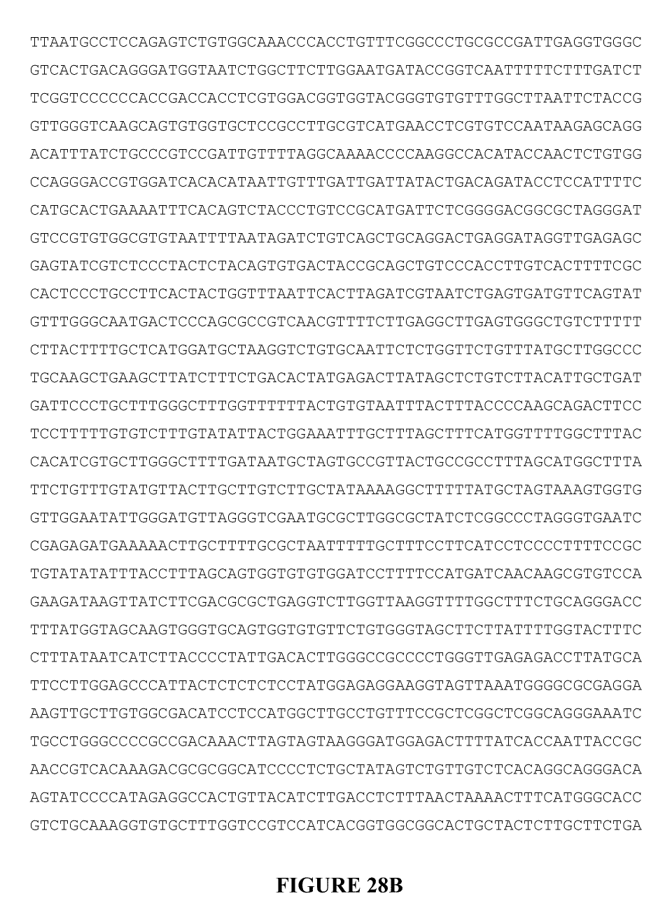

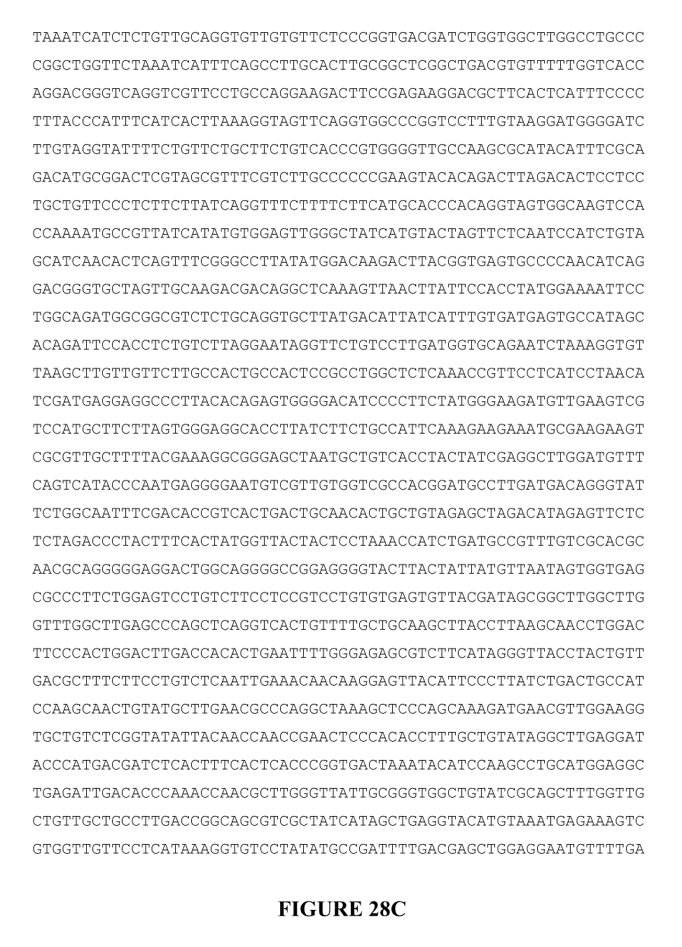

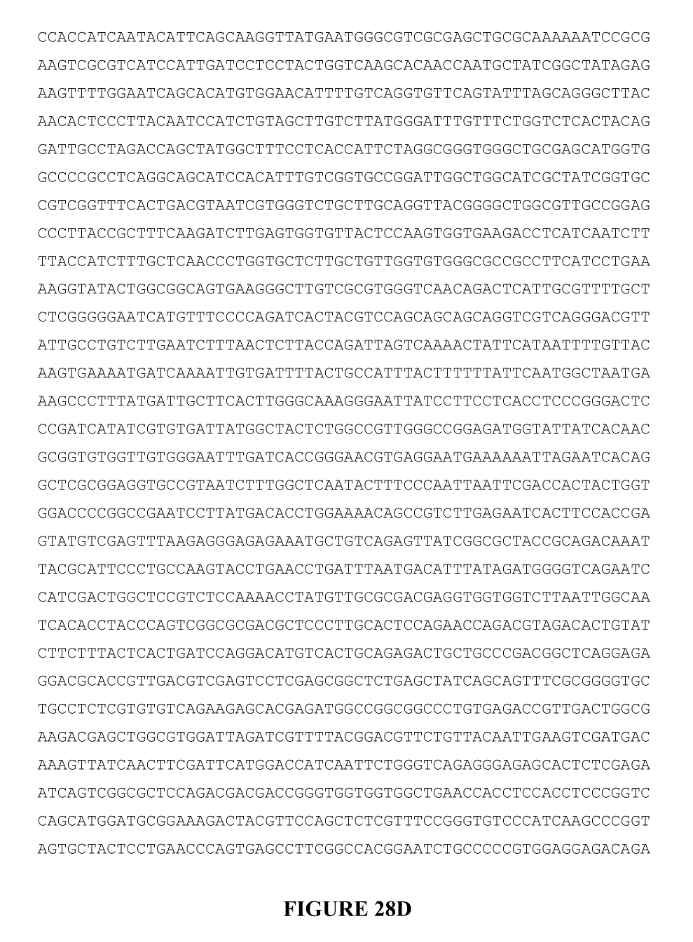

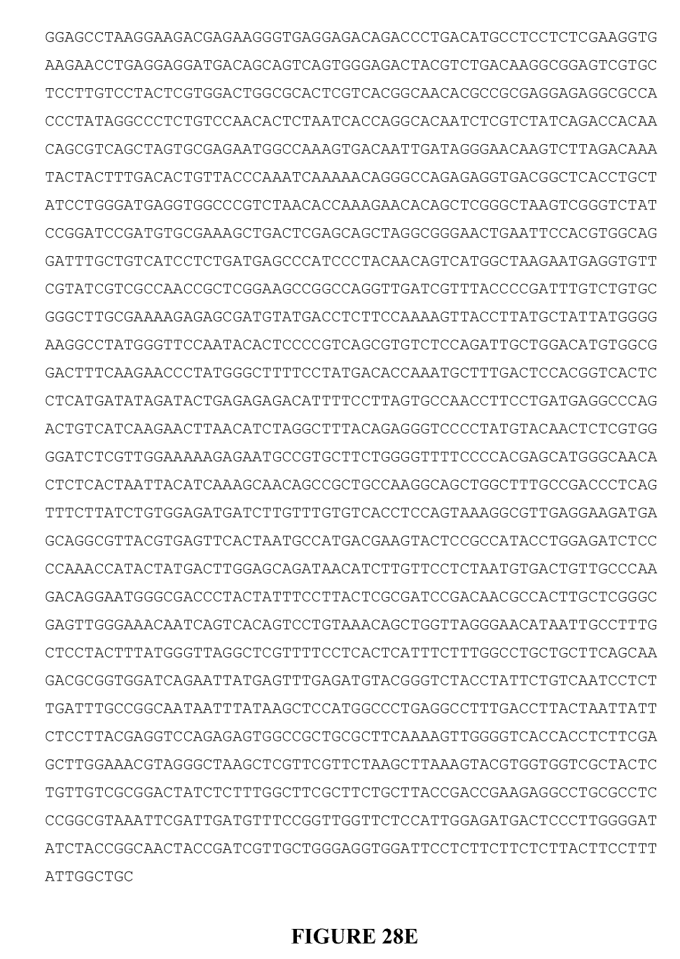

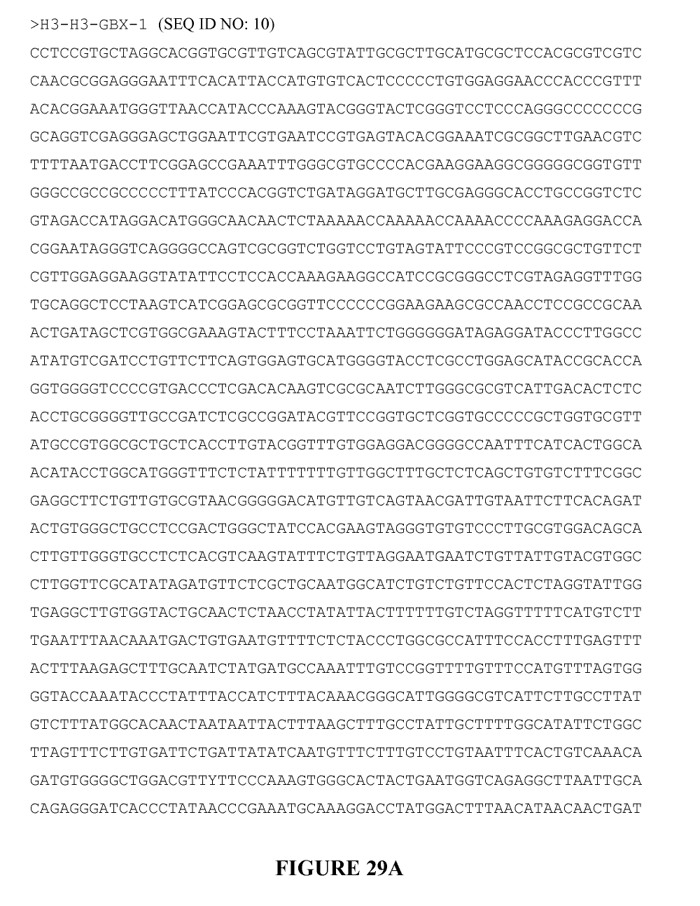

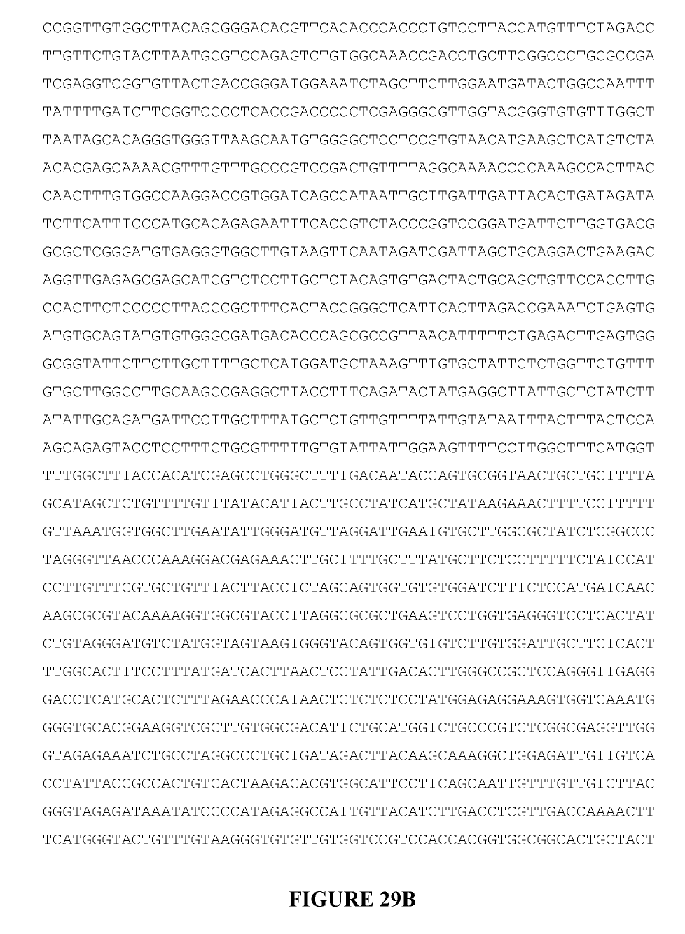

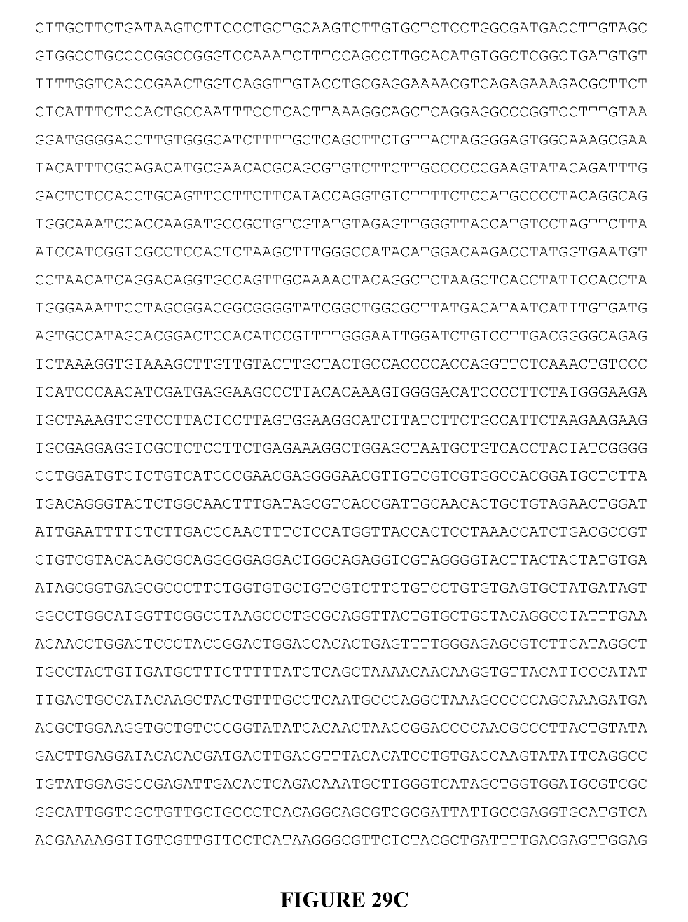

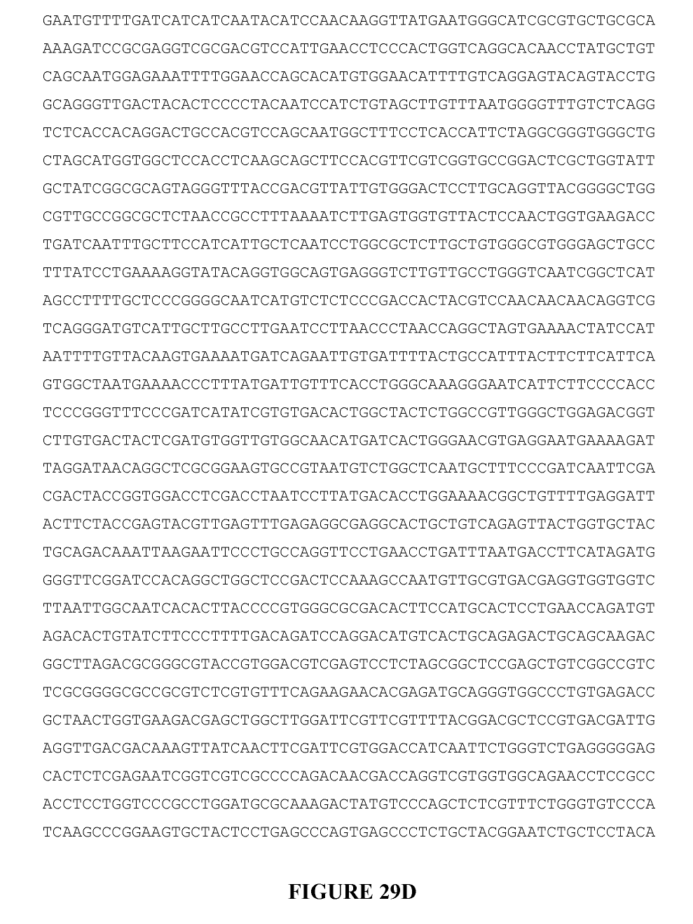

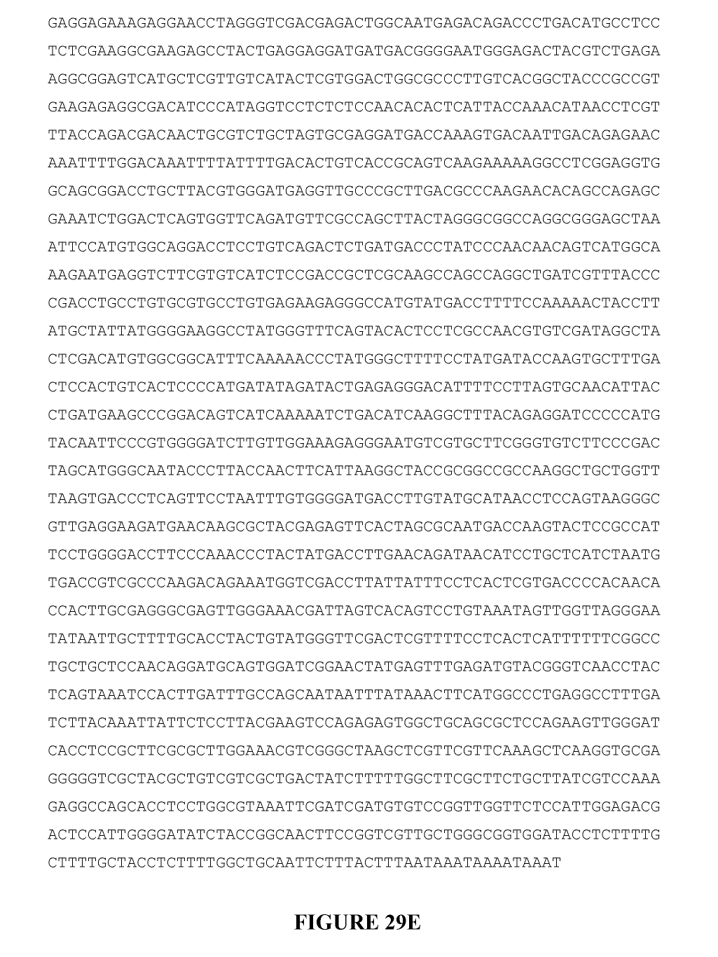

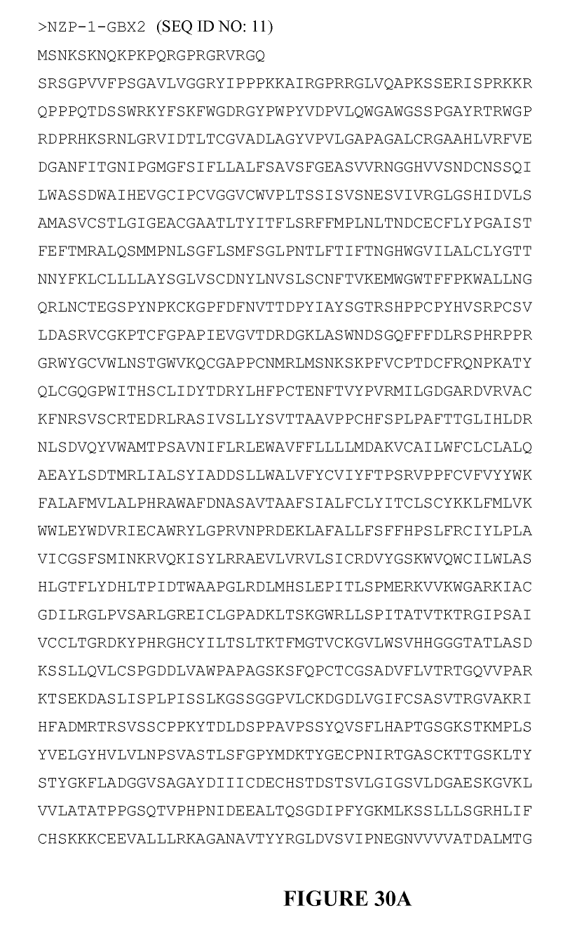

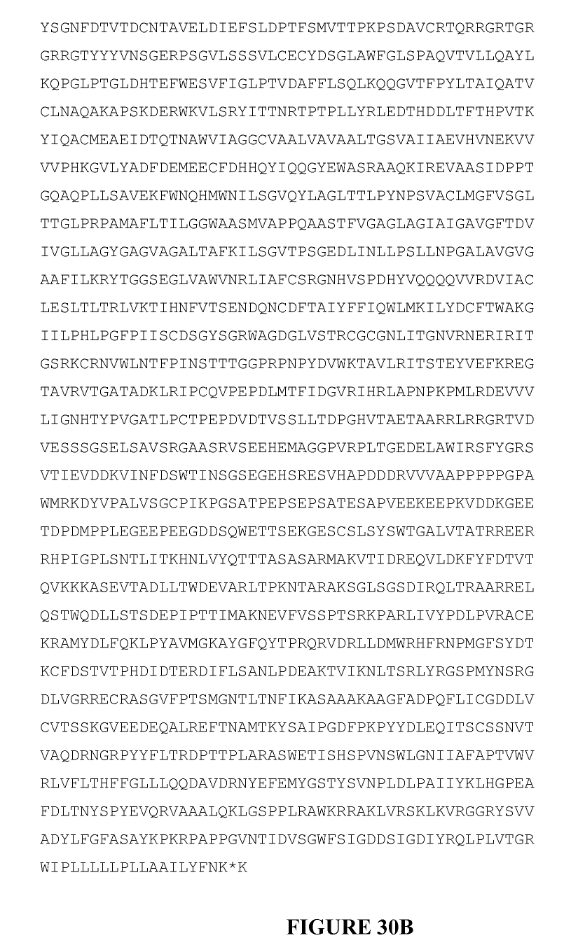

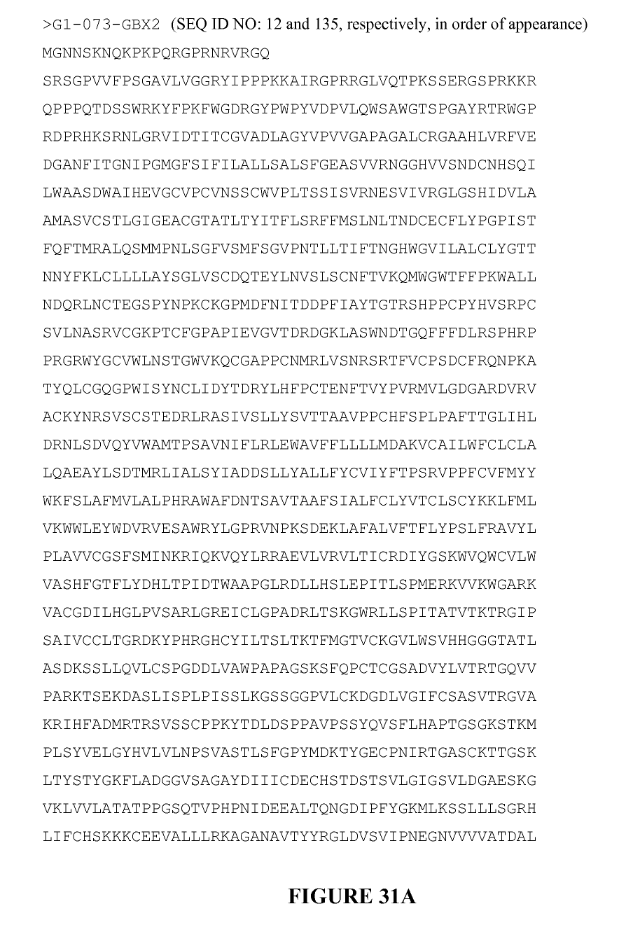

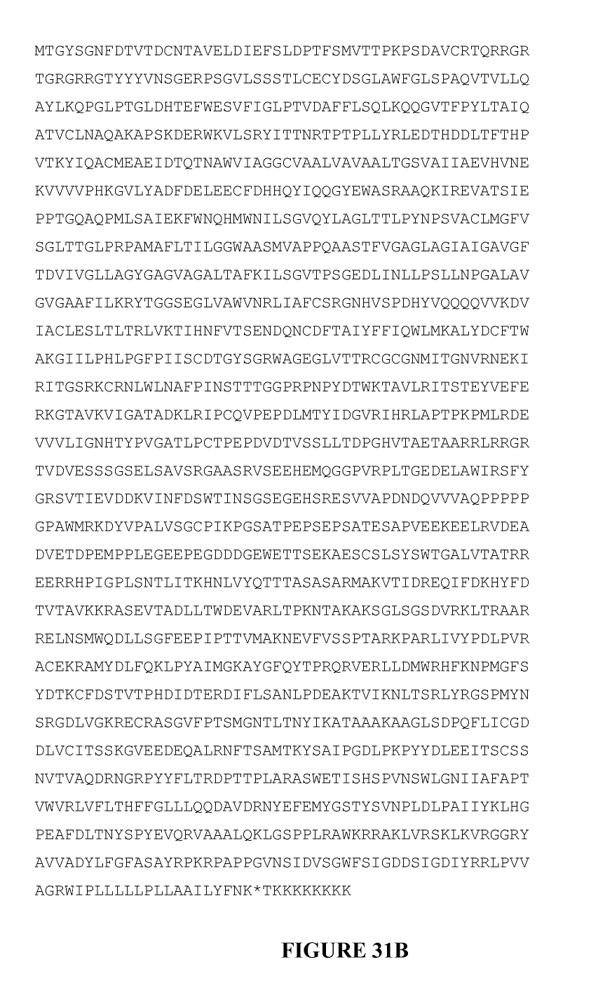

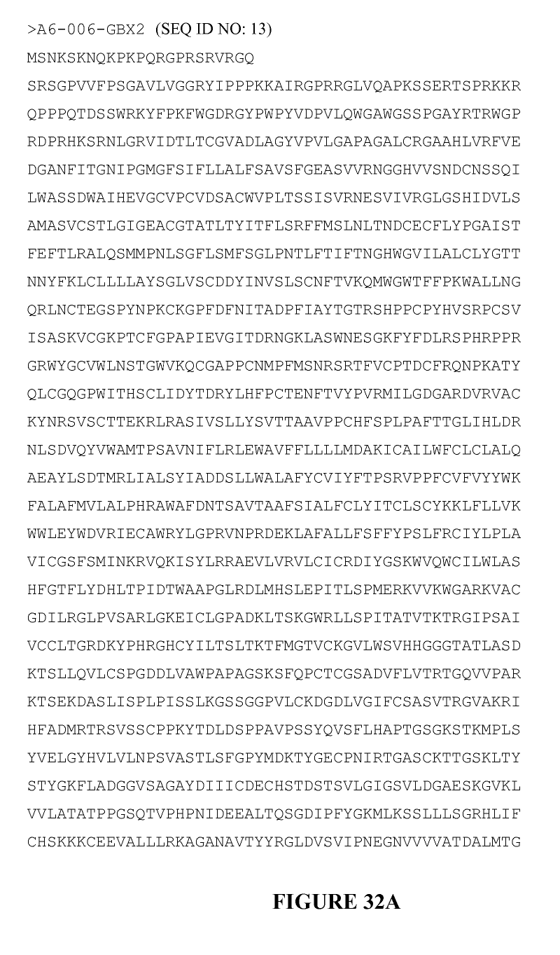

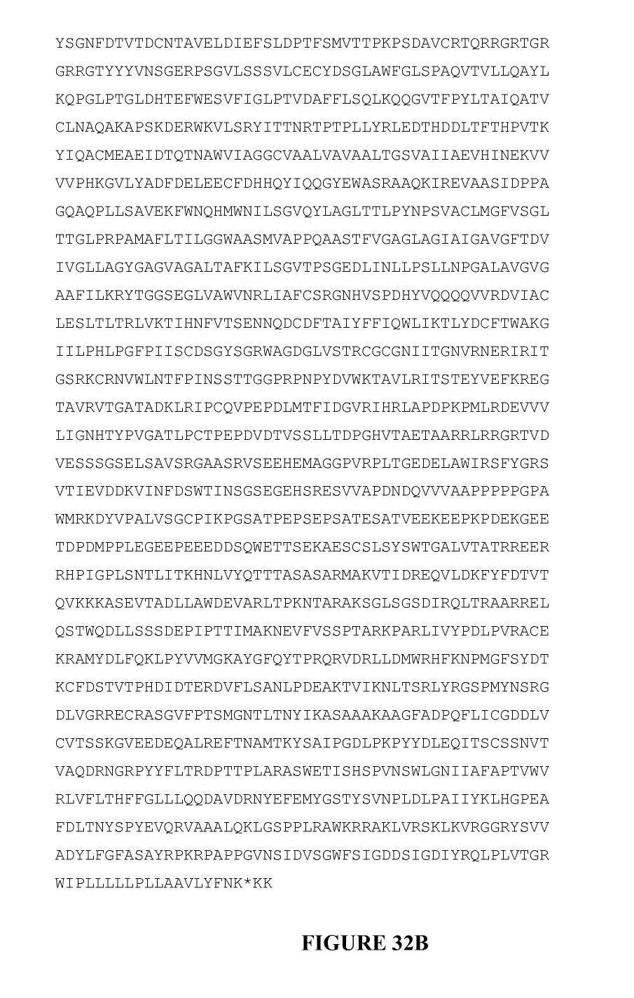

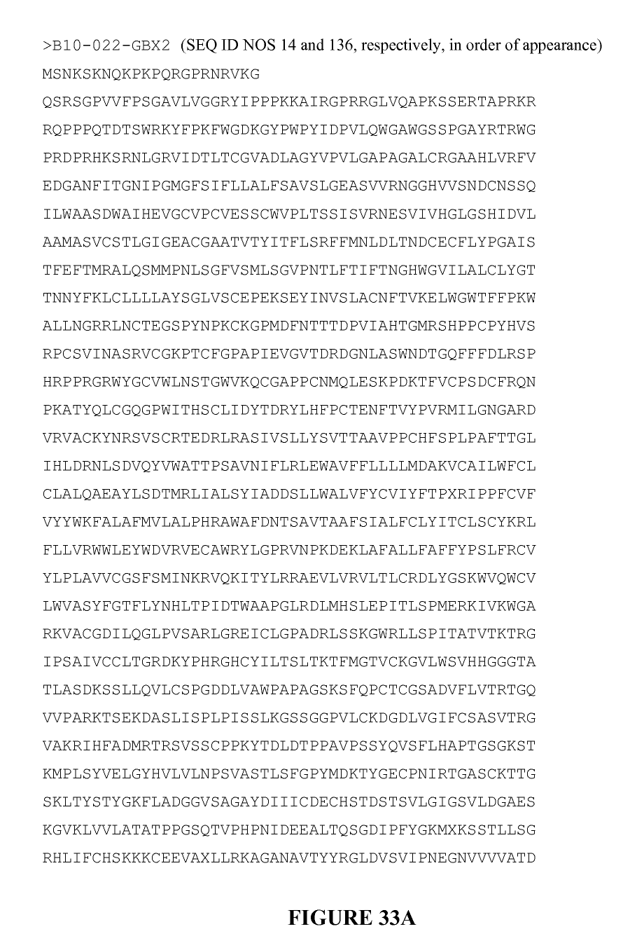

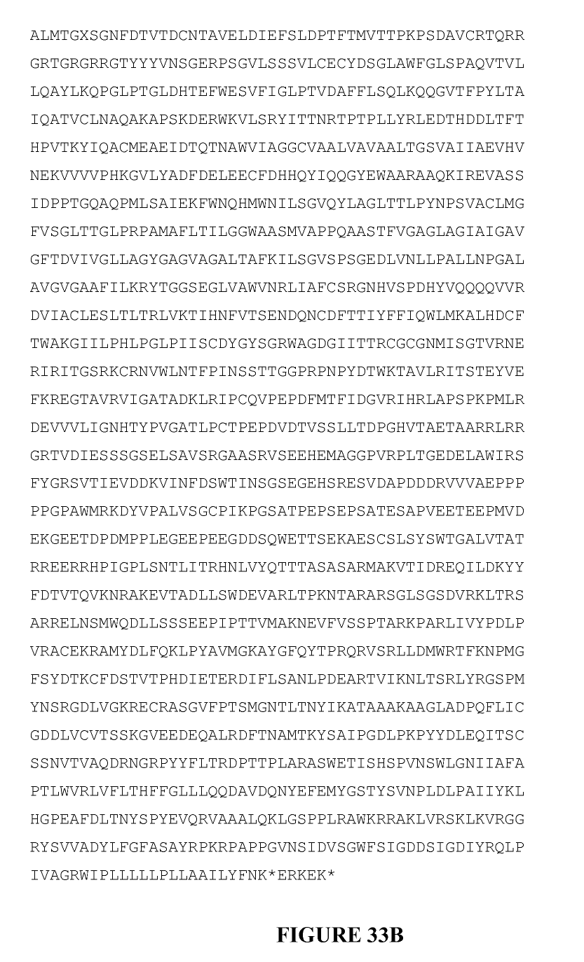

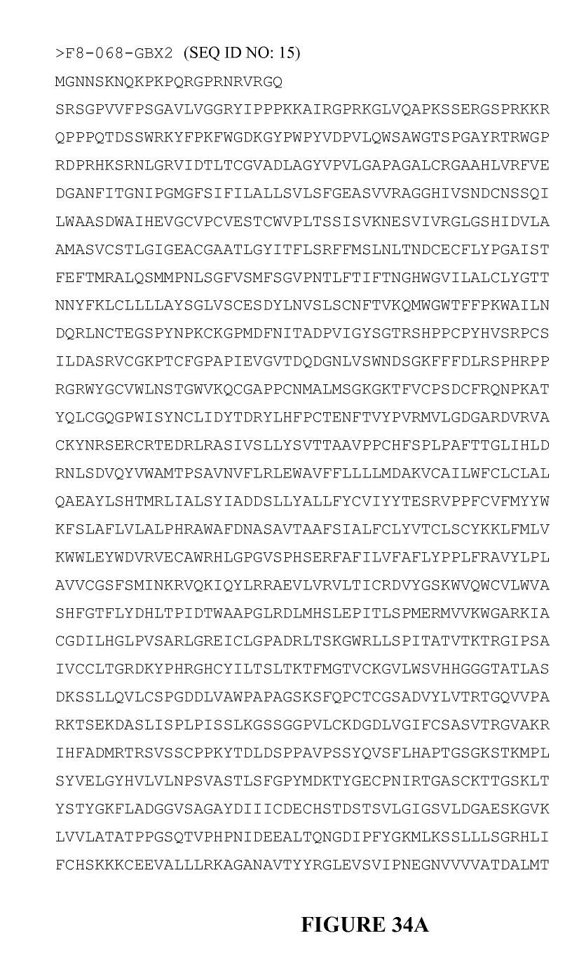

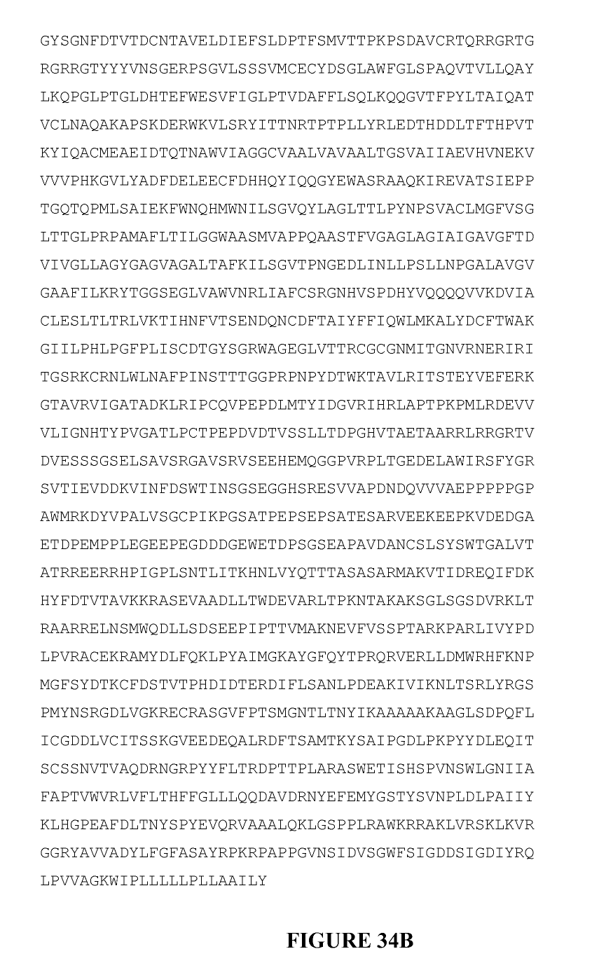

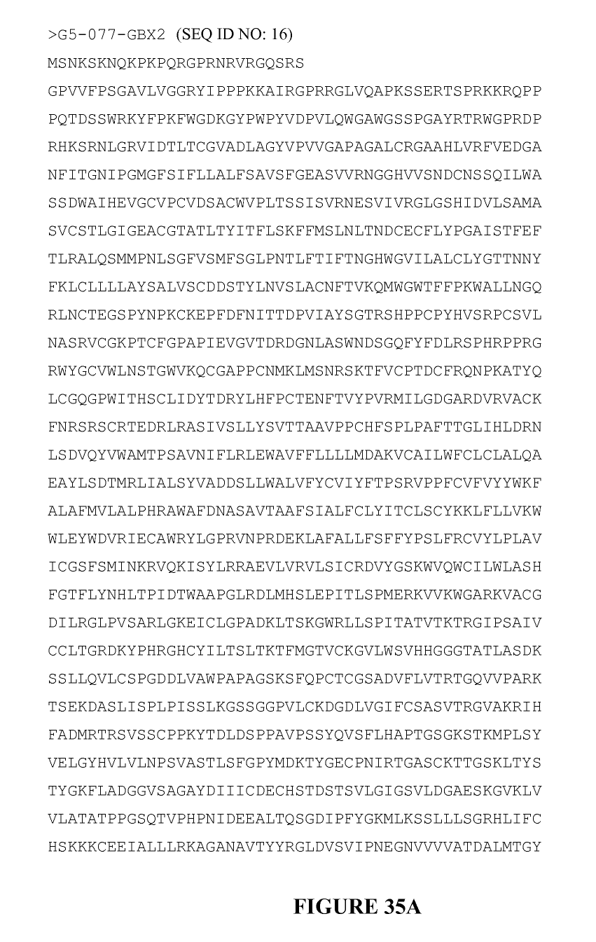

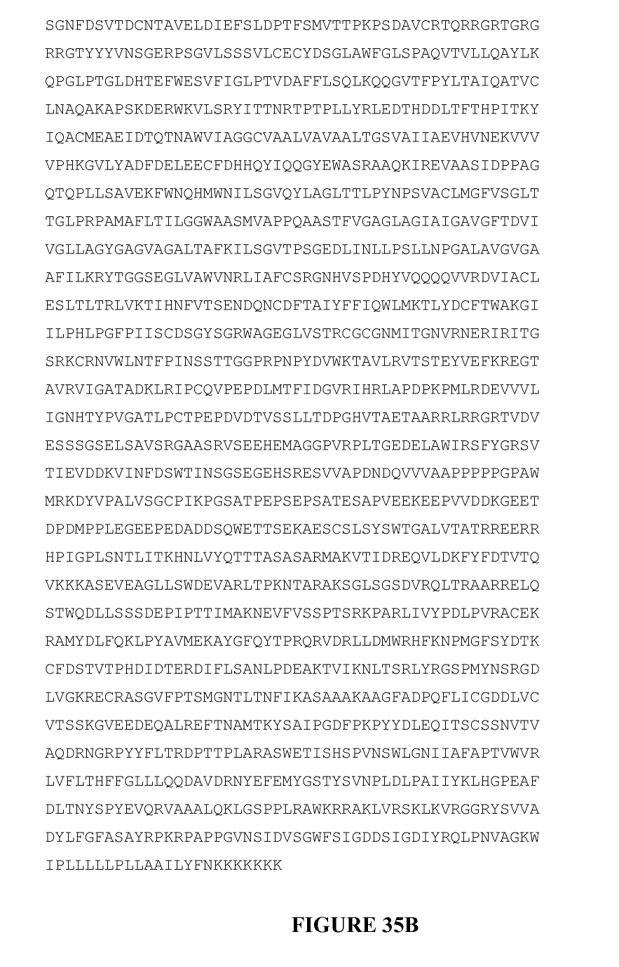

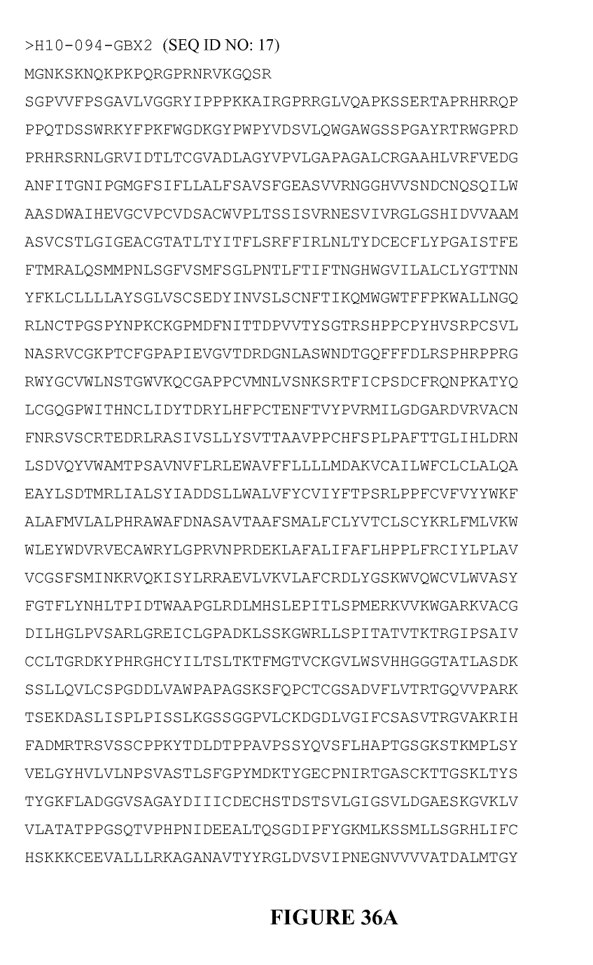

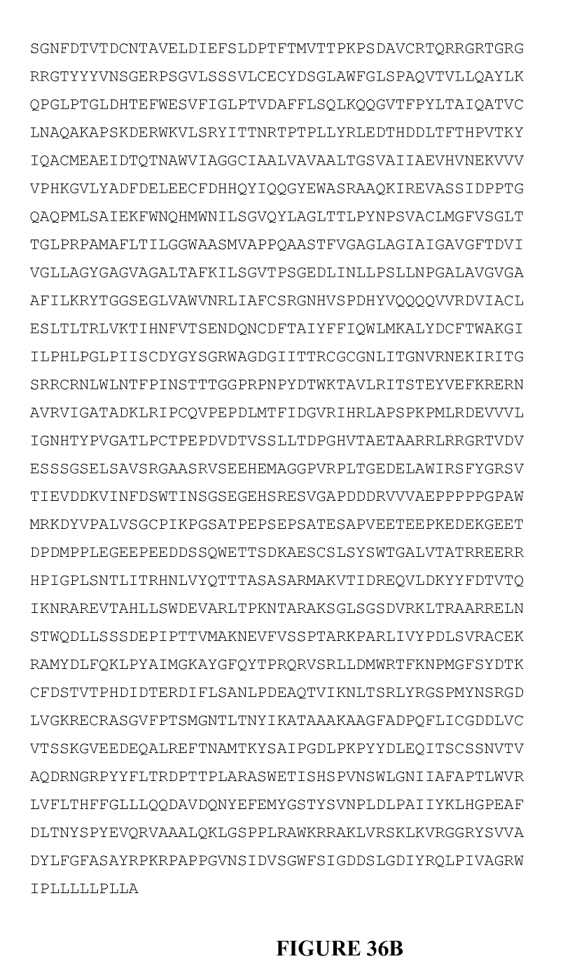

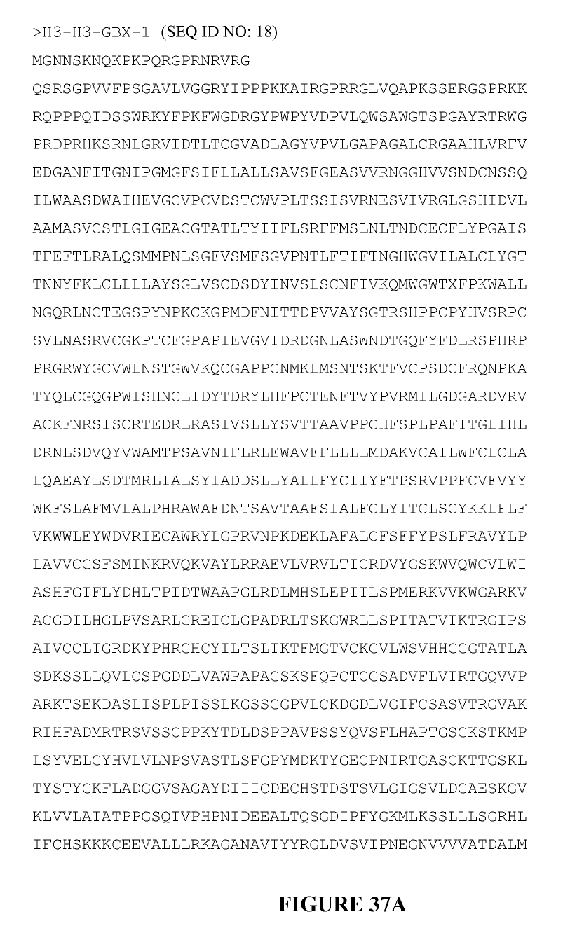

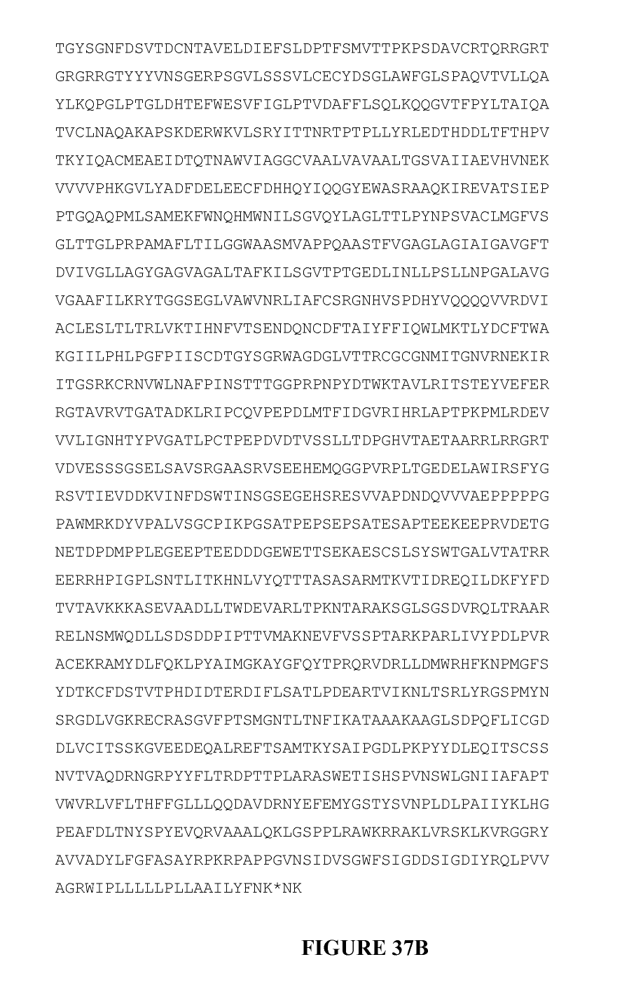

Described herein is a highly divergent Hepacivirus species found in several dogs. The novel virus species belongs to genus Hepacivirus of family Flaviviridae. The partial nucleotide sequence, translated protein sequence of this new virus provisionally named NPHV are provided (FIG. 19 and FIG. 20). Genetic analysis also confirms that NPHV is a new species of flavivirus (FIG. 21). The NPHV 3′UTR was unique in that it contains 3 long poly-nucleotide tracts, one having ˜100 Us.

Many cases of serum hepatitis in horses, such as Theiler's disease, are unexplained. The disease association of NPHV is currently being investigated, and it is possible that these viruses are responsible for such cases, as was recently shown for a different novel equine pegivirus, Theiler's disease associated virus (TDAV) (Chandriani et al. (2013) PNAS 110:E1407-15) NPHV is a hepatotropic virus that leads to chronic infection in horses with a prevalence comparable to HCV. NPHV can be a useful model virus for HCV.

### Example 3

### Serology Based Discovery of Genetically Diverse Hepaciviruses and their Natural Host

The ability to study hepacivirus pathogenesis in animals would dramatically enhance hepatitis C virus (HCV) research, which naturally infects only humans and chimpanzees resulting in a paucity of animal models. Animal homolog of HCV includes a recently discovered non-primate hepacivirus (NPHV) and GBV-B, both viruses of unclear natural host range. A versatile serology based approach was used to determine the natural host and infection prevalence of the only known non-primate hepacivirus, NPHV which is also the closest phylogenetic relative of HCV. Of the several non-primate animal species studied, the serum samples of 36% Horses were immunoreactive to the NPHV helicase protein. Expecting that like HCV, NPHV can cause persistent infection, all horse sera were tested by PCR and detected viral genomic RNA in 8 of 36 sero-positive animals. Described herein is the natural host, infection prevalence, complete genomes and genetic analysis of eight novel and diverse non-primate hepaciviruses (NPHV). Sequence diversity among NPHV variants is greater than the intra-subtype diversity reported for HCV and Pegiviruses indicating existence of different NPHV subtypes. Genetic analysis of the complete coding sequences, 5′UTR and their predicted secondary structures, reveals several unique genomic features of hepaciviruses. The results described herein can be used to design the complete genome clone, animal model and in-vivo pathogenesis studies for hepaciviruses.

Identification and characterization of animal virus homologs provide insights into the pathogenesis of human pathogenic viruses, and, in some instances, in vivo models for investigating prevention and treatment of human disease (Wobus et al., 2006). A few of these well-characterized animal viruses include simian immunodeficiency virus, animal poxviruses, herpesviruses and mouse norovirus. Hepatitis C virus (HCV), in contrast, has few known animal relatives (Bukh et al., 2011; Kapoor et al., 2011). Moreover, HCV naturally infects only humans and chimpanzees, resulting in a paucity of animal models for mechanistic studies. Most of what researchers know about HCV therefore comes from cell culture systems that often fail to recapitulate the studies on virus infection, spread, immunity and pathogenesis (Lindenbach et al., 2005; Pietschmann et al., 2005; Wakita et al., 2005; Dolgin et al., 2011). An estimated 2% of the world's population is chronically infected with HCV. The ability to study hepacivirus pathogenesis in animals would dramatically enhance resources for HCV research (Dolgin et al., 2011; Murray and Rice, 2011).

The genus Hepacivirus, one of four genera in the family Flaviviridae, comprises HCV and GBV-B (Stapelton et al., 2010). GBV-B, which diverges significantly from HCV, was isolated by passage of hepatitis patient serum in tamarins but was never again recovered from a human sample. The natural host of GBV-B remains elusive (Stapelton et al., 2010; Bukh et al., 1999; Nam et al, 2004; Bukh et al., 2001). Non-primate hepacivirus (NPHV) was recently identified in respiratory samples of domestic dogs (Kapoor et al., 2011). NPHV is the first non-primate hepacivirus discovered and comparative phylogenetic analysis confirmed it as the closest genetic relative of HCV described to date (Bukh et al., 2011; Kapoor et al., 2011). The envelope protein E2 of HCV, for example, is among the most variable portions of its genome, yet it has remarkable sequence similarity to NPHV (Kapoor et al., 2011. Furthermore, NPHV was detected in canine hepatocytes, although its link with hepatitis and the persistence of infection was not studied (Bukh et al., 2011).

Recent advances in sequencing technologies helped in identification of many highly divergent human and animal viruses, including NPHV (Kapoor et al., 2011a; Kapoor et al., 2009; Kapoor et al, 2010; Bruderer et al., 2004; Kapoor et al, 2010; Kapoor et al., 2008a; Kapoor et al., 2008b; Kapoor et al., 2011b). However detection of viral nucleic acid alone, particularly in feces or respiratory samples where they may simply represent ingested contaminants, is insufficient to establish infection let alone disease (Lipkin 2010; Burbelo et al, 2011). Although the demonstration of specific adaptive immune responses against the virus structural and non-structural proteins cannot in itself prove a causal relationship it does provide definitive evidence of host infection (Bruderer et al., 2004; Burbelo et al, 2011a). Described herein is the usefulness of serological assays to identify the natural host tropism of a highly divergent and uncharacterized virus like NPHV. The serology data described herein guided investigation of the presence of NPHV-like viruses in a specific animal species and lead to identification of eight novel and genetically diverse non-primate hepaciviruses (NPHV).

Investigation of the natural host range of hepaciviruses related to NPHV using luciferase immunoprecipitation system (LIPS) assay. NPHV, the only known non-primate hepacivirus and phylogenetically most related to HCV, has the potential to become a valuable model system to study the infection and pathogenesis of hepaciviruses (Bukh et al., 2011). The aim was to identify the natural host of NPHV using a serological approach. In hepaciviruses, the structural and nonstructural proteins are typically generated by proteolytic cleavage by virus- and host encoded proteinases (Murray et al., 2011; Blight et al., 2000).

The hypothetical cleavage map of the NPHV polyprotein was generated using an alignment with representative sequences from the seven HCV genotypes (Kapoor et al., 2011a). The NPHV core and serine protease/helicase (NS3) that represent the capsid and non-structural proteins respectively, were cloned into pREN3 vector for recombinant expression in 293 and Cos cells. Luciferase immunoprecipitation system (LIPS) assays were performed using NPHV proteins fused with Renilla luciferase protein (Burbelo et al., 2011a; Burbelo et al., 2011b; Burbelo et al., 2007; Burbelo et al., 2005). Antibodies specifically bound to the NPHV proteins were measured as the luciferase unit retained on protein A/G beads.

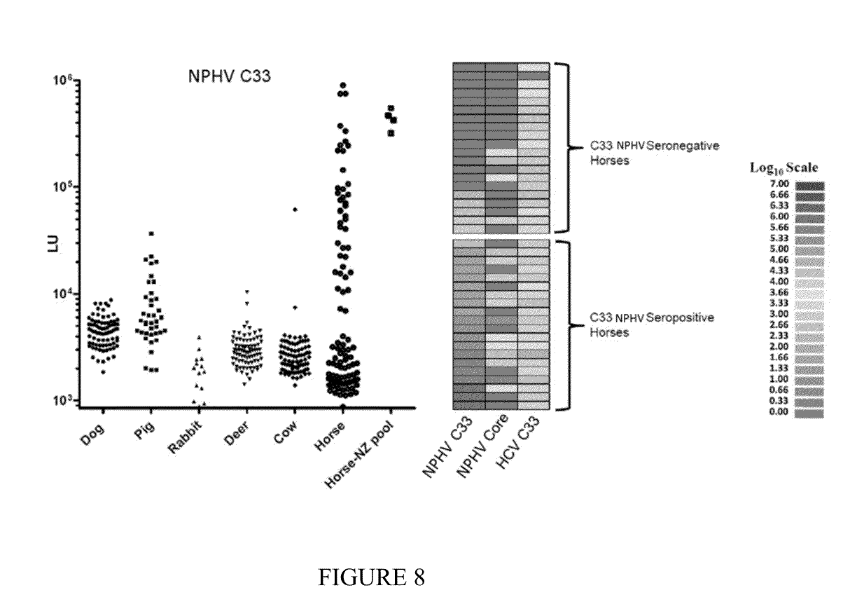

Given the high genetic diversity observed in other RNA viruses including HCV, the evolutionary conserved NPHV helicase protein was used as antigen in LIPS assays. Serum samples of 100 dogs, 38 pigs, 15 rabbits, 100 deer, 100 cows and 100 horses were tested for presence of anti-NPHV helicase IgG. Results showed presence of high titer IgG antibodies in 36% of serum samples from horses, while one each of cow and pig serum sample also showed intermediate reactivity (FIG. 8A). To rule out antigenic cross-reactivity, the helicase proteins of hepatitis C virus were used and the NPHV reactive samples were tested and found as non-reactive in all these samples (FIG. 8B). These serological assays indicated infection of horses by hepacivirus/es that should be genetically more related to NPHV than HCV in the helicase protein.

Expecting that like HCV infection in humans the hepacivirus infection in animals will be persistent (Murray et al., 2011), two broadly reactive PCR assays targeting the highly conserved motifs in NPHV 5′UTR and helicase were developed to detect the genetically related hepacivirus genomic RNA in serums samples of horses and cows. Of the 100 each horse and cows sera tested, only 8 horse samples showed presence of hepacivirus RNA. Initial sequencing of these hepaciviruses found in horse sera indicated presence of genetically diverse viruses. Comparison of serological data and PCR results showed that all 8 samples positive for hepacivirus RNA were those found highly reactive to NPHV helicase protein in LIPS assay. Primer walking approach was then used to acquire additional genomic sequences of all 8 hepacivirus variants. Since these viruses were found in a different natural host and had substantial genetic diversity compared to NPHV, they were named non-primate hepaciviruses (NPHV 1-8). The results described herein confirm that NPHV causes persistent infection in horses.

The complete genomic sequences of all 8 NPHV variants was acquired directly from horse serum samples for the purpose of phylogenetic classification and estimation of genome wide diversity. Complete genome sequences of NPHV were almost completely co-linear, with three sites of 1-3 base insertion among some variants in the 5′UTR and three regions of single amino acid insertions in the coding region. Compared to the original NPHV sequence, all NPHV variants were 17 bases longer in the 5′UTR, indicating the original NPHV genomic sequence (JF744991) may have been incomplete at 5′ end.

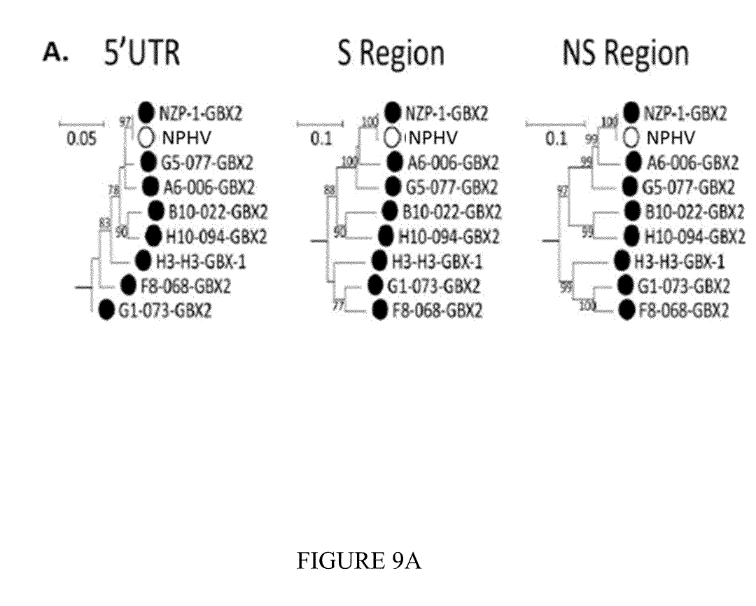

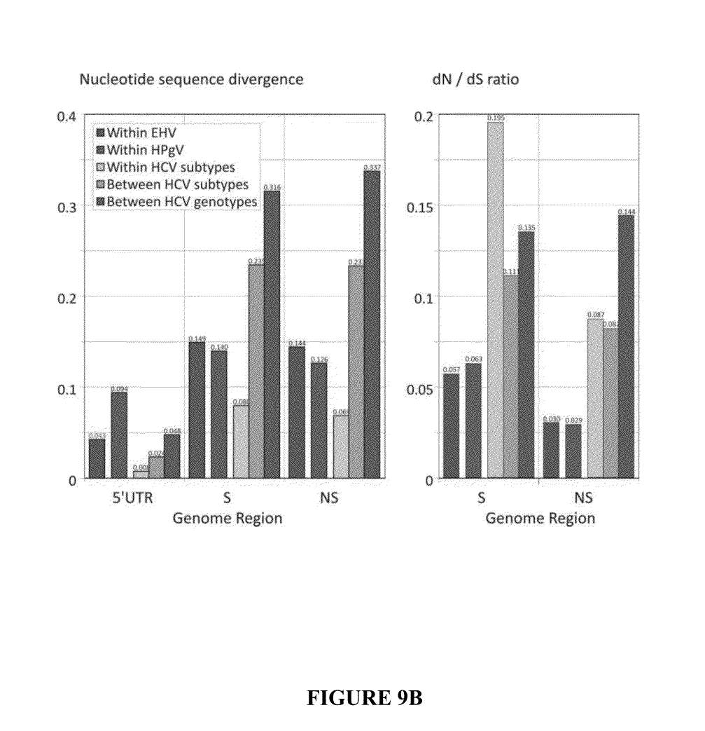

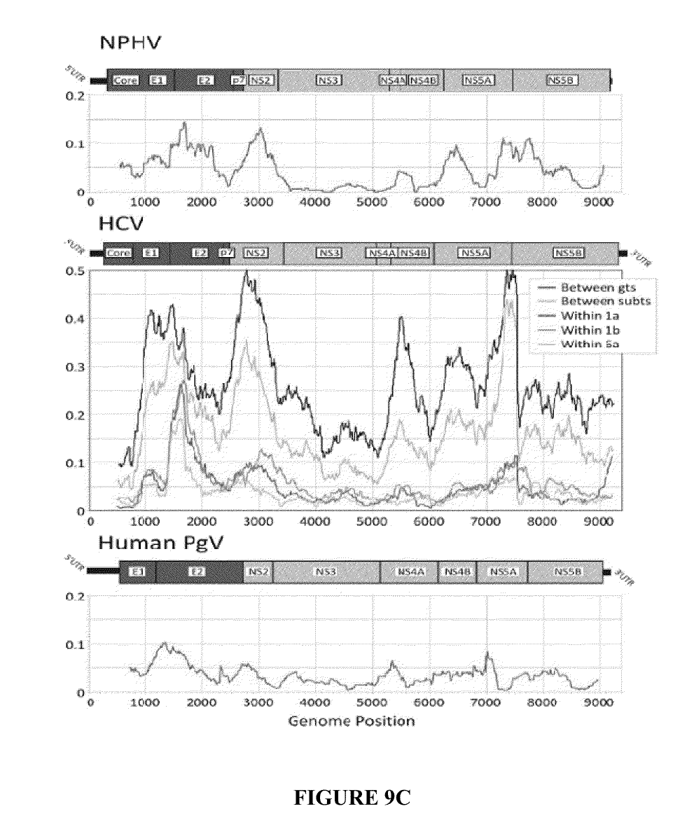

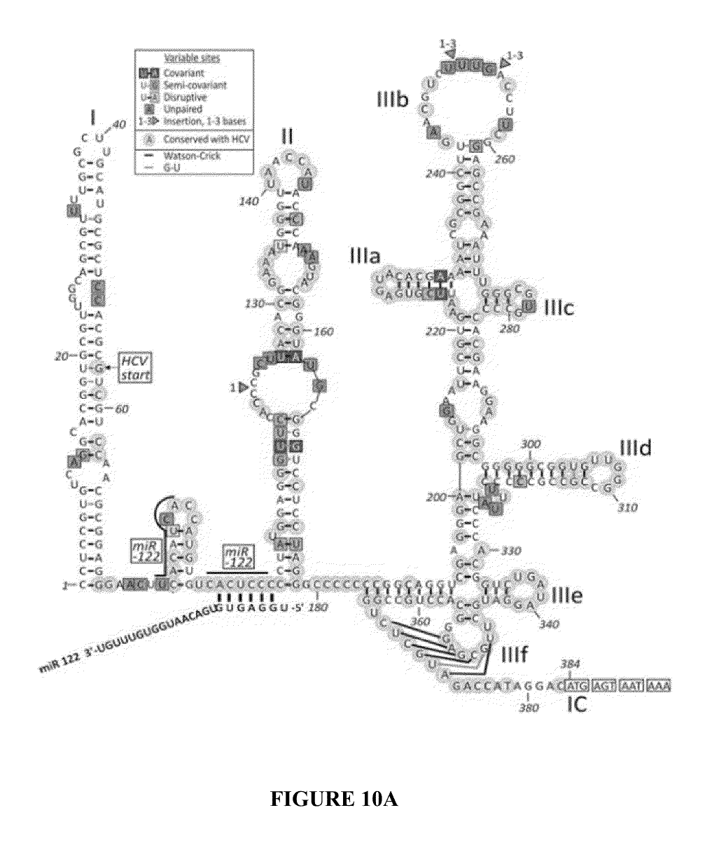

With the exception of the original NPHV variants which were highly similar to NZP-1-GBX2 (maximum of 0.35% divergence), the 8 horse-derived NPHV sequences showed moderate sequence divergence from each other (6.4%-17.2%) over the length of the genome (mean 14.0%). At the nucleotide level, sequences were more divergent in the structural (S) and non-structural (NS) gene regions (encoding core, E1 and E2 proteins and NS2-NSSB respectively) than the 5′UTR (FIG. 9), although the S region showed greater amino acid sequence divergence than NS genes (6.7% compared to 4.0%). However, most sequence diversity between NPHV variants occurred at synonymous sites with extremely low dN/dS ratios in both coding regions (0.057 and 0.030) (FIG. 9). These figures are conservative estimates because calculated Jukes-Cantor corrected synonymous distances of between 1 and 2 may underestimate the frequency of multiple substitutions. Despite the differences in sequence variability between sequence regions, phylogenetic relationships between the 8 equine sequences and NPHV were consistent across the genome (FIG. 9) with no bootstrap-supported changes in branching order indicative of recombination within NPHV and NPHV groups (Tamura et al., 2011).

Sequence diversity within NPHV was greater than subtype diversity within HCV (mean pairwise distances in S and NS regions ranged from 6-10% and 5-12% in representative subtypes 1a, 1b and 6a, compared to 15% and 14% in NPHV). NPHV diversity in the two regions was however substantially less than the mean divergence between HCV subtypes and genotypes (24%/23% and 32%/34%). HCV additionally differed from NPHV in its greater frequency of non-synonymous substitutions; although less divergent overall, mean within subtype amino acid sequence of 1a, 1b and 6a in the two regions (7.2% and 6.5%) was greater than within NPHV (6.7% and 4.0%). The pattern of diversity was indeed more similar to that of human pegiviruses (formerly referred to as GB virus-C or hepatitis G virus; (Pietschmann et al., 2005)). Diversity among human variants occurred at a similar level (14% and 12.5% nucleotide sequence divergence in S and NS regions) and similarly low dN/dS ratios (0.063 and 0.029) (FIG. 9).

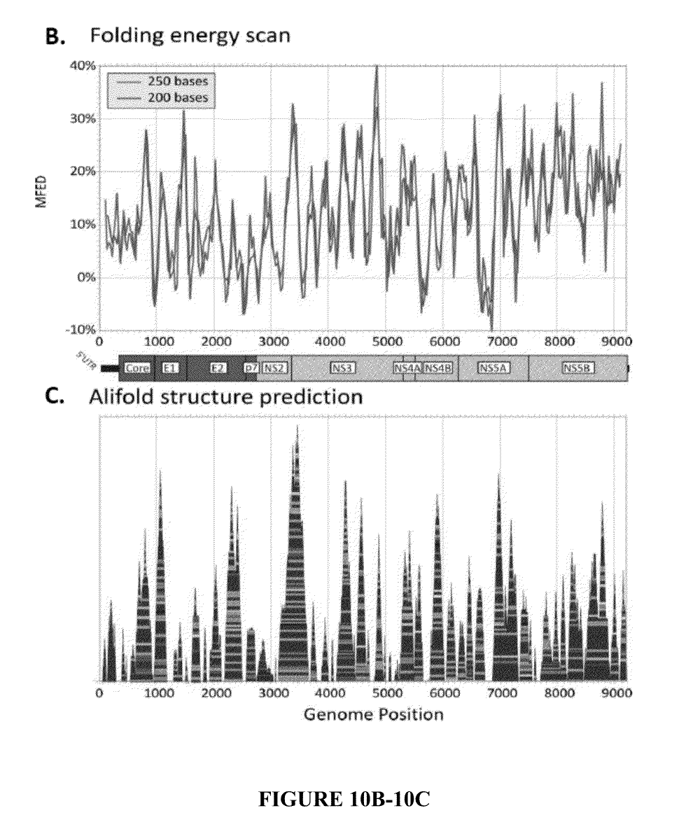

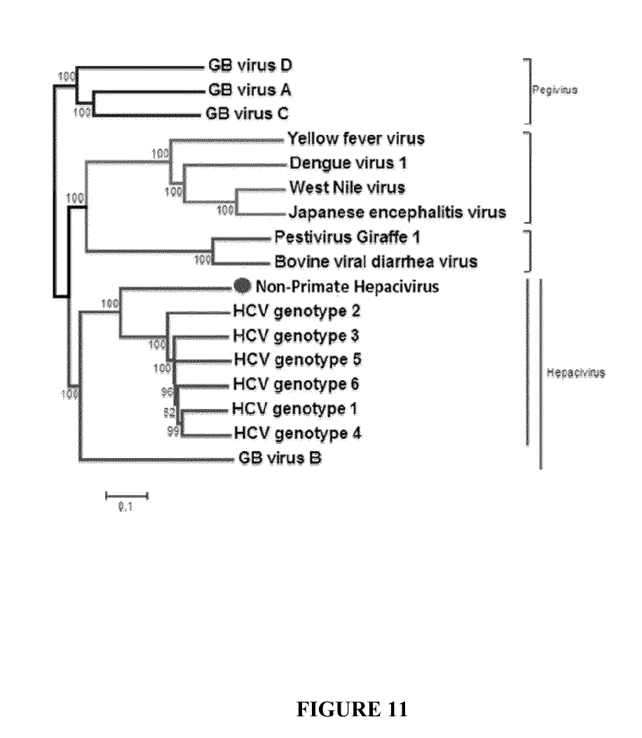

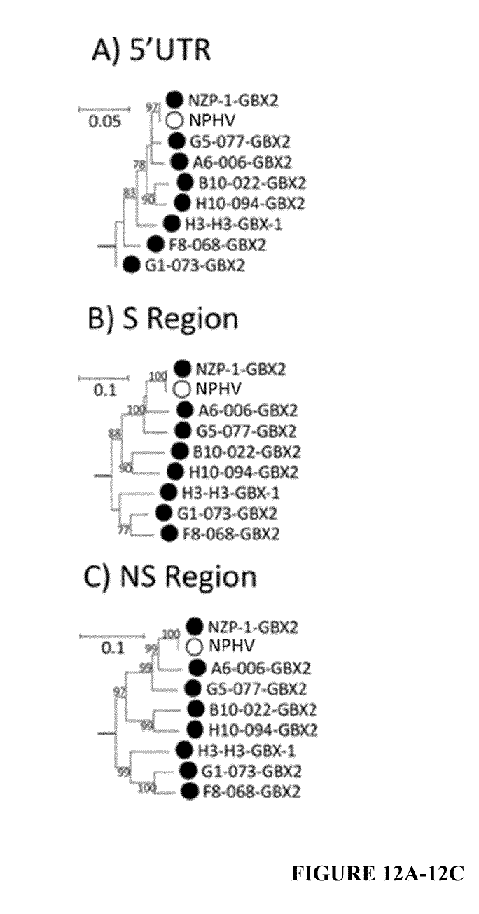

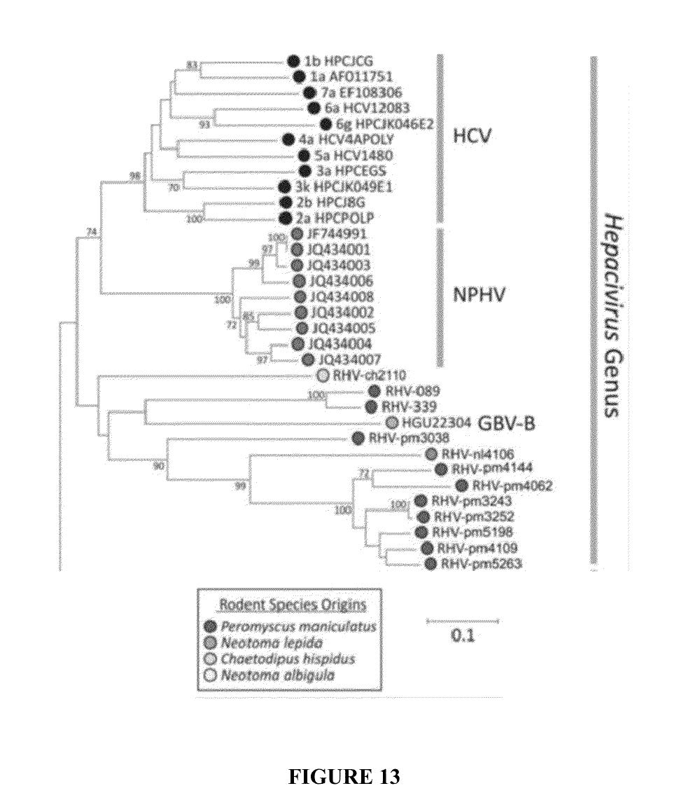

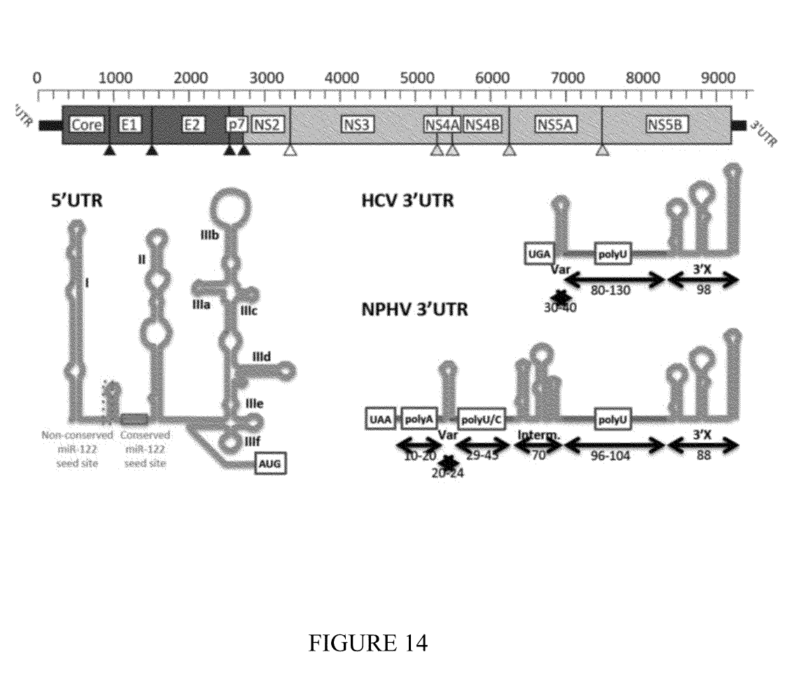

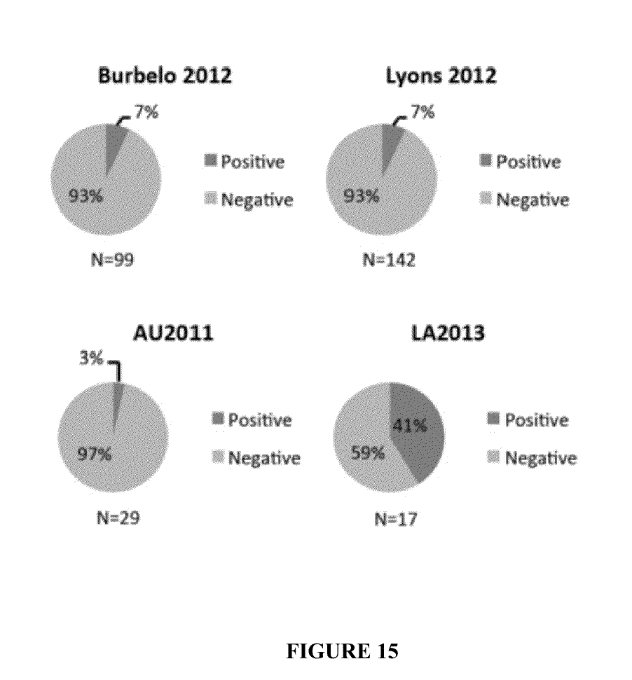

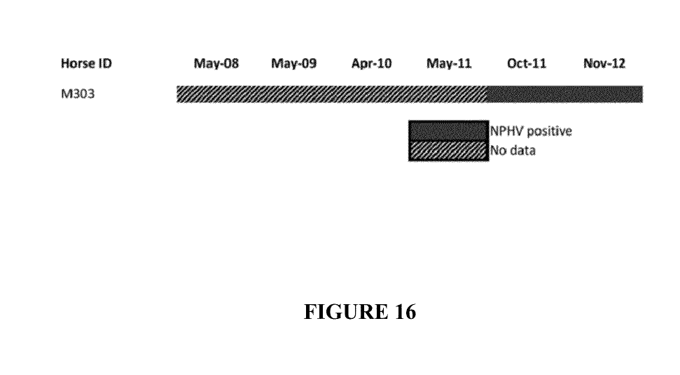

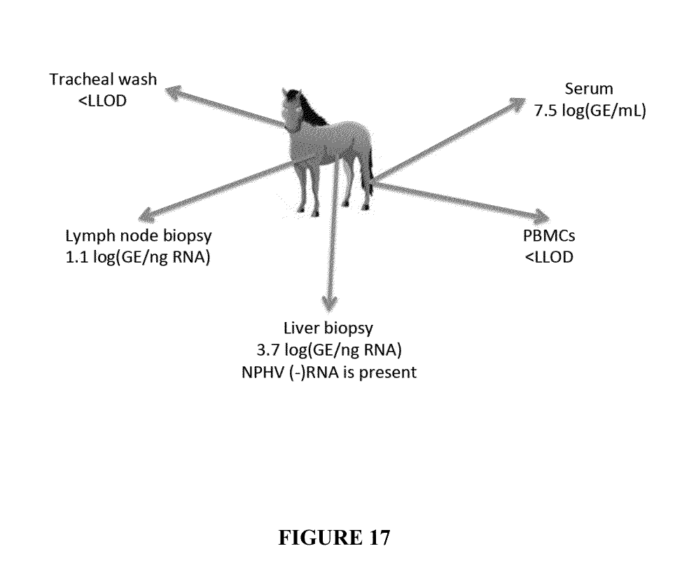

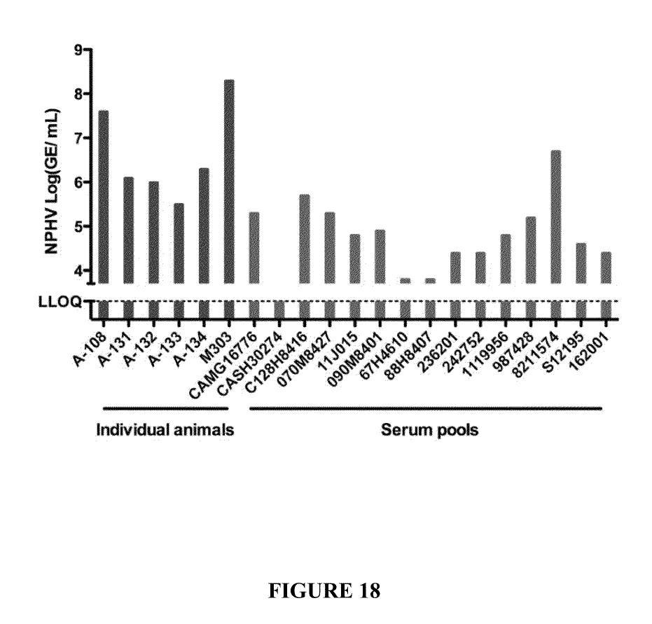

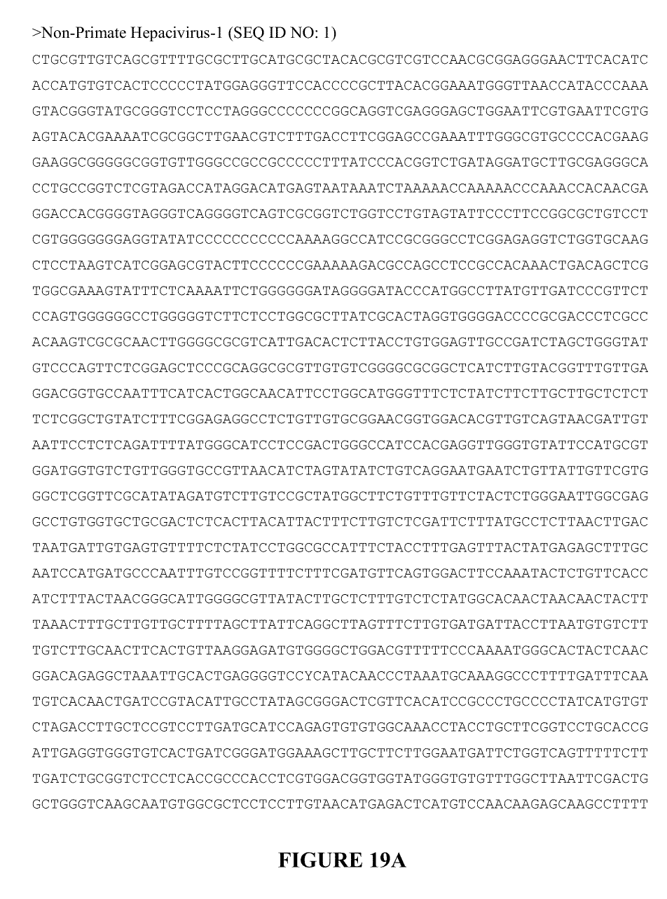

The availability of multiple sequences from NPHV enabled verification and refinement of the 5′UTR secondary structure prediction as well as an exploration of the nature of large scale RNA structure in the coding part of the genome. The additional 17 bases at the 5′ end of the 5′UTR extends the terminal loop creating a highly conserved, thermodynamically supported structure that is both larger and more conserved than structure found within the homologous region in HCV. 5′UTR sequences showed a mean divergence of approximately 4% between horse-derived NPHV variants and the distribution of this variability was investigated to determine whether substitutions could be accommodated within the previously proposed secondary structure (FIG. 10A) (Kapoor et al., 2011a). Most of the 44 polymorphic sites occurred in regions of no predicted base-paring (n=26; 59%—green boxes). All but two of the remainder were covariant (i.e. substitutions occurred in pairs to maintain base-pairing; n=6) or semi-covariant (G-C<->G-U or A-U<->G-U; n=10). All insertions/deletions (triangles) occurred in unpaired loop regions (in stem-loops II and IIIb) (FIG. 10A).

Variability in the 5′UTR sequences was concentrated in stem-loop II, IIIb and the homologous region to the miRNA-122 binding site 1 in HCV (Kapoor et al., 2011a; Jopling et al., 2005). In general, regions that were conserved between NPHV and HCV (blue circles) were invariant between NPHV variants, while other regions such as the IIIb terminal were variable in both sequence and length in both viruses (FIG. 10A). Regions of the IRES with known or suspected functional roles in ribosome binding/translation initiation were invariant in NPHV and mostly identical in sequence to homologous regions in HCV (Honda et al., 2009). The exception was the base-paired region between position 5′-185-193 and 5′-357-365 which was non-homologous to paired base regions in HCV.

Sequence variability and the use of phylogenetic information (such as the occurrence of co-variant sites) were used to explore RNA secondary structure in the coding region of the genome (FIG. 10B). Previous thermodynamic folding analysis of the NPHV genome revealed a 14% free energy difference between its minimum folding energy (MFE) with that of sequence order-randomized controls, observations consistent with the presence of genome-scale ordered RNA structure in the NPHV genome (Kapoor et al., 2011a). MFE differences (MFEDs) in the coding region of the 8 horse-derived hepacivirus sequences ranged from 12.5% to 13.9% (mean 13.0%). MFEDs of successive fragments of length between 250 and 400 bases revealed the presence of 27 regularly spaced stem-loops running through the coding part of the genome (FIG. 10B). Mean stem-loop separations (between peak MFED values) were 295 (standard deviation±80) and 306 (±71) bases in separate analyses using fragment lengths of 250 and 200 base fragments respectively for scanning Positions and spacing of structures predicted by MFED scanning were consistent with ALIFOLD (Gruber et al., 2008) which computes pairing likelihoods based on phylogenetic conservation and covariance weighted structure prediction on an underlying thermodynamic model (FIG. 10C). Through analysis of the predicted pairings, the substantial sequence diversity between NPHV sequences in coding regions (14%) was compensated by semi- and fully covariant sites and concentration of polymorphisms in predicted unpaired terminal loop regions analogous to the pattern observed in the 5′UTR. A full analysis of the coding region of NPHV and other viruses with large-scale RNA secondary is in preparation.

HCV and its genetically related viruses were considered to be restricted to primates until the recent discovery of NPHV indicated a wider host range (Bukh et al., 2011; Kapoor et al., 2011a). Initially NPHV was found in dogs but subsequent efforts to find similar viruses in dogs remained largely unsuccessful and therefore to determine its natural host evidence of viral infection was examined in other non-primate animal species. Serology for advantages of tolerance for sequence divergence, capacity to detect historical as well as current infection and high throughput was pursued. It was expected that like other RNA viruses including HCV, different NPHV variants will be genetically diverse and therefore to increase the sensitivity of LIPS assay a conserved viral protein, viral helicase, was used.

The serological studies described herein showed NPHV-like virus infection of horses. Infection was confirmed by detecting diverse viral genome in the multiple serum samples. Horses are also known to support replication of several other flaviviruses including WNV and SLEV and that can infect other animal species including humans. Most of the NPHV variants detected in horses were genetically distinct from the NPHV suggesting their species specificity. However, one NPHV variant found in a commercial horse serum pool (from New Zealand) was almost identical to NPHV indicating cross-species transmission potential of these viruses. The results described herein indicate that NPHV causes persistent infection in horses, more like HCV infection in humans than GBV-B.

Comparative sequence analysis of related viruses can be used to identity evolutionary conserved and therefore functionally important genomic regions. Despite their high nucleotide diversity over the entire genome, the HCV 5′UTR contains two miR-122 binding sites that are highly conserved among all genotypes and required for replication in liver cells. The predicted secondary structure of the NPHV 5′UTR shows occlusion of these mir122 binding sites and variation in the seed sites for miR-122 (Kapoor et al., 2011a). The availability of multiple sequences from NPHV enabled verification and refinement of the 5′UTR secondary structure prediction and notably the revised structure showed that despite genetic variability, the 5′UTR of all NPHV variants contain an open and completely conserved miR122 seed site (FIG. 10A). This observation indicates potential for hepatotropism.

RNA viruses containing genome-scale ordered RNA structure (GORS) with high mean folding energies (MFE) are more likely to cause persistent infection (Simmonds et al., 2004). The NPHV genome sequences were analyzed for evidence of GORS by comparing folding energies of consecutive fragments of nucleotide sequence with random sequence order controls (Simmonds et al., 2004). Similar to HCV, all NPHV genomes have high MFE differences (12.5% to 13.9% (mean 13.0%). Although it is possible that the failure to detect viral sequences in more than 22% of seropositive horses (8 of 36) reflects sequence divergence that confounded consensus PCR, NPHV may be cleared in the majority of equine hosts.

Similarities and differences between the HCV and NPHV will be equally informative with respect to hepacivirus biology. If NPHV resembles HCV in its pathogenesis, it could lead to a tractable in vivo model for the human virus. Where the species diverge, it will provide a unique opportunity to compare the molecular and cellular basis for those differences. The ability to compare closely related hepaciviruses in vitro will provide insights into the molecular biology of both viruses. Features such as entry factors, interactions of viral and host proteins, and the regulation of replication by genomic elements can be pursued. Moreover an infectious clone for NPHV will pave the way for experimental animal infections. The data presented here will help in generating a NPHV consensus sequence from multiple isolated sequences. As for HCV, a consensus clone will be useful in recapitulating replication and potentially infectious virus production in cultured cells. Ultimately, these NPHV can provide an ideal backbone for the development of recombinant HCV immunogenic compositions. Together, the results described herein can be used to design studies to define hepacivirus biology from a new angle. The availability of genetically distinct NPHV genomes and their comparative genetic analysis with HCV genotypes will advance understanding of the role these genetic elements and proteins play in viral life cycle.

Serum Samples.

Serum samples from different animal's species included sera of 100 dogs, 38 pigs, 15 rabbits, 100 deer, 100 cows and 100 horses. All were residual samples collected for diagnostic or commercial use and investigators have no other sample identifiers, except that all animal were living in New York state area. All serum samples were stored at −80° C., thawed, and then left at 4° C. prior to processing for LIPS analysis.

Generation of Ruc-Astrovirus Antigen Fusion Constructs.

Templates for NS3 serine protease/helicase coding sequences of NPHV was generated by RT-PCR amplification from respiratory sample of a dog (Kapoor et al., 2011a). Due to the possibility of antibody cross-reactivity with the HCV helicase gene, a fragment encompassing the corresponding helicase region of HCV was also generated as an antigen control. The primer adapter sequences used to clone each protein fragment are as follows: for NPHV, 5′-GAGGGATC CATGGCTGGTAAACAGCCC-3′ (SEQ ID NO: 47) and 5′-GAGCTCGAGTCAAGGGCCTGTGTTAGGTGC-3′ (SEQ ID NO: 48) and for HCV, 5′-GAGGGATCCAACACCACTACAGGGTCA-3′ (SEQ ID NO: 49) and 5′-GAGCTCGAGTCAATCCAGTGGGGTCAATCT-3′ (SEQ ID NO: 50). Both protein fragments were subcloned downstream of Renilla luciferase (Ruc) using the pREN2 vector (Burbelo et al., 2005). DNA sequencing was used to confirm the integrity of the DNA constructs. The sequences for the helicase fragment of NPHV have been deposited in GenBank (accession no. ______). Plasmid DNA was then prepared from these two different pREN2 expression vectors using a Qiagen Midi preparation kit. Following transfection of mammalian expression vectors, crude protein extracts were obtained as described for use as antigen (Burbelo et al., 2009).

LIPS assays. Briefly, animal sera were processed in a 96-well format at room temperature as previously described (Burbelo et al., 2011b; Burbelo et al., 2007). Serum samples were first diluted 1:10 in assay buffer A (50 mM Tris, pH 7.5, 100 mM NaCl, 5 mM MgCl₂, 1% Triton X-100) using a 96-well polypropylene microtiter plate. Antibody titers were measured by adding 40 μl of buffer A, 10 μl of diluted sera (1 μl equivalent), and 1×10⁷ light units (LU) of each of the Ruc-NPHV and HCV helicase antigen fragments containing crude Cos1 cell extract to wells of a polypropylene plate and incubated for 60 minutes at room temperature on a rotary shaker. Next, 5 μl of a 30% suspension of Ultralink protein A/G beads (Pierce Biotechnology, Rockford, Ill.) in PBS were added to the bottom of each well of a 96-well filter HTS plate (Millipore, Bedford, Mass.). To this filter plate, the 100 μl antigen-antibody reaction mixture was transferred and incubated for 60 minutes at room temperature on a rotary shaker. The washing steps of the retained protein A/G beads were performed on a Biomek Workstation or Tecan plate washer with a vacuum manifold. After the final wash, LU were measured in a Berthold LB 960 Centro microplate luminometer (Berthold Technologies, Bad Wilbad, Germany) using coelenterazine substrate mix (Promega, Madison, Wis.). All LU data were obtained from the average of at least two separate experiments.

GraphPad Prism software (San Diego, Calif.) was used for statistical analysis of LIPS data. For the calculation of sensitivity and specificity, a cut-off limit was used, which was derived from the combined value of the mean value plus 3 standard deviations (SD) of the replica samples containing only buffer, Ruc-extract and protein A/G beads. Horse serum samples highly positive for anti-NPHV helicase antibodies were used as internal positive controls to standardize the LIPS parameters for testing of all serum samples.

Screening for NPHV-Like Viruses and Quantitative PCR.

All respiratory and tissue samples were extracted using Qiagen viral RNA extraction kit and RNAeasy tissue DNA/RNA extraction kit. RNA was converted to cDNA using random primers and used in nested PCR with primers for first round (NPHV-0F1: 5′-TCCACCTATGGTAAGTTCTTAGC-3′ (SEQ ID NO: 51) and NPHV-0R1: 5′-ACCCTGTCATAAGGGCGTC-3′ (SEQ ID NO: 52)) and second round (NPHV-0F2: CCTATGGTAAGTTCTTAGCTGAC-3′ (SEQ ID NO: 53) and NPHV-0R2: 5′-CCTGTCATAAGGGCGTCCGT-3′ (SEQ ID NO: 54)). Details are available on request. All PCR products were sequenced to confirm the presence of NPHV in samples. Quantitative PCR to determine the NPHV genome copy number in respiratory samples was done using Syber green chemistry and as a copy number standard a plasmid containing HCV helicase gene. The primers used were NPHV-SG-F1 (5′GCCATAGCACAGACTCCAC3′ (SEQ ID NO: 55)) and NPHV-SG-2R1 (5′GACGGAAACATCCAAACCCCG3′ (SEQ ID NO: 56)) with ready to use PCR mix (Applied Biosystems).

Genome Sequencing and Phylogenetic Analysis.

Sequences with similarity to flaviviruses were assembled against prototype HCV strains. Gaps were filled by primer walking, using specific and degenerate flavivirus primers. Both termini of the genome were acquired using rapid amplification of cDNA ends (RACE) (Kapoor et al., 2008). Thereafter, sequence validity was tested in 4× genome coverage by classical dideoxy Sanger sequencing. Nucleotide compositions of different flaviviruses and NPHV were determined using EMBOSS compseq (http://emboss.bioinformatics.nl/cgi-bin/emboss/compseq). Translated amino acid sequences were aligned using ClustalW. Trees were constructed by neighbor-joining of pairwise amino acid distances using the program, MEGA5 (Kumar et al., 2008), employing bootstrap re-sampling to determine robustness.

RNA Structure and GORS Predictions.

Independent of phylogenetic information, the secondary structure of the NPHV 5′ UTR RNA was modeled with MFOLD. Labeling of the predicted structures in the 5′ UTR followed numbering used for reported homologous structures in HCV and GBV-B. The NPHV genome sequence was analyzed for evidence of GORS by comparing folding energies of consecutive fragments of nucleotide sequence with random sequence-order controls (35). MFEs of NPHV were calculated by using default setting in the program ZIPFOLD. MFE results were expressed as MFE differences, i.e., the percentage difference between the MFE of the native sequence from that of the mean value of the 50 sequence order-randomized controls.

### Example 4

### Eight Novel and Genetically Diverse Non-Primate Hepaciviruses

The ability to study hepacivirus pathogenesis in animals would dramatically enhance hepatitis C virus (HCV) research, which naturally infects only humans and chimpanzees resulting in a paucity of animal models. Animal homolog of HCV includes a recently discovered non-primate hepacivirus (NPHV) and GBV-B, both viruses of unclear natural host range. A versatile serology based approach was used to determine the natural host and infection prevalence of the only known non-primate hepacivirus, NPHV which is also the closest phylogenetic relative of HCV. Of the several non-primate animal species studied, the serum samples of 36% Horses showed distinctively high reactivity against NPHV helicase protein. Based on the serological studies described herein and expecting that like HCV, NPHV can cause persistent infection, all horse sera were tested by PCR and detected viral genomic RNA in 8 of 36 sero-positive animals. Described herein is the natural host, infection prevalence and complete genomes of eight novel and genetically diverse non-primate hepaciviruses (NPHV) (FIG. 12). Sequence diversity among NPHV variants is greater than the intra-genotypic diversity reported for HCV indicating existence of different NPHV genotypes. Genetic analysis of the complete coding sequences, 5′ untranslated regions and their predicted secondary structures, reveals several unique genomic features of hepaciviruses. The results described herein can be used to design of the complete genome clone, animal model and in-vivo pathogenesis studies for hepaciviruses.

### Example 5

### Cultivation of NPHV

NPHV can be isolated from frozen and/or fresh tissues. Supernatants from those cells are frozen and evaluated for the presence of NPHV nucleic acid or amino acid sequences. Primers suitable for identification of NPHV are described herein and can be generated by one of skill in the art to detect NPHV I a sample. Cells suitable for culturing the cells described herein can be any non-primate cell including but not limited to MDBK cells, BHK 21 cells, CHO cells, 3T3 cells, C2C12 cells, RAW 264.7 cells, Mouse embryonic fibroblasts and the like. The cells can be cultivated in a medium suitable for propagation and grown to reach an optimal cell density prior infection with the respective virus. Non-limiting examples of cell culture media suitable for use with the methods described herein include, MEM, DMEM, DMEM/F12, IMDM, alpha-MEM, MESENCULT. One of skill in the art will appreciate that additional factors or supplements can be added to cell culture media to support cell growth including FBS, LIF, IGF-1, FGF, Wnt3a, PDGF-B as well as any combination of cell culture factors known in the art.

### Example 6

### Attenuation of NPHV

In certain embodiments, the immunogenic compositions described herein can comprise attenuated or inactivated NPHV. Methods for attenuating the viruses further are well known in the art and include such methods as serial passage in cell culture on a suitable cell line, or ultraviolet or chemical mutagenesis. The inactivated viruses described herein can made by methods well known in the art. For example, once the virus is propagated to high titers, it would be readily apparent by those skilled in the art that the virus antigenic mass could be obtained by methods well known in the art. For example, the virus antigenic mass may be obtained by dilution, concentration, or extraction. All of these methods can be employed to obtain appropriate viral antigenic mass to produce vaccines.

The NPHV descried herein can also be inactivated by treatment with formalin, betapropriolactone (BPL), or with binary ethyleneimine (BEI), or other methods known to those skilled in the art. Inactivation by formalin can be performed by mixing the NPHV suspension with 37% formaldehyde to a final formaldehyde concentration of 0.05%. The NPHV-formaldehyde mixture can be mixed by constant stirring for approximately 24 hours at room temperature. The inactivated NPHV mixture can then tested for residual live virus by assaying for growth on a suitable cell line.

### Example 7

### Administration of NPHV Immunogenic Compositions to Animals

The immunogenic compositions described herein can be used as a prophylactic treatment to immunizes animals against NPHV. The immunogenic compositions can also be used for the therapeutic treatment of animals already infected with NPHV.

The route of administration for any one of the embodiments of immunogenic compositions described herein includes, but is not limited to, oronasal, intramuscular, intraperitoneal, intradermal, subcutaneous, intravenous, intraarterial, intraocular, and oral as well as transdermal or by inhalation or suppository. Routes of administration include oronasal, intramuscular, intraperitoneal, intradermal, and subcutaneous injection. The immunogenic compositions described herein can be administered by any means that includes, but is not limited to, syringes, nebulizers, misters, needleless injection devices, or microprojectile bombardment gene guns.

The immunogenic compositions described herein can be formulated in a pharmaceutically accepted carrier according to the mode of administration to be used. One skilled in the art can readily formulate a vaccine that comprises a live or killed NPHV, a NPHV protein, or an immunogenic fragment thereof, a recombinant virus vector encoding NPHV, an NPHV protein or an immunogenic fragment thereof, or a DNA molecule encoding a NPHV, a NPHV protein or an immunogenic fragment thereof. In cases where intramuscular injection is used, an isotonic formulation is can be used. Additives for isotonicity can include, but are not limited to sodium chloride, dextrose, mannitol, sorbitol, and lactose. In certain embodiments, isotonic solutions such as phosphate buffered saline can be used. The formulations can further provide stabilizers such as gelatin and albumin. In some embodiments, a vaso-constrictive agent can be added to the formulation. The immunogenic compositions described herein can also include vaccine-compatible pharmaceutically acceptable (i.e., sterile and non-toxic) liquid, semisolid, or solid diluents that serve as pharmaceutical vehicles, excipients, or media. Diluents can include water, saline, dextrose, ethanol, glycerol, and the like. Isotonic agents can include sodium chloride, dextrose, mannitol, sorbitol, and lactose, among others. Stabilizers include albumin, among others.

The immunogenic compositions described herein can also be mixed with an adjuvant. In certain formulations of the immunogenic compositions described herein can be combined with other vaccines to produce a polyvalent vaccine product that can protect animals against a wide variety of diseases caused by other pathogens.

Inoculation of a non-primate animal can be by a single administration that produces a full, broad immunogenic response. In another embodiment of the present invention, the animals can be subjected to a series of administrations to produce a full, broad immune response.

Any adjuvant known in the art may be used in the immunogenic compositions described herein, including, but not limited to, oil-based adjuvants such as Freund's Complete Adjuvant and Freund's Incomplete Adjuvant, mycolate-based adjuvants, bacterial lipopolysaccharide, peptidoglycans, proteoglycans, Biostim™, aluminum hydroxide, saponin, DEAE-dextran, neutral oils, vegetable oils, liposomes, cholesterol, oil-in water emulsions, water-in-oil emulsions, block co-polymer, SAF-M, AMPHIGEN adjuvant, saponin, Quil A, QS-21, GPI-0100 or other saponin fractions, monophosphoryl lipid A, Avridine lipid-amine adjuvant, heat-labile enterotoxin from E. coli, cholera toxin, or muramyl dipeptide, among many others. The immunogenic compositions can further include one or more other immunomodulatory agents such as, e.g., interleukins, interferons, or other cytokines. The immunogenic compositions can also include gentamicin and Merthiolate.

### REFERENCES

Alter H J (1989) Discovery of the non-A, non-B hepatitis virus: The end of the beginning or the beginning of the end. Transfus Med Rev 3:77-81.
Belyi V A, Levine A J, Skalka A M (2010) Unexpected inheritance: Multiple integrations of ancient bornavirus and ebolavirus/marburgvirus sequences in vertebrate genomes. PLoS Pathog 6:e1001030.

Blight K J, Kolykhalov A A, & Rice C M (2000) Efficient initiation of HCV RNA replication in cell culture. Science 290(5498):1972-1974.
Branch A D, Stump D D, Gutierrez J A, Eng F, Walewski J L (2005) The hepatitis C virus alternate reading frame (ARF) and its family of novel products: The alternate reading frame protein/F-protein, the double-frameshift protein, and others. Semin Liver Dis 25:105-117.
Bruderer U, et al. (2004) Differentiating infection from immunization in foot-and-mouth-disease: evaluation of an ELISA based on recombinant 3ABC. Veterinary microbiology 101(3): 187-197.
Bukh J (2011) Hepatitis C homolog in dogs with respiratory illness. Proc Natl Acad Sci USA 108(31):12563-12564.
Bukh J, Apgar C L, & Yanagi M (1999) Toward a surrogate model for hepatitis C virus: An infectious molecular clone of the GB virus-B hepatitis agent. Virology 262(2):470-478.
Bukh J, Apgar C L, Govindarajan S, & Purcell R H (2001) Host range studies of GB virus-B hepatitis agent, the closest relative of hepatitis C virus, in New World monkeys and chimpanzees. J Med Virol 65(4):694-697.
Bukh J, Apgar C L, Yanagi M (1999) Toward a surrogate model for hepatitis C virus: An infectious molecular clone of the GB virus-B hepatitis agent. Virology 262:470-478.
Burbelo P D, Ching K H, Klimavicz C M, & Iadarola M J (2009) Antibody profiling by Luciferase Immunoprecipitation Systems (LIPS). J Vis Exp (32).
Burbelo P D, et al. (2007) Rapid antibody quantification and generation of whole proteome antibody response profiles using LIPS (luciferase immunoprecipitation systems). Biochemical and biophysical research communications 352(4):889-895.
Burbelo P D, et al. (2011a) Serological studies confirm the novel astrovirus HMOAstV-C as a highly prevalent human infectious agent. PLoS One 6(8):e22576.
Burbelo P D, et al. (2011b) LIPS arrays for simultaneous detection of antibodies against partial and whole proteomes of HCV, HIV and EBV. Mol Biosyst 7(5):1453-1462.
Burbelo P D, Goldman R, & Mattson T L (2005) A simplified immunoprecipitation method for quantitatively measuring antibody responses in clinical sera samples by using mammalian-produced Renilla luciferase-antigen fusion proteins. BMC Biotechnol 5:22.
Choo Q L, et al. (1989) Isolation of a cDNA clone derived from a blood-borne non-A, non-B viral hepatitis genome. Science 244:359-362.
Dolgin E (2011) Research technique: the murine candidate. Nature 474(7350):514-15.
Drummond A J, Rambaut A (2007) BEAST: Bayesian evolutionary analysis by sampling trees. BMC Evol Biol 7:214.
Drummond A J, Rambaut A, Shapiro B, Pybus O G (2005) Bayesian coalescent inference of past population dynamics from molecular sequences. Mol Biol Evol 22: 1185-1192.
Epstein J H, et al. (2010) Identification of GBV-D, a novel GB-like flavivirus from old world frugivorous bats (Pteropus giganteus) in Bangladesh. PLoS Pathog 6:e1000972.
Ferreiro M C, Dios P D, Scully C (2005) Transmission of hepatitis C virus by saliva? Oral Dis 11:230-235.
Gruber A R, Lorenz R, Bernhart S H, Neubock R, & Hofacker I L (2008) The Vienna RNA websuite. Nucleic acids research 36(Web Server issue):W70-74.
Hatziioannou T, et al. (2009) A macaque model of HIV-1 infection. Proc Natl Acad Sci USA 106:4425-4429.
Ho S Y, Phillips M J, Cooper A, Drummond A J (2005) Time dependency of molecular rate estimates and systematic overestimation of recent divergence times. Mol Biol Evol 22:1561-1568.
Honda M, Brown E A, Lemon S M (1996) Stability of a stem-loop involving the initiator AUG controls the efficiency of internal initiation of translation on hepatitis C virus RNA. RNA 2:955-968.
Horie M, et al. (2010) Endogenous non-retroviral RNA virus elements in mammalian genomes. Nature 463:84-87.
Jangra R K, Yi M, Lemon S M (2010) Regulation of hepatitis C virus translation and infectious virus production by the microRNA miR-122. J Virol 84:6615-6625.
Jopling C L, Yi M, Lancaster A M, Lemon S M, Sarnow P (2005) Modulation of hepatitis C virus RNA abundance by a liver-specific MicroRNA. Science 309:1577-1581.
Kapoor A, et al. (2008) A highly divergent picornavirus in a marine mammal J Virol 82:311-320.
Kapoor A, et al. (2008) A highly prevalent and genetically diversified Picornaviridae genus in South Asian children. Proc Natl Acad Sci USA 105:20482-20487.
Kapoor A, et al. (2009) Multiple novel astrovirus species in human stool. J Gen Virol 90:2965-2972.
Kapoor A, et al. (2010) Identification and characterization of a new bocavirus species in gorillas. PLoS ONE 5:e11948.
Kapoor A, et al. (2011a) Characterization of a canine homolog of hepatitis C virus. Proc Natl Acad Sci USA 108(28):11608-11613.
Kapoor A, et al. (2011b) Characterization of Novel Canine Bocaviruses and their Association with Respiratory Disease. The Journal of general virology.
Kapoor A, Simmonds P, Lipkin W I (2010) Discovery and characterization of mammalian endogenous parvoviruses. J Virol 84:12628-12635.
Kapoor A, Simmonds P, Lipkin W I, Zaidi S, & Delwart E (2010) Use of nucleotide composition analysis to infer hosts for three novel picorna-like viruses. J Virol 84(19):10322-10328.
Katzourakis A, Gifford R J (2010) Endogenous viral elements in animal genomes. PLoS Genet 6:e1001191.
Keckesova Z, Ylinen L M, Towers G J, Gifford R J, Katzourakis A (2009) Identification of a RELIK orthologue in the European hare (Lepus europaeus) reveals a minimum age of 12 million years for the lagomorph lentiviruses. Virology 384:7-11.
Kieft J S (2008) Viral IRES RNA structures and ribosome interactions. Trends Biochem Sci 33:274-283.
Klatt N R, et al. (2010) CD8+ lymphocytes control viral replication in SIVmac239-infected rhesus macaques without decreasing the lifespan of productively infected cells. PLoS Pathog 6:e1000747.
Kolykhalov A A, Feinstone S M, Rice C M (1996) Identification of a highly conserved sequence element at the 3′ terminus of hepatitis C virus genome RNA. J Virol 70: 3363-3371.
Krey T, et al. (2010) The disulfide bonds in glycoprotein E2 of hepatitis C virus reveal the tertiary organization of the molecule. PLoS Pathog 6:e1000762.
Kumar S, Nei M, Dudley J, Tamura K (2008) MEGA: A biologist-centric software for evolutionary analysis of DNA and protein sequences. Brief Bioinform 9:299-306.
Lindenbach B D, et al. (2005) Complete replication of hepatitis C virus in cell culture. Science 309:623-626.
Lindenbach B D, Rice C M (2005) Unravelling hepatitis C virus replication from genome to function. Nature 436:933-938.
Lipkin W I (2010) Microbe hunting. Microbiol Mol Biol Rev 74(3):363-377.
Lohmann V, et al. (1999) Replication of subgenomic hepatitis C virus RNAs in a hepatoma cell line. Science 285:110-113.
Magiorkinis G, et al. (2009) The global spread of hepatitis C virus 1a and 1b: A phylodynamic and phylogeographic analysis. PLoS Med 6:e1000198.
Minin V N, Bloomquist E W, Suchard M A (2008) Smooth skyride through a rough skyline: Bayesian coalescent-based inference of population dynamics. Mol Biol Evol 25:1459-1471.
Mohr E L, Stapleton J T (2009) GB virus type C interactions with HIV: The role of envelope glycoproteins. J Viral Hepat 16:757-768.
Murray C L & Rice C M (2011) Turning hepatitis C into a real virus. Annu Rev Microbiol 65:307-327.
Nam J H, et al. (2004) In vivo analysis of the 3′ untranslated region of GB virus B after in vitro mutagenesis of an infectious cDNA clone: persistent infection in a transfected tamarin. J Virol 78(17):9389-9399.
Parrish C R, et al. (2008) Cross-species virus transmission and the emergence of new epidemic diseases. Microbiol Mol Biol Rev 72:457-470.
Parrish C R, Kawaoka Y (2005) The origins of new pandemic viruses: The acquisition of new host ranges by canine parvovirus and influenza A viruses. Annu Rev Microbiol 59: 553-586.
Pietschmann T, et al. (2002) Persistent and transient replication of full-length hepatitis C virus genomes in cell culture. J Virol 76(8):4008-4021.
Posada D, Crandall K A (1998) MODELTEST: Testing the model of DNA substitution. Bioinformatics 14:817-818.
Protsch R, Berger R (1973) Earliest radiocarbon dates for domesticated animals: Europe is added to the Near East as another early center of domestication. Science 179:235-239.
Pybus O G, et al. (2009) Genetic history of hepatitis C virus in East Asia. J Virol 83: 1071-1082.
Ray Kim W (2002) Global epidemiology and burden of hepatitis C. Microbes Infect 4: 1219-1225.
Rijnbrand R, Abell G, Lemon S M (2000) Mutational analysis of the GB virus B internal ribosome entry site. J Virol 74:773-783.
Simmonds P (2004) Genetic diversity and evolution of hepatitis C virus-15 years on. J Gen Virol 85:3173-3188.
Simmonds P, Tuplin A, Evans D J (2004) Detection of genome-scale ordered RNA structure (GORS) in genomes of positive-stranded RNA viruses: Implications for virus evolution and host persistence. RNA 10:1337-1351.
Stapleton J T, Foung S, Muerhoff A S, Bukh J, Simmonds P (2011) The GB viruses: A review and proposed classification of GBV-A, GBV-C (HGV), and GBV-D in genus Pegivirus within the family Flaviviridae. J Gen Virol 92:233-246.
Tamura K, Dudley J, Nei M, Kumar S (2007) MEGA4: Molecular Evolutionary Genetics Analysis (MEGA) software version 4.0. Mol Biol Evol 24:1596-1599.
Tanaka T, Kato N, Cho M J, Shimotohno K (1995) A novel sequence found at the 3′ terminus of hepatitis C virus genome. Biochem Biophys Res Commun 215:744-749.
Tsai C C, et al. (1995) Prevention of SIV infection in macaques by (R)-9-(2-phosphonylmethoxypropyl)adenine. Science 270:1197-1199.
Van Rompay K K (2010) Evaluation of antiretrovirals in animal models of HIV infection. Antiviral Res 85:159-175.
Victoria J G, et al. (2009) Metagenomic analyses of viruses in stool samples from children with acute flaccid paralysis. J Virol 83:4642-4651.
Wakita T, et al. (2005) Production of infectious hepatitis C virus in tissue culture from a cloned viral genome. Nat Med 11(7):791-796.
Whidby J, et al. (2009) Blocking hepatitis C virus infection with recombinant form of envelope protein 2 ectodomain. J Virol 83:11078-11089.
Wobus C E, Thackray L B, & Virgin H Wt (2006) Murine norovirus: a model system to study norovirus biology and pathogenesis. J Virol 80(11):5104-5112.
Worobey M, et al. (2010) Island biogeography reveals the deep history of SIV. Science 329:1487.
Xu Z, et al. (2001) Synthesis of a novel hepatitis C virus protein by ribosomal frameshift. EMBO J 20:3840-3848.
Kapoor et al. (2011) PNAS 108:11608-11613
Burbelo et al. (2012) J Virol. 86:6171-6178.
Lyons et al. (2012) Emerg Infect Dis 18:1976-82
Kapoor et al. (2013) mBio 4:e00216-13
Chandriani et al. (2013) PNAS 110:E1407---15

What is claimed is:

1. An isolated nucleic acid having a sequence of any of SEQ ID NO: 1 or SEQ ID NO: 3-10.

2. An isolated nucleic acid which comprises 10 consecutive nucleotides having a sequence of any of SEQ ID NO: 1 or SEQ ID NO: 3-10.

3. An isolated nucleic acid which is a variant of any of SEQ ID NO: 1 or SEQ ID NO: 3-10 and has at least about 60% identity to any of SEQ ID NO: 1 or SEQ ID NO: 3-10.

4. (canceled)

5. An isolated nucleic acid complementary to a sequence of any of SEQ ID NO: 1 or SEQ ID NO: 3-10.

6. An isolated nucleic acid comprising 10 consecutive nucleotides complementary to a sequence of any of SEQ ID NO: 1 or SEQ ID NO: 3-10.

7. An isolated nucleic acid which is a complementary to a variant of any of SEQ ID NO: 1 or SEQ ID NO: 3-10 and wherein the variant has at least about 60% identity to any of SEQ ID NO: 1 or SEQ ID NO: 3-10.

8. (canceled)

9. (canceled)

10. (canceled)

11. An isolated polypeptide having a sequence of any of SEQ ID NO: 2 or SEQ ID NO: 11-18.

12. An isolated polypeptide comprising 8 consecutive amino acids having a sequence of any of SEQ ID NO: 2 or SEQ ID NO: 11-18.

13. An isolated polypeptide which is a variant of any of SEQ ID NO: 2 or SEQ ID NO: 11-18 and has at least about 70% identity to any of SEQ ID NO: 2 or SEQ ID NO: 11-18.

14. (canceled)

15. (canceled)

16. (canceled)

17. An isolated diagnostic antibody that specifically binds to a polypeptide encoded by the nucleotide sequence shown in any of SEQ ID NO: 1 or SEQ ID NO: 3-10.

18. An isolated diagnostic antibody that specifically binds to a polypeptide having the sequence of any of SEQ ID NO: 2 or SEQ ID NO: 11-18.

19. (canceled)

20. (canceled)

21. (canceled)

22. (canceled)

23. (canceled)

24. A synthetic nucleic acid which has a sequence consisting of from about 10 to about 30 consecutive nucleotides from a nucleic acid sequence selected from the group consisting of any of SEQ ID NO: 1 or SEQ ID NO: 3-10.

25. (canceled)

26. A synthetic nucleic acid which has a sequence consisting of from about 10 to about 30 consecutive nucleotides from a nucleic acid sequence which is complementary to a nucleic acid sequence selected from the group of sequences consisting of any of SEQ ID NO: 1 or SEQ ID NO: 3-10.

27. A method for determining the presence or absence of NPHV in a biological sample, the method comprising:
a) contacting nucleic acid from a biological sample with at least one primer which is a nucleic acid of claim 24 or 26, and
b) subjecting the nucleic acid and the primer to amplification conditions, and
c) determining the presence or absence of amplification product, wherein the presence of amplification product indicates the presence of RNA associated with NPHV in the sample.

28. (canceled)

29. A method for determining whether or not a sample contains NPHV, the method comprising:
a) contacting a biological sample with an antibody that specifically binds a polypeptide encoded by the nucleic sequence acid of any of SEQ ID NO: 1 or SEQ ID NO: 3-10, and
b) determining whether or not the antibody binds to an antigen in the biological sample, wherein binding indicates that the biological sample contains NPHV.

30. (canceled)

31. (canceled)

32. (canceled)

33. An interfering RNA (iRNA) comprising a sense strand having at least 15 contiguous nucleotides complementary to the anti-sense strand of a gene from a virus comprising a nucleic acid sequence selected from the group of sequences consisting of any of SEQ ID NO: 1 or SEQ ID NO: 3-10, or comprising an anti-sense strand having at least 15 contiguous nucleotides complementary to the sense strand of gene from a virus comprising a nucleic acid sequence selected from the group of sequences consisting of any of SEQ ID NO: 1 or SEQ ID NO: 3-10.

34. (canceled)

35. A method for reducing the levels of a viral protein, viral mRNA or viral titer in a cell in an animal comprising: administering an iRNA agent to an animal, wherein the iRNA agent comprises a sense strand having at least 15 contiguous nucleotides complementary to gene from a NPHV comprising a nucleic acid sequence selected from the group of sequences consisting of any of SEQ ID NO: 1 or SEQ ID NO: 3-10 and an antisense strand having at least 15 contiguous nucleotides complementary to the sense strand.

36. (canceled)

37. (canceled)

38. (canceled)

39. (canceled)

40. (canceled)

41. (canceled)

42. A NPHV immunogenic composition comprising a NPHV nucleic acid selected from the group consisting of: a nucleic acid sequence of any of SEQ ID NO: 1 or SEQ ID NO: 3-10; a nucleic acid comprising least 24 consecutive nucleic acids of any of SEQ ID NO: 1 or SEQ ID NO: 3-10; a nucleic acid substantially identical to the nucleic acid sequence of any of SEQ ID NO: 1 or SEQ ID NO: 3-10; and a nucleic acid that is a variant of any of SEQ ID NO: 1 or SEQ ID NO: 3-10 having at least about 60% identity to any of SEQ ID NO: 1 or SEQ ID NO: 3-10

43. (canceled)

44. (canceled)

45. (canceled)

46. (canceled)

47. (canceled)

48. A NPHV immunogenic composition comprising a NPHV polypeptide selected from the group consisting of: a polypeptide encoded by any of SEQ ID NO: 1 or SEQ ID NO: 3-10; a polypeptide encoded by a nucleic acid comprising least 24 consecutive nucleic acids of any of SEQ ID NO: 1 or SEQ ID NO: 3-10; a polypeptide encoded by a nucleic acid that is substantially identical to the nucleic acid sequence of any of SEQ ID NO: 1 or SEQ ID NO: 3-10; a polypeptide encoded by a nucleic acid that is a variant of any of SEQ ID NO: 1 or SEQ ID NO: 3-10 having at least about 60% identity to any of SEQ ID NO: 1 or SEQ ID NO: 3-10; a polypeptide comprising the amino acid sequence of any of SEQ ID NO: 2 or SEQ ID NO: 11-18; a polypeptide comprising least 8 consecutive amino acids of any of SEQ ID NO: 2 or SEQ ID NO: 11-18; a polypeptide substantially identical to the amino acid sequence of any of SEQ ID NO: 2 or SEQ ID NO: 11-18; and a polypeptide that is a variant of an of SEQ ID NO: 2 or SEQ ID NO: 11-18 having at least about 70% identity to any of SEQ ID NO: 2 or SEQ ID NO: 11-18

49. (canceled)

50. (canceled)

51. (canceled)

52. (canceled)

53. (canceled)

54. (canceled)

55. (canceled)

56. (canceled)

57. (canceled)

58. (canceled)

59. An antibody that binds a NPHV or a NPHV polypeptide and inhibits, neutralizes or reduces the function or activity of the NPHV or NPHV polypeptide.

60. (canceled)

61. (canceled)

62. (canceled)

63. (canceled)

64. (canceled)

65. (canceled)
